(19) United States
(12) Patent Application Publication
(10) Pub. No.: US 2022/0204539 A1
(43) Pub. Date: Jun. 30, 2022

(54) CD73 INHIBITORS

(71) Applicant: BioArdis LLC, San Diego, CA (US)

(72) Inventors: Simon BAILEY, San Diego, CA (US); Dianjun CHEN, Beijing (CN); Jianxia FENG, Beijing (CN); Frank KAYSER, San Rafael, CA (US); Chong LIU, Beijing (CN); Hongbin YUAN, Shanghai (CN)

(21) Appl. No.: 17/603,553

(22) PCT Filed: Apr. 3, 2020
(86) PCT No.: PCT/CN2020/083233

§ 371 Date: Oct. 13, 2021

(30) Foreign Application Priority Data
Apr. 16, 2019 (CN) PCT/CN2019/082816

(51) Int. Cl. C07F 9/6561; A61P 35/00

(52) U.S. Cl. CPC C07F 9/6561; A61P 35/00

(57) ABSTRACT

The present disclosure relates generally to compounds that are inhibitors of CD73 and are useful in treating CD73-associated diseases or conditions. Compositions containing the compounds of the present disclosure are also provided.

### CROSS REFERENCE TO RELATED APPLICATION

This application claims priority to International Patent Application No. PCT/CN2019/082816, filed on Apr. 16, 2019, the content of which is incorporated herein by reference in its entirety.

### FIELD

The present disclosure relates generally to compounds that are inhibitors of CD73 and are useful in treating CD73-associated diseases or conditions. Compositions containing the compounds of the present disclosure are also provided.

### BACKGROUND

CD73 is a 70-kDa glycosylphosphatidylinositol (GPI)-anchored protein normally expressed on endothelial cells and subsets of hematopoietic cells. CD73 is up-regulated by hypoxia-inducible factor (HIF)-1α and after exposure to type I interferons. In steady state, CD73 regulates vascular barrier function, restricts lymphocyte migration to draining lymph nodes, and stimulates mucosal hydration.

CD73 expression on tumor cells has been reported in several types of cancer, including bladder cancer, leukemia, glioma, glioblastoma, melanoma, ovarian cancer, thyroid cancer, esophageal cancer, prostate cancer, and breast cancer. (Stagg, et al., Proc. Natl. Acad. Sci. USA 107(4): 1547-1552). Notably, CD73 expression has been associated with a prometastatic phenotype in melanoma and breast cancer.

There is still a need for new CD73 inhibitors. In this regard, the compounds provided herein address the need.

### BRIEF SUMMARY

In one aspect, provided herein is a compound of formula (I):

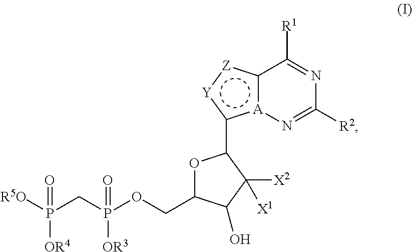

or a stereoisomer, tautomer, or a pharmaceutically acceptable salt of any of the foregoing, wherein, [symbol] , A, Z, Y, X¹, X², and R¹-R⁵ are as described herein.

In another aspect, provided herein is a composition comprising a compound of formula (I), or a stereoisomer, tautomer or a pharmaceutically acceptable salt of any of the foregoing and a pharmaceutically acceptable excipient.

In another aspect, provided herein is a kit comprising a compound of formula (I), or a stereoisomer, tautomer, prodrug, or a pharmaceutically acceptable salt of any of the foregoing. In some embodiments, provided herein is a medicament comprising a compound of formula (I), or a stereoisomer, tautomer, prodrug, or a pharmaceutically acceptable salt of any of the foregoing.

In another aspect, provided herein is a method of treating a disease mediated by CD73 in an individual in need thereof, comprising administering to the individual a therapeutically effective amount of a compound of formula (I), or a stereoisomer, tautomer, prodrug, or a pharmaceutically acceptable salt of any of the foregoing. In some embodiments, the disease is cancer. In some embodiments, the disease is bladder cancer, leukemia, glioma, glioblastoma, melanoma, ovarian cancer, thyroid cancer, esophageal cancer, prostate cancer, lung cancer, colorectal cancer, pancreatic cancer, skin cancer, liver cancer, gastric cancer, head & neck cancer, or breast cancer.

In another aspect, provided herein is a method of inhibition CD73, comprising contacting CD73 with a compound of formula (I), or a stereoisomer, tautomer, prodrug, or a pharmaceutically acceptable salt of any of the foregoing.

In another aspect, provided herein is a compound of formula (I), or a stereoisomer, tautomer, prodrug, or a pharmaceutically acceptable salt of any of the foregoing, in the manufacture of a medicament for use in therapy.

In another aspect, provided herein are methods of preparing a compound of formula (I), or a stereoisomer, tautomer, prodrug, or a pharmaceutically acceptable salt of any of the foregoing, according to the procedures detailed herein.

### BRIEF DESCRIPTION OF THE DRAWINGS

FIG. 1 shows the reduction in tumor volume in a combination study involving Compound No. 65.

### DETAILED DESCRIPTION

Described herein are compounds, including therapeutic agents, that can inhibit CD73. These compounds could be used in the prevention and/or treatment of certain pathological conditions as described herein.

### Definitions

For use herein, unless clearly indicated otherwise, use of the terms “a”, “an” and the like refers to one or more.

Reference to “about” a value or parameter herein includes (and describes) embodiments that are directed to that value or parameter per se. For example, description referring to “about X” includes description of “X”.

“Alkyl” as used herein refers to and includes, unless otherwise stated, a saturated linear (i.e., unbranched) or branched univalent hydrocarbon chain or combination thereof, having the number of carbon atoms designated (i.e., C₁₋₁₀ means one to ten carbon atoms). Particular alkyl groups are those having 1 to 20 carbon atoms (a “C₁₋₂₀ alkyl”), having 1 to 10 carbon atoms (a “C₁₋₁₀ alkyl”), having 6 to 10 carbon atoms (a “C₆₋₁₀ alkyl”), having 1 to 6 carbon atoms (a “C₁₋₆ alkyl”), having 2 to 6 carbon atoms (a “C₂₋₆ alkyl”), or having 1 to 4 carbon atoms (a “C₁₋₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

“Alkoxy” refers to an —O-alkyl. Examples of alkoxy include, but are not limited to, methoxy, ethoxy, n-propoxy, isopropoxy, n-butoxy, iso-butoxy, sec-butoxy, and tert-butoxy.

“Alkylene” as used herein refers to the same residues as alkyl, but having bivalency. Particular alkylene groups are those having 1 to 20 carbon atoms (a “C₁₋₂₀ alkylene”), having 1 to 10 carbon atoms (a “C₁₋₁₀ alkylene”), having 6 to 10 carbon atoms (a “C₆₋₁₀ alkylene”), having 1 to 6 carbon atoms (a “C₁₋₆ alkylene”), 1 to 5 carbon atoms (a “C₁₋₅ alkylene”), 1 to 4 carbon atoms (a “C₁₋₄ alkylene”) or 1 to 3 carbon atoms (a “C₁₋₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

“Alkenyl” as used herein refers to and includes, unless otherwise stated, an unsaturated linear (i.e., unbranched) or branched univalent hydrocarbon chain or combination thereof, having at least one site of olefinic unsaturation (i.e., having at least one moiety of the formula C═C) and having the number of carbon atoms designated (i.e., C₂₋₁₀ means two to ten carbon atoms). An alkenyl group may have “cis” or “trans” configurations, or alternatively have “E” or “Z” configurations. Particular alkenyl groups are those having 2 to 20 carbon atoms (a “C₂₋₂₀ alkenyl”), having 6 to 10 carbon atoms (a “C₆₋₁₀ alkenyl”), having 2 to 8 carbon atoms (a “C₂₋₈ alkenyl”), having 2 to 6 carbon atoms (a “C₂₋₆ alkenyl”), or having 2 to 4 carbon atoms (a “C₂₋₄ alkenyl”). Examples of alkenyl group include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, pent-1-enyl, pent-2-enyl, hex-1-enyl, hex-2-enyl, hex-3-enyl, and the like.

“Alkynyl” as used herein refers to and includes, unless otherwise stated, an unsaturated linear (i.e., unbranched) or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Particular alkynyl groups are those having 2 to 20 carbon atoms (a “C₂₋₂₀ alkynyl”), having 6 to 10 carbon atoms (a “C₆₋₁₀ alkynyl”), having 2 to 8 carbon atoms (a “C₂₋₈ alkynyl”), having 2 to 6 carbon atoms (a “C₂₋₆ alkynyl”), or having 2 to 4 carbon atoms (a “C₂₋₄ alkynyl”). Examples of alkynyl group include, but are not limited to, groups such as ethynyl (or acetylenyl), prop-1-ynyl, prop-2-ynyl (or propargyl), but-1-ynyl, but-2-ynyl, but-3-ynyl, and the like.

“Cycloalkyl” as used herein refers to and includes, unless otherwise stated, cyclic univalent nonaromatic hydrocarbon structures, which may be fully saturated, mono- or polyunsaturated, but which are non-aromatic, having the number of carbon atoms designated (i.e., C₃₋₁₀ means three to ten carbon atoms). Cycloalkyl can consist of one ring, such as cyclohexyl, or multiple rings, such as adamantyl. A cycloalkyl comprising more than one ring may be fused, spiro or bridged, or combinations thereof. Particular cycloalkyl groups are those having from 3 to 12 annular carbon atoms. A preferred cycloalkyl is a cyclic hydrocarbon having from 3 to 8 annular carbon atoms (a “C₃₋₈ cycloalkyl”), having 3 to 6 carbon atoms (a “C₃₋₆ cycloalkyl”), or having from 3 to 4 annular carbon atoms (a “C₃₋₄ cycloalkyl”). Examples of cycloalkyl include, but are not limited to, cyclopropyl, cyclobutyl, cyclopentyl, cyclohexyl, cycloheptyl, norbornyl, and the like. A cycloalkyl group may be fused with aryl, heteroaryl, or heterocyclyl. In one variation, a cycloalkyl group having more than one ring where at least one ring is aryl, heteroaryl, or heterocyclyl is connected to the parent structure at an atom in the nonaromatic hydrocarbon cyclic group.

“Aryl” or “Ar” as used herein refers to an unsaturated aromatic carbocyclic group having a single ring (e.g., phenyl) or multiple condensed rings (e.g., naphthyl or anthryl) which condensed rings may or may not be aromatic. Particular aryl groups are those having from 6 to 14 annular carbon atoms (a “C₆₋₁₄ aryl”). An aryl group may be fused with heteroaryl, cycloalkyl, or heterocyclyl. In one variation, an aryl group having more than one ring where at least one ring is heteroaryl, cycloalkyl, or heterocyclyl is connected to the parent structure at an atom in the aromatic carbocyclic group.

“Heteroaryl” as used herein refers to an unsaturated aromatic cyclic group having from 1 to 14 annular carbon atoms and at least one annular heteroatom, including but not limited to heteroatoms such as nitrogen, oxygen, and sulfur. A heteroaryl group may have a single ring (e.g., pyridyl, furyl) or multiple condensed rings (e.g., indolizinyl, benzothienyl) which condensed rings may or may not be aromatic. Particular heteroaryl groups are 5 to 14-membered rings having 1 to 12 annular carbon atoms and 1 to 6 annular heteroatoms independently selected from nitrogen, oxygen, and sulfur, 5 to 10-membered rings having 1 to 8 annular carbon atoms and 1 to 4 annular heteroatoms independently selected from nitrogen, oxygen, and sulfur, or 5, 6 or 7-membered rings having 1 to 5 annular carbon atoms and 1 to 4 annular heteroatoms independently selected from nitrogen, oxygen, and sulfur. In one variation, particular heteroaryl groups are monocyclic aromatic 5-, 6- or 7-membered rings having from 1 to 6 annular carbon atoms and 1 to 4 annular heteroatoms independently selected from nitrogen, oxygen and sulfur. In another variation, particular heteroaryl groups are polycyclic aromatic rings having from 1 to 12 annular carbon atoms and 1 to 6 annular heteroatoms independently selected from nitrogen, oxygen, and sulfur. A heteroaryl group may be fused with aryl, cycloalkyl, or heterocyclyl. In one variation, a heteroaryl group having more than one ring where at least one ring is aryl, cycloalkyl, or heterocyclyl is connected to the parent structure at an atom in the aromatic cyclic group having at least one annular heteroatom. A heteroaryl group may be connected to the parent structure at a ring carbon atom or a ring heteroatom.

“Heterocycle”, “heterocyclic”, or “heterocyclyl” as used herein refers to a saturated or an unsaturated non-aromatic cyclic group having a single ring or multiple condensed rings, and having from 1 to 14 annular carbon atoms and from 1 to 6 annular heteroatoms, such as nitrogen, sulfur or oxygen, and the like. A heterocycle comprising more than one ring may be fused, bridged or spiro, or any combination thereof, but excludes heteroaryl. The heterocyclyl group may be optionally substituted independently with one or more substituents described herein. Particular heterocyclyl groups are 3 to 14-membered rings having 1 to 13 annular carbon atoms and 1 to 6 annular heteroatoms independently selected from nitrogen, oxygen and sulfur, 3 to 12-membered rings having 1 to 11 annular carbon atoms and 1 to 6 annular heteroatoms independently selected from nitrogen, oxygen and sulfur, 3 to 10-membered rings having 1 to 9 annular carbon atoms and 1 to 4 annular heteroatoms independently selected from nitrogen, oxygen and sulfur, 3 to 8-membered rings having 1 to 7 annular carbon atoms and 1 to 4 annular heteroatoms independently selected from nitrogen, oxygen and sulfur, or 3 to 6-membered rings having 1 to 5 annular carbon atoms and 1 to 4 annular heteroatoms independently selected from nitrogen, oxygen and sulfur. In one variation, heterocyclyl includes monocyclic 3-, 4-, 5-, 6- or 7-membered rings having from 1 to 2, 1 to 3, 1 to 4, 1 to 5, or 1 to 6 annular carbon atoms and 1 to 2, 1 to 3, or 1 to 4 annular heteroatoms independently selected from nitrogen, oxygen and sulfur. In another variation, heterocyclyl includes polycyclic non-aromatic rings having from 1 to 12 annular carbon atoms and 1 to 6 annular heteroatoms independently selected from nitrogen, oxygen and sulfur. A heterocyclyl group may be fused with aryl, cycloalkyl, or heteroaryl. In one variation, a heterocyclyl group having more than one ring where at least one ring is aryl, cycloalkyl, or heteroaryl is connected to the parent structure at an atom in the non-aromatic cyclic group having at least one heteroatom.

“Halo” or “halogen” refers to elements of the Group 17 series having atomic number 9 to 85. Preferred halo groups include the radicals of fluorine, chlorine, bromine and iodine. A haloalkyl is an alkyl group that is substituted with one or more halogens. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl.

“Carbonyl” refers to the group C═O.

“Acyl” refers to —C(═O)R where R is an aliphatic group, preferably a C₁₋₆ moiety. The term “aliphatic” refers to saturated and unsaturated straight chained, branched chained, or cyclic hydrocarbons. Examples of aliphatic groups include, but are not limited to, C₁₋₆ alkyl, C₂₋₆ alkenyl, C₂₋₆ alkynyl, or C₃₋₆ cycloalkyl.

“Oxo” refers to the moiety ═O.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

Unless clearly indicated otherwise, “an individual” as used herein intends a mammal, including but not limited to a primate, human, bovine, horse, feline, canine, or rodent. In one variation, the individual is a human.

As used herein, “treatment” or “treating” is an approach for obtaining beneficial or desired results including clinical results. For purposes of this disclosure, beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods of the present disclosure contemplate any one or more of these aspects of treatment.

As used herein, the term “effective amount” intends such amount of a compound described herein which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound, or pharmaceutically acceptable salt thereof), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

A “therapeutically effective amount” refers to an amount of a compound or salt thereof sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the present disclosure in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the present disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

When a moiety is indicated as substituted by “at least one” substituent, this also encompasses the disclosure of exactly one substituent.

### Compounds

In one aspect, provided is a compound of formula (I):

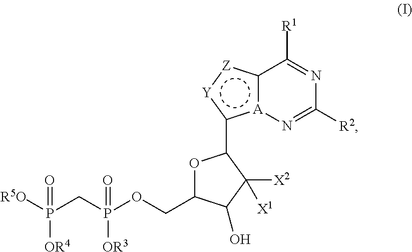

or a stereoisomer, tautomer, prodrug, or a pharmaceutically acceptable salt of any of the foregoing, wherein: [symbol]
means a fully saturated, partially saturated, or aromatic ring;
X¹ and X² are each independently H, —CN, C₁₋₆ alkyl, —OR′ or halogen, wherein R′ is H, C₁₋₆ alkyl, C₃₋₁₂ cycloalkyl, 3- to 12-membered heterocyclyl, 5- to 10-membered heteroaryl, or C₆₋₁₄ aryl;

### Y is CH or N;

### Z is CH, O, or N;

### A is C or N;

R¹ is —NR^{1a}R^{1b} or —OR^{1a}, wherein R^{1a} and R^{1b} are each independently H, C₁₋₆ alkyl, C₃₋₁₂ cycloalkyl, 3- to 12-membered heterocyclyl, 5- to 10-membered heteroaryl, or C₆₋₁₄ aryl, wherein the C₁₋₆ alkyl, C₃₋₁₂ cycloalkyl, 3- to 12-membered heterocyclyl, 5- to 10-membered heteroaryl, and C₆₋₁₄ aryl are each independently optionally substituted with R⁶, or

R^{1a} and R^{1b} are taken together with the nitrogen atom to which they attach to form a 3- to 12-membered heterocyclyl, which is optionally substituted with R⁶;

R² is H, C₁₋₆ alkyl, C₂₋₆ alkenyl, C₂₋₆ alkynyl, halogen, —CN, —OR₂ₐ, —SR₂ₐ, —NR^{2a}R^{2b}, —OC(O)R^{2a}, —NR^{2a}C(O)R^{2b}, —NR^{2a}C(O)OR^{2b}, —NR^{2a}S(O)R^{2b}, —NR^{2a}S(O)₂R^{2b}, —C(O)NR^{2a}R^{2b}, —C(O)NR^{2a}S(O)₂R^{2b}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently optionally substituted with R⁷, and wherein:

R^{2a} and R^{2b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{2a} and R^{2b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
   R³, R⁴, and R are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl of R³, R⁴, and R⁵ are each independently substituted with R⁸, or

R³ and R⁴ or R⁴ and R⁵ are taken together with the atoms to which they attach to form a 3- to 12-membered heterocyclyl, which is optionally substituted with R⁸;

each R⁶ is independently oxo, C₁₋₆ alkyl, C₂₋₆ alkenyl, C₂₋₆ alkynyl, halogen, —CN, —OR^{6a}, —SR^{6a}, —NR^{6b}R^{6b}, —NO₂, —C═NH(OR^{6a}), —C(O)R^{6a}, —OC(O)R^{6a}, —C(O)OR^{6a}, —C(O)NR^{6a}R^{6b}, —OC(O)NR^{6a}R^{6b}, —NR^{6a}C(O)R^{6b}, —NR^{6a}C(O)OR^{6b}, —S(O)R^{6a}, —S(O)₂R^{6a}, —NR^{6a}S(O)R^{6b}, —C(O)NR^{6a}S(O)R^{6b}, —NR^{6a}S(O)₂R^{6b}, —C(O)NR^{6a}S(O)₂R^{6b}, —S(O)NR^{6a}R^{6b}, —S(O)₂NR^{6a}R^{6b}, —P(O)(OR^{6a})(OR^{6b}), C₃₋₆ cycloalkyl, 3- to 12-membered heterocyclyl, 5- to 10-membered heteroaryl, or C₆₋₁₄ aryl, wherein the C₃₋₆ cycloalkyl, 3- to 12-membered heterocyclyl, 5- to 10-membered heteroaryl, and C₆₋₁₄ aryl are each independently optionally substituted with C₁₋₆ alkyl, C₂₋₆ alkenyl, C₂₋₆ alkynyl, halogen, hydroxyl, C₁₋₆ alkoxy, or —CN, and wherein:

R^{6a} and R^{6b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{6a} and R^{6b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
   each R⁷ is independently oxo, C₁₋₆ alkyl, C₂₋₆ alkenyl, C₂₋₆ alkynyl, halogen, —CN, —OR^{7a}, —SR^{7a}, —N^{7a}R^{7b}, —NO₂, —C═NH(OR^{7a}), —C(O)R^{7a}, —OC(O)R^{7a}, —C(O)OR^{7a}, —C(O)NR^{7a}R^{7b}, —OC(O)NR^{7a}R^{7b}, —NR^{7a}C(O)R^{7b}, —NR^{7a}C(O)OR^{7b}, —S(O)R^{7a}, —S(O)₂R^{7a}, —NR^{7a}S(O)R^{7b}, —C(O)NR^{7a}S(O)R^{7b}, —NR^{7a}S(O)₂R, —C(O)NR^{7a}S(O)₂R^{7b}, —S(O)NR^{7a}R^{7b}, —S(O)₂NR^{7a}R^{7b}, —P(O)(OR^{7a}) (OR^{7b}), C₃₋₆ cycloalkyl, 3- to 12-membered heterocyclyl, 5- to 10-membered heteroaryl, or C₆₋₁₄ aryl, wherein:
R⁷ and R^{7b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
   R^{7a} and R^{7b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
      each R is independently oxo, C₁₋₆ alkyl, C₂₋₆ alkenyl, C₂₋₆ alkynyl, halogen, —CN, —OR^{8a}, —SR^{8a}, —NR^{8a}R^{8b}, —NO₂, —C═NH(OR^{8a}), —C(O)R^{8a}, —OC(O)R^{8a}, —C(O)OR^{8a}, —C(O)NR^{8a}R^{8b}, —OC(O)NR^{8a}R^{8b}, —NR^{8a}C(O)R^{8b}, —NR^{8a}C(O)OR^{8b}, —S(O)R^{8a}, —S(O)₂R^{8a}, —NR^{8a}S(O)R^{8b}, —C(O)NR^{8a}S(O)R^{8b}, —NR^{8a}S(O)₂R^{8b}, —C(O)NR^{8a}S(O)₂R^{8b}, —S(O)NR^{8a}R^{8b}, —S(O)₂NR^{8a}R^{8b}, —P(O)(OR^{8a})(OR^{8b}), C₃₋₆ cycloalkyl, 3- to 12-membered heterocyclyl, 5- to 10-membered heteroaryl, or C₆₋₁₄ aryl, each of which is independently optionally substituted with C₁₋₆ alkyl, C₂₋₆ alkenyl, C₂₋₆ alkynyl, halogen, hydroxyl, C₁₋₆ alkoxy, or —CN, and wherein:

R^{8a} and R^{8b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{8a} and R^{8b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN.

In some embodiments, provided is a compound of formula (I):

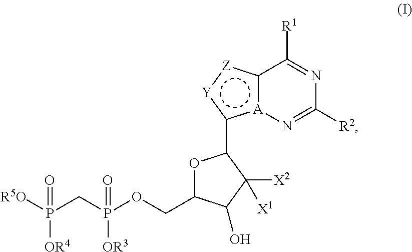

or a stereoisomer, tautomer, prodrug, or a pharmaceutically acceptable salt of any of the foregoing, wherein: [symbol]
means a fully saturated, partially saturated, or aromatic ring;
X¹ and X² are each independently H, —CN, C₁₋₆ alkyl, —OR′ or halogen, wherein R′ is H, C₁₋₆ alkyl, C₃₋₁₂ cycloalkyl, 3- to 12-membered heterocyclyl, 5- to 10-membered heteroaryl, or C₆₋₁₄ aryl;

### Y is CH or N;

### Z is CH, O, or N;

### A is C or N;

R¹ is —NR^{1a}R^{1b} or —OR^{1a}, wherein R^{1a} and R^{1b} are each independently H, C₁₋₆ alkyl, C₃₋₁₂ cycloalkyl, 3- to 12-membered heterocyclyl, 5- to 10-membered heteroaryl, or C₆₋₁₄ aryl, wherein the C₁₋₆ alkyl, C₃₋₁₂ cycloalkyl, 3- to 12-membered heterocyclyl, 5- to 10-membered heteroaryl, and C₆₋₁₄ aryl are each independently optionally substituted with R⁶, or

R^{1a} and R^{1b} are taken together with the nitrogen atom to which they attach to form a 3 to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;

R² is H, C₁₋₆ alkyl, C₂₋₆ alkenyl, C₂₋₆ alkynyl, halogen, —CN, —OR^{2a}, —SR^{2a},
—NR^{2a}R^{2b}, —OC(O)R^{2a}, —NR^{2a}C(O)R^{2b}, —NR^{2a}C(O)OR^{2b},
—NR^{2a}S(O)R^{2b}, —NR^{2a}S(O)₂R^{2b}, —C(O)NR^{2a}R^{2b}, —C(O)NR^{2a}S(O)₂R^{2b}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently optionally substituted with R⁷, and wherein:

R² and R^{2b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{2a} and R^{2b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
   R³, R⁴, and R are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl;
   each R⁶ is independently oxo, C₁₋₆ alkyl, C₂₋₆ alkenyl, C₂₋₆ alkynyl, halogen, —CN, —OR^{6a}, —SR^{6a}, —NR^{6a}R^{6b}, —NO₂, —C═NH(OR^{6a}), —C(O)R^{6a}, —OC(O)R^{6a}, —C(O)OR^{6a}, —C(O)NR^{6a}R^{6b}, —OC(O)NR^{6a}R^{6b}, —NR^{6a}C(O)R^{6b}, —NR^{6a}C(O)OR^{6b}, —S(O)R^{6a}, —S(O)₂R^{6a}, —NR^{6a}S(O)R^{6b}, —C(O)NR^{6a}S(O)R^{6b}, —NR^{6a}S(O)₂R^{6b}, —C(O)NR^{6a}S(O)₂R^{6b}, —S(O)NR^{6a}R^{6b}, —S(O)₂NR^{6a}R^{6b}, —P(O)(OR^{6a})(OR^{6b}), C₃₋₆ cycloalkyl, 3- to 12-membered heterocyclyl, 5- to 10-membered heteroaryl, or C₆₋₁₄ aryl, wherein the C₃₋₆ cycloalkyl, 3- to 12-membered heterocyclyl, 5- to 10-membered heteroaryl, and C₆₋₁₄ aryl are each independently optionally substituted with C₁₋₆ alkyl, C₂₋₆ alkenyl, C₂₋₆ alkynyl, halogen, hydroxyl, C₁₋₆ alkoxy, or —CN, and wherein:

R^{6a} and R^{6b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{6a} and R^{6b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
   each R⁷ is independently oxo, C₁₋₆ alkyl, C₂₋₆ alkenyl, C₂₋₆ alkynyl, halogen, —CN, —OR^{7a}, —SR^{7a}, —NR^{7a}R⁷, —NO₂, —C═NH(OR^{7a}), —C(O)R^{7a}, —OC(O)R^{7a}, —C(O)OR^{7a}, —C(O)NR^{7a}R^{7b}, —OC(O)NR^{7a}R^{7b}, —NR^{7a}C(O)R^{7b}, —NR^{7a}C(O)OR^{7b}, —S(O)R^{7a}, —S(O)₂R^{7a}, —NR^{7a}S(O)R^{7b}, —C(O)NR^{7a}S(O)R^{7b}, —NR^{7a}S(O)₂R^{7b}, —C(O)NR^{7a}S(O)₂R^{7b}, —S(O)NR^{7a}R^{7b}, —S(O)₂NR^{7a}R^{7b}, —P(O)(OR^{7a})(OR^{7b}), C₃₋₆ cycloalkyl, 3- to 12-membered heterocyclyl, 5- to 10-membered heteroaryl, or C₆₋₁₄ aryl, wherein:

R^{7a} and R^{7b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{7a} and R^{7b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN.

In some embodiments, the compound of formula (I) is of formula (I-a), or a stereoisomer, tautomer, prodrug, or a pharmaceutically acceptable salt of any of the foregoing,

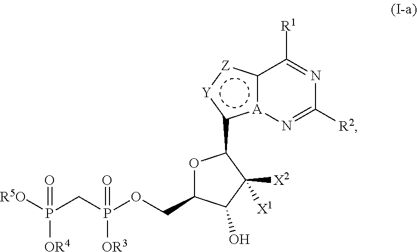

wherein [symbol] , A, Z, Y, X¹, X², and R¹-R⁵ are as defined herein for any embodiment of a compound of formula (I).

In some embodiments, the compound of formula (I) is of formula (I-b), or a stereoisomer, tautomer, prodrug, or a pharmaceutically acceptable salt of any of the foregoing,

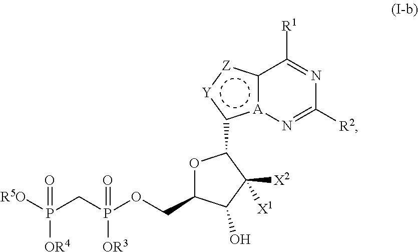

wherein [symbol] , A, Z, Y, X¹, X², and R¹-R⁵ are as defined herein for any embodiment of a compound of formula (I).

In some embodiments of a compound of formula (I), or any related formula, or a stereoisomer, tautomer, prodrug, or a pharmaceutically acceptable salt of any of the foregoing, [symbol] means a partially saturated ring. In some embodiments, [symbol] means an aromatic ring.

In some embodiments of a compound of formula (I), or any related formula, or a stereoisomer, tautomer, prodrug, or a pharmaceutically acceptable salt of any of the foregoing, Y is CH. In some embodiments, Y is N.

In some embodiments of a compound of formula (I), or any related formula, or a stereoisomer, tautomer, prodrug, or a pharmaceutically acceptable salt of any of the foregoing, Z is CH. In some embodiments, Z is O. In some embodiments, Z is N.

In some embodiments of a compound of formula (I), or any related formula, or a stereoisomer, tautomer, prodrug, or a pharmaceutically acceptable salt of any of the foregoing, A is C. In some embodiments, A is N.

In some embodiments of a compound of formula (I), or any related formula, or a stereoisomer, tautomer, prodrug, or a pharmaceutically acceptable salt of any of the foregoing, Y is CH; Z is CH; and A is C. In some embodiments, Y is CH; Z is O; and A is C. In some embodiments, Y is CH; Z is N; and A is N. In some embodiments, Y is N; Z is CH; and A is N.

In some embodiments, the compound of formula (I) is of formula (II), or a stereoisomer, tautomer, prodrug, or a pharmaceutically acceptable salt of any of the foregoing,

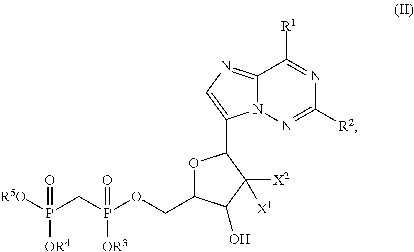

wherein X¹, X², and R¹-R⁵ are as defined herein for any embodiment of a compound of formula (I).

In some embodiments, the compound of formula (II) is of formula (II-a), or a stereoisomer, tautomer, prodrug, or a pharmaceutically acceptable salt of any of the foregoing,

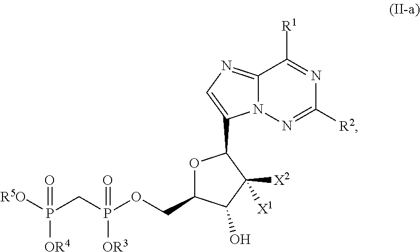

wherein X¹, X², and R¹-R⁵ are as defined herein for any embodiment of a compound of formula (I).

In some embodiments, the compound of formula (II) is of formula (II-b), or a stereoisomer, tautomer, prodrug, or a pharmaceutically acceptable salt of any of the foregoing,

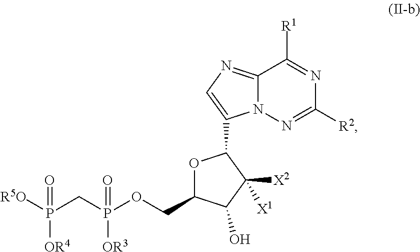

wherein X¹, X², and R¹-R⁵ are as defined herein for any embodiment of a compound of formula (I).

In some embodiments, the compound of formula (I) is of formula (III), or a stereoisomer, tautomer, prodrug, or a pharmaceutically acceptable salt of any of the foregoing,

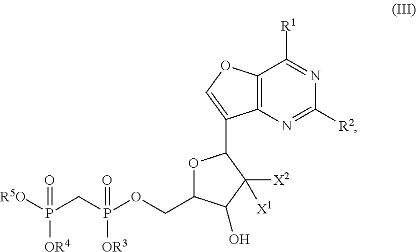

wherein X¹, X², and R¹-R⁵ are as defined herein for any embodiment of a compound of formula (I).

In some embodiments, the compound of formula (III) is of formula (III-a), or a stereoisomer, tautomer, prodrug, or a pharmaceutically acceptable salt of any of the foregoing,

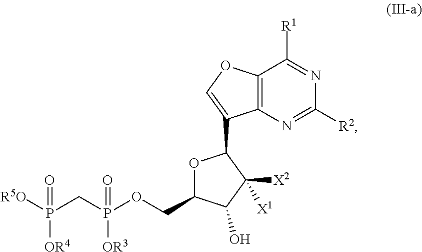

wherein X¹, X², and R¹-R⁵ are as defined herein for any embodiment of a compound of formula (I).

In some embodiments, the compound of formula (III) is of formula (III-b), or a stereoisomer, tautomer, prodrug, or a pharmaceutically acceptable salt of any of the foregoing,

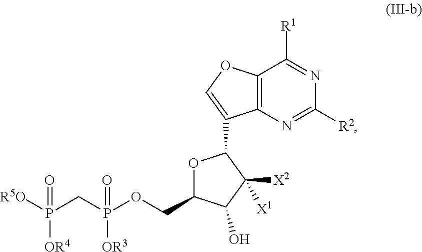

wherein X¹, X², and R¹-R⁵ are as defined herein for any embodiment of a compound of formula (I).

In some embodiments, the compound of formula (I) is of formula (IV), or a stereoisomer, tautomer, prodrug, or a pharmaceutically acceptable salt of any of the foregoing,

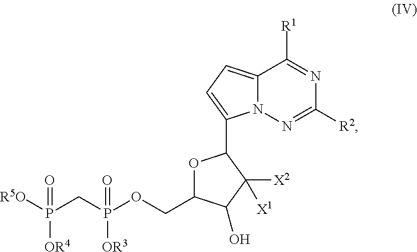

wherein X¹, X², and R¹-R⁵ are as defined herein for any embodiment of a compound of formula (I).

In some embodiments, the compound of formula (IV) is of formula (IV-a), or a stereoisomer, tautomer, prodrug, or a pharmaceutically acceptable salt of any of the foregoing,

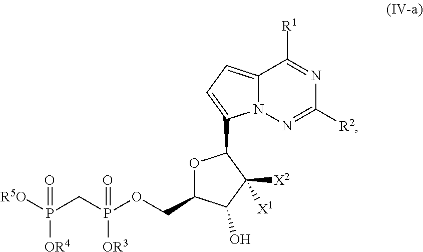

wherein X¹, X², and R¹-R⁵ are as defined herein for any embodiment of a compound of formula (I).

In some embodiments, the compound of formula (IV) is of formula (IV-b), or a stereoisomer, tautomer, prodrug, or a pharmaceutically acceptable salt of any of the foregoing,

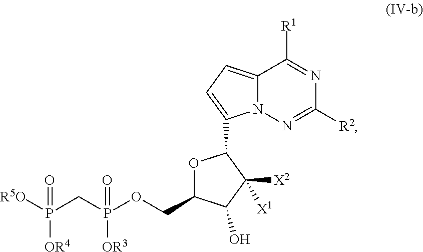

wherein X¹, X², and R¹-R⁵ are as defined herein for any embodiment of a compound of formula (I).

In some embodiments of a compound of formula (I), or any related formula, or a stereoisomer, tautomer, prodrug, or a pharmaceutically acceptable salt of any of the foregoing, R³ is H. In some embodiments, R³ is C₁ alkyl, such as methyl, ethyl, n-propyl, isopropyl, n-butyl, t-butyl, isobutyl, or sec-butyl. In some embodiments, R³ is C₂₋₆ alkenyl, such as ethenyl, prop-1-enyl, prop-2-enyl, 2-methylprop-1-enyl, but-1-enyl, but-2-enyl, or but-3-enyl. In some embodiments, R³ is C₂₋₆ alkynyl, such as ethynyl, prop-1-ynyl, prop-2-ynyl, but-1-ynyl, but-2-ynyl, or but-3-ynyl. In some embodiments, R³ is C₃₋₁₂ cycloalkyl. In some embodiments, R³ is C₃₋₆ cycloalkyl, such as cyclopropyl, cyclobutyl, cyclopentyl, or cyclohexyl. In some embodiments, R³ is C₆₋₁₄ aryl, such as phenyl or naphthyl. In some embodiments, R³ is phenyl. In some embodiments, R³ is 5- to 10-membered heteroaryl. In some embodiments, R³ is 5- or 6-membered heteroaryl, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R³ is 3- to 12-membered heterocyclyl. In some embodiments, R³ is 5- or 6-membered heterocyclyl, such as tetrahydrofuranyl, pyrrolidinyl, piperidinyl, piperazinyl, morpholinyl, or thiomorpholinyl. In some embodiments, R³ is H or C₁₋₆ alkyl. In some embodiments, R³ is H or methyl. In some embodiments, R³ is methyl. In some embodiments, R³ is H or C₁₋₆ alkyl optionally substituted with R⁸.

In some embodiments of a compound of formula (I), or any related formula, or a stereoisomer, tautomer, prodrug, or a pharmaceutically acceptable salt of any of the foregoing, R⁴ is H. In some embodiments, R⁴ is C₁₋₆ alkyl, such as methyl, ethyl, n-propyl, isopropyl, n-butyl, t-butyl, isobutyl, or sec-butyl. In some embodiments, R⁴ is C₂₋₆ alkenyl, such as ethenyl, prop-1-enyl, prop-2-enyl, 2-methylprop-1-enyl, but-1-enyl, but-2-enyl, or but-3-enyl. In some embodiments, R⁴ is C₂₋₆ alkynyl, such as ethynyl, prop-1-ynyl, prop-2-ynyl, but-1-ynyl, but-2-ynyl, or but-3-ynyl. In some embodiments, R⁴ is C₃₋₁₂ cycloalkyl. In some embodiments, R⁴ is C₃₋₆ cycloalkyl, such as cyclopropyl, cyclobutyl, cyclopentyl, or cyclohexyl. In some embodiments, R⁴ is C₆₋₁₄ aryl, such as phenyl or naphthyl. In some embodiments, R⁴ is phenyl. In some embodiments, R⁴ is 5- to 10-membered heteroaryl. In some embodiments, R⁴ is 5- or 6-membered heteroaryl, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R⁴ is 3- to 12-membered heterocyclyl. In some embodiments, R⁴ is 5- or 6-membered heterocyclyl, such as tetrahydrofuranyl, pyrrolidinyl, piperidinyl, piperazinyl, morpholinyl, or thiomorpholinyl. In some embodiments, R⁴ is H or C₁₋₆ alkyl. In some embodiments, R⁴ is H or methyl. In some embodiments, R⁴ is methyl. In some embodiments, R⁴ is H or C₁₋₆ alkyl optionally substituted with R⁸.

In some embodiments of a compound of formula (I), or any related formula, or a stereoisomer, tautomer, prodrug, or a pharmaceutically acceptable salt of any of the foregoing, R⁵ is H. In some embodiments, R⁵ is C₁₋₆ alkyl, such as methyl, ethyl, n-propyl, isopropyl, n-butyl, t-butyl, isobutyl, or sec-butyl. In some embodiments, R⁵ is C₂₋₆ alkenyl, such as ethenyl, prop-1-enyl, prop-2-enyl, 2-methylprop-1-enyl, but-1-enyl, but-2-enyl, or but-3-enyl. In some embodiments, R⁵ is C₂₋₆ alkynyl, such as ethynyl, prop-1-ynyl, prop-2-ynyl, but-1-ynyl, but-2-ynyl, or but-3-ynyl. In some embodiments, R⁵ is C₃₋₁₂ cycloalkyl. In some embodiments, R⁵ is C₃₋₆ cycloalkyl, such as cyclopropyl, cyclobutyl, cyclopentyl, or cyclohexyl. In some embodiments, R⁵ is C₆₋₁₄ aryl, such as phenyl or naphthyl. In some embodiments, R⁵ is phenyl. In some embodiments, R⁵ is 5- to 10-membered heteroaryl. In some embodiments, R⁵ is 5- or 6-membered heteroaryl, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R⁵ is 3- to 12-membered heterocyclyl. In some embodiments, R⁵ is 5- or 6-membered heterocyclyl, such as tetrahydrofuranyl, pyrrolidinyl, piperidinyl, piperazinyl, morpholinyl, or thiomorpholinyl. In some embodiments, R⁵ is H or C₁₋₆ alkyl. In some embodiments, R⁵ is H or methyl. In some embodiments, R⁵ is methyl. In some embodiments, R⁵ is H or C₁₋₆ alkyl optionally substituted with R⁸.

In some embodiments of a compound of formula (I), or any related formula such as formula (II) or (III), or a stereoisomer, tautomer, prodrug, or a pharmaceutically acceptable salt of any of the foregoing, R³ is H; R⁴ is H; and R⁵ is H. In some embodiments, R³, R⁴, and R⁵ are each independently H or C₁₋₆alkyl optionally substituted with R⁸, or R³ and R⁴ or R⁴ and R⁵ are taken together with the atoms to which they attach to form a 3- to 12-membered heterocyclyl, which is optionally substituted with R⁸. In some embodiments, R³, R⁴, and R⁵ are each independently H or C₁₋₆alkyl.

In some embodiments, the compound of formula (I) is of any one of the following formulae, or a stereoisomer, tautomer, prodrug, or a pharmaceutically acceptable salt of any of the foregoing.

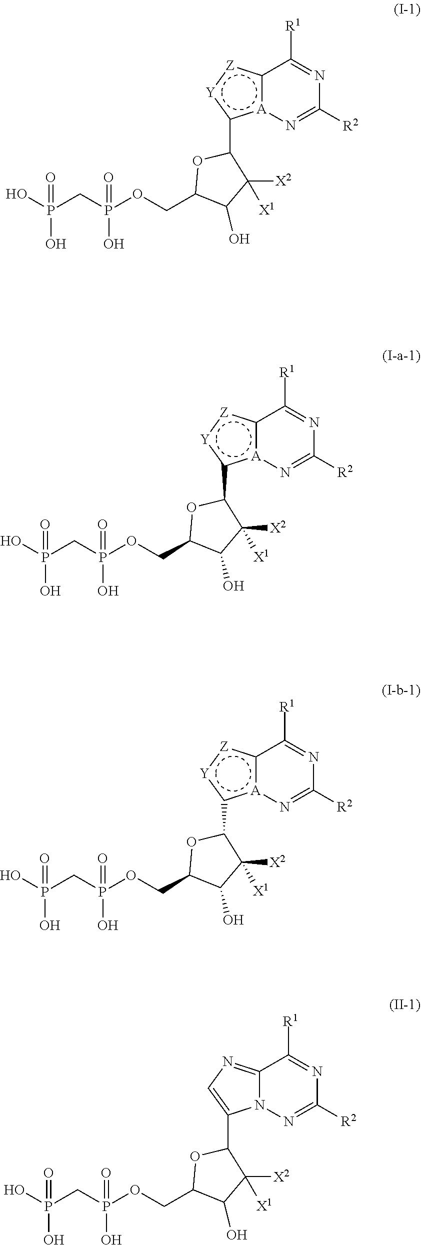

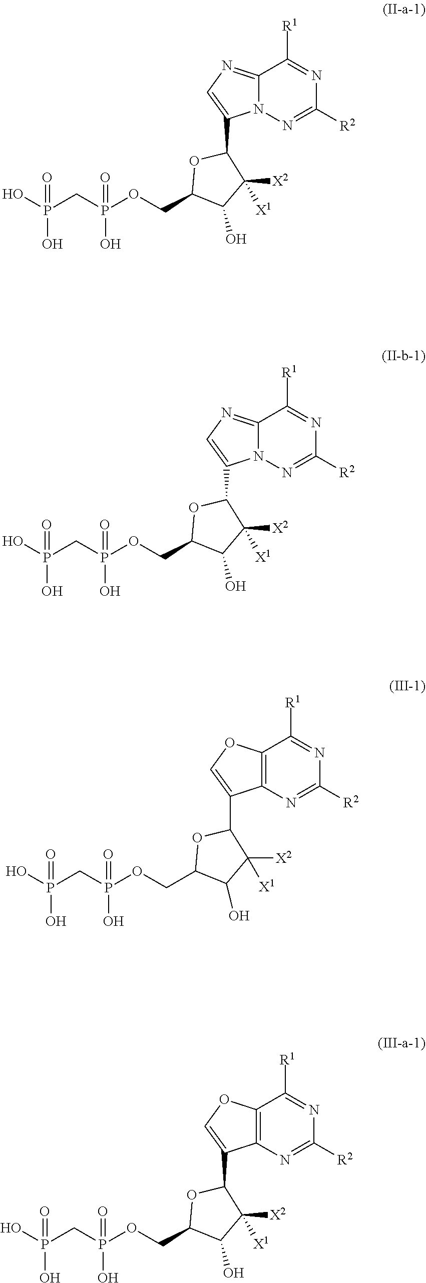

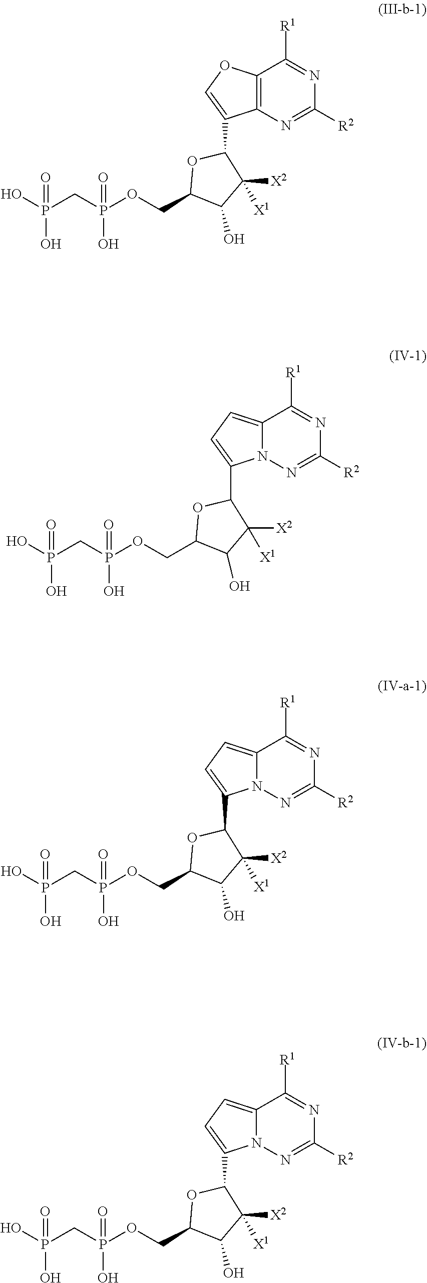

In some embodiments of a compound of formula (I), or any related formula, or a stereoisomer, tautomer, prodrug, or a pharmaceutically acceptable salt of any of the foregoing, X¹ is H. In some embodiments, X¹ is —CN. In some embodiments, X¹ is C₁₋₆alkyl such as methyl, ethyl, n-propyl, isopropyl, n-butyl, t-butyl, isobutyl, or sec-butyl. In some embodiments, X¹ is —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X¹ is —OH. In some embodiments, X¹ is halogen such as fluoro, chloro, or bromo. In some embodiments, X¹ is H or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X¹ is H or halogen. In some embodiments, X¹ is H or —OH.

In some embodiments of a compound of formula (I), or any related formula, or a stereoisomer, tautomer, prodrug, or a pharmaceutically acceptable salt of any of the foregoing, X² is H. In some embodiments, X² is —CN. In some embodiments, X² is C₁₋₆ alkyl such as methyl, ethyl, n-propyl, isopropyl, n-butyl, t-butyl, isobutyl, or sec-butyl. In some embodiments, X² is —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X² is —OH. In some embodiments, X² is halogen such as fluoro, chloro, or bromo. In some embodiments, X² is fluoro. In some embodiments, X² is H or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl. In some embodiments, X² is H or halogen. In some embodiments, X² is H or fluoro.

In some embodiments of a compound of formula (I), or any related formula, or a stereoisomer, tautomer, prodrug, or a pharmaceutically acceptable salt of any of the foregoing, X¹ is H or —OR′, wherein R′ is H, C₁₋₆ alkyl, C₃₋₁₂ cycloalkyl, 3- to 12-membered heterocyclyl, 5- to 10-membered heteroaryl, or C₆₋₁₄ aryl; and X² is H or halogen. In some embodiments, X¹ is H or —OH; and X² is H or halogen. In some embodiments, X¹ is H or —OH; and X² is H or fluoro.

In some embodiments of a compound of formula (I), or any related formula, or a stereoisomer, tautomer, prodrug, or a pharmaceutically acceptable salt of any of the foregoing, R¹ is —NR^{1a}R^{1b}. In some embodiments, R¹ is —OR^{1a}.

In some embodiments of a compound of formula (I), or any related formula, or a stereoisomer, tautomer, prodrug, or a pharmaceutically acceptable salt of any of the foregoing, R^{1a} is H. In some embodiments, R^{1a} is C₁₋₆ alkyl optionally substituted with R⁶, such as methyl, ethyl, n-propyl, isopropyl, n-butyl, t-butyl, isobutyl, or sec-butyl, each of which is independently optionally substituted with R⁶. In some embodiments, R^{1a} is C₁₋₆ alkyl such as methyl, ethyl, n-propyl, isopropyl, n-butyl, t-butyl, isobutyl, or sec-butyl. In some embodiments, R^{1a} is C₃₋₁₂ cycloalkyl optionally substituted with R⁶, such as cyclopropyl, cyclobutyl, cyclopentyl, or cyclohexyl, each of which is independently optionally substituted with R⁶. In some embodiments, R^{1a} is C₃₋₁₂ cycloalkyl which is unsubstituted, such as cyclopropyl, cyclobutyl, cyclopentyl, or cyclohexyl. In some embodiments, R^{1a} is C₆₋₁₄ aryl optionally substituted with R⁶, such as phenyl or naphthyl, each of which is independently optionally substituted with R⁶. In some embodiments, R^{1a} is C₆₋₁₄ aryl, which is unsubstituted, such as phenyl or naphthyl. In some embodiments, R^{1a} is phenyl optionally substituted with R⁶. In some embodiments, R^{1a} is phenyl. In some embodiments, R^{1a} is 5- to 10-membered heteroaryl optionally substituted with R⁶. In some embodiments, R^{1a} is 5- or 6-membered heteroaryl optionally substituted with R⁶, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl, each of which is independently optionally substituted with R⁶. In some embodiments, R^{1a} is 5- or 6-membered heteroaryl which is unsubstituted, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R^{1a} is 3 to 12-membered heterocyclyl optionally substituted with R⁶. In some embodiments, R^{1a} is 5- or 6-membered heterocyclyl optionally substituted with R⁶, such as tetrahydrofuranyl, pyrrolidinyl, piperidinyl, piperazinyl, morpholinyl, or thiomorpholinyl, each of which is independently optionally substituted with R⁶. In some embodiments, R^{1a} is 5- or 6-membered heterocyclyl which is unsubstituted, such as tetrahydrofuranyl, pyrrolidinyl, piperidinyl, piperazinyl, morpholinyl, or thiomorpholinyl. In some embodiments, R^{1a} is C₁₋₆ alkyl, C₃₋₁₂ cycloalkyl, or 3- to 12-membered heterocyclyl, each of which is independently optionally substituted with R⁶.

In some embodiments of a compound of formula (I), or any related formula, or a stereoisomer, tautomer, prodrug, or a pharmaceutically acceptable salt of any of the foregoing, R⁶ is 3- to 12-membered heterocyclyl or C₆₋₁₄ aryl, each of which is independently optionally substituted with halogen. In some embodiments, each R⁶ is independently C₃₋₆ cycloalkyl, 3- to 12-membered heterocyclyl, 5- to 10-membered heteroaryl, C₆₋₁₄ aryl, —OR^{6a}, oxo, or —NR^{6a}R^{6b}, wherein the C₃₋₆ cycloalkyl, 3- to 12-membered heterocyclyl, 5- to 10-membered heteroaryl, and C₆₋₁₄ aryl are each independently substituted with halogen or hydroxyl. In some embodiments, R⁶ is 5- or 6-membered heterocyclyl or phenyl, each of which is independently optionally substituted with halogen. In some embodiments, R⁶ is 3- to 12-membered heterocyclyl optionally substituted with halogen. In some embodiments, R⁶ is 5- or 6-membered heterocyclyl optionally substituted with halogen, such as tetrahydrofuranyl, pyrrolidinyl, piperidinyl, piperazinyl, morpholinyl, or thiomorpholinyl, each of which is independently optionally substituted with halogen. In some embodiments, R⁶ is 5- or 6-membered heterocyclyl which is unsubstituted, such as tetrahydrofuranyl, pyrrolidinyl, piperidinyl, piperazinyl, morpholinyl, or thiomorpholinyl. In some embodiments, R⁶ is tetrahydrofuranyl. In some embodiments, R⁶ is C₆₋₁₄ aryl, which is unsubstituted, such as phenyl or naphthyl. In some embodiments, R⁶ is phenyl optionally substituted with halogen. In some embodiments, R⁶ is phenyl.

In some embodiments of a compound of formula (I), or any related formula such as formula (II) or (III), or a stereoisomer, tautomer, prodrug, or a pharmaceutically acceptable salt of any of the foregoing, R^{1a} is

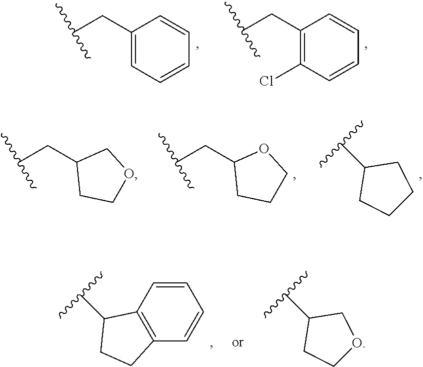

In some embodiments, R^{1a} is

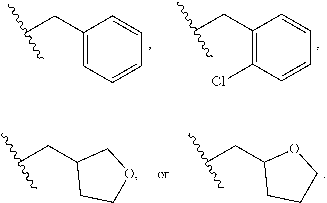 [SMILES: *Cc1ccccc1 ; *Cc1ccccc1Cl ; *CC1CCOC1 ; *CC1CCCO1]

In some embodiments, R^{1a} is

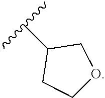 [SMILES: *C1CCOC1]

In some embodiments, R^{1a} is

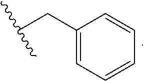 [SMILES: *Cc1ccccc1]

In some embodiments, R^{1a} is

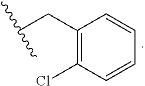 [SMILES: *Cc1ccccc1Cl]

In some embodiments, R^{1a} is

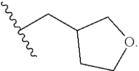 [SMILES: *CC1CCOC1]

In some embodiments, R^{1a} is

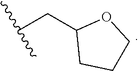 [SMILES: *CC1CCCO1]

In some embodiments, R^{1a} is

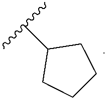 [SMILES: *C1CCCC1]

In some embodiments, R^{1a} is

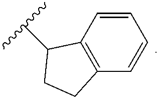 [SMILES: *C1CCc2ccccc21]

In some embodiments, R^{1a} is

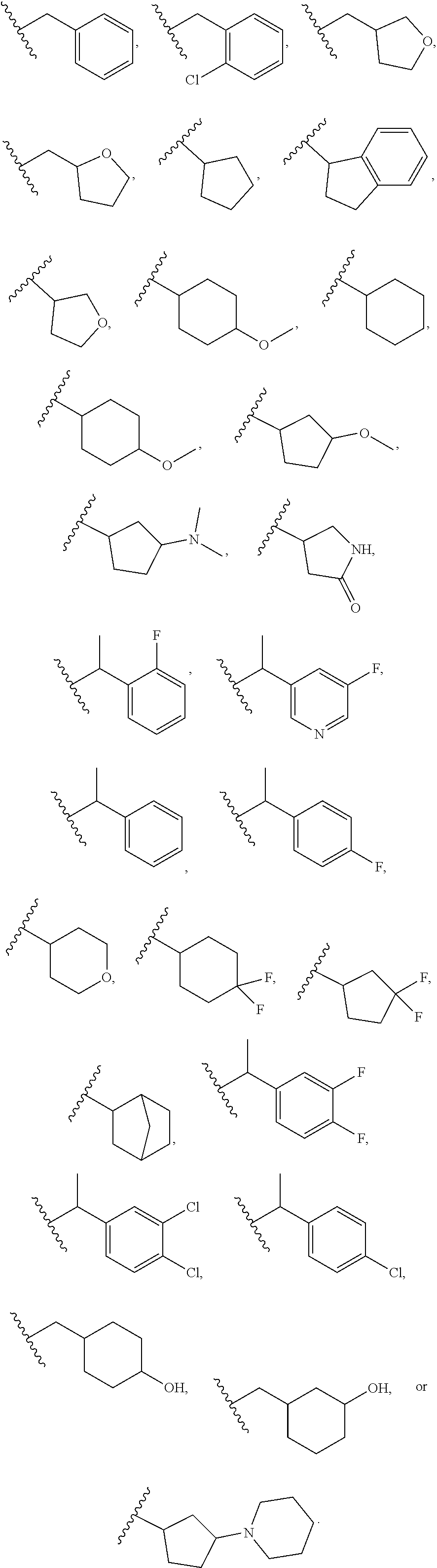

In some embodiments of a compound of formula (I), or any related formula, or a stereoisomer, tautomer, prodrug, or a pharmaceutically acceptable salt of any of the foregoing, R^{1b} is H. In some embodiments, R^{1b} is C₁₋₆alkyl optionally substituted with R⁶, such as methyl, ethyl, n-propyl, isopropyl, n-butyl, t-butyl, isobutyl, or sec-butyl, each of which is independently optionally substituted with R⁶. In some embodiments, R^{1b} is C₁₋₆ alkyl such as methyl, ethyl, n-propyl, isopropyl, n-butyl, t-butyl, isobutyl, or sec-butyl. In some embodiments, R^{1b} is C₃₋₁₂ cycloalkyl optionally substituted with R⁶, such as cyclopropyl, cyclobutyl, cyclopentyl, or cyclohexyl, each of which is independently optionally substituted with R⁶. In some embodiments, R^{1b} is C₃₋₁₂ cycloalkyl which is unsubstituted, such as cyclopropyl, cyclobutyl, cyclopentyl, or cyclohexyl. In some embodiments, R^{1b} is C₆₋₁₄ aryl optionally substituted with R⁶, such as phenyl or naphthyl, each of which is independently optionally substituted with R⁶. In some embodiments, R^{1b} is C₆₋₁₄ aryl, which is unsubstituted, such as phenyl or naphthyl. In some embodiments, R^{1b} is phenyl optionally substituted with R⁶. In some embodiments, R^{1b} is phenyl. In some embodiments, R^{1b} is 5- to 10-membered heteroaryl optionally substituted with R⁶. In some embodiments, R^{1b} is 5- or 6-membered heteroaryl optionally substituted with R⁶, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl, each of which is independently optionally substituted with R⁶. In some embodiments, R^{1b} is 5- or 6-membered heteroaryl which is unsubstituted, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R^{1b} is 3 to 12-membered heterocyclyl optionally substituted with R⁶. In some embodiments, R^{1b} is 5- or 6-membered heterocyclyl optionally substituted with R⁶, such as tetrahydrofuranyl, pyrrolidinyl, piperidinyl, piperazinyl, morpholinyl, or thiomorpholinyl, each of which is independently optionally substituted with R⁶. In some embodiments, R^{1b} is 5- or 6-membered heterocyclyl which is unsubstituted, such as tetrahydrofuranyl, pyrrolidinyl, piperidinyl, piperazinyl, morpholinyl, or thiomorpholinyl. In some embodiments, R^{1b} is C₁₋₆ alkyl, C₃₋₁₂ cycloalkyl, or 3- to 12-membered heterocyclyl, each of which is independently optionally substituted with R⁶. In some embodiments, R^{1b} is H or C₁₋₆ alkyl. In some embodiments, R^{1b} is H or methyl. In some embodiments, R^{1b} is C₁₋₆ alkyl. In some embodiments, R^{1b} is methyl.

In some embodiments of a compound of formula (I), or any related formula, or a stereoisomer, tautomer, prodrug, or a pharmaceutically acceptable salt of any of the foregoing, R^{1a} and R^{1b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN. In some embodiments, R^{1a} and R^{1b} are taken together with the nitrogen atom to which they attach to form a 3- to 12-membered heterocyclyl, which is optionally substituted with R⁶, wherein R⁶ is C₁₋₆ alkyl or C₆₋₁₄ aryl, each of which is optionally substituted with halogen. In some embodiments, R^{1a} and R^{1b} are taken together with the nitrogen atom to which they attach to form a 3- to 12-membered heterocyclyl which is unsubstituted. In some embodiments, R^{1a} and R^{1b} are taken together with the nitrogen atom to which they attach to form

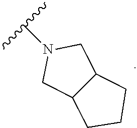 [SMILES: *N1CC2CCCC2C1]

In some embodiments, R^{1a} and R^{1b} are taken together with the nitrogen atom to which they attach to form

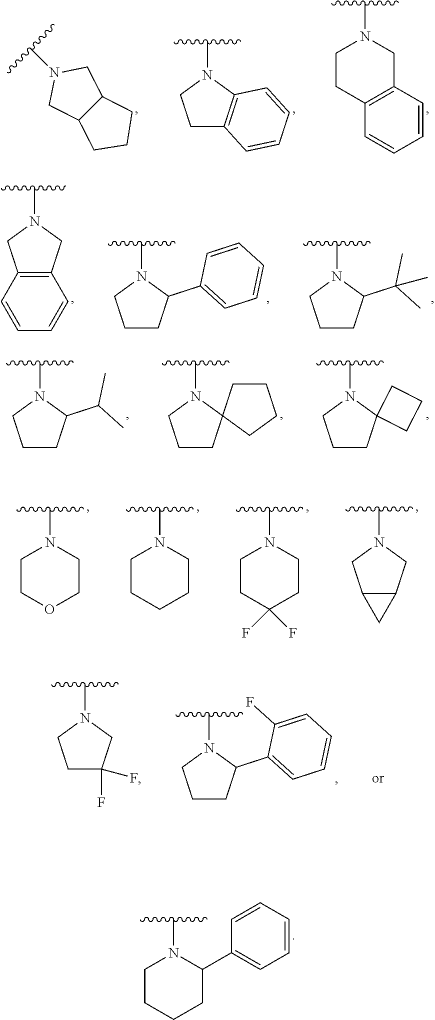

In some embodiments of a compound of formula (I), or any related formula, or a stereoisomer, tautomer, prodrug, or a pharmaceutically acceptable salt of any of the foregoing, R² is H, C₁₋₆ alkyl, C₂₋₆ alkenyl, C₂₋₆ alkynyl, halogen, —CN, —OR^{2a}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently optionally substituted with R⁷. In some embodiments, R² is C₁₋₆ alkyl, C₂₋₆ alkenyl, C₂₋₆ alkynyl, C₃₋₁₂ cycloalkyl, C₆₋₁₄ aryl, 5- to 10-membered heteroaryl, or 3- to 12-membered heterocyclyl, each of which is independently optionally substituted with R⁷. In some embodiments, R² is C₁₋₆ alkyl optionally substituted with R⁷, such as methyl, ethyl, n-propyl, isopropyl, n-butyl, t-butyl, isobutyl, or sec-butyl, each of which is independently optionally substituted with R⁷. In some embodiments, R² is C₁₋₆ alkyl such as methyl, ethyl, n-propyl, isopropyl, n-butyl, t-butyl, isobutyl, or sec-butyl. In some embodiments, R² is C₂₋₆ alkenyl optionally substituted with R⁷, such as ethenyl, prop-1-enyl, prop-2-enyl, 2-methylprop-1-enyl, but-1-enyl, but-2-enyl, or but-3-enyl, each of which is independently optionally substituted with R⁷. In some embodiments, R² is C₂₋₆ alkenyl, such as ethenyl, prop-1-enyl, prop-2-enyl, 2-methylprop-1-enyl, but-1-enyl, but-2-enyl, or but-3-enyl. In some embodiments, R² is C₂₋₆ alkynyl optionally substituted with R⁷, such as ethynyl, prop-1-ynyl, prop-2-ynyl, but-1-ynyl, but-2-ynyl, or but-3-ynyl, each of which is independently optionally substituted with R⁷. In some embodiments, R² is C₂₋₄ alkynyl, such as ethynyl, prop-1-ynyl, prop-2-ynyl, but-1-ynyl, but-2-ynyl, or but-3-ynyl. In some embodiments, R² is halogen, such as fluoro, chloro, or bromo. In some embodiments, R² is chloro. In some embodiments, R² is C₃₋₁₂ cycloalkyl optionally substituted with R⁷. In some embodiments, R² is C₃₋₆ cycloalkyl optionally substituted with R⁷, such as cyclopropyl, cyclobutyl, cyclopentyl, or cyclohexyl, each of which is independently optionally substituted with R⁷. In some embodiments, R² is C₃₋₆ cycloalkyl which is unsubstituted, such as cyclopropyl, cyclobutyl, cyclopentyl, or cyclohexyl. In some embodiments, R² is C_{6_14} aryl optionally substituted with R⁷, such as phenyl or naphthyl, each of which is independently optionally substituted with R⁷. In some embodiments, R² is C₆₋₁₄ aryl, which is unsubstituted, such as phenyl or naphthyl. In some embodiments, R² is phenyl optionally substituted with R⁷. In some embodiments, R² is phenyl. In some embodiments, R² is 5- to 10-membered heteroaryl optionally substituted with R⁷. In some embodiments, R² is 5- or 6-membered heteroaryl optionally substituted with R⁷, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl, each of which is independently optionally substituted with R⁷. In some embodiments, R² is 5- or 6-membered heteroaryl which is unsubstituted, such as pyridinyl, pyrazinyl, pyridazinyl, pyrimidinyl, triazinyl, pyrrolyl, pyrazolyl, imidazolyl, triazolyl, tetrazolyl, oxazolyl, thiazolyl, thiazolyl, or furanyl. In some embodiments, R² is 3- to 12-membered heterocyclyl optionally substituted with R⁷. In some embodiments, R² is 5- or 6-membered heterocyclyl optionally substituted with R⁷, such as tetrahydrofuranyl, pyrrolidinyl, piperidinyl, piperazinyl, morpholinyl, or thiomorpholinyl, each of which is independently optionally substituted with R⁷. In some embodiments, R² is 5- or 6-membered heterocyclyl which is unsubstituted, such as tetrahydrofuranyl, pyrrolidinyl, piperidinyl, piperazinyl, morpholinyl, or thiomorpholinyl. In some embodiments, R² is H, halogen, C₂₋₆ alkenyl, —C(O)NR^{2a}R^{2b}, or C₃₋₁₂ cycloalkyl. In some embodiments, R² is H or halogen. In some embodiments, R² is H. In some embodiments, R² is halogen. In some embodiments, R² is chloro or fluoro. In some embodiments, R² is chloro. In some embodiments, R² is H, chloro, vinyl, —C(O)NH₂, or cyclopropyl.

In the descriptions herein, it is understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R¹ of formula (I) may be combined with every description, variation, embodiment or aspect of [symbol] , A, Z, Y, X¹, X², and R²-R⁵ the same as if each and every combination were specifically and individually listed. It is also understood that all descriptions, variations, embodiments or aspects of formula (I), where applicable, apply equally to other formulae detailed herein, and are equally described, the same as if each and every description, variation, embodiment or aspect were separately and individually listed for all formulae. For example, in some embodiments of a compound of formula (I) or any related formula where applicable, or a stereoisomer, tautomer, prodrug, or a pharmaceutically acceptable salt of any of the foregoing, [symbol] means an aromatic ring; Y is CH; Z is N; A is N; X¹ is H or —OH; X² is H or halogen; R¹ is —NR^{1a}R^{1b}; R^{1a} is C₁₋₆ alkyl, C₃₋₁₂ cycloalkyl, or 3- to 12-membered heterocyclyl, each of which is independently optionally substituted with R⁶, wherein R⁶ is 3- to 12-membered heterocyclyl or C₆₋₁₄ aryl, wherein the 3- to 12-membered heterocyclyl and C₆₋₁₄ aryl of R⁶ are each independently optionally substituted with halogen; R^{1b} is H or C₁₋₆ alkyl, or R^{1a} and R^{1b} are taken together with the nitrogen atom to which they attach to form a 3- to 12-membered heterocyclyl; R² is H or halogen; R³ is H; R⁴ is H; and R⁵ is H.

In some embodiments, provided is compound selected from the compounds in Table 1, or a stereoisomer, tautomer, solvate, prodrug or salt thereof. In some embodiments, provided is compound selected from the compounds in Table 1, or a stereoisomer, tautomer, prodrug, or a pharmaceutically acceptable salt of any of the foregoing. In some embodiments, provided is a compound selected from the compounds in Table 1, or a pharmaceutically acceptable salt thereof. In some embodiments, provided is a compound selected from the compounds in Table 1. Although certain compounds described in Table 1 are presented as specific stereoisomers and/or in a non-stereochemical form, it is understood that any or all stereochemical forms, including any enantiomeric or diastereomeric forms, and any tautomers or other forms of any of the compounds of Table 1 are herein described.

TABLE 1

| Compound No. | Structure |
|---|---|
| 1 | 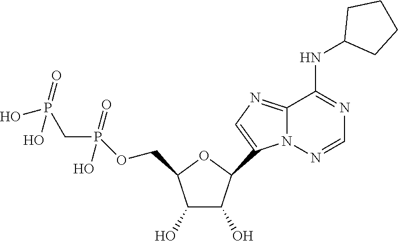 [SMILES: O=P(O)(O)CP(=O)(O)OC[C@H]1O[C@@H](c2cnc3c(NC4CCCC4)ncnn23)[C@H](O)[C@@H]1O] |
| 2 | 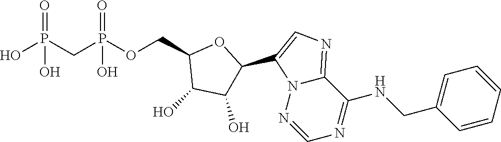 [SMILES: O=P(O)(O)CP(=O)(O)OC[C@H]1O[C@@H](c2cnc3c(NCc4ccccc4)ncnn23)[C@H](O)[C@@H]1O] |
| 3 | 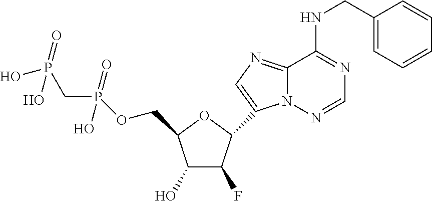 [SMILES: O=P(O)(O)CP(=O)(O)OC[C@H]1O[C@H](c2cnc3c(NCc4ccccc4)ncnn23)[C@@H](F)[C@@H]1O] |
| 4 | 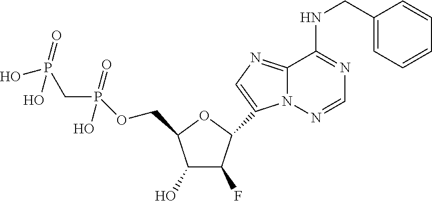 [SMILES: O=P(O)(O)CP(=O)(O)OC[C@H]1O[C@H](c2cnc3c(NCc4ccccc4)ncnn23)[C@@H](F)[C@@H]1O] |
| 5 | 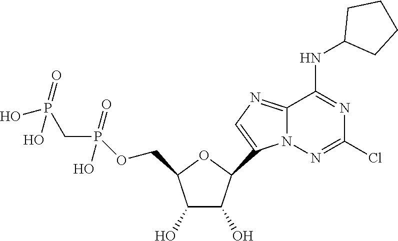 [SMILES: O=P(O)(O)CP(=O)(O)OC[C@H]1O[C@@H](c2cnc3c(NC4CCCC4)nc(Cl)nn23)[C@H](O)[C@@H]1O] |
| 6 | 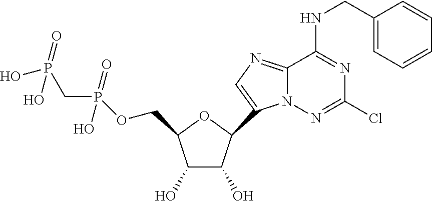 [SMILES: O=P(O)(O)CP(=O)(O)OC[C@H]1O[C@@H](c2cnc3c(NCc4ccccc4)nc(Cl)nn23)[C@H](O)[C@@H]1O] |
| 7 | 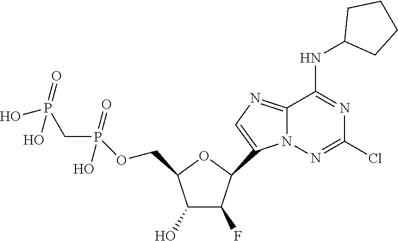 [SMILES: O=P(O)(O)CP(=O)(O)OC[C@H]1O[C@@H](c2cnc3c(NC4CCCC4)nc(Cl)nn23)[C@@H](F)[C@@H]1O] |
| 8 | 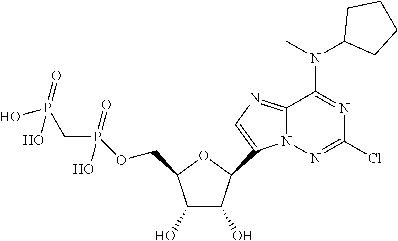 [SMILES: CN(c1nc(Cl)nn2c([C@@H]3O[C@H](COP(=O)(O)CP(=O)(O)O)[C@@H](O)[C@H]3O)cnc12)C1CCCC1] |
| 9 | 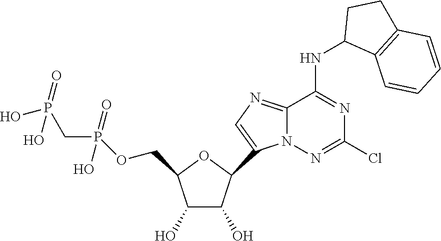 [SMILES: O=P(O)(O)CP(=O)(O)OC[C@H]1O[C@@H](c2cnc3c(NC4CCc5ccccc54)nc(Cl)nn23)[C@H](O)[C@@H]1O] |
| 10 | 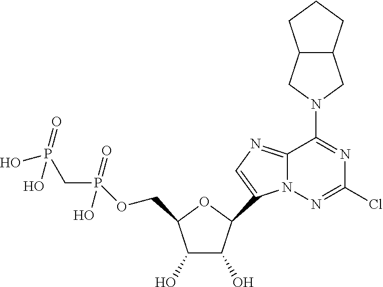 [SMILES: O=P(O)(O)CP(=O)(O)OC[C@H]1O[C@@H](c2cnc3c(N4CC5CCCC5C4)nc(Cl)nn23)[C@H](O)[C@@H]1O] |
| 11 | 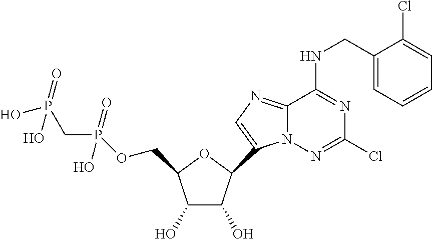 [SMILES: O=P(O)(O)CP(=O)(O)OC[C@H]1O[C@@H](c2cnc3c(NCc4ccccc4Cl)nc(Cl)nn23)[C@H](O)[C@@H]1O] |
| 12 | 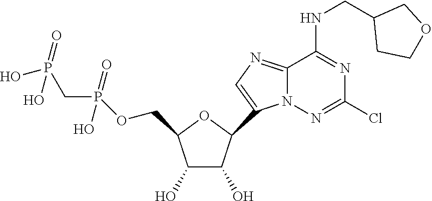 [SMILES: O=P(O)(O)CP(=O)(O)OC[C@H]1O[C@@H](c2cnc3c(NCC4CCOC4)nc(Cl)nn23)[C@H](O)[C@@H]1O] |
| 13 | 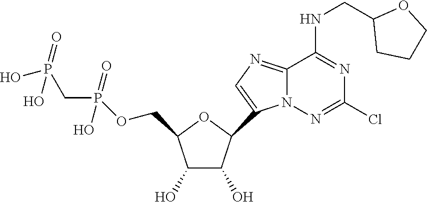 [SMILES: O=P(O)(O)CP(=O)(O)OC[C@H]1O[C@@H](c2cnc3c(NCC4CCCO4)nc(Cl)nn23)[C@H](O)[C@@H]1O] |
| 14 | 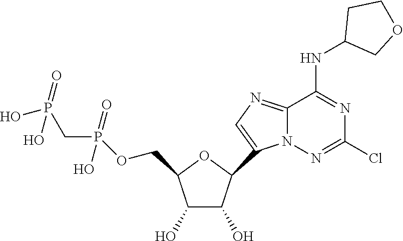 [SMILES: O=P(O)(O)CP(=O)(O)OC[C@H]1O[C@@H](c2cnc3c(NC4CCOC4)nc(Cl)nn23)[C@H](O)[C@@H]1O] |
| 15 | 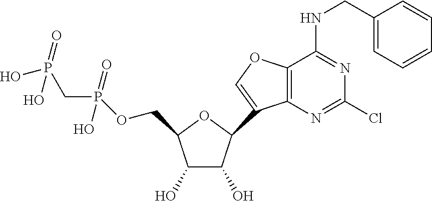 [SMILES: O=P(O)(O)CP(=O)(O)OC[C@H]1O[C@@H](c2coc3c(NCc4ccccc4)nc(Cl)nc23)[C@H](O)[C@@H]1O] |
| 16 | 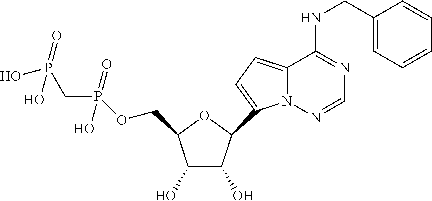 [SMILES: O=P(O)(O)CP(=O)(O)OC[C@H]1O[C@@H](c2ccc3c(NCc4ccccc4)ncnn23)[C@H](O)[C@@H]1O] |
| 17 | 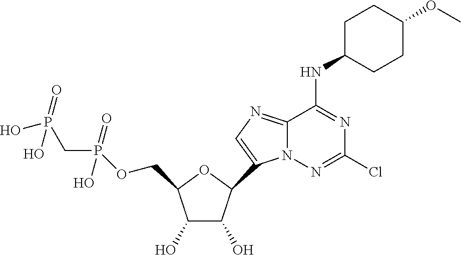 [SMILES: CO[C@H]1CC[C@H](Nc2nc(Cl)nn3c([C@@H]4O[C@H](COP(=O)(O)CP(=O)(O)O)[C@@H](O)[C@H]4O)cnc23)CC1] |
| 18 | 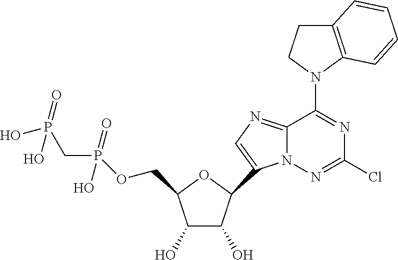 [SMILES: O=P(O)(O)CP(=O)(O)OC[C@H]1O[C@@H](c2cnc3c(N4CCc5ccccc54)nc(Cl)nn23)[C@H](O)[C@@H]1O] |
| 19 | 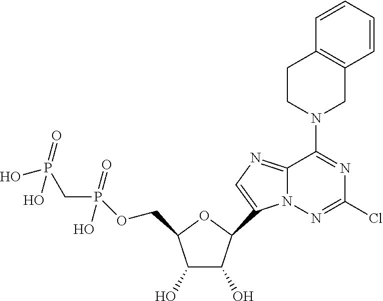 [SMILES: O=P(O)(O)CP(=O)(O)OC[C@H]1O[C@@H](c2cnc3c(N4CCc5ccccc5C4)nc(Cl)nn23)[C@H](O)[C@@H]1O] |
| 20 | 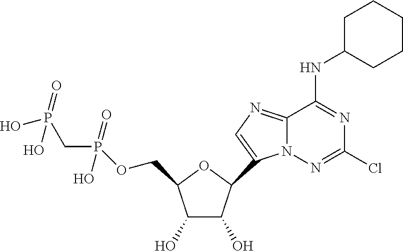 [SMILES: O=P(O)(O)CP(=O)(O)OC[C@H]1O[C@@H](c2cnc3c(NC4CCCCC4)nc(Cl)nn23)[C@H](O)[C@@H]1O] |
| 21 | 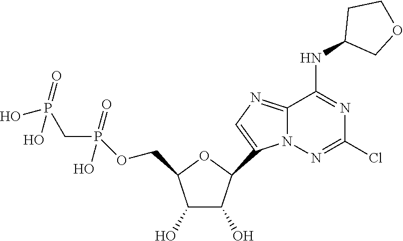 [SMILES: O=P(O)(O)CP(=O)(O)OC[C@H]1O[C@@H](c2cnc3c(N[C@H]4CCOC4)nc(Cl)nn23)[C@H](O)[C@@H]1O] |
| 22 | 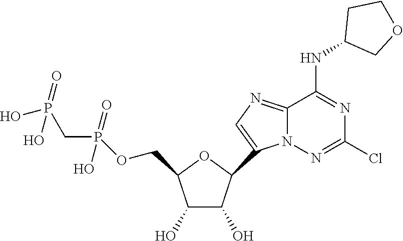 [SMILES: O=P(O)(O)CP(=O)(O)OC[C@H]1O[C@@H](c2cnc3c(N[C@@H]4CCOC4)nc(Cl)nn23)[C@H](O)[C@@H]1O] |
| 23 | 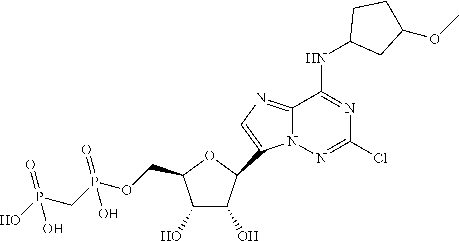 [SMILES: COC1CCC(Nc2nc(Cl)nn3c([C@@H]4O[C@H](COP(=O)(O)CP(=O)(O)O)[C@@H](O)[C@H]4O)cnc23)C1] |
| 24 | 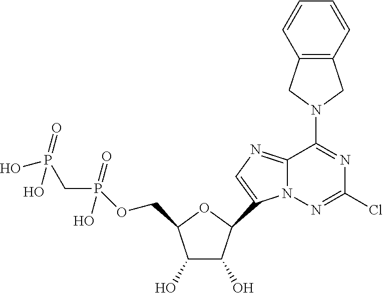 [SMILES: O=P(O)(O)CP(=O)(O)OC[C@H]1O[C@@H](c2cnc3c(N4Cc5ccccc5C4)nc(Cl)nn23)[C@H](O)[C@@H]1O] |
| 25 | 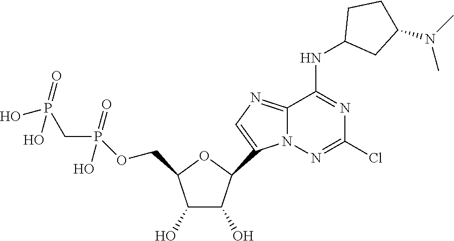 [SMILES: CN(C)[C@H]1CCC(Nc2nc(Cl)nn3c([C@@H]4O[C@H](COP(=O)(O)CP(=O)(O)O)[C@@H](O)[C@H]4O)cnc23)C1] |
| 26 | 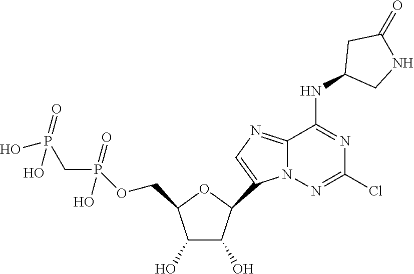 [SMILES: O=C1C[C@H](Nc2nc(Cl)nn3c([C@@H]4O[C@H](COP(=O)(O)CP(=O)(O)O)[C@@H](O)[C@H]4O)cnc23)CN1] |
| 27 | 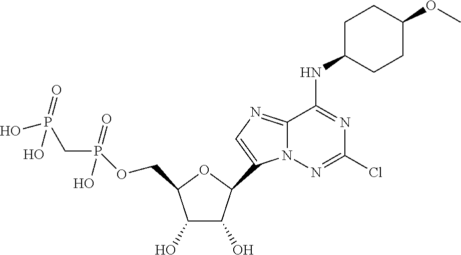 [SMILES: CO[C@H]1CC[C@@H](Nc2nc(Cl)nn3c([C@@H]4O[C@H](COP(=O)(O)CP(=O)(O)O)[C@@H](O)[C@H]4O)cnc23)CC1] |
| 28 | 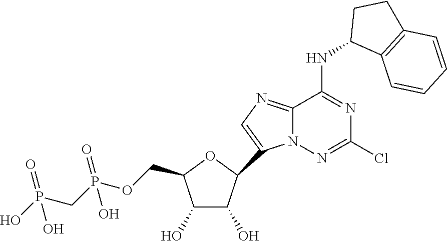 [SMILES: O=P(O)(O)CP(=O)(O)OC[C@H]1O[C@@H](c2cnc3c(N[C@@H]4CCc5ccccc54)nc(Cl)nn23)[C@H](O)[C@@H]1O] |
| 29 | 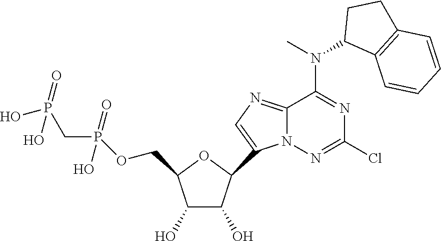 |
| 30 | 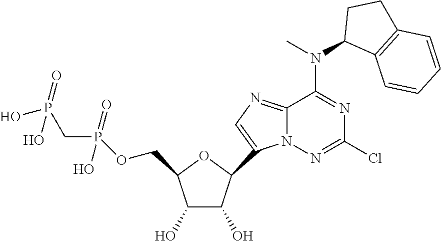 [SMILES: CN(c1nc(Cl)nn2c([C@@H]3O[C@H](COP(=O)(O)CP(=O)(O)O)[C@@H](O)[C@H]3O)cnc12)[C@H]1CCc2ccccc21] |
| 31 | 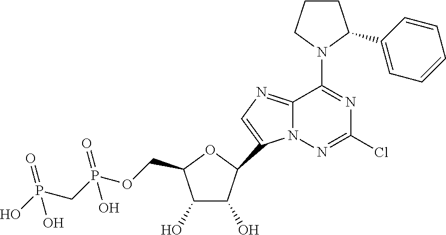 [SMILES: O=P(O)(O)CP(=O)(O)OC[C@H]1O[C@@H](c2cnc3c(N4CCC[C@@H]4c4ccccc4)nc(Cl)nn23)[C@H](O)[C@@H]1O] |
| 32 | 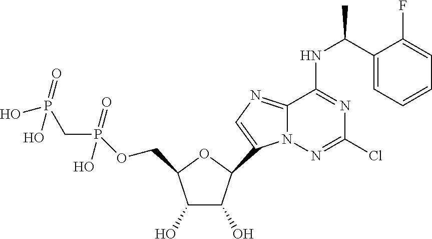 [SMILES: C[C@H](Nc1nc(Cl)nn2c([C@@H]3O[C@H](COP(=O)(O)CP(=O)(O)O)[C@@H](O)[C@H]3O)cnc12)c1ccccc1F] |
| 33 | 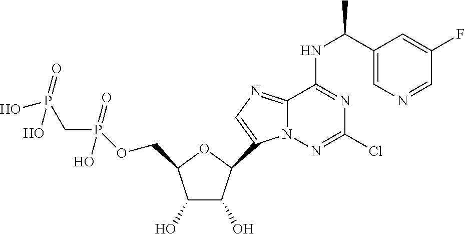 [SMILES: C[C@H](Nc1nc(Cl)nn2c([C@@H]3O[C@H](COP(=O)(O)CP(=O)(O)O)[C@@H](O)[C@H]3O)cnc12)c1cncc(F)c1] |
| 34 | 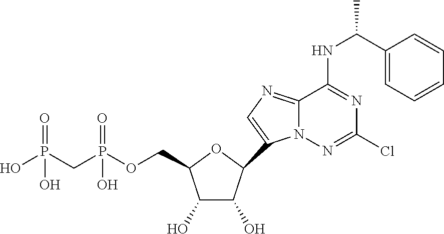 [SMILES: C[C@@H](Nc1nc(Cl)nn2c([C@@H]3O[C@H](COP(=O)(O)CP(=O)(O)O)[C@@H](O)[C@H]3O)cnc12)c1ccccc1] |
| 35 | 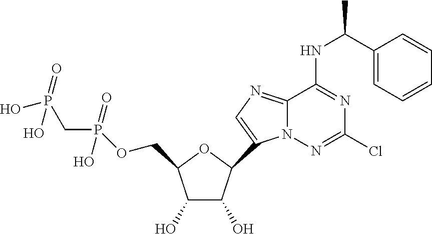 [SMILES: C[C@H](Nc1nc(Cl)nn2c([C@@H]3O[C@H](COP(=O)(O)CP(=O)(O)O)[C@@H](O)[C@H]3O)cnc12)c1ccccc1] |
| 36 | 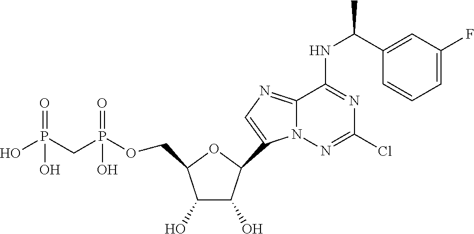 [SMILES: C[C@H](Nc1nc(Cl)nn2c([C@@H]3O[C@H](COP(=O)(O)CP(=O)(O)O)[C@@H](O)[C@H]3O)cnc12)c1cccc(F)c1] |
| 37 | 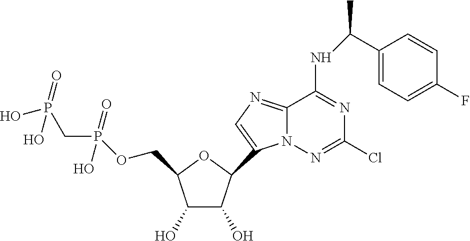 [SMILES: C[C@H](Nc1nc(Cl)nn2c([C@@H]3O[C@H](COP(=O)(O)CP(=O)(O)O)[C@@H](O)[C@H]3O)cnc12)c1ccc(F)cc1] |
| 38 | 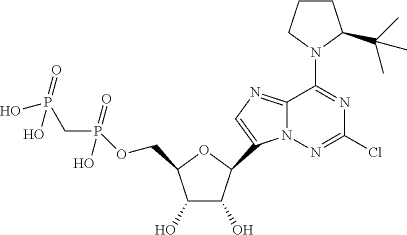 [SMILES: CC(C)(C)[C@@H]1CCCN1c1nc(Cl)nn2c([C@@H]3O[C@H](COP(=O)(O)CP(=O)(O)O)[C@@H](O)[C@H]3O)cnc12] |
| 39 | 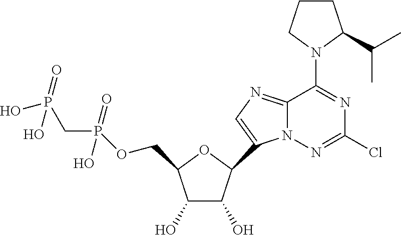 [SMILES: CC(C)[C@@H]1CCCN1c1nc(Cl)nn2c([C@@H]3O[C@H](COP(=O)(O)CP(=O)(O)O)[C@@H](O)[C@H]3O)cnc12] |
| 40 | 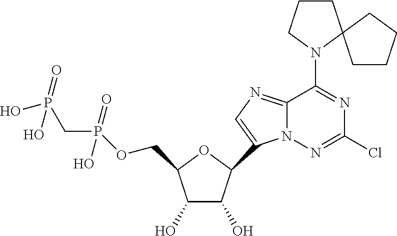 [SMILES: O=P(O)(O)CP(=O)(O)OC[C@H]1O[C@@H](c2cnc3c(N4CCCC45CCCC5)nc(Cl)nn23)[C@H](O)[C@@H]1O] |
| 41 | 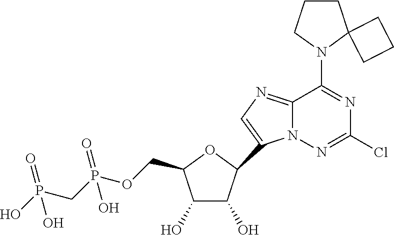 [SMILES: O=P(O)(O)CP(=O)(O)OC[C@H]1O[C@@H](c2cnc3c(N4CCCC45CCC5)nc(Cl)nn23)[C@H](O)[C@@H]1O] |
| 42 | 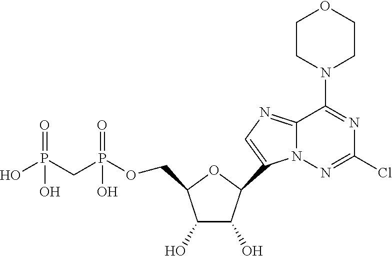 [SMILES: O=P(O)(O)CP(=O)(O)OC[C@H]1O[C@@H](c2cnc3c(N4CCOCC4)nc(Cl)nn23)[C@H](O)[C@@H]1O] |
| 43 | 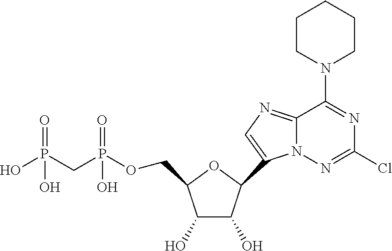 [SMILES: O=P(O)(O)CP(=O)(O)OC[C@H]1O[C@@H](c2cnc3c(N4CCCCC4)nc(Cl)nn23)[C@H](O)[C@@H]1O] |
| 44 | 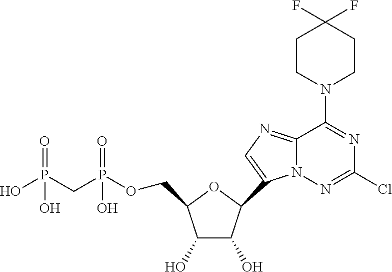 [SMILES: O=P(O)(O)CP(=O)(O)OC[C@H]1O[C@@H](c2cnc3c(N4CCC(F)(F)CC4)nc(Cl)nn23)[C@H](O)[C@@H]1O] |
| 45 | 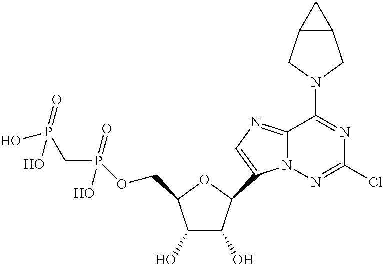 [SMILES: O=P(O)(O)CP(=O)(O)OC[C@H]1O[C@@H](c2cnc3c(N4CC5CC5C4)nc(Cl)nn23)[C@H](O)[C@@H]1O] |
| 46 | 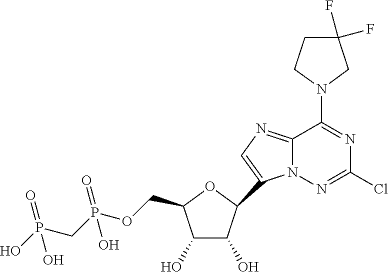 [SMILES: O=P(O)(O)CP(=O)(O)OC[C@H]1O[C@@H](c2cnc3c(N4CCC(F)(F)C4)nc(Cl)nn23)[C@H](O)[C@@H]1O] |
| 47 | 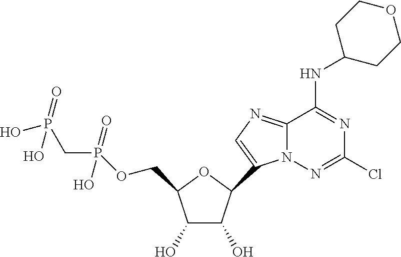 [SMILES: O=P(O)(O)CP(=O)(O)OC[C@H]1O[C@@H](c2cnc3c(NC4CCOCC4)nc(Cl)nn23)[C@H](O)[C@@H]1O] |
| 48 | 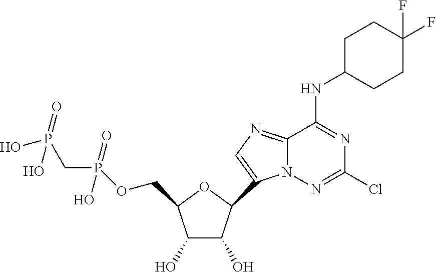 [SMILES: O=P(O)(O)CP(=O)(O)OC[C@H]1O[C@@H](c2cnc3c(NC4CCC(F)(F)CC4)nc(Cl)nn23)[C@H](O)[C@@H]1O] |
| 49 | 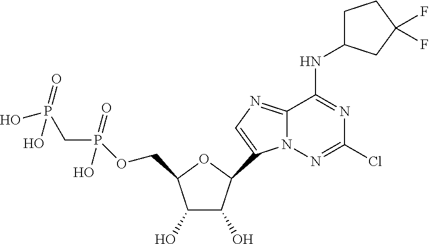 [SMILES: O=P(O)(O)CP(=O)(O)OC[C@H]1O[C@@H](c2cnc3c(NC4CCC(F)(F)C4)nc(Cl)nn23)[C@H](O)[C@@H]1O] |
| 50 | 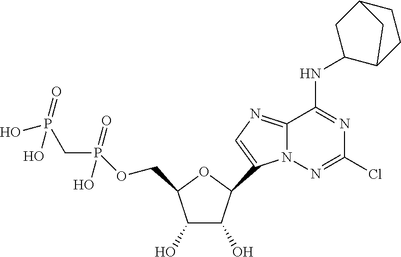 [SMILES: O=P(O)(O)CP(=O)(O)OC[C@H]1O[C@@H](c2cnc3c(NC4CC5CCC4C5)nc(Cl)nn23)[C@H](O)[C@@H]1O] |
| 51 | 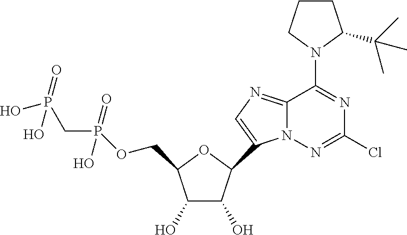 [SMILES: CC(C)(C)[C@H]1CCCN1c1nc(Cl)nn2c([C@@H]3O[C@H](COP(=O)(O)CP(=O)(O)O)[C@@H](O)[C@H]3O)cnc12] |
| 52 | 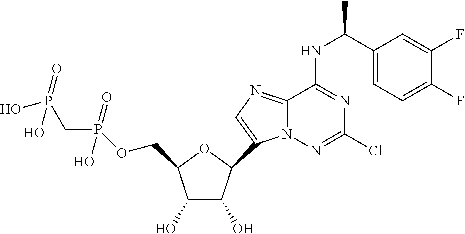 [SMILES: C[C@H](Nc1nc(Cl)nn2c([C@@H]3O[C@H](COP(=O)(O)CP(=O)(O)O)[C@@H](O)[C@H]3O)cnc12)c1ccc(F)c(F)c1] |
| 53 | 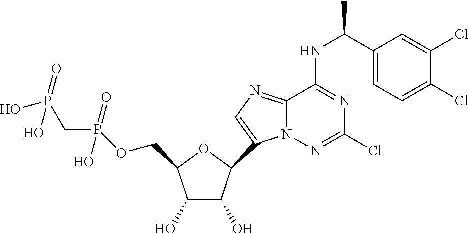 [SMILES: C[C@H](Nc1nc(Cl)nn2c([C@@H]3O[C@H](COP(=O)(O)CP(=O)(O)O)[C@@H](O)[C@H]3O)cnc12)c1ccc(Cl)c(Cl)c1] |
| 54 | 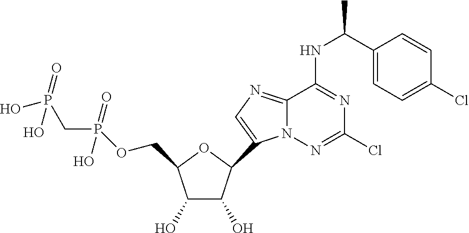 [SMILES: C[C@H](Nc1nc(Cl)nn2c([C@@H]3O[C@H](COP(=O)(O)CP(=O)(O)O)[C@@H](O)[C@H]3O)cnc12)c1ccc(Cl)cc1] |
| 55 | 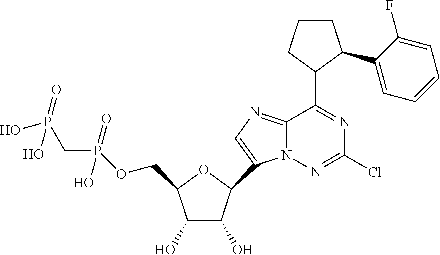 [SMILES: O=P(O)(O)CP(=O)(O)OC[C@H]1O[C@@H](c2cnc3c(C4CCC[C@H]4c4ccccc4F)nc(Cl)nn23)[C@H](O)[C@@H]1O] |
| 56 | 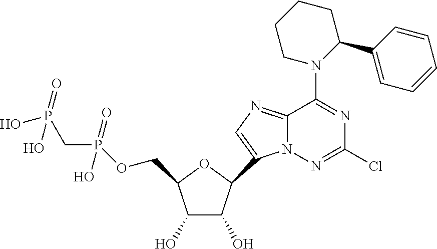 [SMILES: O=P(O)(O)CP(=O)(O)OC[C@H]1O[C@@H](c2cnc3c(N4CCCC[C@H]4c4ccccc4)nc(Cl)nn23)[C@H](O)[C@@H]1O] |
| 57 | 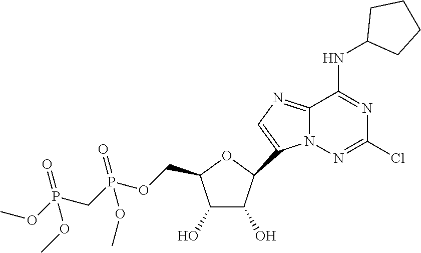 [SMILES: COP(=O)(CP(=O)(OC)OC[C@H]1O[C@@H](c2cnc3c(NC4CCCC4)nc(Cl)nn23)[C@H](O)[C@@H]1O)OC] |
| 58 | 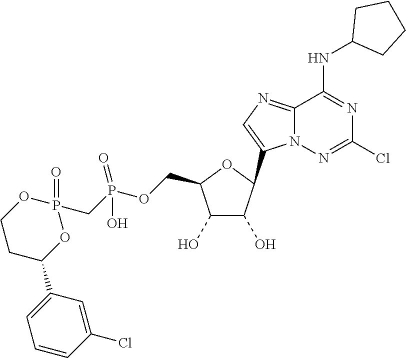 [SMILES: O=P(O)(CP1(=O)OCC[C@@H](c2cccc(Cl)c2)O1)OC[C@H]1O[C@@H](c2cnc3c(NC4CCCC4)nc(Cl)nn23)[C@H](O)[C@@H]1O] |
| 59 | 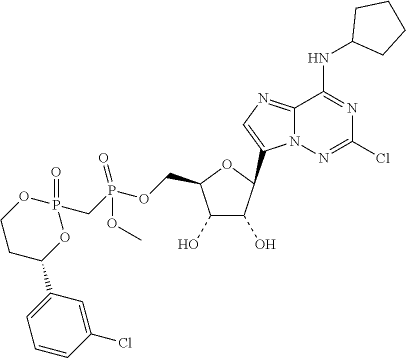 [SMILES: COP(=O)(CP1(=O)OCC[C@@H](c2cccc(Cl)c2)O1)OC[C@H]1O[C@@H](c2cnc3c(NC4CCCC4)nc(Cl)nn23)[C@H](O)[C@@H]1O] |
| 60 | 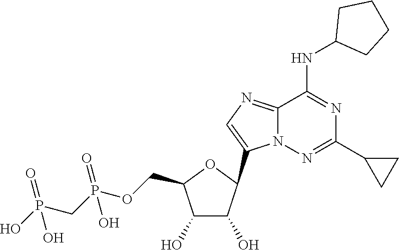 [SMILES: O=P(O)(O)CP(=O)(O)OC[C@H]1O[C@@H](c2cnc3c(NC4CCCC4)nc(C4CC4)nn23)[C@H](O)[C@@H]1O] |
| 61 | 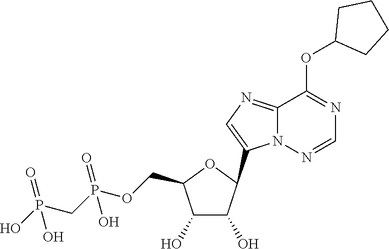 [SMILES: O=P(O)(O)CP(=O)(O)OC[C@H]1O[C@@H](c2cnc3c(OC4CCCC4)ncnn23)[C@H](O)[C@@H]1O] |
| 62 | 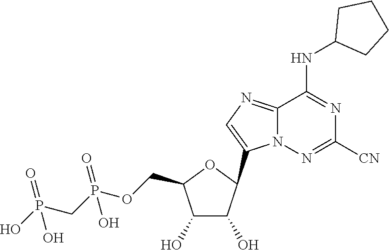 [SMILES: N#Cc1nc(NC2CCCC2)c2ncc([C@@H]3O[C@H](COP(=O)(O)CP(=O)(O)O)[C@@H](O)[C@H]3O)n2n1] |
| 63 | 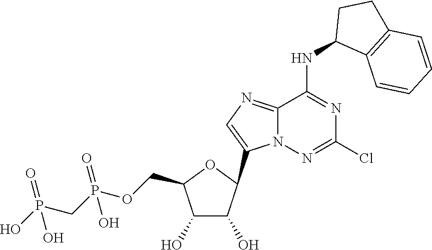 [SMILES: O=P(O)(O)CP(=O)(O)OC[C@H]1O[C@@H](c2cnc3c(N[C@H]4CCc5ccccc54)nc(Cl)nn23)[C@H](O)[C@@H]1O] |
| 64 | 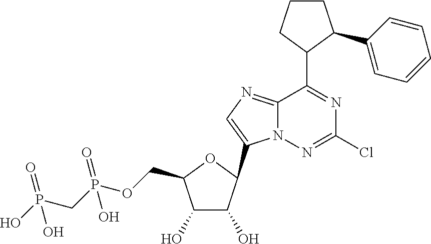 [SMILES: O=P(O)(O)CP(=O)(O)OC[C@H]1O[C@@H](c2cnc3c(C4CCC[C@H]4c4ccccc4)nc(Cl)nn23)[C@H](O)[C@@H]1O] |
| 65 | 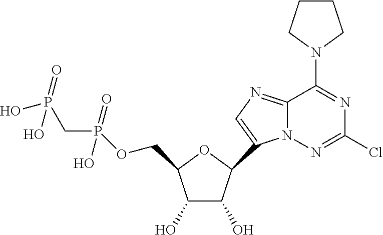 [SMILES: O=P(O)(O)CP(=O)(O)OC[C@H]1O[C@@H](c2cnc3c(N4CCCC4)nc(Cl)nn23)[C@H](O)[C@@H]1O] |
| 66 | 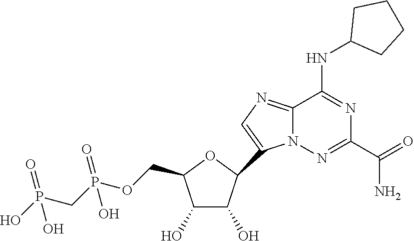 [SMILES: NC(=O)c1nc(NC2CCCC2)c2ncc([C@@H]3O[C@H](COP(=O)(O)CP(=O)(O)O)[C@@H](O)[C@H]3O)n2n1] |
| 67 | 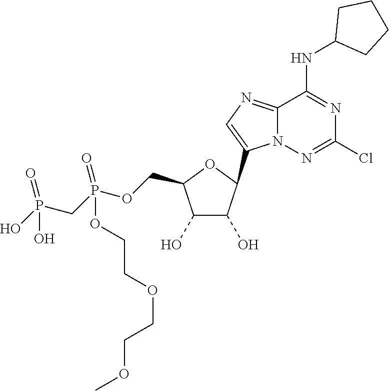 [SMILES: COCCOCCOP(=O)(CP(=O)(O)O)OC[C@H]1O[C@@H](c2cnc3c(NC4CCCC4)nc(Cl)nn23)[C@H](O)[C@@H]1O] |
| 68 | 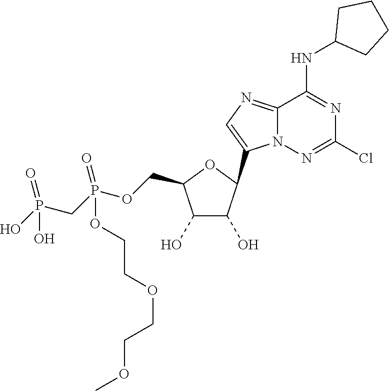 [SMILES: COCCOCCOP(=O)(CP(=O)(O)O)OC[C@H]1O[C@@H](c2cnc3c(NC4CCCC4)nc(Cl)nn23)[C@H](O)[C@@H]1O] |
| 69 | 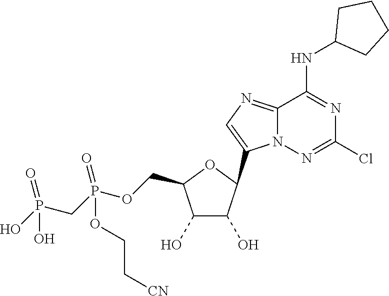 [SMILES: N#CCCOP(=O)(CP(=O)(O)O)OC[C@H]1O[C@@H](c2cnc3c(NC4CCCC4)nc(Cl)nn23)[C@H](O)[C@@H]1O] |
| 70 | 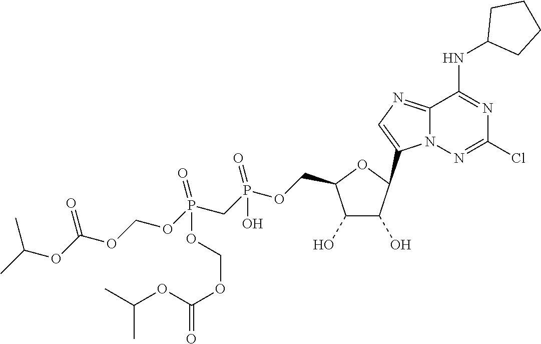 [SMILES: CC(C)OC(=O)OCOP(=O)(CP(=O)(O)OC[C@H]1O[C@@H](c2cnc3c(NC4CCCC4)nc(Cl)nn23)[C@H](O)[C@@H]1O)OCOC(=O)OC(C)C] |
| 71 | 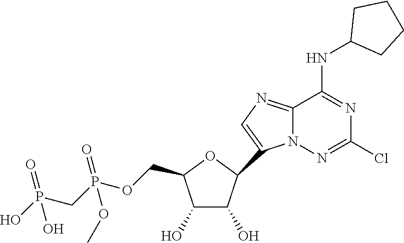 [SMILES: COP(=O)(CP(=O)(O)O)OC[C@H]1O[C@@H](c2cnc3c(NC4CCCC4)nc(Cl)nn23)[C@H](O)[C@@H]1O] |
| 72 | 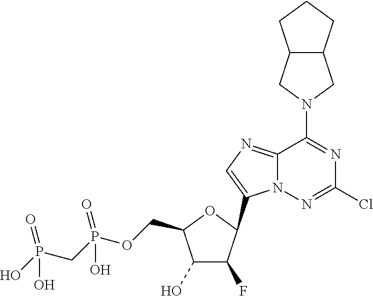 [SMILES: O=P(O)(O)CP(=O)(O)OC[C@H]1O[C@@H](c2cnc3c(N4CC5CCCC5C4)nc(Cl)nn23)[C@@H](F)[C@@H]1O] |
| 73 | 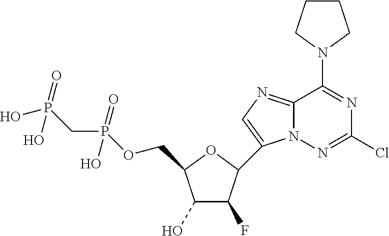 [SMILES: O=P(O)(O)CP(=O)(O)OC[C@H]1OC(c2cnc3c(N4CCCC4)nc(Cl)nn23)[C@@H](F)[C@@H]1O] |
| 74 | 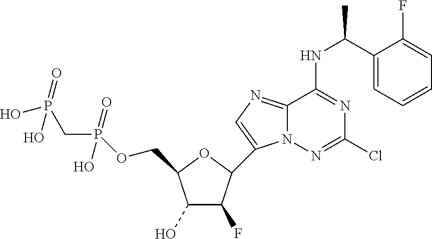 [SMILES: C[C@H](Nc1nc(Cl)nn2c(C3O[C@H](COP(=O)(O)CP(=O)(O)O)[C@@H](O)[C@@H]3F)cnc12)c1ccccc1F] |
| 75 | 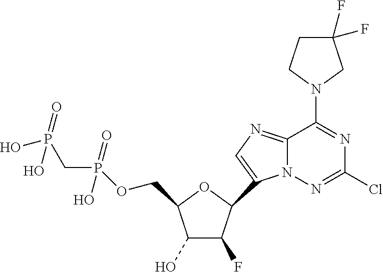 [SMILES: O=P(O)(O)CP(=O)(O)OC[C@H]1O[C@@H](c2cnc3c(N4CCC(F)(F)C4)nc(Cl)nn23)[C@@H](F)[C@@H]1O] |
| 76 | 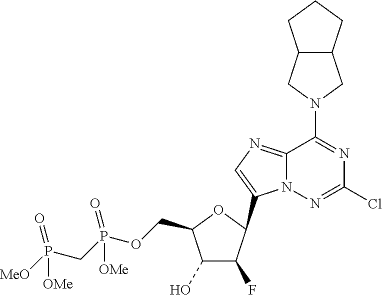 [SMILES: COP(=O)(CP(=O)(OC)OC[C@H]1O[C@@H](c2cnc3c(N4CC5CCCC5C4)nc(Cl)nn23)[C@@H](F)[C@@H]1O)OC] |
| 77 | 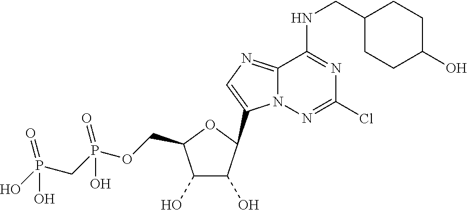 [SMILES: O=P(O)(O)CP(=O)(O)OC[C@H]1O[C@@H](c2cnc3c(NCC4CCC(O)CC4)nc(Cl)nn23)[C@H](O)[C@@H]1O] |
| 78 | 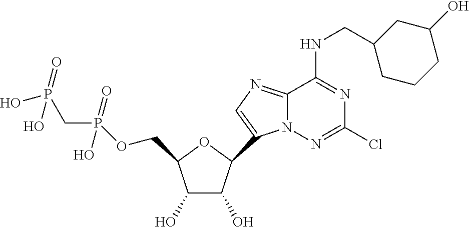 [SMILES: O=P(O)(O)CP(=O)(O)OC[C@H]1O[C@@H](c2cnc3c(NCC4CCCC(O)C4)nc(Cl)nn23)[C@H](O)[C@@H]1O] |
| 79 | 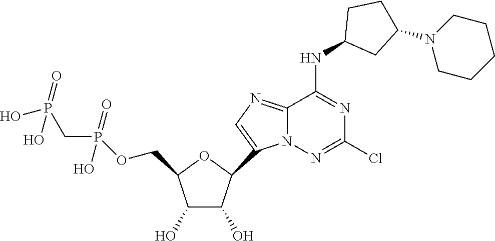 [SMILES: O=P(O)(O)CP(=O)(O)OC[C@H]1O[C@@H](c2cnc3c(N[C@H]4CC[C@H](N5CCCCC5)C4)nc(Cl)nn23)[C@H](O)[C@@H]1O] |
| 80 | 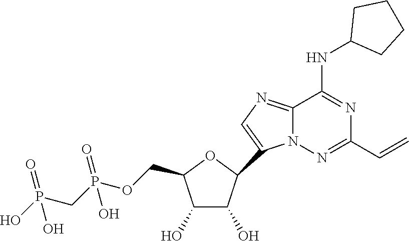 [SMILES: C=Cc1nc(NC2CCCC2)c2ncc([C@@H]3O[C@H](COP(=O)(O)CP(=O)(O)O)[C@@H](O)[C@H]3O)n2n1] |
| 81 | 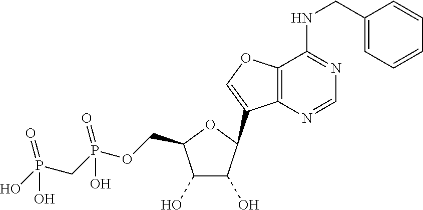 [SMILES: O=P(O)(O)CP(=O)(O)OC[C@H]1O[C@@H](c2coc3c(NCc4ccccc4)ncnc23)[C@H](O)[C@@H]1O] |

Also provided are salts of compounds referred to herein, such as pharmaceutically acceptable salts. The present disclosure also includes any or all of the stereochemical forms, including any enantiomeric or diastereomeric forms, and any tautomers or other forms of the compounds described. Thus, if a particular stereochemical form, such as a specific enantiomeric form or diastereomeric form, is depicted for a given compound, then it is understood that any or all stereochemical forms, including any enantiomeric or diastereomeric forms, and any tautomers or other forms of any of that same compound are herein described. Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The disclosure also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds described herein include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances. Isotopically-labeled compounds described herein can generally be prepared by standard methods and techniques known to those skilled in the art or by procedures similar to those described in the accompanying Examples substituting appropriate isotopically-labeled reagents in place of the corresponding non-labeled reagent.

The disclosure also includes any or all metabolites of any of the compounds described. The metabolites may include any chemical species generated by a biotransformation of any of the compounds described, such as intermediates and products of metabolism of the compound, such as would be generated in vivo following administration to a human.

Solvates and/or polymorphs of a compound provided herein or a salt thereof are also contemplated. Solvates contain either stoichiometric or non-stoichiometric amounts of a solvent and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 35% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### Pharmaceutical Compositions and Formulations

Pharmaceutical compositions of any of the compounds detailed herein are embraced by this disclosure. Thus, the present disclosure includes pharmaceutical compositions comprising a compound as detailed herein, or a stereoisomer, tautomer, prodrug, or a pharmaceutically acceptable salt of any of the foregoing, and a pharmaceutically acceptable carrier or excipient. In one aspect, the pharmaceutically acceptable salt is an acid addition salt, such as a salt formed with an inorganic or organic acid. Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein, or a stereoisomer, tautomer, prodrug, or a pharmaceutically acceptable salt of any of the foregoing, may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

A compound detailed herein, or a stereoisomer, tautomer, prodrug, or a pharmaceutically acceptable salt of any of the foregoing, can be used in the preparation of a formulation, such as a pharmaceutical formulation, by combining the compound or compounds, or a salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington's Pharmaceutical Sciences, Mack Publishing Company, Philadelphia, Pa., 20^{th} ed. (2000), which is incorporated herein by reference.

A compound detailed herein, or a stereoisomer, tautomer, prodrug, or a pharmaceutically acceptable salt of any of the foregoing, may be administered to individuals in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Any of the compounds described herein can be formulated in a tablet in any dosage form described, for example, a compound as described herein, or a salt thereof can be formulated as a 10 mg tablet.

Compositions comprising a compound provided herein are also described. In one variation, the composition comprises a compound or salt thereof and a pharmaceutically acceptable carrier or excipient. In another variation, a composition of substantially pure compound is provided. In some embodiments, the composition is for use as a human or veterinary medicament. In some embodiments, the composition is for use in a method described herein. In some embodiments, the composition is for use in the treatment of a disease or disorder described herein.

### Methods of Use

Compounds and compositions detailed herein, such as a pharmaceutical composition containing a compound of any formula provided herein, or a stereoisomer, tautomer, prodrug, or a pharmaceutically acceptable salt of any of the foregoing, and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease or disorder in an individual in need thereof comprising administering a compound describes herein or any embodiment, variation, or aspect thereof, or a pharmaceutically acceptable salt thereof. In some embodiments, the compound, pharmaceutically acceptable salt thereof, or composition is administered to the individual according to a dosage and/or method of administration described herein.

Compounds and compositions detailed herein can inhibit the activity of the CD73. For example, the compounds of the disclosure can be used to inhibit activity of CD73 in a cell or in an individual or patient in need of inhibition of the enzyme by administering an inhibiting amount of a compound of the disclosure to the cell, individual, or patient.

Compounds and compositions detailed herein are useful in the treatment of cancer. Examples of cancers include, without limitation, bladder cancer, leukemia, glioma, glioblastoma, melanoma, ovarian cancer, thyroid cancer, esophageal cancer, prostate cancer, lung cancer, colorectal cancer, pancreatic cancer, skin cancer, liver cancer, gastric cancer, head & neck cancer, and breast cancer.

Compounds and compositions detailed herein are useful in the treatment of immune-related disease. The term “immune-related disease” means a disease in which a component of the immune system causes, mediates or otherwise contributes to a morbidity. Also included are diseases in which stimulation or intervention of the immune response has an ameliorative effect on progression of the disease. Examples of immune-related diseases include, without limitation, immune-mediated inflammatory diseases, non-immune-mediated inflammatory diseases, infectious diseases, immunodeficiency diseases, and neoplasia, etc.

### Combinations

In certain aspects, compounds or compositions described herein are administered to an individual for treatment of a disease in combination with one or more additional pharmaceutical agents that can treat the disease. For example, in some embodiments, an effective amount of the compound of formula (I) or any related formula, or a stereoisomer, tautomer, prodrug, or a pharmaceutically acceptable salt of any of the foregoing, is administered to an individual for the treatment of a disease such as cancer in combination with one or more additional therapeutic agents. In some embodiments, the additional therapeutic agent comprises a checkpoint inhibitor. In some embodiments, the checkpoint inhibitor comprises a cytotoxic T lymphocyte associated protein 4 (CTLA-4) inhibitor, programmed cell death protein 1 (PD-1) inhibitor, or programmed death ligand 1 (PD-L1) inhibitor. In some embodiments, the checkpoint inhibitor comprises a CTLA-4 inhibitor such as ipilimumab. In some embodiments, the checkpoint inhibitor comprises a PD-1 inhibitor such as nivolumab or pembrolizumab. In some embodiments, the checkpoint inhibitor comprises a PD-L1 inhibitor such as atezolizumab. In some embodiments, the combination can be used for treating a cancer. In some embodiments, the combination can be used for treating a cancer, wherein the cancer is bladder cancer, leukemia, glioma, glioblastoma, melanoma, ovarian cancer, thyroid cancer, esophageal cancer, prostate cancer, colon cancer, or breast cancer.

### Dosing and Method of Administration

The dose of a compound administered to an individual (such as a human) may vary with the particular compound or salt thereof, the method of administration, and the particular disease, such as type and stage of cancer, being treated. In some embodiments, the amount of the compound or salt thereof is a therapeutically effective amount.

The effective amount of the compound may in one aspect be a dose of between about 0.01 and about 100 mg/kg. Effective amounts or doses of the compounds of the present disclosure may be ascertained by routine methods, such as modeling, dose escalation, or clinical trials, taking into account routine factors, e.g., the mode or route of administration or drug delivery, the pharmacokinetics of the agent, the severity and course of the disease to be treated, the subject's health status, condition, and weight. An exemplary dose is in the range of about from about 0.7 mg to 7 g daily, or about 7 mg to 350 mg daily, or about 350 mg to 1.75 g daily, or about 1.75 to 7 g daily.

Any of the methods provided herein may in one aspect comprise administering to an individual a pharmaceutical composition that contains an effective amount of a compound provided herein or a salt thereof and a pharmaceutically acceptable excipient.

A compound or composition provided herein may be administered to an individual in accordance with an effective dosing regimen for a desired period of time or duration, such as at least about one month, at least about 2 months, at least about 3 months, at least about 6 months, or at least about 12 months or longer, which in some variations may be for the duration of the individual's life. In one variation, the compound is administered on a daily or intermittent schedule. The compound can be administered to an individual continuously (for example, at least once daily) over a period of time. The dosing frequency can also be less than once daily, e.g., about a once weekly dosing. The dosing frequency can be more than once daily, e.g., twice or three times daily. The dosing frequency can also be intermittent, including a ‘drug holiday’ (e.g., once daily dosing for 7 days followed by no doses for 7 days, repeated for any 14-day time period, such as about 2 months, about 4 months, about 6 months or more). Any of the dosing frequencies can employ any of the compounds described herein together with any of the dosages described herein.

### Articles of Manufacture and Kits

The present disclosure further provides articles of manufacture comprising a compound described herein or a salt thereof, a composition described herein, or one or more unit dosages described herein in suitable packaging. In certain embodiments, the article of manufacture is for use in any of the methods described herein. Suitable packaging is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises one or more compounds described herein or a composition comprising a compound described herein. The kits may employ any of the compounds disclosed herein. In one variation, the kit employs a compound described herein or a salt thereof. The kits may be used for any one or more of the uses described herein, and, accordingly, may contain instructions for the treatment of any disease or described herein, for example for the treatment of cancer.

Kits generally comprise suitable packaging. The kits may comprise one or more containers comprising any compound described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound as disclosed herein and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### General Synthetic Methods

The compounds of the present disclosure may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High-Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

Solvates and/or polymorphs of a compound provided herein, or a salt thereof are also contemplated. Solvates contain either stoichiometric or non-stoichiometric amounts of a solvent and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and/or solubility. Various factors such as the recrystallization solvent, rate of crystallization, and storage temperature may cause a single crystal form to dominate.

General methods of preparing compounds according to the present disclosure are depicted in the schemes below, wherein L is a protecting group; and X¹, X², A, G, Y, Z, W, R¹, R², R^{1a}, R^{1b}, R⁴, R⁵, and R⁶ are as detailed herein.

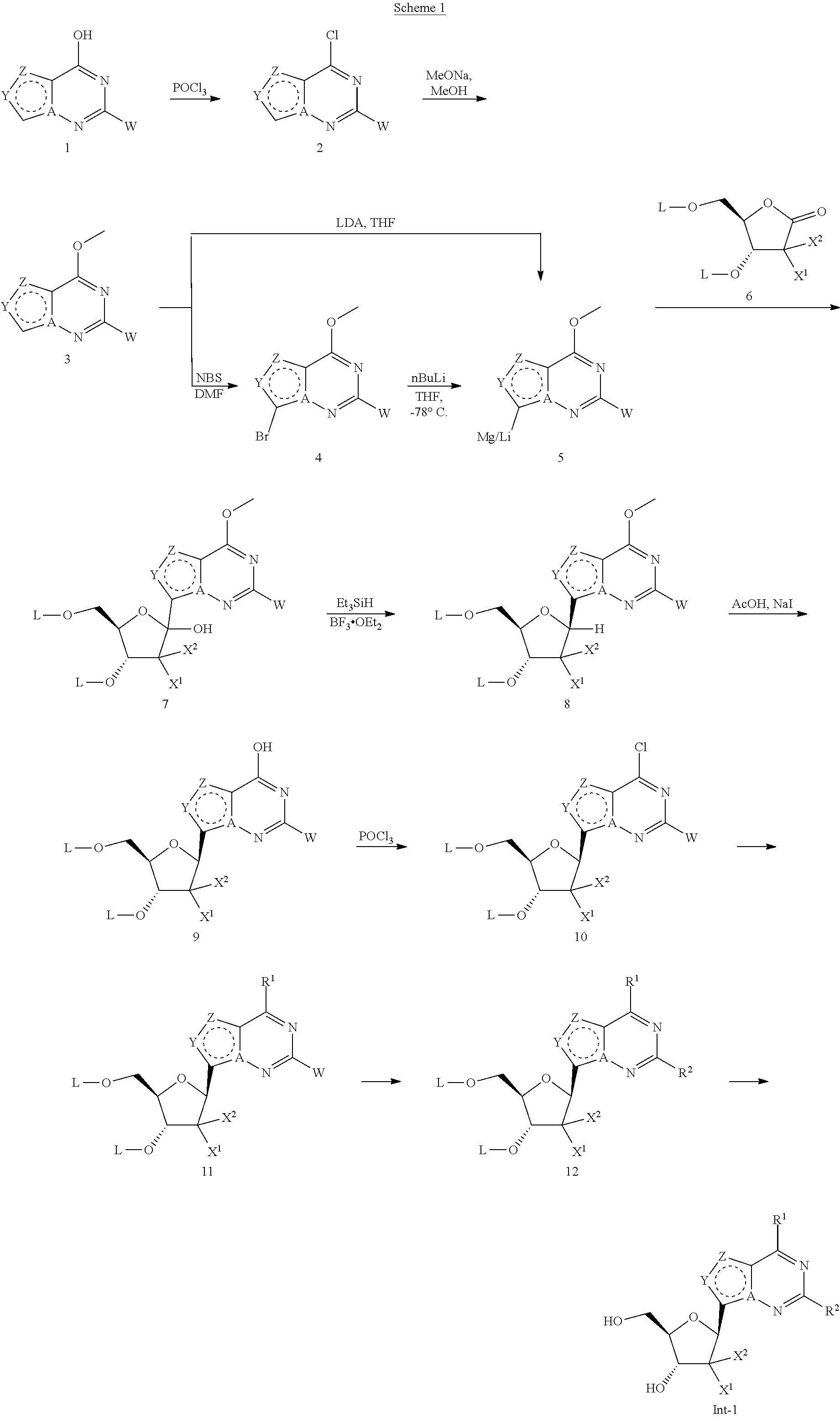

As shown in Scheme 1, some compounds of this invention can be prepared from 1. 1 is commercially available or can be prepared by procedures described in the literature. 1 can be converted into the chloro derivative 2 with e.g. POCl₃, PCl₃, PCl₅, or SOCl₂. A base, such as PhNMe₂ can be added during the reaction. Compound 3 can be prepared from 2 by reacting 2 with sodium methoxide. Compound 3 can be converted into the bromo-derivative 4 by reacting with NBS in an appropriate solvent such as, for example, CHCl₃ or CCl₄. 4 can be treated with an organometallic compound to give the metalated species 5. This can be accomplished, for example, with n-BuLi, sec-BuLi or tert-Buli or with MeMgBr and iPrMgBr in a solvent such as diethylether, dimethoxyethane, or THF. In some cases, 3 can be lithiated directly with, e.g. LDA or LHMDS to give 5. The organometallic species 5 can be added to the appropriately protected lactone 6, to give 7. Appropriate protecting groups (L) are known to those skilled in the art and are described, for example, in “Greene's Protective Groups in Organic Synthesis”, John Wiley & Sons, Inc., 2014.

Compound 7 can be reduced to 8 with a silane in presence of a Lewis acid. For example, Et₃SiH in presence of BF₃.OEt₂ will accomplish this reaction. De-methylation of 8 to give 9 can be accomplished, for example, by NaI in AcOH. 9 can be converted into the chloro derivative 10 by reacting with e.g. POCl₃, as described above. 10 can be reacted with an alcohol in the presence of a base, such as sodium hydride in a solvent, such as THF, to give 11 (R¹=—OR^{1a}). Alternatively, 10 can be reacted with a primary or secondary amine in the presence of a base, such as, for example, Et₃N or DIEA in a solvent, such as THF or EtOH to give 11 (R¹=—NR^{1a}R^{1b}). 11 can be converted into 12 by methods described in the individual Examples below. 12 can be deprotected to give Int-1. Deprotection will be accomplished by methods known to the skilled practitioner and are also described in “Greene's Protective Groups in Organic Synthesis”, John Wiley & Sons, Inc., 2014. For example, if the protection group is a benzyl ether (L=Bn), hydrogen in presence of a catalyst, such as Pd on carbon, or BCl₃ in DCM will achieve the deprotection. If the protection group is a silyl ether, the deprotection can be accomplished, for example, by using Bu₄NF in THF. Many other protecting groups and methods for removing them are known to those skilled in the art.

Alternatively, 11 can also be obtained as shown in Scheme 2, below. 13 can be converted into the chloro derivative 14 with e.g. POCl₃, PCl₃, PCl₅, or SOCl₂. Compound 15 can be obtained by treating 14 with either lithium tert-butoxide or sodium methanethiolate in an inert solvent, such as THF. Lithiation, with, for example, with n-BuLi, sec-BuLi or tert-Buli in a solvent such as diethylether, dimethoxyethane, or THF will give 16. 16 can be added to the appropriately protected lactone 6, to give 17. 17 can be reacted with an alcohol in the presence of a base, such as sodium hydride in a solvent, such as THF, to give 18 (R¹=—OR₁ₐ). Alternatively, 17 can be reacted with a primary or secondary amine in the presence of a base, such as, for example, Et₃N or DIEA in a solvent, such as THF or EtOH to give 18 (R¹=—NR^{1a}R^{1b}) Reduction of 18 with a silane in presence of a Lewis acid, such as Et₃SiH in presence of BF₃.OEt₂ as described above, will yield 11.

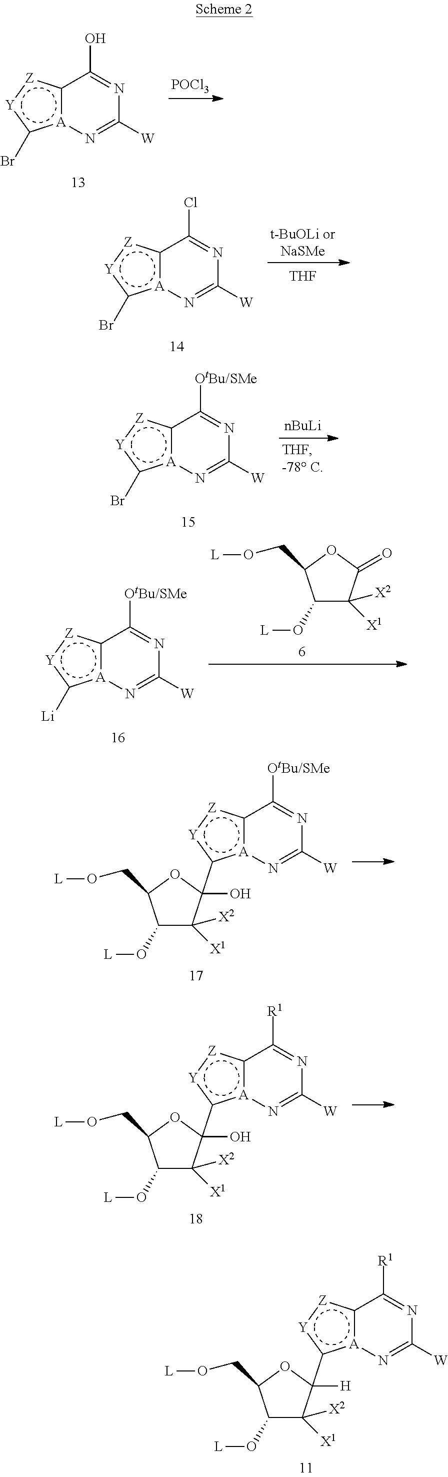

Scheme 3 below exemplifies the synthesis of compounds of the general structure 26. Briefly, di-tert-butyl phosphonate (19) is alkylated with MeI in presence of a base, such as NaH or BuLi, in a suitable solvent. Deprotonation of 20 with a base, such as LDA, followed by reaction with 1-chloro-N,N,N,N′-tetraisopropylphosphanediamine, yields compound 21. One of the diisopropylamino groups can be displaced by an alcohol (R—OH) or 25 by water (R═H) to give 22. Reaction of 22 with alcohol 23 in the presence of a coupling reagent, such as DCI in a suitable solvent, such as ACN will furnish 24. 24 can be oxidized to by an organic peroxide, such as, for example, ^{t}butyl hydroperoxide. Hydrolysis of the tert-butyl ester groups of 25 under acidic conditions and removal of the protecting group L will furnish 26. Appropriate protecting groups (L) are known to those skilled in the art and their introduction and removal are described, for example, in “Greene's Protective Groups in Organic Synthesis”, John Wiley & Sons, Inc., 2014.

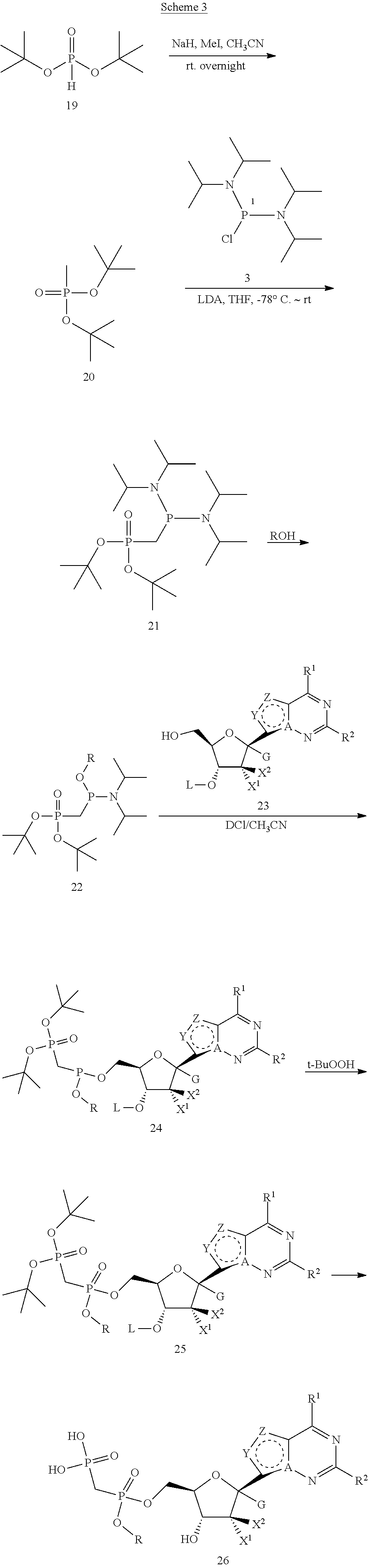

Yet, other compounds of this invention can be prepared as shown in Scheme 4. 2,4-Dichlorofuro[3,2-d]pyrimidine (27) can be treated with meOH in methanol to give 28. 28 can be brominated by using bromine in presence of KOAc or acetic acid. Treatment with a suitable base, such as, for example, KOH in EtOH will give 30. 30 can be treated with an organometallic compound to give the metalated species 31. This halogen-metal exchange can be accomplished, for example, with n-BuLi, sec-BuLi or tert-Buli or with MeMgBr and iPrMgBr in a solvent such as diethylether, dimethoxyethane, or THF. The organometallic species 31 can be added to the appropriately protected lactone 6, to give 32. Clevage of the methyl ether groups with, e.g. NaI in acetone and chlorination of the product with SOCl₂, PCl₅ or POCl₃ will give compound 33. 33 can be reacted with an alcohol in the presence of a base, such as sodium hydride in a solvent, such as THF, to give 34 (R¹=—OR^{1a}) Alternatively, 33 can be reacted with a primary or secondary amine in the presence of a base, such as, for example, Et₃N or DIEA in a solvent, such as THF or EtOH to give 34 (R¹=—NR^{1a}R^{1b}). 34 can be converted into 35 by methods described above and known to a person skilled in the art. Deprotection of 35, as described above will give Int-2.

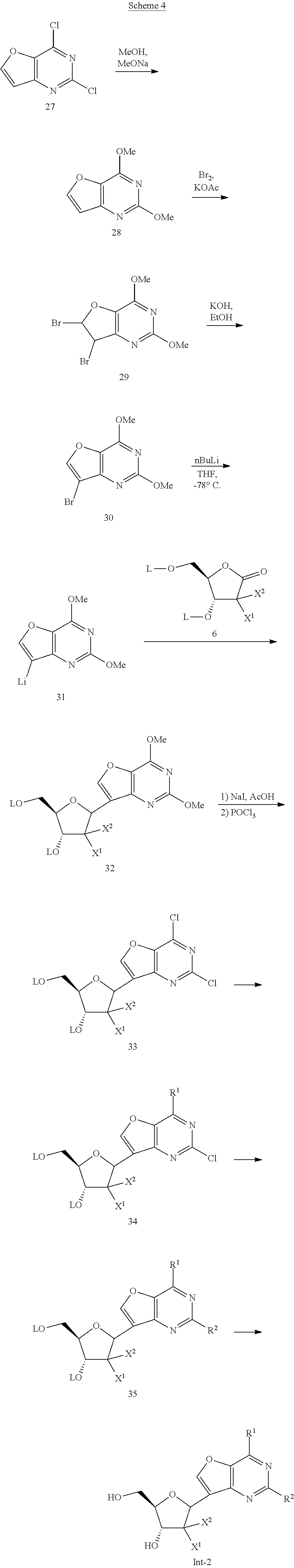

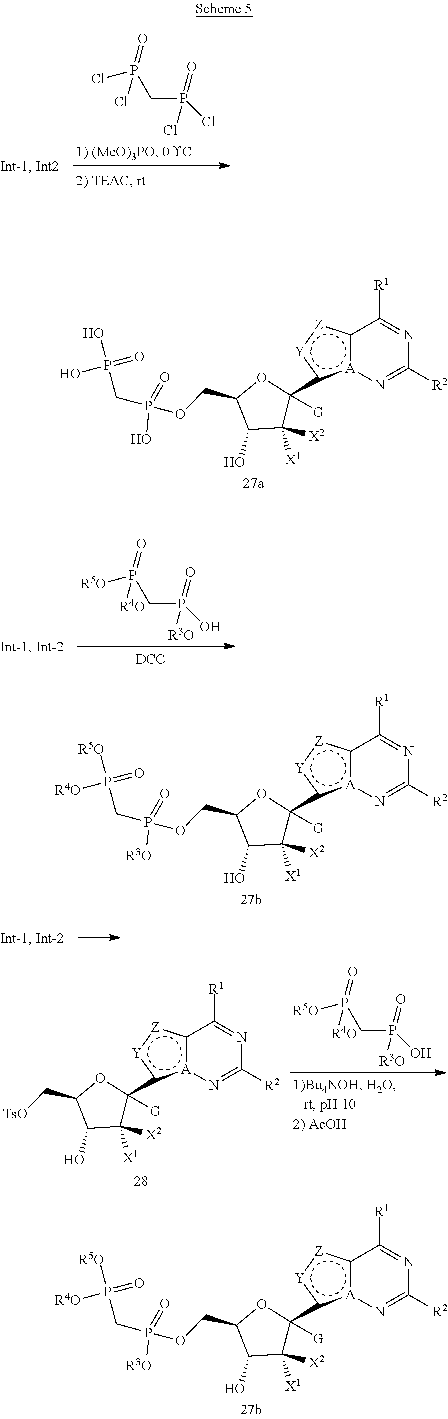

Compounds of formula 27a and 27b may be prepared according to steps outlined in Scheme 5. For example, reaction of Int-1 or Int-2 with methylenebis(phosphonic dichloride) followed by hydrolysis with a suitable base, such as TEAC, can provide 27a. Alternatively, reaction of Int-1 or Int-2 with methylenebis(phosphonic acid) or a suitable methylenebis(phosphonic acid) ester in presence of a coupling reagent, such as DCC, will provide 27b. Int-1 or Int-2 can also be converted into a mesylate, tosylate or triflate (28) by methods known to a person skilled in the art. Reaction of 28 with methylenebis(phosphonic acid) or a suitable methylenebis(phosphonic acid) ester in presence of a coupling reagent, such as DCC, will provide 27b which may be hydrolyzed using an acid, such as formic acid or acetic acid.

In some embodiments a compound of the present invention, for example a compound of a formula given in Table 1, is synthesized according to one of the general routes outlined in Schemes 1-5, Examples S1-S81 or by methods generally known to those skilled in the art.

### EXAMPLES

It is understood that the present disclosure has been made only by way of example, and that numerous changes in the combination and arrangement of parts can be resorted to by those skilled in the art without departing from the spirit and scope of the present disclosure.

The chemical reactions in the Examples described can be readily adapted to prepare a number of other compounds disclosed herein, and alternative methods for preparing the compounds of this disclosure are deemed to be within the scope of this disclosure. For example, the synthesis of non-exemplified compounds according to the present disclosure can be successfully performed by modifications apparent to those skilled in the art, e.g., by appropriately protecting interfering groups, by utilizing other suitable reagents known in the art other than those described, or by making routine modifications of reaction conditions, reagents, and starting materials. Alternatively, other reactions disclosed herein or known in the art will be recognized as having applicability for preparing other compounds of the present disclosure.

The following abbreviations may be used herein:
˜ about
+ve or pos. ion positive ion
Δ heat
Ac Acetyl
ACN Acetonitrile
Ac₂O Acetic anhydride
AcOH Acetic acid
AMP Adenosine monophosphate
anh. anhydrous
aq aqueous
Bn benzyl
Boc tert-butyloxycarbonyl
BSA bovine serum albumin
Bz benzoyl
Calcd or Calc'd calculated
CombiFlash® CombiFlash®, Teledyne ISCO Inc., Lincoln Nebr., USA
Conc. concentrated
d day(s) or doublet (NMR)
DCC dicyclohexylcarbodiimide
DCE Dichloroethane
DCI 1H-Imidazole-4,5-dicarbonitrile
DCM Dichloromethane
dd Dublet of doublets (NMR)
DEA Diethylamine
DIEA or DIPEA Diisopropylethylamine
DME 1,2-Dimethoxyethane
DMF N,N-Dimethylformamide
DMSO Dimethyl sulfoxide
EA Ethyl acetate
EHNA erythro-9-(2-Hydroxy-3-nonyl)adenine
eq equivalent
ESI electrospray ionization
Et ethyl
Et₂O Diethyl ether
Et₃N Triethylamine
EtOAc Ethyl acetate
EtOH Ethyl alcohol
FA Formic acid
g gram(s)
h hour(s)
Hex Hexanes
HMPA Hexamethylphosphoramide
HPLC high performance liquid chromatography
Hz Hertz
IPA or iPrOH Isopropyl alcohol
J Coupling constant (NMR) in Hz
KOAc Potassium acetate
LCMS, LC-MS or LC/MS liquid chromatography mass spectrometry
LDA lithium diisopropylamide
LHMDS or LiHMDS lithium hexamethyldisilazide
m Multiplet (NMR)
M molar (mol L⁻¹)
Me methyl
MeCN acetonitrile
MeI iodomethane
MeOH methyl alcohol
mg milligram(s)
min minute(s)
mL milliliter(s)
M mole(s)
MS mass spectrometry
MsCl methanesulfonyl chloride
MTBE or MtBE methyl tert-butyl ether
m/z mass-to-charge ratio
NaHMDS sodium hexamethyldisilazide
NaOtBu sodium tert-butoxide
nBuLi n-butyl lithium
NBS N-bromo succinimide
nm Nanometer (wavelength)
NMR nuclear magnetic resonance
P1 Product one; faster eluting isomer
P2 Product two; slower eluting isomer
PCC Pyridinium chlorochromate, CAS Number: 26299-14-9
PE Petroleum ether, CAS Number: 101316-46-5
PBS phosphate buffered saline
PMB para-methoxybenzyl, 4-methoxybenzyl
Pr propyl
ppm parts per million
p-tol para-toluoyl
rac racemic
RP-HPLC or RPHPLC reversed phase high performance liquid chromatography
RT or rt or r.t. room temperature
s singlet (NMR)
sat. or sat'd or satd saturated
SFC Supercritical fluid chromatography
t triplet (NMR)
TBSCl tert-Butyldimethylsilyl chloride
tBuOH tert-butyl alcohol
TEA triethylamine
TEAC triethylammonium chloride
tert or t tertiary
TFA triflouroacetic acid
THF tetrahydrofuran
TLC thin layer chromatography
TMS trimethylsilyl or trimethylsilane
TNAP Tissue-nonspecific alkaline phosphatase
Tris tris(hydroxymethyl)aminomethane
v/v volume per volume

### Example S1

### Synthesis of (((((2R,3S,4R,1S)-5-(4-(cyclopentylamino)imidazo[2,1-f][1,2,4]triazin-7-yl)-3,4-dihydroxytelmhydrofuran-2-yl)methoxy)(hydroxy)phosphoryl)methyl)phosphonic Acid

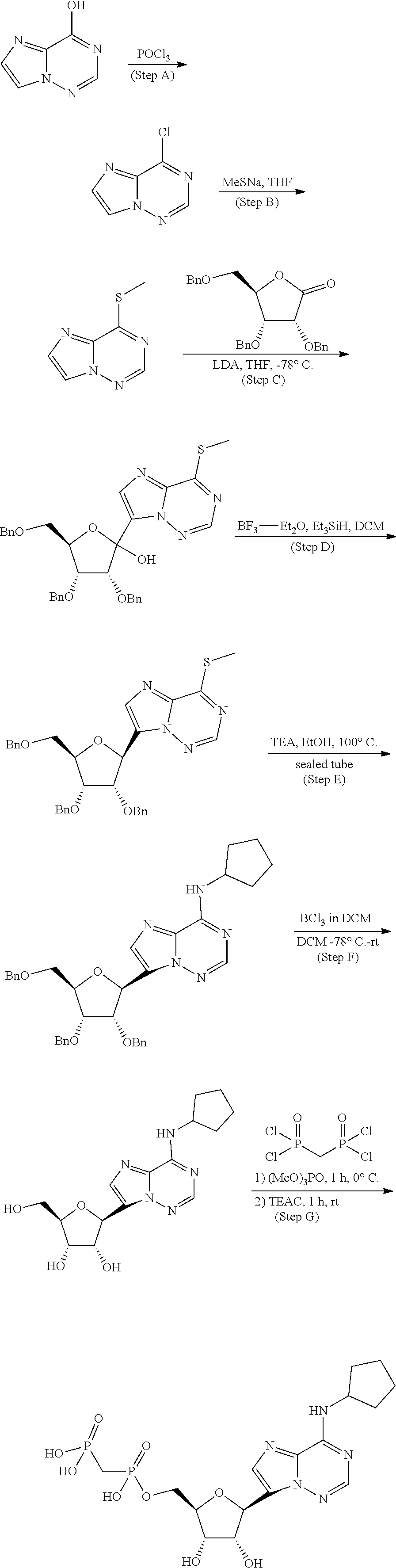

Step A: To a solution of imidazo[2,1-f][1,2,4]triazin-4-ol (1.85 g, 13.6 mmol) in POCl₃ (20 mL) was added PhNMe₂ (1.31 g, 12 mmol) dropwise at 0° C. Then the mixture was heated to reflux for 4 h. The solvent was removed under reduced pressure. The residue was dissolved in DCM and the organic layer was washed with sat. aq. NaHCO₃ solution and brine, dried over Na₂SO₄, filtered, the filtrate was concentrated and purified by CombiFlash® (PE:EA=5:1) to give 4-chloroimidazo[2,1-f][1,2,4]triazine (1.8 g, 86% yield) as yellow solid. Mass Spectrum (ESI) m/z=155.1 (M+23).

Step B: NaSCH₃ (818 mg, 11.68 mmol) was added to a solution of 4-chloroimidazo[2,1-f][1,2,4]triazine (1.8 g, 11.68 mmol) in THF (50 mL) portionwise at 0° C. The mixture was stirred at 50° C. for 18 h. The reaction was quenched with sat. aq. NH₄Cl solution and extracted with EA (50 mL×2). The organic layer was washed with brine, dried over Na₂SO₄ and filtered. The filtrate was concentrated and purified by CombiFlash® (PE:EA=5:1) to give 4-(methylsulfanyl)imidazo[2,1-f][1,2,4]triazine (887 mg, 45% yield). Mass Spectrum (ESI) m/z=167.1 (M+1).

Step C: To a solution of 4-(methylsulfanyl)imidazo[2,1-f][1,2,4]triazine (700 mg, 4.21 mmol) in THF (10 ml) under a N₂ atmosphere was added LDA (2 M, 4.2 mL, 8.4 mmol) dropwise at −78° C. The mixture was stirred at the same temperature for 30 min, then a solution of (3R,4R,5R)-3,4-bis(benzyloxy)-5-[(benzyloxy)methyl]oxolan-2-one (1.76 g, 4.21 mmol) in THF (10 mL) was added dropwise. The reaction was stirred at −78° C. for another 2 h. The reaction was quenched with sat. aq. NH₄Cl solution and extracted with EA. The organic layer was washed with brine, concentrated and purified by CombiFlash® (PE:EA=5:1) to give (3R,4R,5R)-3,4-bis(benzyloxy)-5-[(benzyloxy)methyl]-2-[4-(methylsulfanyl)imidazo[2,1-f][1,2,4]triazin-7-yl]oxolan-2-ol as a yellow oil (2 g, 81% yield). Mass Spectrum (ESI) m/z=586.1 (M+1).

Step D: To a solution of (3R,4R,5R)-3,4-bis(benzyloxy)-5-[(benzyloxy)methyl]-2-[4-(methylsulfanyl)imidazo[2,1-f][1,2,4]triazin-7-yl]oxolan-2-ol (2 g, 4.95 mmol) in DCM (20 mL) was added BF₃.Et₂O (2.8 g, 19.8 mmol) and Et₃SiH (2.3 g, 19.8 mmol) dropwise at −78° C. under a N₂ atmosphere. The resulting mixture was stirred at rt for 16 h. The reaction was quenched with sat. aq. NaHCO₃ solution and extracted with DCM. The organic layer was concentrated and purified by CombiFlash® (PE/EA=5:1) to give 7-[(3S,4R,5R)-3,4-bis(benzyloxy)-5-[(benzyloxy)methyl]oxolan-2-yl]-4-(methylsulfanyl)imidazo[2,1-f][1,2,4]triazine (790 mg, 40% yield). Mass Spectrum (ESI) m/z=569.1 (M+1).

Step E: A solution of 7-[(3S,4R,5R)-3,4-bis(benzyloxy)-5-[(benzyloxy)methyl]oxolan-2-yl]-4-(methylsulfanyl)imidazo[2,1-f][1,2,4]triazine (530 mg, 0.93 mmol), Et₃N (188 mg, 1.87 mmol) and cyclopentylamine (119 mg, 1.4 mmol) in EtOH (10 mL) was stirred at 70° C. for 5 h. The reaction was concentrated and purified by CombiFlash® (PE/EA=5:1) to give 7-[(3S,4R,5R)-3,4-bis(benzyloxy)-5-[(benzyloxy)methyl]oxolan-2-yl]-N-cyclopentylimidazo[2,1-f][1,2,4]triazin-4-amine (230 mg, 40% yield). Mass Spectrum (ESI) m/z=606.1 (M+1).

Step F: BCb (1M in DCM, 3.8 mL, 3.8 mmol) was added to a solution of 7-[(3S,4R,5R)-3,4-bis(benzyloxy)-5-[(benzyloxy)methyl]oxolan-2-yl]-N-cyclopentylimidazo[2,1-f][1,2,4]triazin-4-amine (230 mg, 0.38 mmol) in DCM (10 mL) dropwise at −78° C. The mixture was stirred at the same temperature for 2 h. Then the reaction was brought to −30° C. over a period of 30 min and quenched by adding a mixture of methanol:chloroform (2:1, 10 mL). After the reaction mixture reached rt, it was neutralized with NH₃ in methanol (10%, 10 mL) and concentrated. The residue was purified by CombiFlash® (DCM/MeOH=10:1) to give (3R,4S,5R)-2-[4-(cyclopentylamino)imidazo[2,1-f][1,2,4]triazin-7-yl]-5-(hydroxymethyl)oxolane-3,4-diol (100 mg, 78% yield). Mass Spectrum (ESI) m/z=336.1 (M+1).

Step G: To a solution of (3R,4S,5R)-2-[4-(cyclopentylamino)imidazo[2,1-f][1,2,4]triazin-7-yl]-5-(hydroxymethyl)oxolane-3,4-diol (100 mg, 0.3 mmol) in trimethylphosphate (1 mL) at 0° C. was added a cold solution of methylenebis(phosphonic dichloride) (374 mg, 1.5 mmol) in trimethylphosphate (1 mL) dropwise. Then the reaction solution was stirred at 0° C. for 1 h. TEAC (0.5 M, 2.1 mL) was added to the reaction carefully, and the reaction was stirred at this temperature for 15 min, then warmed to room temperature and stirring was continued for 1 h. Trimethylphosphate was extracted using tert-butyl methyl ether (5 mL×2) and the aqueous layer was basified with ammonium hydroxide to pH ˜7-8. Then purified by Prep-HPLC using a gradient of 0.2% formic acid/ACN from 90:10 to 70:30, and suitable fractions were pooled and lyophilized to give (((((2R,3S,4R,5S)-5-(4-(cyclopentylamino)imidazo[2,1-f][1,2,4]triazin-7-yl)-3,4-dihydroxytetrahydrofuran-2-yl)methoxyxhydroxy)phosphoryl)methyl)phosphonic acid (11 mg, 7% yield) as a white solid.

¹H NMR (400 MHz, D₂O) δ 8.19 (s, 1H), 7.87 (s, 1H), 5.32 (d, J=6.4 Hz, 1H), 4.74 (d, J=4.5 Hz, 1H), 4.69-4.62 (m, 1H), 4.45-4.39 (m, 1H), 4.30-4.24 (m, 1H), 4.12-4.02 (m, 2H), 2.25-2.00 (m, 5H), 1.84-1.63 (m, 5H). Mass Spectrum (ESI) m/z=491.7 (M−1).

### Example S2

### Synthesis of (((((2R,3S,4R,1S)-5-(4-(benzylamino)imidazo[2,1-f][1,2,4]triazin-7-yl)-3,4-dihydroxytetrahydrofuran-2-yl)methoxy)(hydroxy)phosphoryl)methyl)phosphonic Acid

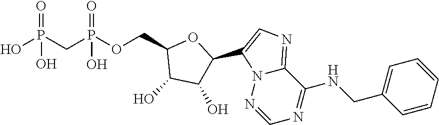 [SMILES: O=P(O)(O)CP(=O)(O)OC[C@H]1O[C@@H](c2cnc3c(NCc4ccccc4)ncnn23)[C@H](O)[C@@H]1O]

(((((2R,3S,4R,5S)-5-(4-(cyclopentylamino)imidazo[2,1-f][1,2,4]triazin-7-yl)-3,4-dihydroxytetrahydrofuran-2-yl)methoxyxhydroxy)phosphoryl)methyl)phosphonic acid was synthesized by procedures similar to the ones described in Example S1, replacing cyclopentylamine in Step E with benzylamine.

¹H NMR (400 MHz, DMSO) δ 9.37 (s, 1H), 8.15 (s, 1H), 7.70 (s, 1H), 7.42-7.27 (m, 4H), 7.26-7.20 (m, 1H), 5.09 (d, J=5.5 Hz, 1H), 4.73 (d, J=4.4 Hz, 2H), 4.38-4.29 (m, 1H), 4.14-3.82 (m, 4H), 2.09 (t, J=17.6 Hz, 2H). Mass Spectrum (ESI) m/z=514.0 (M−1).

### Example S3

### Synthesis of [({[(2R,3R,4S,5S)-5-[4-(benzylamino)imidazo[2,1-f][1,2,4]triazin-7-yl]-4-fluoro-3-hydroxyoxolan-2-yl]methoxy}(hydroxy)phosphoryl)methyl]phosphonic Acid

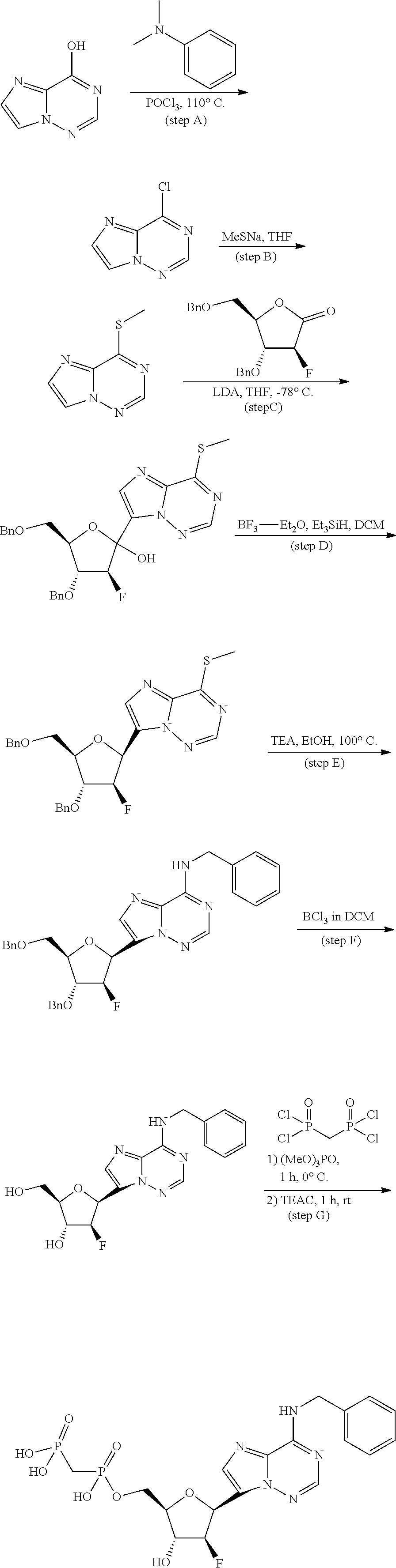

Step A: To a suspension of imidazo[2,1-f][1,2,4]triazin-4-ol (5 g, 36.8 mmol) in POCl₃ (150 mL) was added N,N-dimethylaniline (3.6 g, 29.4 mmol). Then the mixture was heated to reflux for 4 h. The solvent was removed under reduced pressure. The residue was dissolved in DCM, and the organic layer was washed with sat. aq. NaHCO₃ solution and brine, dried over Na₂SO₄, filtered and the filtrate was concentrated and purified by CombiFlash® (eluting with PE/EA=5:1) to give 4-chloroimidazo[2,1-f][1,2,4]triazine (3.5 g, 62% yield) as a yellow solid. Mass Spectrum (ESI) m/z=154.6 (M+1).

Step B: To a solution of 4-chloroimidazo[2,1-f][1,2,4]triazine (2 g, 10 mmol) in THF (60 mL) was added sodium thiomethoxide (1.4 g, 20 mmol). The reaction was stirred at 50° C. for 18 h. Then water was added, and the mixture was extracted with EA (3×120 mL). The organic layer was dried over anhydrous Na₂SO₄, filtered, and the filtrate was concentrated and purified by silica gel column chromatography (PE/EA=9:1) to give 4-(methylsulfanyl)imidazo[2,1-f][1,2,4]triazine (900 mg, 43% yield) as a yellow solid. Mass Spectrum (ESI) m/z=167.1 (M+1).

Step C: To a solution of 4-(methylsulfanyl)imidazo[2,1-f][1,2,4]triazine (800 mg, 4.8 mmol) in 15 mL anhydrous THF (25 mL) stirring at −78° C., 3.6 mL of 2.0 M lithium diisopropylamide (3.6 mL, 7.2 mmol) was added dropwise over a period of 20 min. The reaction mixture was stirred for 30 min. at −78° C., then a solution of (3S,4R,5R)-4-(benzyloxy)-5-[(benzyloxy)methyl]-3-fluorooxolan-2-one (1.6 g, 4.81 mmol) in 5 mL of THF was added dropwise over 30 min. The reaction was stirred for 6 h at −78° C., then for 2 h at −30° C. After quenching with sat. aq. NH₄Cl solution, it was extracted with Et₂O. The organic layer was dried over anhydrous Na₂SO₄, filtered and the filtrate was concentrated and purified by silica gel column chromatography (PE/EA=9:1) to give (3R,4R,5R)-3,4-bis(benzyloxy)-5-[(benzyloxy)methyl]-2-[4-(methylsulfanyl)imidazo[2,1-f][1,2,4]triazin-7-yl]oxolan-2-ol (800 mg, 15% yield) as a yellow oil. Mass Spectrum (ESI) m/z=496.6 (M+1).

Step D: To a solution of (3S,4R,5R)-4-(benzyloxy)-5-[(benzyloxy)methyl]-3-fluoro-2-[4-(methylsulfanyl)imidazo[2,1-f][1,2,4]triazin-7-yl]oxolan-2-ol (650 mg, 1.13 mmol) in anhydrous CH₂Cl₂ stirred at −78° C., triethylsilane (524 mg, 4.52 mmol) was added dropwise, followed by 0.5 mL of boron trifluoride diethyl etherate (642 mg, 4.52 mmol). The reaction was stirred at −78° C. and allowed to slowly warm up to rt overnight. Then the mixture was quenched with sat. aq. NaHCO₃ solution and extracted with Et₂O. The organic layer was dried over anhydrous Na₂SO₄, filtered and the filtrate was concentrated and purified by silica gel column chromatography (hexanes/EA 9:1) to give 7-[(3S,4R,5R)-4-(benzyloxy)-5-[(benzyloxy)methyl]-3-fluorooxolan-2-yl]-4-(methylsulfanyl)imidazo[2,1-f][1,2,4]triazine (560 mg, 86% yield) as a yellow oil. Mass Spectrum (ESI) m/z=481.1 (M+1).

Step E: To a solution of 7-[(3S,4R,5R)-4-(benzyloxy)-5-[(benzyloxy)methyl]-3-fluorooxolan-2-yl]-4-(methylsulfanyl)imidazo[2,1-f][1,2,4]triazine (400 mg, 0.83 mmol) in ethanol (10 mL) was added trimethylamine (252 mg, 2.49 mmol) and benzylamine (116 mg, 1.08 mmol). The reaction mixture was stirred at 60° C. for 24 h, then allowed to cool to room temperature. The reaction solution was concentrated and purified by silica gel column chromatography (PE/EA=85:15) to give 4-(benzyloxy)-5-[(benzyloxy)methyl]-3-fluorooxolan-2-yl]imidazo[2,1-f][1,2,4]triazin-4-amine (380 mg, 80% yield) as a yellow oil. Mass Spectrum (ESI) m/z=539.8 (M+1).

Step F: To a solution of N-benzyl-7-[(2R,3R,4R,5R)-4-(benzyloxy)-5-[(benzyloxy)methyl]-3-fluorooxolan-2-yl]imidazo[2,1-f][1,2,4]triazin-4-amine (350 mg, 0.65 mmol) in DCM (10 mL) was added boron trichloride (1 M in DCM, 6.5 ML, 6.5 mmol) at −78° C. The reaction was stirred at −78° C. for 2 h. Then the reaction was brought to −30° C. over a period of 30 min, and quenched by adding a mixture of methanol:chloroform (2:1, 10 mL). After the reaction mixture reached rt, it was neutralized with NH₃ in methanol (10%, 10 mL) and concentrated. The residue was purified by CombiFlash® (eluting with MeOH/DCM=5:95) to give (2R,3R,4S,5S)-5-[4-(benzylamino)imidazo[2,1-f][1,2,4]triazin-7-yl]-4-fluoro-2-(hydroxymethyl)oxolan-3-ol (44 mg, 73% yield) as a yellow solid. Mass Spectrum (ESI) m/z=360.1 (M+1).

Step G: To a solution of (2R,3R,4S,5S)-5-[4-(benzylamino)imidazo[2,1-f][1,2,4]triazin-7-yl]-4-fluoro-2-(hydroxymethyl)oxolan-3-ol (44 mg, 0.12 mmol) in trimethyl phosphate (0.6 mL) was added methylenebis(phosphonic dichloride) (150 mg, 0.6 mmol) in trimethylphosphate (0.7 mL) dropwise at 0° C. The reaction was stirred for 4 h. TEAC (0.5 M, 0.9 mL) was added to the reaction carefully and the reaction was stirred at this temperature for 15 min, then warmed to room temperature and stirring was continued for 1 h. Trimethylphosphate was extracted using tert-butyl methyl ether (5 mL×2) and the aqueous layer was basified with ammonium hydroxide to pH ˜7-8, then purified by Prep-HPLC using a gradient of 0.2% formic acid in water/ACN from 80:20 to 70:30. Product containing fractions were pooled and lyophilized to give [({[(2R,3R,4S,5S)-5-[4-(benzylamino)imidazo[2,1-f][1,2,4]triazin-7-yl]-4-fluoro-3-hydroxyoxolan-2-yl]methoxy}(hydroxy)phosphoryl)methyl]phosphonic acid (3 mg, 8% yield) as a white solid.

¹H NMR (400 MHz, DMSO) δ 9.42 (s, 1H), 8.23-8.12 (m, 1H), 7.73-7.66 (m, 1H), 7.45-7.15 (m, 5H), 5.55-5.45 (m, 1H), 5.43-5.32 (m, 1H), 4.81-4.64 (m, 2H), 4.43-4.31 (m, 1H), 4.11-4.04 (m, 1H), 3.97-3.87 (m, 2H), 2.07-1.68 (m, 2H). Mass Spectrum (ESI) m/z=517.7 (M+1).

### Example S4

### Synthesis of [({[(2R,3R,4S,5R)-5-[4-(benzylamino)imidazo[2,1-f][1,2,4]triazin-7-yl]-4-fluoro-3-hydroxyoxolan-2-yl]methoxy}(hydroxy)phosphoryl)methyl]phosphonic Acid

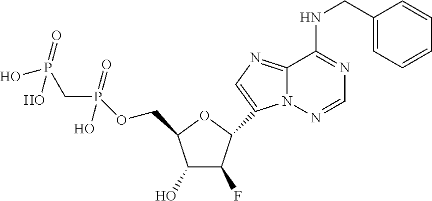 [SMILES: O=P(O)(O)CP(=O)(O)OC[C@H]1O[C@H](c2cnc3c(NCc4ccccc4)ncnn23)[C@@H](F)[C@@H]1O]

[({[(2R,3R,4S,5R)-5-[4-(benzylamino)imidazo[2,1-f][1,2,4]triazin-7-yl]-4-fluoro-3-hydroxyoxolan-2-yl]methoxy}(hydroxy)phosphoryl)methyl]phosphonic acid was obtained as another isomer from Step G in Example S3.

¹H NMR (400 MHz, DMSO) δ 9.48-9.41 (m, 1H), 8.21-8.16 (m, 1H), 7.61-7.58 (m, 1H), 7.38-7.25 (m, 5H), 5.56-5.53 (m, 1H), 5.51-5.46 (m, 1H), 5.25-5.00 (m, 2H), 4.76-4.73 (m, 1H), 4.39-3.92 (m, 3H), 2.10-1.90 (m, 2H). Mass Spectrum (ESI) m/z=517.7 (M+1).

### Example 5S

### Synthesis of (((((2R,3S,4R,1S)-5-(2-chloro-4-(cyclopentylamino)imidazo[2,1-f][1,2,4]triazin-7-yl)-3,4-dihydroxytetrahydrofuran-2-yl)methoxy)(hydroxy)phosphoryl)methyl)phosphonic acid

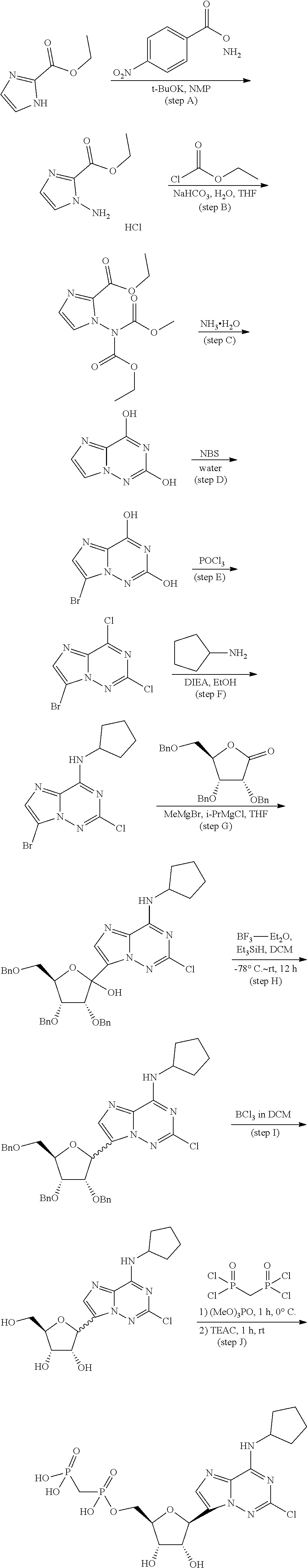

Step A: To a suspension of ethyl 1H-imidazole-2-carboxylate (150 g, 1.07 mol) in NMP (2 L) was added (tert-butoxy)potassium (1.32 L, 1.17 mol). The mixture was stirred at rt for 15 min, then a solution of o-(4-nitrobenzoyl)hydroxylamine (214 g, 1.17 mol) in NMP (1 L) was added slowly. The reaction mixture was stirred at rt for 2 h. A solution of HCl in ether (2M, 35 mL) was added and the mixture was stirred for 20 min. Another 500 mL of ether was added and stirring was continued for another 30 min. Then the mixture was filtered to give ethyl 1-amino-1H-imidazole-2-carboxylate (120 g, 72% yield) as a brown solid. Mass Spectrum (ESI) m/z=156.1 (M+1).

Step B: To a solution of ethyl 1-aminoimidazole-2-carboxylate (190 g, 1.26 mol) in THF (2 L) and water (2 L) was added sodium bicarbonate (775 g, 9.23 mol), followed by chloro(ethoxy)methanone (400 mL, 12.3 mol). The mixture was stirred at rt overnight. The reaction mixture was concentrated and extracted with EA (2 L×2). The combined organic layers were washed with brine, dried, filtered and the filtrate was concentrated and purified by silica gel column chromatography (eluting with PE/EA=10:1-4:1) to give ethyl 1-[bis(ethoxycarbonyl)amino]imidazole-2-carboxylate (150 g, 41% yield) as a yellow oil. Mass Spectrum (ESI) m/z=300.1 (M+1).

Step C: A mixture of ethyl 1-[bis(ethoxycarbonyl)amino]imidazole-2-carboxylate (40 g, 133.73 mmol) in i-PrOH (100 mL) and ammonium hydroxide (300 mL) was stirred in a sealed tube overnight at 120° C. Then the reaction was concentrated, washed with MeOH:diethyl ether (1:10, 100 mL) and filtered to give imidazo[2,1-f][1,2,4]triazine-2,4-diol (20 g, 98% yield) as a brown solid. Mass Spectrum (ESI) m/z=153.2 (M+1).

Step D: To a suspension of imidazo[2,1-f][1,2,4]triazine-2,4-diol (20 g, 130.72 mmol) in water (200 mL) was added NBS (16.3 g, 91.50 mmol) in several portions. Then the mixture was stirred at rt for 1 h. The mixture was filtered and the filtrate was concentrated, washed with methanol and toluene to give 7-bromoimidazo[2,1-f][1,2,4]triazine-2,4-diol (8 g, 27% yield) as an off-white solid. Mass Spectrum (ESI) m/z=230.9 (M+1).

Step E: To a suspension of 7-bromoimidazo[2,1-f][1,2,4]triazine-2,4-diol (8.0 g, 34.6 mmol) in POCl₃ (100 mL) was added triethylamine hydrochloride (3.34 g, 24.24 mmol) at 0° C. Then the mixture was stirred at 110° C. for 8 h in a sealed tube. The solvent was removed under reduced pressure and the residue was dissolved in DCM (100 mL) and poured into ice-water (100 mL). The organic layer was washed with brine, dried, filtered and the filtrate was concentrated and purified by CombiFlash® (40 g, eluting with EA/PE=0-6%) to give 7-bromo-2,4-dichloroimidazo[2,1-f][1,2,4]triazine (4.5 g, 44% yield) as a brown solid.

¹H NMR (301 MHz, CDCl₃) δ ppm 7.98 (s, 1H).

Step F: To a solution of 7-bromo-2,4-dichloroimidazo[2,1-f][1,2,4]triazine (4.5 g, 16.8 mmol) in THF (50 mL) was added DIEA (3.8 g, 33.6 mmol) and cyclopentanamine (1.57 g, 18.48 mmol). The mixture was stirred at rt for 30 mins. Then the reaction mixture was concentrated and purified by CombiFlash® (20 g, eluting with EA/PE=0-10%) to give 7-bromo-2-chloro-N-cyclopentylimidazo[2,1-f][1,2,4]triazin-4-amine (3.7 g, 59% yield) as a brown solid. Mass Spectrum (ESI) m/z=316.0 (M+1).

Step G: To a solution of 7-bromo-4-(tert-butoxy)-2-chloroimidazo[2,1-f][1,2,4]triazine (3.7 g, 11.69 mmol) in THF (80 mL) at −78° C. was added methylmagnesium bromide (3 M, 3.9 mL, 11.69 mmol), followed by isopropylmagnesium chloride-lithium chloride complex (1.3 M, 9.89 mL, 12.86 mmol), then a solution of (3R,4R,5R)-3,4-bis(benzyloxy)-5-[(benzyloxy)methyl]oxolan-2-one (4.89 g, 11.69 mmol) in THF (20 mL) was added slowly. The reaction solution was stirred at −78° C. for 2 h. The resulting solution was quenched with saturated aq. NH₄Cl solution (50 mL) and extracted with EA (100 mL×2). The combined organic layers were washed with brine, dried, filtered and the filtrate was concentrated and purified by CombiFlash® (40 g, eluting with EA/PE=0-40%) to give (3R,4R,5R)-3,4-bis(benzyloxy)-5-((benzyloxy)methyl)-2-(2-chloro-4-(cyclopentylamino)imidazo[2,1-f][1,2,4]triazin-7-yl)tetrahydrofuran-2-ol (4.5 g, 52% yield) as a yellow oil. Mass Spectrum (ESI) m/z=656.1 (M+1).

Step H: To a solution of (3R,4R,5R)-3,4-bis(benzyloxy)-5-[(benzyloxy)methyl]-2-[2-chloro-4-(cyclopentylamino)imidazo[2,1-f][1,2,4]triazin-7-yl]oxolan-2-ol (4.5 g, 6.86 mmol) in DCM (50 mL) was added triethylsilane (7.98 g, 68.6 mmol) and boron trifluoride diethyl etherate (9.74 g, 10.35 mmol) at −78° C. The mixture was stirred at rt for 1 h. Saturated aqueous NaHCO₃ solution was added to the reaction slowly. Then it was extracted with DCM (20 mL×2). The combined organic layers were washed with brine, dried, filtered and the filtrate was concentrated and purified by CombiFlash® (40 g, eluting with EA/PE=0-15%) to give 7-((3S,4R,5R)-3,4-bis(benzyloxy)-5-((benzyloxy)methyl)tetrahydrofuran-2-yl)-2-chloro-N-cyclopentylimidazo[2,1-f][1,2,4]triazin-4-amine (3.7 g, 67% yield) as a colorless oil. Mass Spectrum (ESI) m/z=639.8 (M+1).

Step I: To a solution of 7-[(3S,4R,5R)-3,4-bis(benzyloxy)-5-[(benzyloxy)methyl]oxolan-2-yl]-2-chloro-N-cyclopentylimidazo[2,1-f][1,2,4]triazin-4-amine (3.7 g, 5.78 mmol) in DCM (40 mL) was added trichloroborane in DCM (1M, 57.8 mL) at −70° C. The mixture was stirred at −70° C. for 1 h. Then the reaction was brought to −30° C. over a period of 30 min and quenched by adding a mixture of methanol:chloroform (2:1, 10 mL). After the reaction mixture reached rt, it was neutralized with NH₃ in methanol (10%, 10 mL) and concentrated. The residue was purified by CombiFlash® (20 g, eluting with MeOH/DCM=0-10%) to give (3R,4S,5R)-2-(2-chloro-4-(cyclopentylamino)imidazo[2,1-f][1,2,4]triazin-7-yl)-5-(hydroxymethyl)tetrahydrofuran-3,4-diol (1.8 g, 75% yield) as an off-white solid. Mass Spectrum (ESI) m/z=370.1 (M+1).

Step J: To a solution of (3R,4S,5R)-2-[2-chloro-4-(cyclopentylamino)imidazo[2,1-f][1,2,4]triazin-7-yl]-5-(hydroxymethyl)oxolane-3,4-diol (800 mg, 2.16 mmol) in trimethylphosphate (7 mL) at 0° C. was added a cold solution of methylenebis(phosphonic dichloride) (2.7 g, 10.8 mmol) in trimethylphosphate (3.8 mL) dropwise. Then the reaction solution was stirred at 0° C. for 5 h. TEAC (0.5 M, 10 mL) was added to the reaction carefully and the reaction was stirred at this temperature for 15 mins, then warmed to rt and continued to stir for 1 h. Trimethylphosphate was extracted using tert-butyl methyl ether (20 mL×2) and the aqueous layer was basified with ammonium hydroxide to pH ˜7-8. Then purified by Prep-HPLC using a gradient of (0.2% formic acid in water)/ACN from 90:10 to 70:30. Product containing fractions were pooled and lyophilized to give (((((2R,3S,4R,5S)-5-(2-chloro-4-(cyclopentylamino)imidazo[2,1-f][1,2,4]triazin-7-yl)-3,4-dihydroxytetrahydrofuran-2-yl)methoxyxhydroxy)phosphoryl)methyl)phosphonic acid (150 mg, 13% yield) as a white solid.

¹H NMR (400 MHz, D₂O) δ 7.81 (s, 1H), 5.17 (d, J=5.1 Hz, 1H), 4.53-4.48 (m, 1H), 4.42-4.34 (m, 1H), 4.32-4.26 (m, 1H), 4.19-4.14 (m, 1H), 4.08-3.96 (m, 2H), 2.21 (t, J=19.7 Hz, 2H), 2.02-1.91 (m, 2H), 1.73-1.53 (m, 6H). Mass Spectrum (ESI) m/z=527.6 (M+1).

### Example S6

### Synthesis of (((((2R,3S,4R,1S)-5-(4-(benzylamino)-2-chloroimidazo[2,1-f][1,2,4]triazin-7-yl)-3,4-dihydroxytetrahydrofuran-2-yl)methoxy)(hydroxy)phosphoryl)methyl)phosphonic Acid

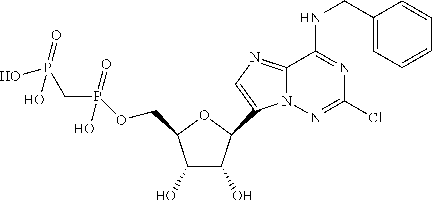 [SMILES: O=P(O)(O)CP(=O)(O)OC[C@H]1O[C@@H](c2cnc3c(NCc4ccccc4)nc(Cl)nn23)[C@H](O)[C@@H]1O]

(((((2R,3S,4R,5S)-5-(4-(benzylamino)-2-chloroimidazo[2,1-f][1,2,4]triazin-7-yl)-3,4-dihydroxytetrahydrofuran-2-yl)methoxyxhydroxy)phosphoryl)methyl)phosphonic acid was synthesized by procedures similar to the ones described in Example S5, replacing cyclopentanamine in Step F with benzylamine.

¹H NMR (400 MHz, D₂O) δ 7.72 (s, 1H), 7.33-7.21 (m, 5H), 5.16 (d, J=6.1 Hz, 1H), 4.54 (t, J=5.3 Hz, 1H), 4.30 (t, J=4.5 Hz, 1H), 4.19-4.13 (m, 1H), 4.05-3.95 (m, 2H), 2.17 (t, J=19.2 Hz, 2H). Mass Spectrum (ESI) m/z=547.7 (M−1).

### Example S7

### Synthesis of (((((2R,3R,4S,5S)-5-(2-chloro-4-(cyclopentylamino)imidazo[2,1-f][1,2,4]triazin-7-yl)-4-fluoro-3-hydroxytetrahydrofuran-2-yl)methoxy)(hydroxy)phosphoryl)methyl)phosphonic acid

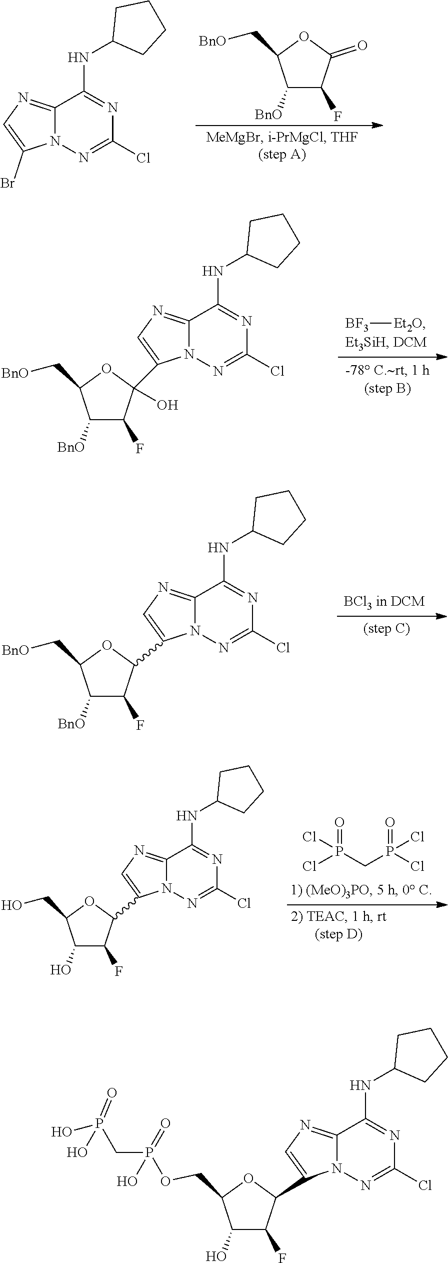

Step A: To a solution of 7-bromo-2-chloro-N-cyclopentylimidazo[2,1-f][1,2,4]triazin-4-amine (2 g, 6.3 mmol) in THF (10 mL) at 0° C. was added methylmagnesium bromide (3 M, 2.1 mL, 6.3 mmol), followed by isopropylmagnesium chloride lithium chloride complex (1.3 M, 5.8 mL, 7.56 mmol), then a solution of (3S,4R,5R)-4-(benzyloxy)-5-((benzyloxy)methyl)-3-fluorodihydrofuran-2(3H)-one (2.08 g, 6.3 mmol) in THF (20 mL) was added slowly. The reaction solution was stirred at 0° C. for 2 h. The resulting solution was quenched with saturated aq. NH₄Cl solution (50 mL) and extracted with EA (100 mL×2). The combined organic layers were washed with brine, dried, filtered and the filtrate was concentrated and purified by CombiFlash® (24 g, eluting with EA/PE=0-40%) to give (3S,4R,5R)-4-(benzyloxy)-5-((benzyloxy)methyl)-2-(2-chloro-4-(cyclopentylamino)imidazo[2,1-f][1,2,4]triazin-7-yl)-3-fluorotetrahydrofuran-2-ol (2 g, 44% yield) as a yellow oil. Mass Spectrum (ESI) m/z=568.1 (M+1).

Step B: To a solution of (3S,4R,5R)-4-(benzyloxy)-5-((benzyloxy)methyl)-2-(2-chloro-4-(cyclopentylamino)imidazo[2,1-f][1,2,4]triazin-7-yl)-3-fluorotetrahydrofuran-2-ol (966 mg, 1.7 mmol) in DCM (10 mL) was added triethylsilane (788 mg, 6. 8 mmol) and BF₃.OEt₂ (965 g, 6.8 mmol) at −78° C. The mixture was stirred at rt for 1 h. Saturated aq. NaHCO₃ solution was added to the reaction slowly, then it was extracted with DCM (50 mL×2). The combined organic layers were washed with brine, dried, filtered and the filtrate was concentrated and purified by CombiFlash® (12 g, eluting with EA/PE=0-15%) to give 7-((3R,4R,5R)-4-(benzyloxy)-5-((benzyloxy)methyl)-3-fluorotetrahydrofuran-2-yl)-2-chloro-N-cyclopentylimidazo[2,1-f][1,2,4]triazin-4-amine (400 mg, 38% yield) as a colorless oil. Mass Spectrum (ESI) m/z=552.0 (M+1).

Step C: To a solution of 7-((3R,4R,5R)-4-(benzyloxy)-5-((benzyloxy)methyl)-3-fluorotetrahydrofuran-2-yl)-2-chloro-N-cyclopentylimidazo[2,1-f][1,2,4]triazin-4-amine (170 mg, 0.31 mmol) in DCM (5 mL) was added trichloroborane in DCM (1M, 3.1 mL) at −70° C. The mixture was stirred at −70° C. for 1 h. Then the reaction was brought to −30° C. over a period of 30 min, and quenched by adding a mixture of methanol:chloroform (2:1, 10 mL). After the reaction mixture had warmed to rt, it was neutralized with NH₃ in methanol (10%, 10 mL) and concentrated. The residue was purified by CombiFlash® (12 g, eluting with MeOH/DCM=0-10%) to give (2R,3R,4S)-5-(2-chloro-4-(cyclopentylamino)imidazo[2,1-f][1,2,4]triazin-7-yl)-4-fluoro-2-(hydroxymethyl)tetrahydrofuran-3-ol (60 mg, 48% yield) as an off-white solid. Mass Spectrum (ESI) m/z=371.8 (M+1).

Step D: To a solution of ((2R,3R,4S)-5-(2-chloro-4-(cyclopentylamino)imidazo[2,1-f][1,2,4]triazin-7-yl)-4-fluoro-2-(hydroxymethyl)tetrahydrofuran-3-ol (50 mg, 0.13 mmol) in trimethylphosphate (1 mL) at 0° C. was added a cold solution of methylenebis(phosphonic dichloride) (167 mg, 0.67 mmol) in trimethylphosphate (0.5 mL) dropwise. Then the reaction solution was stirred at 0° C. for 5 h. TEAC (0.5 M, 2 mL) was added to the reaction carefully and the reaction was stirred at this temperature for 15 mins, then warmed to room temperature and continued to stir for 1 h. Trimethylphosphate was extracted using tert-butyl methyl ether (20 mL×2) and the aqueous layer was basified with ammonium hydroxide to pH ˜7-8. The aq. solution was purified by prep-HPLC using a gradient of (0.2% formic acid in water)/ACN from 90:10 to 70:30. Product containing fractions were pooled and lyophilized to give (((((2R,3R,4S,5S)-5-(2-chloro-4-(cyclopentylamino)imidazo[2,1-f][1,2,4]triazin-7-yl)-4-fluoro-3-hydroxytetrahydrofuran-2-yl)methoxyxhydroxy)phosphoryl)methyl)phosphonic acid (4.5 mg, 6% yield) as a white solid.

¹H NMR (400 MHz, D₂O) δ 7.81 (s, 1H), 5.17 (d, J=5.1 Hz, 1H), 4.53-4.48 (m, 1H), 4.42-4.34 (m, 1H), 4.32-4.26 (m, 1H), 4.19-4.14 (m, 1H), 4.08-3.96 (m, 2H), 2.21 (t, J=19.7 Hz, 2H), 2.02-1.91 (m, 2H), 1.73-1.53 (m, 6H). Mass Spectrum (ESI) m/z=528.0 (M−1).

### Example S8

### Synthesis of (((((2R,3S,4R,1S)-5-(2-chloro-4-(cyclopentyl(methyl)amino)imidazo[2,1-f][1,2,4]triazin-7-yl)-3,4-dihydroxytetrahydrofuran-2-yl)methoxy)(hydroxy)phosphoryl)methyl)phosphonic acid

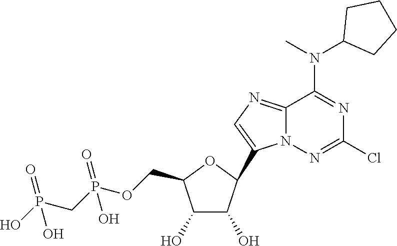 [SMILES: CN(c1nc(Cl)nn2c([C@@H]3O[C@H](COP(=O)(O)CP(=O)(O)O)[C@@H](O)[C@H]3O)cnc12)C1CCCC1]

(((((2R,3S,4R,5S)-5-(2-chloro-4-(cyclopentyl(methyl)amino)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with N-methylcyclopentanamine.

¹H NMR (400 MHz, D₂O) δ 7.68-7.60 (m, 1H), 5.19-5.13 (m, 1H), 4.59-4.52 (m, 1H), 4.43-4.32 (m, 1H), 4.23-4.15 (m, 1H), 4.05-3.95 (m, 2H), 3.62-3.49 (m, 1H), 3.20-3.13 (m, 3H), 2.05-1.92 (m, 2H), 1.92-1.54 (m, 8H). Mass Spectrum (ESI) m/z=540.0 (M−1).

### Example S9

### Synthesis of (((((2R,3S,4R,1S)-5-(2-chloro-4-((2,3-dihydro-1H-inden-1-yl)amino)imidazo[2,1-f][1,2,4]triazin-7-yl)-3,4-dihydroxytetrahydrofuran-2-yl)methoxy)(hydroxy)phosphoryl) methy)phosphonic acid

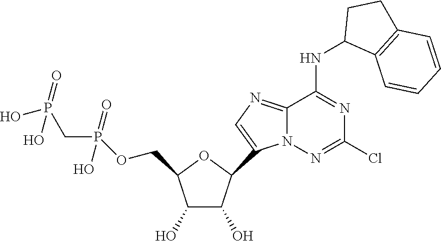 [SMILES: O=P(O)(O)CP(=O)(O)OC[C@H]1O[C@@H](c2cnc3c(NC4CCc5ccccc54)nc(Cl)nn23)[C@H](O)[C@@H]1O]

(((((2R,3S,4R,5S)-5-(2-chloro-4-((2,3-dihydro-1H-inden-1-yl)amino)imidazo[2,1-f][1,2,4]triazin-7-yl)-3,4-dihydroxytetrahydrofuran-2-yl)methoxyxhydroxy)phosphoryl) methyl)phosphonic acid was synthesized by procedures similar to the ones described in Example S5, replacing cyclopentanamine in Step F with 2,3-dihydro-1H-inden-1-amine.

¹H NMR (400 MHz, D₂O) δ 7.68 (s, 1H), 7.44-7.11 (m, 4H), 5.74 (s, 1H), 5.28-5.08 (m, 1H), 4.62-4.54 (m, 1H), 4.38 (t, J=5.0 Hz, 1H), 4.19 (d, J=4.2 Hz, 1H), 4.09-3.92 (m, 2H), 3.06-3.00 (m, 1H), 2.95-2.87 (m, 1H), 2.64-2.58 (m, 1H), 2.09-1.90 (m, 3H).

Mass Spectrum (ESI) m/z=576.0 (M+1).

### Example S10

### Synthesis of (((((2R,3S,4R,1S)-5-(2-chloro-4-(hexahydrocyclopenta[c]pyrrol-2(1H)-yl)imidazo[2,1-f][1,2,4]triazin-7-yl)-3,4-dihydroxytetrahydrofuran-2-yl)methoxy)(hydroxy)phosphoryl) methy)phosphonic acid

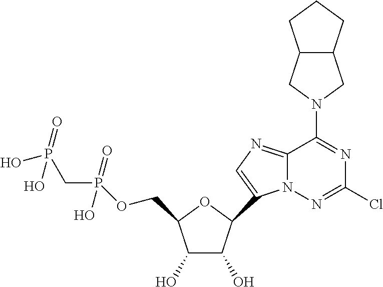 [SMILES: O=P(O)(O)CP(=O)(O)OC[C@H]1O[C@@H](c2cnc3c(N4CC5CCCC5C4)nc(Cl)nn23)[C@H](O)[C@@H]1O]

(((((2R,3S,4R,5S)-5-(2-chloro-4-(hexahydrocyclopenta[c]pyrrol-2(1H)-yl)imidazo[2,1-f][1,2,4]triazin-7-yl)-3,4-dihydroxytetrahydrofuran-2-yl)methoxyxhydroxy)phosphoryl)methyl) phosphonic acid was synthesized by procedures similar to the ones described in Example S5, replacing cyclopentanamine in Step F with octahydrocyclopenta [c]pyrrole.

¹H NMR (400 MHz, D₂O) δ 7.68 (s, 1H), 5.16 (d, J=6.8 Hz, 1H), 4.61-4.54 (m, 1H), 4.39-4.30 (m, 2H), 4.19 (d, J=4.0 Hz, 1H), 4.10 (d, J=13.3 Hz, 1H), 4.02-3.94 (m, 2H), 3.90-3.80 (m, 1H), 3.60-3.50 (m, 1H), 2.88-2.82 (m, 1H), 2.79-2.72 (m, 1H), 2.17-2.00 (m, 2H), 1.89-1.80 (m, 2H), 1.76-1.67 (m, 1H), 1.66-1.56 (m, 1H), 1.52-1.43 (m, 2H). Mass Spectrum (ESI) m/z=552.0 (M−1).

### Example S11

### Synthesis of [({[(2R,3S,4R,1S)-5-(2-chloro-4-{[(2-chlorophenyl)methyl]amino}imidazo[2,1-f][1,2,4]triazin-7-yl)-3,4-dihydroxyoxolan-2-yl]methoxy}(hydroxy)phosphoryl)methyl]phosphonic Acid

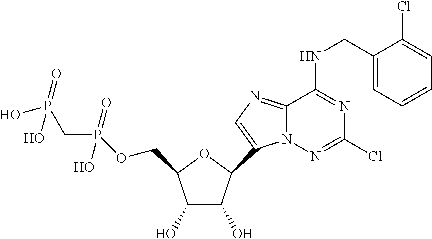 [SMILES: O=P(O)(O)CP(=O)(O)OC[C@H]1O[C@@H](c2cnc3c(NCc4ccccc4Cl)nc(Cl)nn23)[C@H](O)[C@@H]1O]

[({[(2R,3S,4R,5S)-5-(2-chloro-4-{[(2-chlorophenyl)methyl]amino}imidazo[2,1-f][1,2,4]triazin-7-yl)-3,4-dihydroxyoxolan-2-yl]methoxy}(hydroxy)phosphoryl)methyl]phosphonic acid was synthesized by procedures similar to the ones described in Example S5, replacing cyclopentanamine in Step F with 2-chlorobenzylamine.

¹H NMR (400 MHz, D₂O) δ 7.70 (s, 1H), 7.48-7.36 (m, 2H), 7.29-7.22 (m, 2H), 5.18 (d, J=6.4 Hz, 1H), 4.83 (s, 2H), 4.59-4.55 (m, 1H), 4.40-4.33 (m, 1H), 4.18-4.13 (m, 1H), 3.99-3.90 (m, 2H), 1.96 (t, J=19.7 Hz, 2H). Mass Spectrum (ESI) m/z=581.9 (M−1).

### Example S12

### Synthesis of (((((2R,3S,4R,1S)-5-(2-chloro-4-(((tetrahydrofuran-3-yl)methyl)amino)imidazo[2,1-f][1,2,4]triazin-7-yl)-3,4-dihydroxytetrahydrofuran-2-yl)methoxy)(hydroxy)phosphoryl)methyl) phosphonic Acid

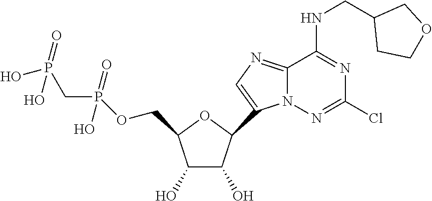 [SMILES: O=P(O)(O)CP(=O)(O)OC[C@H]1O[C@@H](c2cnc3c(NCC4CCOC4)nc(Cl)nn23)[C@H](O)[C@@H]1O]

(((((2R,3S,4R,5S)-5-(2-chloro-4-(((tetrahydrofuran-3-yl)methyl)amino)imidazo[2,1-f][1,2,4]triazin-7-yl)-3,4-dihydroxytetrahydrofuran-2-yl)methoxyxhydroxy)phosphoryl)methyl) phosphonic acid was synthesized by procedures similar to the ones described in Example S5, replacing cyclopentanamine in Step F with (tetrahydrofuran-3-yl) methanamine.

¹H NMR (400 MHz, D₂O) δ ppm 7.67 (s, 1H), 5.15 (d, J=6.3 Hz, 1H), 4.57-4.52 (m, 1H), 4.36 (m, 1H), 4.16 (m, 1H), 4.06-3.93 (m, 2H), 3.87-3.79 (m, 2H), 3.75-3.69 (m, 1H), 3.59-3.56 (m, 1H), 3.54 (d, J=7.4 Hz, 2H), 2.73-2.64 (m, 1H), 2.10-2.02 (m, 1H), 1.93 (t, J=19.6 Hz, 2H), 1.75-1.63 (m, 1H). Mass Spectrum (ESI) m/z=542.0 (M−1).

### Example S13

### Synthesis of (((((2R,3S,4R,1S)-5-(2-chloro-4-(((tetrahydrofuran-2-yl)methyl)amino)imidazo[2,1-f][1,2,4]triazin-7-yl)-3,4-dihydroxytetrahydrofuran-2-yl)methoxy)(hydroxy)phosphoryl)methyl) phosphonic Acid

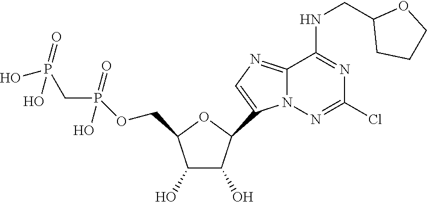 [SMILES: O=P(O)(O)CP(=O)(O)OC[C@H]1O[C@@H](c2cnc3c(NCC4CCCO4)nc(Cl)nn23)[C@H](O)[C@@H]1O]

(((((2R,3S,4R,5S)-5-(2-chloro-4-(((tetrahydrofuran-2-yl)methyl)amino)imidazo[2,1-f][1,2,4]triazin-7-yl)-3,4-dihydroxytetrahydrofuran-2-yl)methoxyxhydroxy)phosphoryl)methyl) phosphonic acid was synthesized by procedures similar to the ones described in Example S5, replacing cyclopentanamine in Step F with (tetrahydrofuran-2-yl) methanamine.

¹H NMR (400 MHz, D₂O) δ ppm 7.67 (s, 1H), 5.16 (d, J=6.6 Hz, 1H), 4.59-4.53 (m, 1H), 4.34 (t, J=4.8 Hz, 1H), 4.21-4.16 (m, 2H), 4.03-3.94 (m, 2H), 3.85-3.80 (m, 1H), 3.77-3.70 (m, 1H), 3.67-3.61 (m, 2H), 2.08-1.95 (m, 3H), 1.92-1.83 (m, 2H), 1.69-1.61 (m, 1H). Mass Spectrum (ESI) m/z=542.0 (M−1).

### Example S14

### Synthesis of (((((2R,3S,4R,1S)-5-(2-chloro-4-(((tetrahydrofuran-2-yl)methyl)amino)imidazo[2,1-f][1,2,4]triazin-7-yl)-3,4-dihydroxytetrahydrofuran-2-yl)methoxy)(hydroxy)phosphoryl)methyl) phosphonic Acid

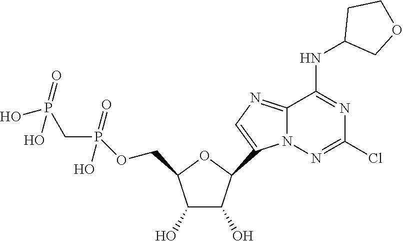 [SMILES: O=P(O)(O)CP(=O)(O)OC[C@H]1O[C@@H](c2cnc3c(NC4CCOC4)nc(Cl)nn23)[C@H](O)[C@@H]1O]

[({[(2R,3S,4R,5S)-5-[2-chloro-4-(oxolan-3-ylamino)imidazo[2,1-f][1,2,4]triazin-7-yl]-3,4-dihydroxyoxolan-2-yl]methoxy}(hydroxy)phosphoryl)methyl]phosphonic acid was synthesized by procedures similar to the ones described in Example S5, replacing cyclopentanamine in Step F with 3-aminotetrahydrofuran.

¹H NMR (400 MHz, D₂O) δ 7.67 (s, 1H), 5.16 (d, J=6.3 Hz, 1H), 4.76-4.75 (m, 1H), 4.55-4.53 (m, 1H), 4.36-4.30 (m, 1H), 4.16-4.10 (m 1H), 3.97-3.90 (m, 4H), 3.88-3.82 (m, 2H), 2.34-2.30 (m, 1H), 2.06-2.00 (m, 1H), 1.93-1.86 (m, 2H). Mass Spectrum (ESI) m/z=528.0 (M+1)

### Example S15

### Synthesis of [({[(2R,3S,4R,5S)-5-[4-(benzylamino)-2-chlorofuro[3,2-d]pyrimidin-7-yl]-3,4-dihydroxyoxolan-2-yl]methoxy}(hydroxy)phosphoryl)methyl]phosphonic Acid

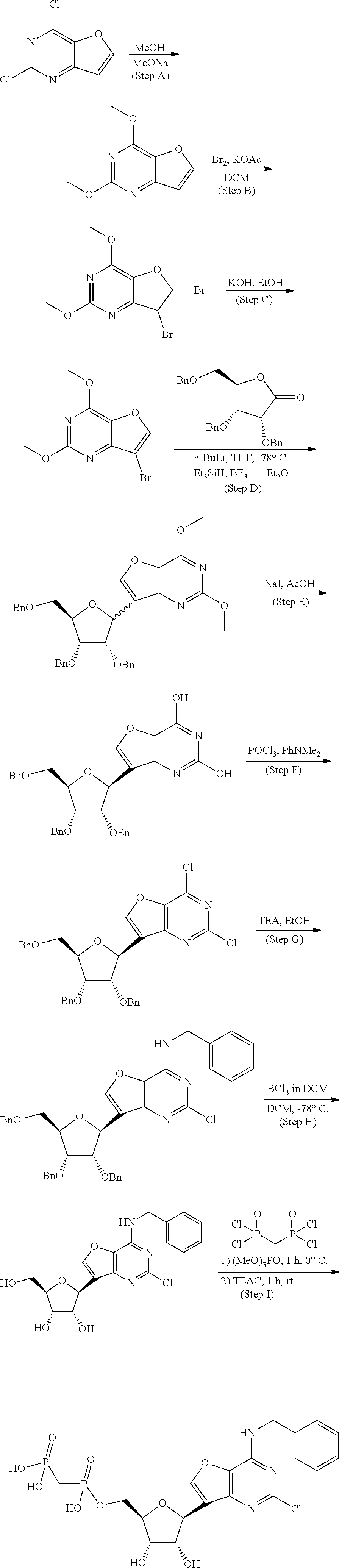

Step A: To a solution of 2,4-dichlorofuro[3,2-d]pyrimidine (6 g, 31.9 mmol) in MeOH was added sodium methoxide (17 g, 319 mmol). The reaction mixture was stirred and refluxed for 3 hours. The mixture was concentrated under vacuo and the residue was purified by silica gel column chromatography (PE/EA=4:1) to obtain 2,4-dimethoxyfuro[3,2-d]pyrimidine as a white solid (3.6 g, 65% yield). Mass Spectrum (ESI) m/z=181.0 (M+1).

Step B: To a suspension of 2,4-dimethoxyfuro[3,2-d]pyrimidine (3.6 g, 20 mmol) and potassium acetate (3.9 g, 40 mmol) in DCM was added a solution of bromine (6.4 g, 40 mmol) in DCM dropwise. The reaction mixture was stirred and refluxed for 4 hours. Then the aq. Na₂SO₃ solution was added carefully until the Br₂ was quenched completely. The mixture was extracted with DCM (100 mL×3). The combined organic phases were concentrated under vacuo to afford the residue as a yellow oil (5.8 g, 85% yield), which was used for the next step without further purification. Mass Spectrum (ESI) m/z=338.8 (M+1).

Step C: To a solution of 6,7-dibromo-2,4-dimethoxy-6H,7H-furo[3,2-d]pyrimidine (5.8 g, 17.2 mmol) in EtOH was added potassium hydroxide (1.47 g, 25.8 mmol). The reaction mixture was stirred at rt for 1 h and diluted with water, then extracted with EA (100 mL×3). The combined organic phases were concentrated and purified by silica gel column chromatography (PE/EA=4:1) to get 7-bromo-2,4-dimethoxyfuro[3,2-d]pyrimidine as a white solid (2.4 g, 54% yield). Mass Spectrum (ESI) m/z=259.0 (M+1).

Step D: To a solution of 7-bromo-2,4-dimethoxyfuro[3,2-d]pyrimidine (2.4 g, 9.27 mmol) in 30 mL anhydrous THF was carefully added n-BuLi (2.4 M, 5.8 mL, 14 mmol) dropwise at −78° C. under a nitrogen atmosphere. The reaction mixture was stirred for 30 min at −78° C., then a solution of (3R,4R,5R)-3,4-bis(benzyloxy)-5-[(benzyloxy)methyl]oxolan-2-one (3.88 g, 9.27 mmol) in 20 mL anhydrous THF was added dropwise over 30 min. The reaction was stirred for 2 h at −78° C., then for 4 h at −30° C. After quenching with 30 mL sat. aq. NH₄Cl solution, the mixture was extracted with EA (80 mL×3), the combined organic layers were dried over anhydrous Na₂SO₄, filtered, and the filtrate was concentrated. The dry residue was dissolved in anhydrous CH₂Cl₂ and stirred at −78° C. To this mixture, triethylsilane (4.31 g, 37.1 mmol) was added dropwise, followed by boron trifluoride diethyl etherate (5.26 g, 37.1 mmol). The reaction was stirred overnight at −78° C. and allowed to warm up to room temperature. After quenching with 50 mL sat. aq. NaHCO₃ solution, the mixture was extracted with EA (80 mL×3), and the combined organic layers were dried over anhydrous Na₂SO₄, filtered, and the filtrate was concentrated. The residue was purified by silica gel column chromatography (PE/EA=5:1) to give 7-[(2S,3S,4R,5R)-3,4-bis(benzyloxy)-5-[(benzyloxy)methyl]oxolan-2-yl]-2,4-dimethoxyfuro[3,2-d]pyrimidine as a colorless oil (1.4 g, 26% yield). Mass Spectrum (ESI) m/z=583.1 (M+1).

Step E: To a solution of 7-[(2S,3S,4R,5R)-3,4-bis(benzyloxy)-5-[(benzyloxy)methyl]oxolan-2-yl]-2,4-dimethoxyfuro[3,2-d]pyrimidine (1.4 g, 2.4 mmol) in glacial acetic acid (20 mL) was added sodium iodide (1.8 g, 12 mmol). The reaction mixture was heated to 60° C. for 45 min, and then the volatiles were removed in vacuo. The residue was dissolved in EtOAc and washed with aq. saturated Na₂SO₃ (20 mL×3) and saturated sodium bicarbonate solution (20 mL×3). The aqueous layers were extracted with EtOAc (20 mL×3). The combined organics were dried over Na₂SO₄ and concentrated in vacuo. The residue was purified by silica gel column chromatography (PE/EA=5:1) to give 7-((3S,4R,5R)-3,4-bis(benzyloxy)-5-((benzyloxy)methyl)tetrahydrofuran-2-yl)furo[3,2-d]pyrimidine-2,4-diol as a white solid (642 mg, 48% yield). Mass Spectrum (ESI) m/z=555.1 (M+1).

Step F: To a suspension of 7-[(2S,3S,4R,5R)-3,4-bis(benzyloxy)-5-[(benzyloxy)methyl]oxolan-2-yl]furo[3,2-d]pyrimidine-2,4-diol (230 mg, 0.42 mmol), benzyltriethylammonium chloride (189 mg, 0.83 mmol) and N,N-dimethylaniline (75 mg, 0.22 mmol) in acetonitrile (5 ml) was added phosphorus oxychloride (777 mg, 4.98 mmol). Then the reaction mixture was stirred at 80° C. for 16 h. The solvent was removed, and the residue was dissolved in DCM, washed with sat. NaHCO₃ solution and brine, dried over Na₂SO₄, and filtered. The filtrate was concentrated and the residue was purified by CombiFlash® (PE:EA=3:1) to afford 7-[(2S,3S,4R,5R)-3,4-bis(benzyloxy)-5-[(benzyloxy)methyl]oxolan-2-yl]-2,4-dichlorofuro[3,2-d]pyrimidine (140 mg, 55% yield) as a yellow oil. Mass Spectrum (ESI) m/z=590.9 (M+1).

Step G: A solution of 7-[(2S,3S,4R,5R)-3,4-bis(benzyloxy)-5-[(benzyloxy)methyl]oxolan-2-yl]-2,4-dichlorofuro[3,2-d]pyrimidine (140 mg, 0.23 mmol), BnNH₂ (25 mg, 0.23 mmol) and Et₃N (48 mg, 0.47 mmol) in EtOH (5 mL) was stirred at 70° C. for 2 h. Then the solvent was removed under reduced pressure and the residue was purified by CombiFlash® (PE/EA=2:1) to give N-benzyl-7-[(3S,4R,5R)-3,4-bis(benzyloxy)-5-[(benzyloxy)methyl]oxolan-2-yl]-2-chlorofuro[3,2-d]pyrimidin-4-amine (80 mg, 51% yield). Mass Spectrum (ESI) m/z=662.1 (M+1).

Step H and Step I: N-benzyl-7-[(3S,4R,5R)-3,4-bis(benzyloxy)-5-[(benzyloxy)methyl]oxolan-2-yl]-2-chlorofuro[3,2-d]pyrimidin-4-amine was converted to the title compound by procedures similar to the ones described in Example S5, Step I and Step J.

¹H NMR (400 MHz, D₂O) δ 8.07 (s, 1H), 7.39-7.20 (m, 5H), 5.00-4.90 (m, 1H), 4.67 (s, 2H), 4.36-4.22 (m, 2H), 4.16-3.98 (m, 3H), 2.25-2.09 (m, 2H). Mass Spectrum (ESI) m/z=547.6 (M−1).

### Example S16

### Synthesis of (((((2R,3S,4R,1S)-5-(4-(benzylamino)pyrrolo[2,1-f][1,2,4]triazin-7-yl)-3,4-dihydroxytetrahydrofuran-2-yl)methoxy)(hydroxy)phosphoryl)methyl)phosphonic acid

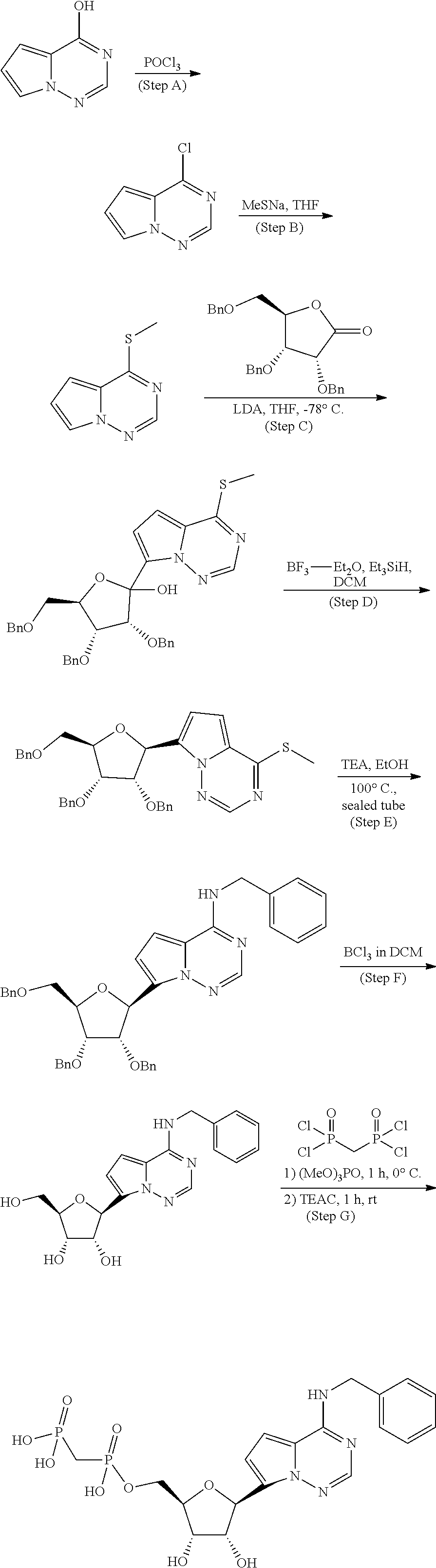

Step A: To a suspension of pyrrolo[2,1-f][1,2,4]triazin-4-ol (5.0 g, 37 mmol) in POCl₃ (50 mL) was added N,N-dimethylaniline (3.6 g, 29.6 mmol). The mixture was stirred at 100° C. for 4 h. The solvent was removed under reduced pressure. The residue was dissolved in DCM (100 mL) and poured into ice water. The aqueous layer was extracted with DCM (100 mL×2). The combined organic layers were washed with brine, dried, filtered and the filtrate was concentrated and purified by CombiFlash® (40 g, eluting with EA/PE=0:100 to 9:1) to give 4-chloropyrrolo[2,1-f][1,2,4]triazine (3.4 g, 54% yield) as a yellow oil. Mass Spectrum (ESI) m/z=153 (M+1).

Step B: To a solution of 4-chloropyrrolo[2,1-f][1,2,4]triazine (3.4 g, 22 mmol) in THF (60 mL) was added (methylsulfanyl)sodium (3.1 g, 44.16 mmol). The mixture was stirred at 50° C. for 2 h. Water (100 mL) was added to the reaction and the mixture was extracted with EA (100 mL×2). The combined organic layers were washed with brine, dried, filtered and the filtrate was concentrated and purified by CombiFlash® (40 g, eluting with EA/PE=0:100 to 2:3) to give 4-(methylthio)pyrrolo[2,1-f][1,2,4]triazine (2.7 g, 74% yield) as a white solid. Mass Spectrum (ESI) m/z=166.1 (M+1).

Step C: To a solution of 4-(methylsulfanyl)pyrrolo[2,1-f][1,2,4]triazine (2.5 g, 15.15 mmol) in THF (55 mL) was added n-butyllithium (2.4 M, 9.5 mL, 22.73 mmol) at −78° C. under nitrogen atmosphere. After stirring at −78° C. for 30 min, a solution of (3R,4R,5R)-3,4-bis(benzyloxy)-5-[(benzyloxy)methyl]oxolan-2-one (6.34 g, 15.15 mmol) in THF (5 mL) was added slowly. Then the mixture was stirred at −78° C. for 2 h. The resulting reaction was carefully quenched with saturated aq. NH₄Cl solution, and the mixture was extracted with EA (100 mL×2). The combined organic layers were washed with brine, dried, filtered and the filtrate was concentrated and purified by CombiFlash® (80 g, EA:PE=0-30%) to give (3R,4R,5R)-3,4-bis(benzyloxy)-5-((benzyloxy)methyl)-2-(4-(methylthio)pyrrolo[2,1-f][1,2,4]triazin-7-yl)tetrahydrofuran-2-ol (1.4 g, 16% yield) as a yellow oil. Mass Spectrum (ESI) m/z=584.1 (M+1).

Step D: To a solution of (3R,4R,5R)-3,4-bis(benzyloxy)-5-[(benzyloxy)methyl]-2-[4-(methylsulfanyl)pyrrolo[2,1-f][1,2,4]triazin-7-yl]oxolan-2-ol (1.4 g, 2.4 mmol) in DCM (14 mL) was added triethylsilane (1.63 g, 24 mmol) and boron trifluoride diethyl etherate (5.8 g, 24 mmol) at 0° C. The mixture was stirred at 0° C. for 1 h. Then the reaction was quenched with saturated aqueous NaHCO₃ slowly, extracted with DCM (100 mL×2). The combined organic layers were washed with brine, dried, filtered and the filtrate was concentrated and purified by CombiFlash® (20 g, EA/PE=0-15%) to give 7-((2S,3S,4R,5R)-3,4-bis(benzyloxy)-5-((benzyloxy)methyl)tetrahydrofuran-2-yl)-4-(methylthio)pyrrolo[2,1-f][1,2,4]triazine (780 mg, 57% yield) as a yellow oil. Mass Spectrum (ESI) m/z=568 (M+1).

Step E: To a suspension of 7-[(3S,4R,5R)-3,4-bis(benzyloxy)-5-[(benzyloxy)methyl]oxolan-2-yl]-4-(methylsulfanyl)pyrrolo[2,1-f][1,2,4]triazine (780 mg, 1.37 mmol) in EtOH (3 mL) was added triethylamine (556 mg, 5.5 mmol) and benzylamine (293 mg, 2.74 mmol). The mixture was stirred at 100° C. for 24 h in a sealed tube. Then the reaction mixture was concentrated and purified by CombiFlash® (12 g, EA/PE=0-30%) to give N-benzyl-7-((2S,3S,4R,5R)-3,4-bis(benzyloxy)-5-((benzyloxy)methyl)tetrahydrofuran-2-yl)pyrrolo[2,1-f][1,2,4]triazin-4-amine (770 mg, 70% yield) as a white solid. Mass Spectrum (ESI) m/z=627.1 (M+1).

Step F: To a solution of N-benzyl-7-[(2S,3S,4R,5R)-3,4-bis(benzyloxy)-5-[(benzyloxy)methyl]oxolan-2-yl]pyrrolo[2,1-f][1,2,4]triazin-4-amine (770 mg, 1.23 mmol) in DCM (10 mL) was added trichloroborane (1M in DCM, 12.3 mL, 12.3 mmol) dropwise at −78° C. under a nitrogen atmosphere. The mixture was stirred for 1 h at −78° C. Then the reaction was brought to −30° C. over a period of 30 min, and quenched by adding a mixture of methanol:chloroform (2:1, 10 mL). After the reaction mixture reached to rt, it was neutralized with NH₃ in methanol (10%, 10 mL) and concentrated. The residue was purified by CombiFlash® (4 g, MeOH/DCM=0-15%) to give (2S,3R,4S,5R)-2-(4-(benzylamino)pyrrolo[2,1-f][1,2,4]triazin-7-yl)-5-(hydroxymethyl)tetrahydrofuran-3,4-diol (300 mg, 68% yield) as a colorless oil. Mass Spectrum (ESI) m/z=389.1 (M+32).

Step G: To a solution of 2S,3R,4S,5R)-2-[4-(benzylamino)pyrrolo[2,1-f][1,2,4]triazin-7-yl]-5-(hydroxymethyl)oxolane-3,4-diol (100 mg, 0.28 mmol) in trimethylphosphate (2 mL) at 0° C. was added a cold solution of [(dichlorophosphoryl)methyl]phosphonoyl dichloride (350 mg, 1.4 mmol) in trimethylphosphate (0.5 mL) dropwise. Then the reaction solution was stirred at 0° C. for 4 h. TEAC (0.5 M, 6 mL) was added to the reaction carefully and the reaction was stirred at this temperature for 15 min, then warmed to room temperature and continued to stir for 1 h. Trimethylphosphate was extracted using tert-butyl methyl ether (5 mL×2) and the aqueous layer was basified with ammonium hydroxide to pH=7-8. Then purified by Prep-HPLC using a gradient of 0.2% Formic acid in water/ACN from 90:10 to 80:20, and suitable fractions were pooled and lyophilized to give (((((2R,3S,4R,5S)-5-(4-(benzylamino)pyrrolo[2,1-f][1,2,4]triazin-7-yl)-3,4-dihydroxytetrahydrofuran-2-yl)methoxyxhydroxy)phosphoryl)methyl)phosphonic acid (6 mg, 4% yield) as a white solid.

¹H NMR (400 MHz, DMSO) δ ppm 8.76 (s, 1H), 7.91 (s, 1H), 7.41-7.22 (m, 5H), 6.95 (d, J=4.3 Hz, 1H), 6.78-6.67 (m, 1H), 5.23-5.09 (m, 2H), 4.74 (d, J=6.0 Hz, 2H), 4.06 (m, 1H), 3.98 (m, 1H), 3.69-3.61 (m, 2H), 3.60 (m, 1H), 2.25-2.17 (m, 2H). Mass Spectrum (ESI) m/z=514.9 (M+32).

### Example S17

### Synthesis of (((((2R,3S,4R,1S)-5-(2-chloro-4-(((1r,4S)-4-methoxycyclohexyl)amino)imidazo[2,1-f][1,2,4]triazin-7-yl)-3,4-dihydroxytetrahydrofuran-2-yl)methoxy)(hydroxy)phosphoryl) methyl)phosphonic acid

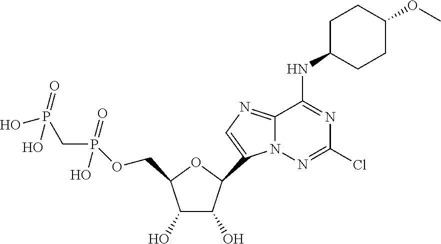 [SMILES: CO[C@H]1CC[C@H](Nc2nc(Cl)nn3c([C@@H]4O[C@H](COP(=O)(O)CP(=O)(O)O)[C@@H](O)[C@H]4O)cnc23)CC1]

(((((2R,3S,4R,5S)-5-(2-chloro-4-(((1R,4S)-4-methoxycyclohexyl)amino)imidazo[2,1-f][1,2,4]triazin-7-yl)-3,4-dihydroxytetrahydrofuran-2-yl)methoxy)(hydroxy)phosphoryl) methyl)phosphonic acid was prepared by procedures similar to the one described in Example S5, replacing cyclopentanamine in Step F with (1R,4R)-4-methoxycyclohexan-1-amine.

¹H NMR (400 MHz, D₂O) δ 7.65 (s, 1H), 5.14 (d, J=6.5 Hz, 1H), 4.62-4.51 (m, 1H), 4.37-4.28 (m, 1H), 4.20-4.15 (m, 1H), 4.12-4.04 (m, 1H), 4.02-3.92 (m, 2H), 3.50 (s, 1H), 3.27 (s, 3H), 2.10-1.92 (m, 2H), 1.85-1.82 (m, 2H), 1.79-1.70 (m, 2H), 1.69-1.63 (m, 4H). Mass Spectrum (ESI) m/z=570.0 (M−1).

### Example S18

### Synthesis of (((((2R,3S,4R,1S)-5-(2-chloro-4-(indolin-1-yl)imidazo[2,1-f][1,2,4]triazin-7-yl)-3,4-dihydroxytetrahydrofuran-2-yl)methoxy)(hydroxy)phosphoryl)methyl)phosphonic Acid

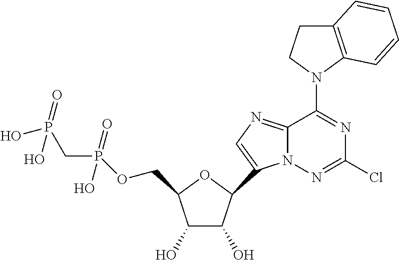 [SMILES: O=P(O)(O)CP(=O)(O)OC[C@H]1O[C@@H](c2cnc3c(N4CCc5ccccc54)nc(Cl)nn23)[C@H](O)[C@@H]1O]

(((((2R,3S,4R,5S)-5-(2-chloro-4-(indolin-1-yl)imidazo[2,1-f][1,2,4]triazin-7-yl)-3,4-dihydroxytetrahydrofuran-2-yl)methoxyxhydroxy)phosphoryl)methyl)phosphonic acid was prepared by procedures similar to the one described in Example S5, replacing cyclopentanamine in Step F with indoline.

¹H NMR (400 MHz, D₂O) δ 8.37-8.31 (m, 1H), 7.77 (s, 1H), 7.34-7.28 (m, 1H), 7.20-7.15 (m, 1H), 7.10 (m, 1H), 5.20 (d, J=6.4 Hz, 1H), 4.83-4.78 (m, 2H), 4.63-4.57 (m, 1H), 4.37-4.32 (m, 1H), 4.22-4.17 (m, 1H), 4.03-3.94 (m, 2H), 3.30-3.22 (m, 2H), 2.10-1.98 (m, 2H). Mass Spectrum (ESI) m/z=559.9 (M−1).

### Example S19

### Synthesis of (((((2R,3S,4R,1S)-5-(2-chloro-4-(3,4-dihydroisoquinolin-2(1H)-yl)imidazo[2,1-f][1,2,4]triazin-7-yl)-3,4-dihydroxytetrahydrofuran-2-yl)methoxy)(hydroxy)phosphoryl)methyl)phosphonic Acid

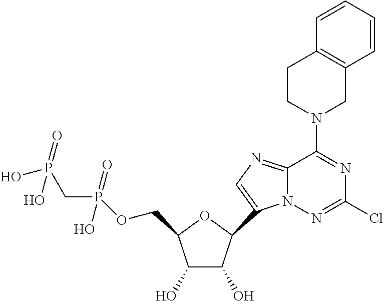 [SMILES: O=P(O)(O)CP(=O)(O)OC[C@H]1O[C@@H](c2cnc3c(N4CCc5ccccc5C4)nc(Cl)nn23)[C@H](O)[C@@H]1O]

(((((2R,3S,4R,5S)-5-(2-chloro-4-(3,4-dihydroisoquinolin-2(1H)-yl)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with 1,2,3,4-tetrahydroisoquinoline.

¹H NMR (400 MHz, DMSO) δ 7.83 (d, J=3.0 Hz, 1H), 7.36-7.20 (m, 4H), 5.91 (d, J=5.8 Hz, 1H), 5.05 (s, 2H), 4.98-4.91 (m, 1H), 4.31-4.21 (m, 1H), 4.17-4.05 (m, 2H), 3.98-3.84 (m, 3H), 3.05-2.97 (m, 2H), 1.83 (t, J=18.4 Hz, 2H). Mass Spectrum (ESI) m/z=576.0 (M+1).

### Example S20

### Synthesis of (((((2R,3S,4R,1S)-5-(2-chloro-4-(cyclohexylamino)imidazo[2,1-f][1,2,4]triazin-7-yl)-3,4-dihydroxytetrahydrofuran-2-yl)methoxy)(hydroxy)phosphoryl)methyl)phosphonic Acid

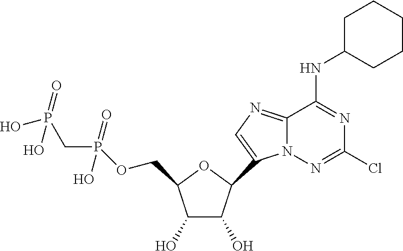 [SMILES: O=P(O)(O)CP(=O)(O)OC[C@H]1O[C@@H](c2cnc3c(NC4CCCCC4)nc(Cl)nn23)[C@H](O)[C@@H]1O]

(((((2R,3S,4R,5S)-5-(2-chloro-4-(cyclohexylamino)imidazo[2,1-f][1,2,4]triazin-7-yl)-3,4-dihydroxytetrahydrofuran-2-yl)methoxyxhydroxy)phosphoryl)methyl)phosphonic acid was prepared by procedures similar to the one described in Example S5, replacing cyclopentanamine in Step F with cyclohexanamine.

¹H NMR (400 MHz, D₂O) δ 7.60 (s, 1H), 5.10 (d, J=6.9 Hz, 1H), 4.53 (m, 1H), 4.28 (m, 1H), 4.13 (d, J=3.6 Hz, 1H), 3.98-3.89 (m, 3H), 2.02-2.00 (m, 2H), 1.89-1.85 (m, 2H), 1.68-1.66 (m, 2H), 1.52 (m, 1H), 1.27-1.25 (m, 5H). Mass Spectrum (ESI) m/z=540.0 (M−1).

### Example S21

### Synthesis of [({[(2R,3S,4R,5S)-5-{2-chloro-4-[(3S)-oxolan-3-ylamino]imidazo[2,1-f][1,2,4]triazin-7-yl}-3,4-dihydroxyoxolan-2-yl]methoxy}(hydroxy)phosphoryl)methyl]phosphonic Acid

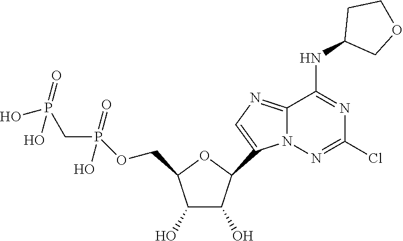 [SMILES: O=P(O)(O)CP(=O)(O)OC[C@H]1O[C@@H](c2cnc3c(N[C@H]4CCOC4)nc(Cl)nn23)[C@H](O)[C@@H]1O]

[({[(2R,3S,4R,5S)-5-{2-chloro-4-[(3S)-oxolan-3-ylamino]imidazo[2,1-f][1,2,4]triazin-7-yl}-3,4-dihydroxyoxolan-2-yl]methoxy}(hydroxy)phosphoryl)methyl]phosphonic acid was prepared by procedures similar to the one described in Example S5, replacing cyclopentanamine in Step F with (S)-tetrahydrofuran-3-amine.

¹H NMR (400 MHz, D₂O) δ 7.67 (s, 1H), 5.16 (d, J=6.9 Hz, 1H), 4.77-4.73 (m, 1H), 4.57 (dd, J=6.8, 5.4 Hz, 1H), 4.36-4.31 (m, 1H), 4.20-4.15 (m, 1H), 4.02-3.90 (m, 4H), 3.90-3.82 (m, 2H), 2.40-2.31 (m, 1H), 2.12-1.98 (m, 3H). Mass Spectrum (ESI) m/z=528.0 (M−1).

### Example S22

### Synthesis of (((((2R,3S,4R,1S)-5-(2-chloro-4-(((R)-tetrahydrofuran-3-yl)amino)imidazo[2,1-f][1,2,4]triazin-7-yl)-3,4-dihydroxytetrahydrofuran-2-yl)methoxy)(hydroxy)phosphoryl)methyl)phosphonic Acid

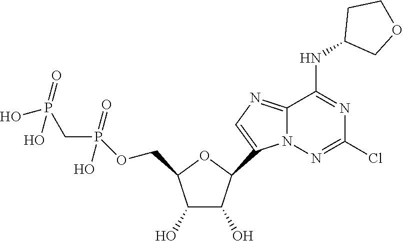 [SMILES: O=P(O)(O)CP(=O)(O)OC[C@H]1O[C@@H](c2cnc3c(N[C@@H]4CCOC4)nc(Cl)nn23)[C@H](O)[C@@H]1O]

(((((2R,3S,4R,5S)-5-(2-chloro-4-(((R)-tetrahydrofuran-3-yl)amino)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with (R)-tetrahydrofuran-3-amine.

¹H NMR (400 MHz, D₂O) δ 7.68 (s, 1H), 5.17 (d, J=6.8 Hz, 1H), 4.58 (m, 1H), 4.36-4.33 (m, 1H), 4.18 (d, J=4.0 Hz, 1H), 4.04-3.83 (m, 7H), 2.41-2.31 (m, 1H), 2.10-1.99 (m, 3H). Mass Spectrum (ESI) m/z=527.9 (M−1).

### Example S23

### Synthesis of (((((2R,3S,4R,1S)-5-(2-chloro-4-((3-methoxycyclopentyl)amino)imidazo[2,1-f][1,2,4]triazin-7-yl)-3,4-dihydroxytetrahydrofuran-2-yl)methoxy)(hydroxy)phosphoryl)methyl)phosphonic acid

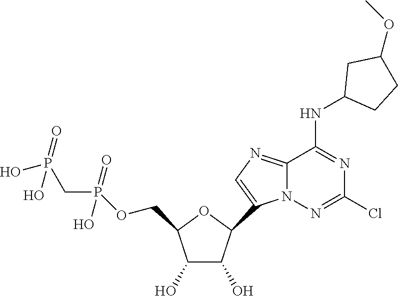 [SMILES: COC1CCC(Nc2nc(Cl)nn3c([C@@H]4O[C@H](COP(=O)(O)CP(=O)(O)O)[C@@H](O)[C@H]4O)cnc23)C1]

(((((2R,3S,4R,5S)-5-(2-chloro-4-((3-methoxycyclopentyl)amino)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with 3-methoxycyclopentan-1-amine.

¹H NMR (400 MHz, D₂O) δ 7.62 (s, 1H), 5.12 (d, J=6.3 Hz, 1H), 4.53-4.48 (m, 2H), 4.36-4.30 (m, 1H), 4.16-4.11 (m, 1H), 4.08-3.89 (m, 3H), 3.23 (s, 3H), 2.15-1.98 (m, 4H), 1.96-1.84 (m, 2H), 1.68-1.58 (m, 2H). Mass Spectrum (ESI) m/z=556.1 (M−1).

### Example S24

### Synthesis of [({[(2R,3S,4R,1S)-5-[2-chloro-4-(1,3-dihydroisoindol-2-yl)imidazo[2,1-f][1,2,4]triazin-7-yl]-3,4-dihydroxyoxolan-2-yl]methoxy}(hydroxy)phosphoryl)methyl]phosphonic Acid

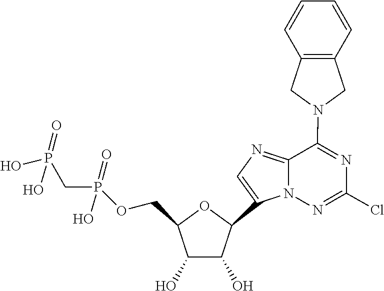 [SMILES: O=P(O)(O)CP(=O)(O)OC[C@H]1O[C@@H](c2cnc3c(N4Cc5ccccc5C4)nc(Cl)nn23)[C@H](O)[C@@H]1O]

[({[(2R,3S,4R,5S)-5-[2-chloro-4-(1,3-dihydroisoindol-2-yl)imidazo[2,1-f][1,2,4]triazin-7-yl]-3,4-dihydroxyoxolan-2-yl]methoxy}(hydroxy)phosphoryl)methyl]phosphonic acid was prepared by procedures similar to the one described in Example S5, replacing cyclopentanamine in Step F with isoindoline.

¹H NMR (400 MHz, D₂O) δ 7.73 (s, 1H), 7.28-7.16 (m, 4H), 5.31 (s, 2H), 5.12 (d, J=6.5 Hz, 1H), 4.83 (s, 2H), 4.61-4.56 (m, 1H), 4.38-4.33 (m, 1H), 4.21-4.17 (m, 1H), 4.04-3.95 (m, 2H), 2.06 (t, J=19.8 Hz, 2H). Mass Spectrum (ESI) m/z=559.9 (M−1).

### Example S25

### Synthesis of (((((2R,3S,4R,1S)-5-(2-chloro-4-(((1S,3S)-3-(dimethylamino)cyclopentyl)amino)imidazo[2,1-f][1,2,4]triazin-7-yl)-3,4-dihydroxytetrahydrofuran-2-yl)methoxy)(hydroxy)phosphoryl)methyl)phosphonic acid

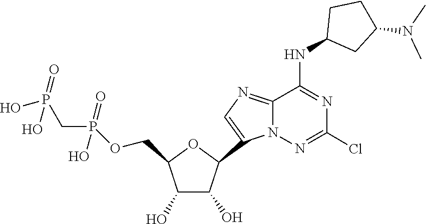 [SMILES: CN(C)[C@H]1CC[C@H](Nc2nc(Cl)nn3c([C@@H]4O[C@H](COP(=O)(O)CP(=O)(O)O)[C@@H](O)[C@H]4O)cnc23)C1]

(((((2R,3S,4R,5S)-5-(2-chloro-4-(((1S,3S)-3-(dimethylamino)cyclopentyl)amino)imidazo[2,1-f][1,2,4]triazin-7-yl)-3,4-dihydroxytetrahydrofuran-2-yl)methoxy)(hydroxy)phosphoryl)methyl)phosphonic acid was prepared by procedures similar to the one described in Example S5, replacing cyclopentanamine in Step F with (1S,3S)—N¹,N¹-dimethylcyclopentane-1,3-diamine.

¹H NMR (400 MHz, D₂O) δ 7.64 (d, J=2.2 Hz, 1H), 5.13 (m, 1H), 4.58-4.53 (m, 1H), 4.50-4.42 (m, 1H), 4.34-4.29 (m, 1H), 4.17-4.12 (m, 1H), 3.97-3.89 (m, 2H), 3.65-3.56 (m, 1H), 2.79 (s, 3H), 2.78 (s, 3H), 2.69-2.62 (m, 1H), 2.19-2.07 (m, 2H), 2.03-1.78 (m, 5H). Mass Spectrum (ESI) m/z=569.0 (M−1).

### Example S26

### Synthesis of [({[(2R,3S,4R,5S)-5-{2-chloro-4-[(5-oxopyrrolidin-3-yl)amino]imidazo[2,1-f][1,2,4]triazin-7-yl}-3,4-dihydroxyoxolan-2-yl]methoxy}(hydroxy)phosphoryl)methyl]phosphonic acid

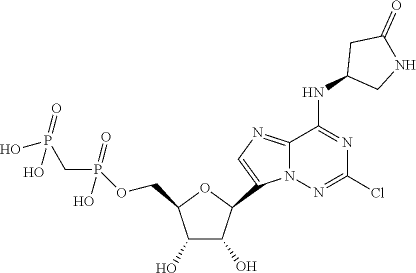 [SMILES: O=C1C[C@H](Nc2nc(Cl)nn3c([C@@H]4O[C@H](COP(=O)(O)CP(=O)(O)O)[C@@H](O)[C@H]4O)cnc23)CN1]

[({[(2R,3S,4R,5S)-5-{2-chloro-4-[(5-oxopyrrolidin-3-yl)amino]imidazo[2,1-f][1,2,4]triazin-7-yl}-3,4-dihydroxyoxolan-2-yl]methoxy}(hydroxy)phosphoryl)methyl]phosphonic acid was prepared by procedures similar to the one described in Example S5, replacing cyclopentanamine in Step F with (S)-4-aminopyrrolidin-2-one.

¹H NMR (400 MHz, D₂O) δ 7.67 (s, 1H), 5.15 (d, J=6.2 Hz, 1H), 4.95-4.90 (m, 1H), 4.57-4.51 (m, 1H), 4.37-4.35 (m, 1H), 4.17-4.15 (m, 1H), 4.10-3.92 (m, 3H), 3.86-3.81 (m, 1H), 3.45-3.42 (m, 1H), 2.51-2.46 (m, 1H), 1.98-1.85 (m, 2H). Mass Spectrum (ESI) m/z=541.9 (M−1).

### Example S27

### Synthesis of (((((2R,3S,4R,1S)-5-(2-chloro-4-(((1s,4R)-4-methoxycyclohexyl)amino)imidazo[2,1-f][1,2,4]triazin-7-yl)-3,4-dihydroxytetrahydrofuran-2-yl)methoxy)(hydroxy)phosphoryl) methyl)phosphonic Acid

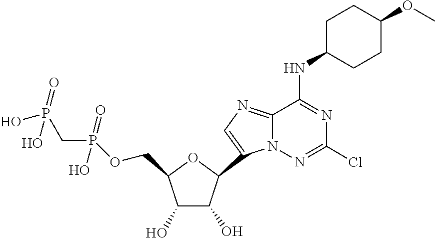 [SMILES: CO[C@H]1CC[C@@H](Nc2nc(Cl)nn3c([C@@H]4O[C@H](COP(=O)(O)CP(=O)(O)O)[C@@H](O)[C@H]4O)cnc23)CC1]

(((((2R,3S,4R,5S)-5-(2-chloro-4-(((1s,4R)-4-methoxycyclohexyl)amino)imidazo [2,1-f][1,2,4]triazin-7-yl)-3,4-dihydroxytetrahydrofuran-2-yl)methoxyxhydroxy)phosphoryl) methyl)phosphonic acid was prepared by procedures similar to the one described in Example S5, replacing cyclopentanamine in Step F with (1S,4S)-4-methoxycyclohexan-1-amine.

¹H NMR (400 MHz, D₂O) δ 7.65 (s, 1H), 5.14 (d, J=6.1 Hz, 1H), 4.54 (t, J=5.6 Hz, 1H), 4.36 (t, J=4.7 Hz, 1H), 4.18-4.13 (m, 1H), 4.05-3.94 (m, 3H), 3.39-3.33 (m, 1H), 3.29 (s, 3H), 2.12-1.99 (m, 4H), 1.96-1.82 (m, 2H), 1.49-1.40 (m, 2H), 1.34-1.25 (m, 2H). Mass Spectrum (ESI) m/z=570.0 (M−1).

### Example S28

### Synthesis of (((((2R,3S,4R,1S)-5-(2-chloro-4-(((R)-2,3-dihydro-1H-inden-1-yl)amino)imidazo[2,1-f][1,2,4]triazin-7-yl)-3,4-dihydroxytetrahydrofuran-2-yl)methoxy)(hydroxy)phosphoryl)methyl)phosphonic Acid

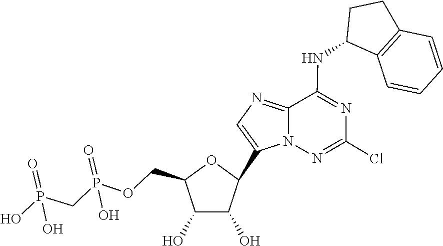 [SMILES: O=P(O)(O)CP(=O)(O)OC[C@H]1O[C@@H](c2cnc3c(N[C@@H]4CCc5ccccc54)nc(Cl)nn23)[C@H](O)[C@@H]1O]

(((((2R,3S,4R,5S)-5-(2-chloro-4-(((R)-2,3-dihydro-1H-inden-1-yl)amino)imidazo[2,1-f][1,2,4]triazin-7-yl)-3,4-dihydroxytetrahydrofuran-2-yl)methoxyxhydroxy)phosphoryl)methyl)phosphonic acid was prepared by procedures similar to the one described in Example S5, replacing cyclopentanamine in Step F with (R)-2,3-dihydro-1H-inden-1-amine.

¹H NMR (400 MHz, D₂O) δ 7.65 (s, 1H), 7.34-7.14 (m, 4H), 5.75-5.68 (m, 1H), 5.18-5.14 (m, 1H), 4.57-4.52 (m, 1H), 4.38-4.32 (m, 1H), 4.19-4.13 (m, 1H), 4.04-3.92 (m, 2H), 2.96-2.82 (m, 2H), 2.62-2.53 (m, 1H), 2.08-2.00 (m, 1H), 2.00-1.88 (m, 2H). Mass Spectrum (ESI) m/z=574.0 (M−1).

### Example S29

### Synthesis of (((((2R,3S,4R,1S)-5-(2-chloro-4-(((R)-2,3-dihydro-1H-inden-1-yl)(methyl)amino)imidazo[2,1-f][1,2,4]triazin-7-yl)-3,4-dihydroxytetrahydrofuran-2-yl)methoxy)(hydroxy)phosphoryl)methyl)phosphonic Acid

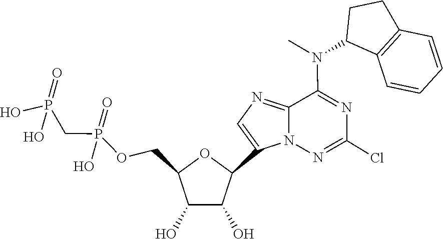

(((((2R,3S,4R,5S)-5-(2-chloro-4-(((R)-2,3-dihydro-1H-inden-1-yl)(methyl)amino)imidazo[2,1-f][1,2,4]triazin-7-yl)-3,4-dihydroxytetrahydrofuran-2-yl)methoxyxhydroxy)phosphoryl)methyl)phosphonic acid was prepared by procedures similar to the one described in Example S5, replacing cyclopentanamine in Step F with (R)—N-methyl-2,3-dihydro-1H-inden-1-amine.

¹H NMR (400 MHz, D₂O) δ 7.69-7.60 (m, 1H), 7.50 (t, J=7.9 Hz, 0.5H), 7.330 (m, 1H), 7.26-7.20 (m, 1H), 7.16-7.10 (m, 2H), 6.56 (t, J=7.7 Hz, 1H), 5.20-5.14 (m, 1H), 4.58-4.50 (m, 1H), 4.32-4.30 (m, 1H), 4.18-4.10 (m, 1H), 3.98-3.90 (m, 2H), 3.33 (s, 1H), 2.94-2.90 (m, 1H), 2.87 (s, 2H), 2.50-2.45 (m, 1H), 2.05-1.70 (m, 4H). Mass Spectrum (ESI) m/z=588.1 (M−1).

### Example S30

### Synthesis of (((((2R,3S,4R,1S)-5-(2-chloro-4-(((S)-2,3-dihydro-1H-inden-1-yl)(methyl)amino)imidazo[2,1-f][1,2,4]triazin-7-yl)-3,4-dihydroxytetrahydrofuran-2-yl)methoxy)(hydroxy)phosphoryl)methyl)phosphonic Acid

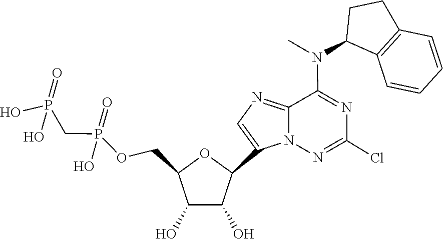 [SMILES: CN(c1nc(Cl)nn2c([C@@H]3O[C@H](COP(=O)(O)CP(=O)(O)O)[C@@H](O)[C@H]3O)cnc12)[C@H]1CCc2ccccc21]

(((((2R,3S,4R,5S)-5-(2-chloro-4-(((S)-2,3-dihydro-1H-inden-1-yl)(methyl)amino)imidazo[2,1-f][1,2,4]triazin-7-yl)-3,4-dihydroxytetrahydrofuran-2-yl)methoxyxhydroxy)phosphoryl)methyl)phosphonic acid was prepared by procedures similar to the one described in Example S5, replacing cyclopentanamine in Step F with (S)—N-methyl-2,3-dihydro-1H-inden-1-amine.

¹H NMR (400 MHz, D₂O) δ 7.66 (d, J=9.7 Hz, 1H), 7.46-7.43 (m, 0.5H), 7.26-7.23 (m, 2H), 7.12-7.10 (m, 2H), 6.54-6.53 (m, 0.5H), 5.15-5.10 (m, 1H), 4.59-4.53 (m, 1H), 4.30 (t, J=4.7 Hz, 1H), 4.15-4.12 (m, 1H), 3.95-3.93 (m, 2H), 3.31-3.29 (m, 1H), 3.03-3.00 (m, 1H), 2.87-2.80 (m, 2H), 2.49-2.40 (m, 1H), 2.05-1.90 (m, 4H). Mass Spectrum (ESI) m/z=587.9 (M−1).

### Example S31

### Synthesis of [({[(2R,3S,4R,5S)-5-{2-chloro-4-[(2R)-2-phenylpyrrolidin-1-yl]imidazo[2,1-f][1,2,4]triazin-7-yl}-3,4-dihydroxyoxolan-2-yl]methoxy}(hydroxy)phosphoryl)methyl]phosphonic Acid

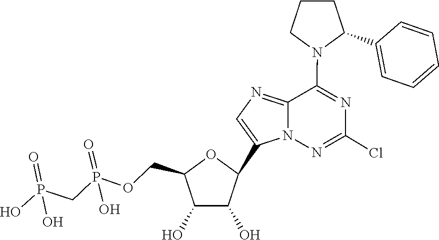 [SMILES: O=P(O)(O)CP(=O)(O)OC[C@H]1O[C@@H](c2cnc3c(N4CCC[C@@H]4c4ccccc4)nc(Cl)nn23)[C@H](O)[C@@H]1O]

[({[(2R,3S,4R,5S)-5-{2-chloro-4-[(2R)-2-phenylpyrrolidin-1-yl]imidazo[2,1-f][1,2,4]triazin-7-yl}-3,4-dihydroxyoxolan-2-yl]methoxy}(hydroxy)phosphoryl)methyl]phosphonic acid was prepared by procedures similar to the one described in Example S5, replacing cyclopentanamine in Step F with (R)-2-phenylpyrrolidine.

¹H NMR (400 MHz, D₂O) δ 7.73 (s, 0.5H), 7.42 (s, 0.5H), 7.32-7.09 (m, 5H), 6.31 (d, J=7.5 Hz, 0.5H), 5.48-5.43 (m, 0.5H), 5.10 (dd, J=23.1, 6.6 Hz, 1H), 4.55-4.47 (m, 1H), 4.44-4.22 (m, 2H), 4.16-4.08 (m, 1H), 4.01-3.89 (m, 2H), 3.87-3.72 (m, 1H), 2.45-2.32 (m, 1H), 2.14-1.80 (m, 5H). Mass Spectrum (ESI) m/z=588.0 (M−1).

### Example S32

### Synthesis of (((((2R,3S,4R,1S)-5-(2-chloro-4-(((S)-1-(2-fluorophenyl)ethyl)amino)imidazo[2,1-f][1,2,4]triazin-7-yl)-3,4-dihydroxytetrahydrofuran-2-yl)methoxy)(hydroxy)phosphoryl)-methyl)phosphonic Acid

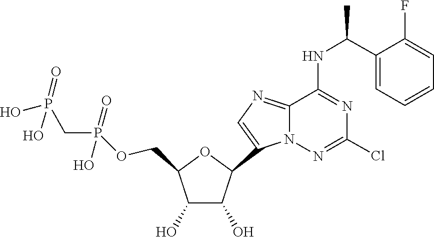 [SMILES: C[C@H](Nc1nc(Cl)nn2c([C@@H]3O[C@H](COP(=O)(O)CP(=O)(O)O)[C@@H](O)[C@H]3O)cnc12)c1ccccc1F]

(((((2R,3S,4R,5S)-5-(2-chloro-4-(((S)-1-(2-fluorophenyl)ethyl)amino)imidazo[2,1-f][1,2,4]triazin-7-yl)-3,4-dihydroxytetrahydrofuran-2-yl)methoxyxhydroxy)phosphoryl)-methyl)phosphonic acid was prepared by procedures similar to the one described in S32, replacing cyclopentanamine in Step F with (S)-1-(2-fluorophenyl)ethan-1-amine.

¹H NMR (400 MHz, D₂O) δ 7.67 (s, 1H), 7.43-7.36 (m, 1H), 7.29-7.21 (m, 1H), 7.13-7.03 (m, 2H), 5.57-5.49 (m, 1H), 5.13 (d, J=6.7 Hz, 1H), 4.57-4.51 (m, 1H), 4.32 (t, J=4.8 Hz, 1H), 4.18-4.13 (m, 1H), 4.02-3.91 (m, 2H), 1.99 (t, J=19.7 Hz, 2H), 1.59 (d, J=6.9 Hz, 3H). Mass Spectrum (ESI) m/z=579.9 (M−1).

### Example S33

### Synthesis of (((((2R,3S,4R,1S)-5-(2-chloro-4-(((S)-1-(S-fluoropyridin-3-yl)ethyl)amino)imidazo[2,1-f][1,2,4]triazin-7-yl)-3,4-dihydroxytetrahydrofuran-2-yl)methoxy)(hydroxy)phosphoryl)methyl)phosphonic Acid

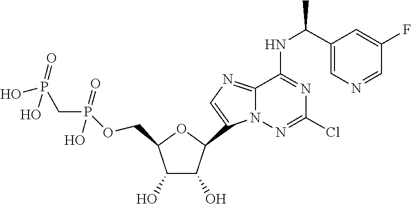 [SMILES: C[C@H](Nc1nc(Cl)nn2c([C@@H]3O[C@H](COP(=O)(O)CP(=O)(O)O)[C@@H](O)[C@H]3O)cnc12)c1cncc(F)c1]

(((((2R,3S,4R,5S)-5-(2-chloro-4-(((S)-1-(5-fluoropyridin-3-yl)ethyl)amino)imidazo[2,1-f][1,2,4]triazin-7-yl)-3,4-dihydroxytetrahydrofuran-2-yl)methoxyxhydroxy)phosphoryl)methyl)phosphonic acid was prepared by procedures similar to the one described in Example S5, replacing cyclopentanamine in Step F with (S)-1-(5-fluoropyridin-3-yl)ethan-1-amine.

¹H NMR (400 MHz, D₂O) δ 8.38 (s, 1H), 8.25 (d, J=2.5 Hz, 1H), 7.68 (s, 1H), 7.64 (d, J=9.6 Hz, 1H), 5.46-5.39 (m, 1H), 5.13 (d, J=6.9 Hz, 1H), 4.59-4.52 (m, 1H), 4.35-4.29 (m, 1H), 4.20-4.11 (m, 1H), 4.00-3.90 (m, 2H), 2.10-1.94 (m, 2H), 1.61 (d, J=7.0 Hz, 3H). Mass Spectrum (ESI) m/z=582.7 (M+1).

### Example S34

### Synthesis of [({[(2R,3S,4R,5S)-5-(2-chloro-4-{[(1R)-1-phenylethyl]amino}imidazo[2,1-f][1,2,4]triazin-7-yl)-3,4-dihydroxyoxolan-2-yl]methoxy}(hydroxy)phosphoryl)methyl]phosphonic Acid

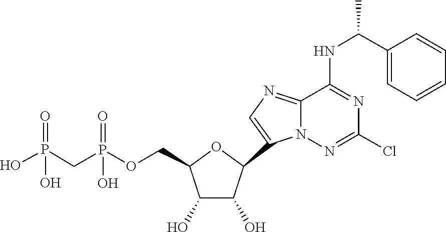 [SMILES: C[C@@H](Nc1nc(Cl)nn2c([C@@H]3O[C@H](COP(=O)(O)CP(=O)(O)O)[C@@H](O)[C@H]3O)cnc12)c1ccccc1]

[({[(2R,3S,4R,5S)-5-(2-chloro-4-{[(1R)-1-phenylethyl]amino}imidazo[2,1-f][1,2,4]triazin-7-yl)-3,4-dihydroxyoxolan-2-yl]methoxy}(hydroxy)phosphoryl)methyl]phosphonic acid was prepared by procedures similar to the one described in Example S5, replacing cyclopentanamine in Step F with (R)-1-phenylethan-1-amine.

¹H NMR (400 MHz, D₂O) δ 7.65 (s, 1H), 7.38-7.35 (m, 2H), 7.31-7.28 (m, 2H), 7.23-7.19 (m, 1H), 5.29-5.25 (t, J=7.0 Hz, 1H), 5.12-5.10 (m, 1H), 4.56-4.50 (m, 1H), 4.32-4.27 (m, 1H), 4.14-4.10 (m, 1H), 3.94-3.90 (m, 2H), 2.00-1.95 (t, J=19.8 Hz, 2H), 1.56 (d, J=7.0 Hz, 3H). Mass Spectrum (ESI) m/z=562.0 (M−1).

### Example S35

### Synthesis of (((((2R,3S,4R,1S)-5-(2-chloro-4-(((S)-1-phenylethyl)amino)imidazo[2,1-f][1,2,4]triazin-7-yl)-3,4-dihydroxytetrahydrofuran-2-yl)methoxy)(hydroxy)phosphoryl)methyl)phosphonic Acid

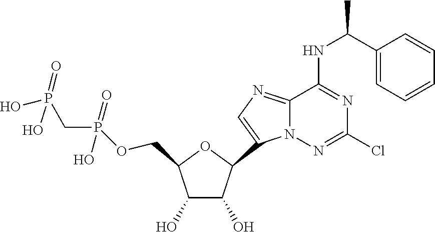 [SMILES: C[C@H](Nc1nc(Cl)nn2c([C@@H]3O[C@H](COP(=O)(O)CP(=O)(O)O)[C@@H](O)[C@H]3O)cnc12)c1ccccc1]

(((((2R,3S,4R,5S)-5-(2-chloro-4-(((S)-1-phenylethyl)amino)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with (S)-1-phenylethan-1-amine.

¹H NMR (400 MHz, D₂O) δ 7.66 (s, 1H), 7.39 (d, J=7.6 Hz, 2H), 7.34-7.29 (m, 2H), 7.27-7.23 (m, 1H), 5.34-5.28 (m, 1H), 5.12 (d, J=6.9 Hz, 1H), 4.56-4.52 (m, 1H), 4.32-4.28 (m, 1H), 4.17-4.13 (m, 1H), 3.95-3.91 (m, 2H), 2.02 (t, J=19.8 Hz, 2H), 1.56 (d, J=7.0 Hz, 3H). Mass Spectrum (ESI) m/z=563.5 (M+1).

### Example S36

### Synthesis of (((((2R,3S,4R,1S)-5-(2-chloro-4-(((S)-1-(3-fluorophenyl)ethyl)amino)imidazo[2,1-f][1,2,4] triazin-7-yl)-3,4-dihydroxytetrahydrofuran-2-yl)methoxy)(hydroxy)phosphoryl)methyl) phosphonic Acid

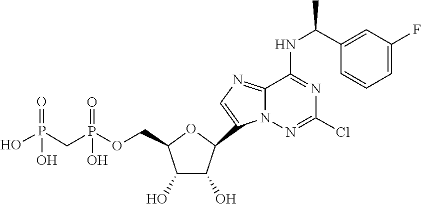 [SMILES: C[C@H](Nc1nc(Cl)nn2c([C@@H]3O[C@H](COP(=O)(O)CP(=O)(O)O)[C@@H](O)[C@H]3O)cnc12)c1cccc(F)c1]

(((((2R,3S,4R,5S)-5-(2-chloro-4-(((S)-1-(3-fluorophenyl)ethyl)amino)imidazo[2,1-f][1,2,4] triazin-7-yl)-3,4-dihydroxytetrahydrofuran-2-yl)methoxy)(hydroxy)phosphoryl)methyl) phosphonic acid was prepared by procedures similar to the one described in Example S5, replacing cyclopentanamine in Step F with (S)-1-(3-fluorophenyl)ethan-1-amine.

¹H NMR (400 MHz, D₂O) δ 7.72 (s, 1H), 7.29-7.27 (dd, J=14.4, 7.7 Hz, 1H), 7.16-7.14 (dd, J=24.0, 9.3 Hz, 2H), 6.96 (t, J=7.7 Hz, 1H), 5.33 (m, 1H), 5.15 (m, 1H), 4.54 (m, 1H), 4.31 (t, J=4.7 Hz, 1H), 4.16 (d, J=3.8 Hz, 1H), 3.98 (s, 2H), 2.14 (m, 2H), 1.57 (d, J=6.9 Hz, 3H). Mass Spectrum (ESI) m/z=579.7 (M−1).

### Example S37

### Synthesis of (((((2R,3S,4R,1S)-5-(2-chloro-4-(((S)-1-(4-fluorophenyl)ethyl)amino)imidazo[2,1-f][1,2,4]triazin-7-yl)-3,4-dihydroxytetrahydrofuran-2-yl)methoxy)(hydroxy)phosphoryl)methyl)phosphonic Acid

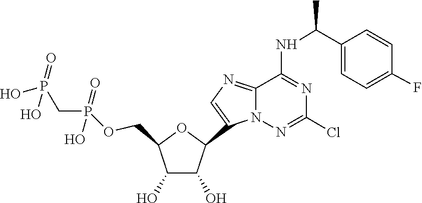 [SMILES: C[C@H](Nc1nc(Cl)nn2c([C@@H]3O[C@H](COP(=O)(O)CP(=O)(O)O)[C@@H](O)[C@H]3O)cnc12)c1ccc(F)cc1]

(((((2R,3S,4R,5S)-5-(2-chloro-4-(((S)-1-(4-fluorophenyl)ethyl)amino)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with (S)-1-(4-fluorophenyl)ethan-1-amine.

¹H NMR (400 MHz, D₂O) δ 7.82-7.73 (s, 1H), 7.42-7.32 (m, 2H), 7.05-6.98 (m, 2H), 5.36-5.27 (m, 1H), 5.16 (d, J=6.2 Hz, 1H), 4.57-4.51 (m, 1H), 4.32-4.27 (m, 1H), 4.19-4.13 (m, 1H), 4.01 (s, 2H), 2.25-2.11 (m, 2H), 1.56 (d, J=6.9 Hz, 3H). Mass Spectrum (ESI) m/z=581.8 (M+1).

### Example S38

### Synthesis of (((((2R,3S,4R,1S)-5-(4-((S)-2-(tert-butyl)pyrrolidin-1-yl)-2-chloroimidazo[2,1-f][1,2,4]triazin-7-yl)-3,4-dihydroxytetrahydrofuran-2-yl)methoxy)(hydroxy)phosphoryl)methyl)phosphonic Acid

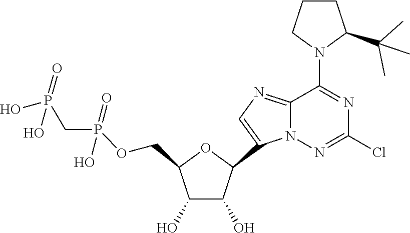 [SMILES: CC(C)(C)[C@@H]1CCCN1c1nc(Cl)nn2c([C@@H]3O[C@H](COP(=O)(O)CP(=O)(O)O)[C@@H](O)[C@H]3O)cnc12]

(((((2R,3S,4R,5S)-5-(4-((S)-2-(tert-butyl)pyrrolidin-1-yl)-2-chloroimidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with (S)-2-(tert-butyl)pyrrolidine.

¹H NMR (400 MHz, D₂O) δ 7.75-7.63 (m, 1H), 5.18-5.10 (m, 1H), 4.69-4.64 (m, 1H), 4.60-4.54 (m, 1H), 4.53-4.40 (m, 1H), 4.35-4.28 (m, 1H), 4.20-4.12 (m, 1H), 4.02-3.88 (m, 3H), 2.26-1.83 (m, 6H), 0.83-0.6 (m, 9H). Mass Spectrum (ESI) m/z=567.7 (M−1).

### Example S39

### Synthesis of (((((2R,3S,4R,1S)-5-(2-chloro-4-((S)-2-isopropylpyrrolidin-1-yl)imidazo[2,1-f][1,2,4]triazin-7-yl)-3,4-dihydroxytetrahydrofuran-2-yl)methoxy)(hydroxy)phosphoryl)methyl)phosphonic acid

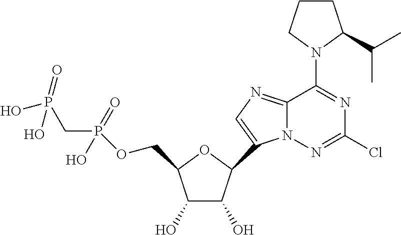 [SMILES: CC(C)[C@@H]1CCCN1c1nc(Cl)nn2c([C@@H]3O[C@H](COP(=O)(O)CP(=O)(O)O)[C@@H](O)[C@H]3O)cnc12]

(((((2R,3S,4R,5S)-5-(2-chloro-4-((S)-2-isopropylpyrrolidin-1-yl)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with (S)-2-isopropylpyrrolidine.

¹H NMR (400 MHz, DMSO) δ 7.79-7.76 (m, 1H), 5.02 (d, J=5.1 Hz, 1H), 4.69-4.59 (m, 1H), 4.40-4.33 (m, 1H), 4.27-4.24 (m, 1H), 4.13-4.04 (m, 2H), 3.94-3.88 (m, 3H), 2.07-1.71 (m, 7H), 0.94-0.82 (m, 4H), 0.80-0.76 (m, 2H). Mass Spectrum (ESI) m/z=556.0 (M+1).

### Example S40

### Synthesis of (((((2R,3S,4R,1S)-5-(2-chloro-4-(1-azaspiro[4.4]nonan-1-yl)imidazo[2,1-f][1,2,4]triazin-7-yl)-3,4-dihydroxytetrahydrofuran-2-yl)methoxy)(hydroxy)phosphoryl)methyl)phosphonic Acid

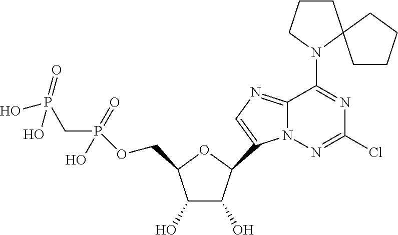 [SMILES: O=P(O)(O)CP(=O)(O)OC[C@H]1O[C@@H](c2cnc3c(N4CCCC45CCCC5)nc(Cl)nn23)[C@H](O)[C@@H]1O]

(((((2R,3S,4R,5S)-5-(2-chloro-4-(1-azaspiro[4.4]nonan-1-yl)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with 1-azaspiro[4.4]nonane.

¹H NMR (400 MHz, D₂O) δ 7.74 (s, 1H), 5.18 (d, J=6.1 Hz, 1H), 4.60-4.49 (m, 1H), 4.38-4.28 (m, 1H), 4.24-4.08 (m, 3H), 4.01 (s, 2H), 2.51-2.33 (m, 2H), 2.15 (t, J=19.7 Hz, 2H), 2.04-1.84 (m, 6H), 1.64-1.36 (m, 4H). Mass Spectrum (ESI) m/z=567.5 (M+1).

### Example S41

### Synthesis of [({[(2R,3S,4R,5S)-5-(4-{5-azaspiro[3.4]octan-5-yl}-2-chloroimidazo[2,1-f][1,2,4]triazin-7-yl)-3,4-dihydroxyoxolan-2-yl]methoxy}(hydroxy)phosphoryl)methyl]phosphonic Acid

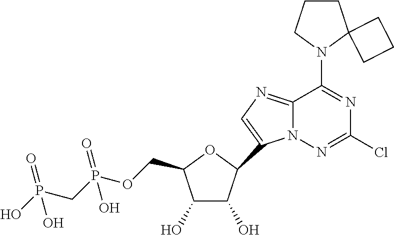 [SMILES: O=P(O)(O)CP(=O)(O)OC[C@H]1O[C@@H](c2cnc3c(N4CCCC45CCC5)nc(Cl)nn23)[C@H](O)[C@@H]1O]

[({[(2R,3S,4R,5S)-5-(4-{5-azaspiro[3.4]octan-5-yl}-2-chloroimidazo[2,1-f][1,2,4]triazin-7-yl)-3,4-dihydroxyoxolan-2-yl]methoxy}(hydroxy)phosphoryl)methyl]phosphonic acid was prepared by procedures similar to the one described in Example S5, replacing cyclopentanamine in Step F with 5-azaspiro[3.4]octane.

¹H NMR (400 MHz, D₂O) δ 7.74 (s, 1H), 5.17-5.15 (m, 1H), 4.55-4.53 (m, 1H), 4.33-4.29 (m, 1H), 4.16-4.14 (m, 1H), 4.08-4.06 (m, 2H), 3.99-3.90 (m, 2H), 3.34-3.40 (m, 2H), 2.13-2.10 (m, 2H), 2.04-1.92 (m, 2H), 1.91-1.64 (m, 6H). Mass Spectrum (ESI) m/z=553.6 (M+1).

### Example S42

### Synthesis of (((((2R,3S,4R,1S)-5-(2-chloro-4-morpholinoimidazo[2,1-f][1,2,4]triazin-7-yl)-3,4-dihydroxytetmhydrofuran-2-yl)methoxy)(hydroxy)phosphoryl)methyl)phosphonic Acid

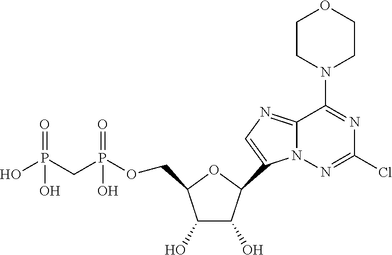 [SMILES: O=P(O)(O)CP(=O)(O)OC[C@H]1O[C@@H](c2cnc3c(N4CCOCC4)nc(Cl)nn23)[C@H](O)[C@@H]1O]

(((((2R,3S,4R,5S)-5-(2-chloro-4-morpholinoimidazo[2,1-f][1,2,4]triazin-7-yl)-3,4-dihydroxytetrahydrofuran-2-yl)methoxyxhydroxy)phosphoryl)methyl)phosphonic acid was prepared by procedures similar to the one described in Example S5, replacing cyclopentanamine in Step F with morpholine.

¹H NMR (400 MHz, D₂O) δ 7.67 (s, 1H), 5.16 (d, J=6.6 Hz, 1H), 4.65 (d, J=1.6 Hz, 4H), 4.58-4.55 (m, 1H), 4.31 (t, J=4.6 Hz, 1H), 4.16 (d, J=3.8 Hz, 1H), 4.01-3.97 (m, 2H), 3.82-3.79 (m, 4H), 2.08-2.12 (m, 2H). Mass Spectrum (ESI) m/z=527.7 (M−1).

### Example S43

### Synthesis of (((((2R,3S,4R,1S)-5-(2-chloro-4-(piperidin-1-yl)imidazo[2,1-f][1,2,4]triazin-7-yl)-3,4-dihydroxytetrahydrofuran-2-yl)methoxy)(hydroxy)phosphoryl)methyl)phosphonic Acid

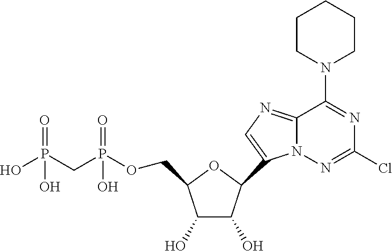 [SMILES: O=P(O)(O)CP(=O)(O)OC[C@H]1O[C@@H](c2cnc3c(N4CCCCC4)nc(Cl)nn23)[C@H](O)[C@@H]1O]

(((((2R,3S,4R,5S)-5-(2-chloro-4-(piperidin-1-yl)imidazo[2,1-f][1,2,4]triazin-7-yl)-3,4-dihydroxytetrahydrofuran-2-yl)methoxyxhydroxy)phosphoryl)methyl)phosphonic acid was prepared by procedures similar to the one described in Example S5, replacing cyclopentanamine in Step F with piperidine.

¹H NMR (400 MHz, D₂O) δ 7.64 (s, 1H), 5.15 (d, J=6.4 Hz, 1H), 4.55 (m, 1H), 4.45-4.42 (m, 2H), 4.31 (m, 1H), 4.17 (m, 1H), 3.98 (m, 2H), 3.83 (m, 2H), 2.15 (m, 2H), 1.65-1.60 (m, 6H). Mass Spectrum (ESI) m/z=525.6 (M−1).

### Example S44

### Synthesis of (((((2R,3S,4R,1S)-5-(2-chloro-4-(4,4-difluoropiperidin-1-yl)imidazo[2,1-f][1,2,4]triazin-7-yl)-3,4-dihydroxytetrahydrofuran-2-yl)methoxy)(hydroxy)phosphoryl)methyl)phosphonic Acid

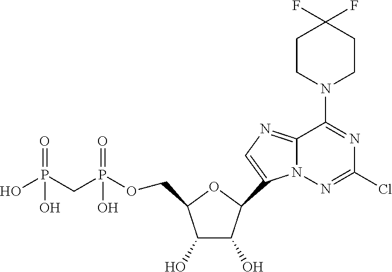 [SMILES: O=P(O)(O)CP(=O)(O)OC[C@H]1O[C@@H](c2cnc3c(N4CCC(F)(F)CC4)nc(Cl)nn23)[C@H](O)[C@@H]1O]

(((((2R,3S,4R,5S)-5-(2-chloro-4-(4,4-difluoropiperidin-1-yl)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with 4,4-difluoropiperidine.

¹H NMR (400 MHz, D₂O) δ 7.68 (s, 1H), 5.16 (d, J=6.5 Hz, 1H), 4.78-4.73 (m, 2H), 4.67 (m, 2H), 4.58 (m, 1H), 4.31 (t, J=4.7 Hz, 1H), 4.16 (d, J=3.7 Hz, 1H), 3.99 (m, 2H), 2.15-2.12 (m, 6H). Mass Spectrum (ESI) m/z=561.5 (M−1).

### Example S45

### Synthesis of [({[(2R,3S,4R,5S)-5-(4-{3-azabicyclo[3.1.0]hexan-3-yl}-2-chloroimidazo[2,1-f][1,2,4]triazin-7-yl)-3,4-dihydroxyoxolan-2-yl]methoxy}(hydroxy)phosphoryl)methyl]phosphonic Acid

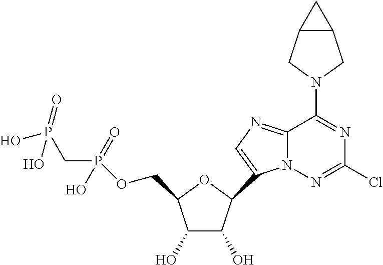 [SMILES: O=P(O)(O)CP(=O)(O)OC[C@H]1O[C@@H](c2cnc3c(N4CC5CC5C4)nc(Cl)nn23)[C@H](O)[C@@H]1O]

[({[(2R,3S,4R,5S)-5-(4-{3-azabicyclo[3.1.0]hexan-3-yl}-2-chloroimidazo[2,1-f][1,2,4]triazin-7-yl)-3,4-dihydroxyoxolan-2-yl]methoxy}(hydroxy)phosphoryl)methyl]phosphonic acid was prepared by procedures similar to the one described in Example S5, replacing cyclopentanamine in Step F with 3-azabicyclo[3.1.0]hexane.

¹H NMR (400 MHz, D₂O) δ 7.66 (s, 1H), 5.12 (d, J=6.1 Hz, 1H), 4.59-4.50 (m, 2H), 4.36-4.31 (m, 1H), 4.16-4.11 (m, 1H), 4.07-3.92 (m, 4H), 3.66-3.59 (m, 1H), 1.91 (t, J=19.6 Hz, 2H), 1.80-1.73 (m, 1H), 1.72-1.64 (m, 1H), 0.79-0.73 (m, 1H), 0.10-0.05 (m, 1H). Mass Spectrum (ESI) m/z=523.7 (M−1).

### Example S46

### Synthesis of [({[(2R,3S,4R,5S)-5-[2-chloro-4-(3,3-difluoropyrrolidin-1-yl)imidazo[2,1-f][1,2,4]triazin-7-yl]-3,4-dihydroxyoxolan-2-yl]methoxy}(hydroxy)phosphoryl)methyl]phosphonic Acid

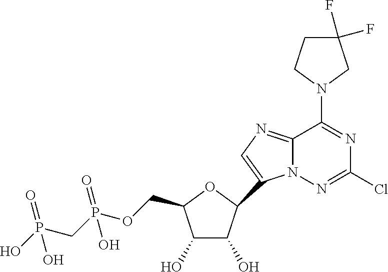 [SMILES: O=P(O)(O)CP(=O)(O)OC[C@H]1O[C@@H](c2cnc3c(N4CCC(F)(F)C4)nc(Cl)nn23)[C@H](O)[C@@H]1O]

[({[(2R,3S,4R,5S)-5-[2-chloro-4-(3,3-difluoropyrrolidin-1-yl)imidazo[2,1-f][1,2,4]triazin-7-yl]-3,4-dihydroxyoxolan-2-yl]methoxy}(hydroxy)phosphoryl)methyl]phosphonic acid was prepared by procedures similar to the one described in Example S5, replacing cyclopentanamine in Step F with 3,3-difluoropyrrolidine.

¹H NMR (400 MHz, D₂O) δ 7.69-7.60 (m, 1H), 5.15 (d, J=6.4 Hz, 1H), 4.57-4.50 (m, 2H), 4.46-4.40 (m, 1H), 4.30 (t, J=4.7 Hz, 1H), 4.16-4.10 (m, 1H), 4.09-3.88 (m, 4H), 2.61-2.45 (m, 2H), 2.10 (t, J=18.6 Hz, 2H). Mass Spectrum (ESI) m/z=547.6 (M−1).

### Example S47

### Synthesis of (((((2R,3S,4R,1S)-5-(2-chloro-4-((tetrahydro-2H-pyran-4-yl)amino)imidazo[2,1-f][1,2,4]triazin-7-yl)-3,4-dihydroxytetrahydrofuran-2-yl)methoxy)(hydroxy)phosphoryl)methyl)phosphonic Acid

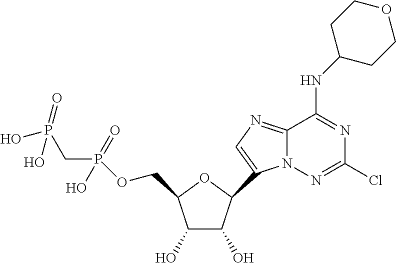 [SMILES: O=P(O)(O)CP(=O)(O)OC[C@H]1O[C@@H](c2cnc3c(NC4CCOCC4)nc(Cl)nn23)[C@H](O)[C@@H]1O]

(((((2R,3S,4R,5S)-5-(2-chloro-4-((tetrahydro-2H-pyran-4-yl)amino)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with tetrahydro-2H-pyran-4-amine.

¹H NMR (400 MHz, D₂O) δ 7.69 (s, 1H), 5.15 (d, J=6.5 Hz, 1H), 4.74 (m, 1H), 4.59-4.53 (m, 1H), 4.33-4.26 (m, 2H), 4.18 (m, 1H), 4.01-3.96 (m, 2H), 3.94 (m, 1H), 3.55 (t, J=10.8 Hz, 2H), 2.18-2.04 (m, 2H), 2.01-1.92 (m, 2H), 1.72-1.62 (m, 2H). Mass Spectrum (ESI) m/z=543.5 (M+1).

### Example S48

### Synthesis of [({[(2R,3S,4R,5S)-5-{2-chloro-4-[(4,4-difluorocyclohexyl)amino]imidazo[2,1-f][1,2,4]triazin-7-yl}-3,4-dihydroxyoxolan-2-yl]methoxy}(hydroxy)phosphoryl)methyl]phosphonic Acid

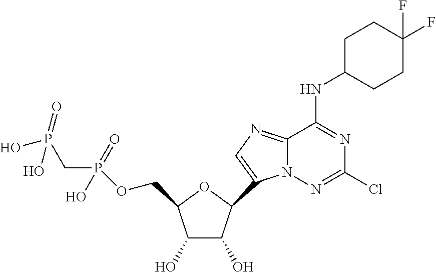 [SMILES: O=P(O)(O)CP(=O)(O)OC[C@H]1O[C@@H](c2cnc3c(NC4CCC(F)(F)CC4)nc(Cl)nn23)[C@H](O)[C@@H]1O]

[({[(2R,3S,4R,5S)-5-{2-chloro-4-[(4,4-difluorocyclohexyl)amino]imidazo[2,1-f][1,2,4]triazin-7-yl}-3,4-dihydroxyoxolan-2-yl]methoxy}(hydroxy)phosphoryl)methyl] phosphonic acid was prepared by procedures similar to the one described in Example S5, replacing cyclopentanamine in Step F with 4,4-difluorocyclohexan-1-amine.

¹H NMR (400 MHz, D₂O) δ 7.69 (s, 1H), 5.16 (d, J=6.5 Hz, 1H), 4.56 (m, 1H), 4.31 (t, J=4.7 Hz, 1H), 4.22-4.14 (m, 2H), 4.04-3.92 (m, 2H), 2.18-1.69 (m, 10H). Mass Spectrum (ESI) m/z=577.6 (M+1).

### Example S49

### Synthesis of (((((2R,3S,4R,1S)-5-(2-chloro-4-((3,3-difluorocyclopentyl)amino)imidazo[2,1-f][1,2,4]triazin-7-yl)-3,4-dihydroxytetrahydrofuran-2-yl)methoxy)(hydroxy)phosphoryl)methyl)phosphonic Acid

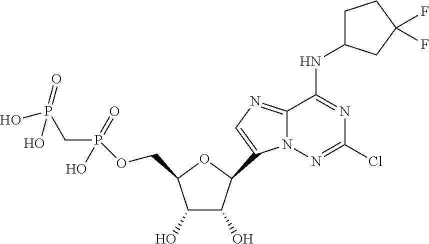 [SMILES: O=P(O)(O)CP(=O)(O)OC[C@H]1O[C@@H](c2cnc3c(NC4CCC(F)(F)C4)nc(Cl)nn23)[C@H](O)[C@@H]1O]

(((((2R,3S,4R,5S)-5-(2-chloro-4-((3,3-difluorocyclopentyl)amino)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with 3,3-difluorocyclopentan-1-amine.

¹H NMR (400 MHz, D₂O) δ 7.68 (s, 1H), 5.15 (d, J=6.5 Hz, 1H), 4.62-4.52 (m, 2H), 4.31 (m, 1H), 4.20-4.13 (m, 1H), 3.99-3.97 (m, 2H), 2.72-2.55 (m, 1H), 2.35-2.03 (m, 6H), 1.97-1.84 (m, 1H). Mass Spectrum (ESI) m/z=563.7 (M+1).

### Example S50

### Synthesis of [({[(2R,3S,4R,1S)-5-(4-{bicyclo[2.2.1]heptan-2-ylamino}-2-chloroimidazo[2,1-f][1,2,4]triazin-7-yl)-3,4-dihydroxyoxolan-2-yl]methoxy}(hydroxy)phosphoryl)methyl]phosphonic Acid

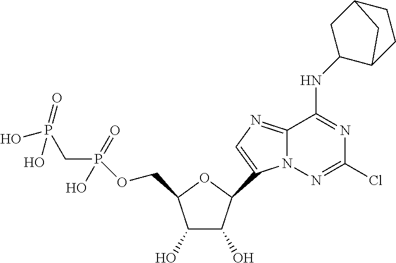 [SMILES: O=P(O)(O)CP(=O)(O)OC[C@H]1O[C@@H](c2cnc3c(NC4CC5CCC4C5)nc(Cl)nn23)[C@H](O)[C@@H]1O]

[({[(2R,3S,4R,5S)-5-(4-{bicyclo[2.2.1]heptan-2-ylamino}-2-chloroimidazo[2,1-f][1,2,4]triazin-7-yl)-3,4-dihydroxyoxolan-2-yl]methoxy}(hydroxy)phosphoryl)methyl]phosphonic acid was prepared by procedures similar to the one described in Example S5, replacing cyclopentanamine in Step F with bicyclo[2.2.1]heptan-2-amine.

¹H NMR (400 MHz, D₂O) δ 7.82 (s, 1H), 5.18 (d, J=5.5 Hz, 1H), 4.55-4.47 (m, 1H), 4.36-4.23 (m, 2H), 4.19-4.11 (m, 1H), 4.08-3.93 (m, 2H), 2.53 (m, 1H), 2.29-2.01 (m, 4H), 1.59-1.18 (m, 6H), 1.15-0.97 (m, 1H). Mass Spectrum (ESI) m/z=553.8 (M+1).

### Example S51

### Synthesis of (((((2R,3S,4R,1S)-5-(4-((R)-2-(tert-butyl)pyrrolidin-1-yl)-2-chloroimidazo[2,1-f][1,2,4]triazin-7-yl)-3,4-dihydroxytetrahydrofuran-2-yl)methoxy)(hydroxy)phosphoryl)methyl)phosphonic Acid

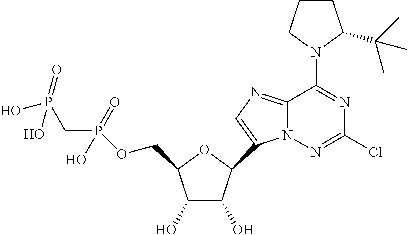 [SMILES: CC(C)(C)[C@H]1CCCN1c1nc(Cl)nn2c([C@@H]3O[C@H](COP(=O)(O)CP(=O)(O)O)[C@@H](O)[C@H]3O)cnc12]

(((((2R,3S,4R,5S)-5-(4-((R)-2-(tert-butyl)pyrrolidin-1-yl)-2-chloroimidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with (R)-2-(tert-butyl)pyrrolidine.

¹H NMR (400 MHz, D₂O) δ 7.75-7.67 (m, 1H), 5.21-5.12 (m, 1H), 4.68 (m, 1H), 4.60-4.45 (m, 2H), 4.36-4.28 (m, 1H), 4.17 (m, 1H), 4.05-3.91 (m, 3H), 2.20-1.83 (m, 6H), 0.84 (d, J=23.2 Hz, 9H). Mass Spectrum (ESI) m/z=567.7 (M−1).

### Example S52

### Synthesis of (((((2R,3S,4R,1S)-5-(2-chloro-4-(((S)-1-(3,4-difluorophenyl)ethyl)amino)imidazo[2,1-f][1,2,4]triazin-7-yl)-3,4-dihydroxytelmhydrofuran-2-yl)methoxy)(hydroxy)phosphoryl)methyl)phosphonic Acid

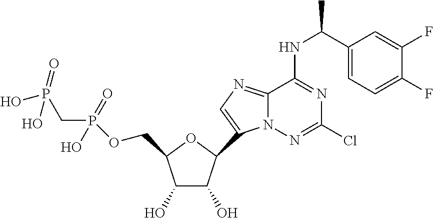 [SMILES: C[C@H](Nc1nc(Cl)nn2c([C@@H]3O[C@H](COP(=O)(O)CP(=O)(O)O)[C@@H](O)[C@H]3O)cnc12)c1ccc(F)c(F)c1]

(((((2R,3S,4R,5S)-5-(2-chloro-4-(((S)-1-(3,4-difluorophenyl)ethyl)amino)imidazo[2,1-f][1,2,4]triazin-7-yl)-3,4-dihydroxytetrahydrofuran-2-yl)methoxy)(hydroxy)phosphoryl)methyl) phosphonic acid was prepared by procedures similar to the one described in Example S5, replacing cyclopentanamine in Step F with (S)-1-(3,4-difluorophenyl)ethan-1-amine.

¹H NMR (400 MHz, D₂O) δ 7.74 (s, 1H), 7.34-7.24 (m, 1H), 7.20-7.10 (m, 2H), 5.31 (d, J=6.8 Hz, 1H), 5.16 (d, J=6.0 Hz, 1H), 4.57-4.52 (m, 1H), 4.34-4.27 (m, 1H), 4.19-4.14 (m, 1H), 4.05-3.95 (m, 2H), 2.24-2.05 (m, 2H), 1.56 (d, J=6.9 Hz, 3H). Mass Spectrum (ESI) m/z=599.8 (M+1).

### Example S53

### Synthesis of (((((2R,3S,4R,1S)-5-(2-chloro-4-(((S)-1-(3,4-dichlorophenyl)ethyl)amino)imidazo[2,1-f][1,2,4]triazin-7-yl)-3,4-dihydroxytetrahydrofuran-2-yl)methoxy)(hydroxy)phosphoryl)methyl)phosphonic Acid

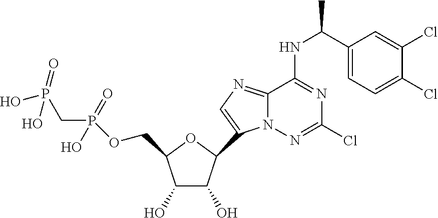 [SMILES: C[C@H](Nc1nc(Cl)nn2c([C@@H]3O[C@H](COP(=O)(O)CP(=O)(O)O)[C@@H](O)[C@H]3O)cnc12)c1ccc(Cl)c(Cl)c1]

(((((2R,3S,4R,5S)-5-(2-chloro-4-(((S)-1-(3,4-dichlorophenyl)ethyl)amino)imidazo[2,1-f][1,2,4]triazin-7-yl)-3,4-dihydroxytetrahydrofuran-2-yl)methoxy)(hydroxy)phosphoryl)methyl)phosphonic acid was prepared by procedures similar to the one described in Example S5, replacing cyclopentanamine in Step F with (S)-1-(3,4-dichlorophenyl)ethan-1-amine.

¹H NMR (400 MHz, D₂O) δ 7.72 (s, 1H), 7.51 (s, 1H), 7.45-7.36 (m, 1H), 7.31-7.21 (m, 1H), 5.31-5.24 (m, 1H), 5.18-5.10 (m, 1H), 4.60-4.48 (m, 1H), 4.35-4.26 (m, 1H), 4.19-4.08 (m, 1H), 4.04-3.94 (m, 2H), 2.25-2.04 (m, 2H), 1.58-1.49 (m, 3H). Mass Spectrum (ESI) m/z=631.6 (M+1).

### Example S54

### Synthesis of (((((2R,3S,4R,1S)-5-(2-chloro-4-(((S)-1-(4-chlorophenyl)ethyl)amino)imidazo[2,1-f][1,2,4]triazin-7-yl)-3,4-dihydroxytetrahydrofuran-2-yl)methoxy)(hydroxy)phosphoryl)methyl)phosphonic Acid

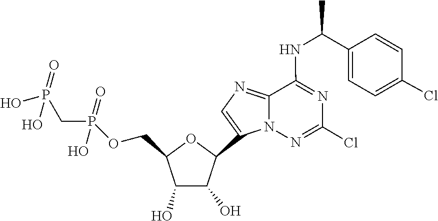 [SMILES: C[C@H](Nc1nc(Cl)nn2c([C@@H]3O[C@H](COP(=O)(O)CP(=O)(O)O)[C@@H](O)[C@H]3O)cnc12)c1ccc(Cl)cc1]

(((((2R,3S,4R,5S)-5-(2-chloro-4-(((S)-1-(4-chlorophenyl)ethyl)amino)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with (S)-1-(4-chlorophenyl)ethan-1-amine.

¹H NMR (400 MHz, D₂O) δ 7.73 (s, 1H), 7.28-7.23 (m, 4H), 5.28-5.25 (m, 1H), 5.12 (m, 1H), 4.53-5.49 (m, 1H), 4.28 (m, 1H), 4.14 (m, 1H), 4.00-3.95 (m, 2H), 2.17 (t, J=19.4 Hz, 2H), 1.55-1.50 (m, 3H). Mass Spectrum (ESI) m/z=597.5 (M+1).

### Example S55

### Synthesis of (((((2R,3S,4R,1S)-5-(2-chloro-4-((S)-2-(2-fluorophenyl)pyrrolidin-1-yl)imidazo[2,1-f][1,2,4]triazin-7-yl)-3,4-dihydroxytetrahydrofuran-2-yl)methoxy)(hydroxy)phosphoryl)methyl)phosphonic Acid

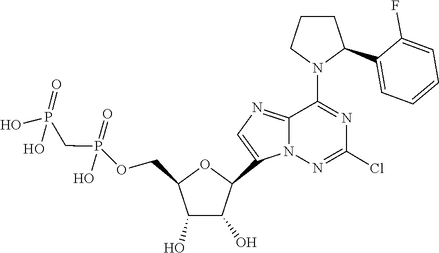 [SMILES: O=P(O)(O)CP(=O)(O)OC[C@H]1O[C@@H](c2cnc3c(N4CCC[C@H]4c4ccccc4F)nc(Cl)nn23)[C@H](O)[C@@H]1O]

(((((2R,3S,4R,5S)-5-(2-chloro-4-((S)-2-(2-fluorophenyl)pyrrolidin-1-yl)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with (S)-2-(2-fluorophenyl)pyrrolidine.

¹H NMR (400 MHz, D₂O) δ 7.82-7.70 (m, 0.5H), 7.92-7.58 (m, 0.5H), 7.43-7.36 (m, 4H), 6.39-6.33 (m, 0.5H), 5.62-5.60 (m, 0.5H), 5.14-5.06 (m, 1H), 4.47-4.45 (m, 1.5H), 4.35-4.31 (m, 1.5H), 4.10-3.99 (m, 1H), 3.90-3.79 (m, 3H), 2.39-2.20 (m, 1H), 2.17-1.80 (m, 5H). Mass Spectrum (ESI) m/z=607.5 (M+1).

### Example S56

### Synthesis of (((((2R,3S,4R,1S)-5-(2-chloro-4-((S)-2-phenylpiperidin-1-yl)imidazo[2,1-f][1,2,4]triazin-7-yl)-3,4-dihydroxytetrahydrofuran-2-yl)methoxy)(hydroxy)phosphoryl)methyl)phosphonic Acid

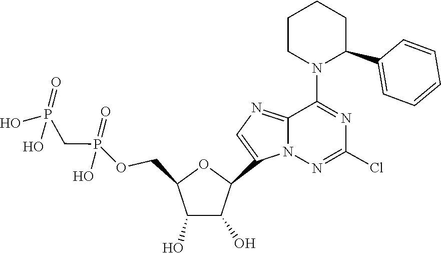 [SMILES: O=P(O)(O)CP(=O)(O)OC[C@H]1O[C@@H](c2cnc3c(N4CCCC[C@H]4c4ccccc4)nc(Cl)nn23)[C@H](O)[C@@H]1O]

(((((2R,3S,4R,5S)-5-(2-chloro-4-((S)-2-phenylpiperidin-1-yl)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with (S)-2-phenylpiperidine.

¹H NMR (400 MHz, DMSO) δ 7.82-7.75 (m, 0.5H), 7.75-7.70 (m, 0.5H), 7.69-7.64 (m, 0.5H), 7.34-7.30 (m, 5H), 6.31-6.28 (m, 0.5H), 6.22-6.20 (m, 0.5H), 5.13-5.05 (m, 1H), 4.86-4.85 (m, 0.5H), 4.36-4.26 (m, 1H), 4.02-3.99 (m, 4H), 3.15-3.10 (m, 0.5H), 2.81-2.80 (m, 0.5H), 2.59 (m, 1H), 2.20-2.18 (m, 2H), 1.99-1.80 (m, 1H), 1.77-1.49 (m, 4H). Mass Spectrum (ESI) m/z=603.6 (M+1).

### Example S57

### Synthesis of ((2R,3S,4R,5S)-5-(2-chloro-4-(cyclopentylamino)imidazo[2,1-f][1,2,4]triazin-7-yl)-3,4-dihydroxytetrahydrofuran-2-yl)methyl methyl ((dimethoxyphosphoryl)methyl)phosphonate

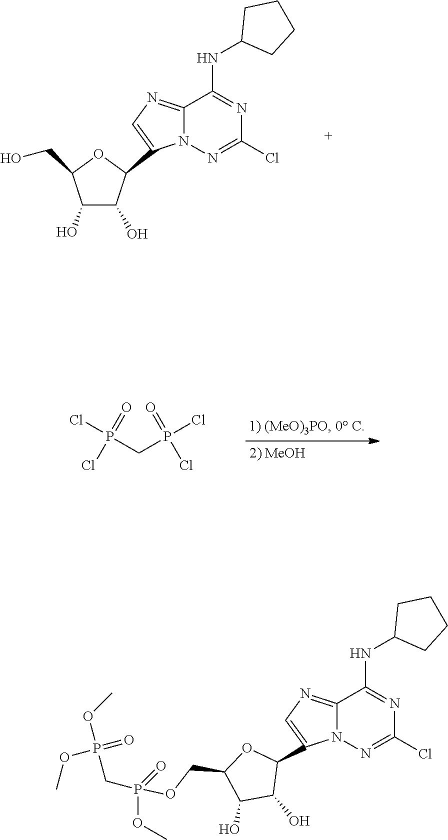 [SMILES: OC[C@H]1O[C@@H](c2cnc3c(NC4CCCC4)nc(Cl)nn23)[C@H](O)[C@@H]1O ; O=P(Cl)(Cl)CP(=O)(Cl)Cl ; COP(=O)(CP(=O)(OC)OC[C@H]1O[C@@H](c2cnc3c(NC4CCCC4)nc(Cl)nn23)[C@H](O)[C@@H]1O)OC]

To a solution of (2S,3R,4S,5R)-2-[2-chloro-4-(cyclopentylamino)imidazo[2,1-f][1,2,4]triazin-7-yl]-5-(hydroxymethyl)oxolane-3,4-diol (100 mg, 0.27 mmol) in trimethylphosphate (1 mL) at 0° C. was added a cold solution of [(dichlorophosphoryl)methyl]phosphonoyl dichloride (337 mg, 1.35 mmol) in trimethylphosphate (1 mL) dropwise. The reaction solution was stirred at 0° C. for 4 h. Then MeOH (6 mL) was added to the reaction and the resulting solution was stirred at rt for another 2 h. The solution was concentrated and purified by Prep-HPLC (Daisogel-C18 250×50 mm, 10 um column, 0.2% FA in water/MeCN=from 85% to 55%) to give ((2R,3S,4R,5S)-5-(2-chloro-4-(cyclopentylamino)imidazo[2,1-f][1,2,4]triazin-7-yl)-3,4-dihydroxytetrahydrofuran-2-yl)methyl methyl ((dimethoxyphosphoryl)methyl)phosphonate (22 mg, 14% yield) as a solid.

¹H NMR (400 MHz, DMSO) δ 9.42 (d, J=7.7 Hz, 1H), 7.68 (d, J=2.8 Hz, 1H), 5.27 (t, J=5.9 Hz, 1H), 5.23-5.20 (m, 1H), 5.05 (d, J=5.9 Hz, 1H), 4.53-4.46 (m, 1H), 4.34-4.30 (m, 1H), 4.21-4.14 (m, 1H), 4.12-4.07 (m, 1H), 4.05-4.01 (m, 2H), 3.71-3.62 (m, 9H), 2.88-2.74 (m, 2H), 1.99-1.91 (m, 2H), 1.74-1.56 (m, 6H). Mass Spectrum (ESI) m/z=569.6 (M+1).

### Example S58

### Synthesis of ((2R,3S,4R,5S)-5-(2-chloro-4-(cyclopentylamino)imidazo[2,1-f][1,2,4]triazin-7-yl)-3,4-dihydroxytetrahydrofuran-2-yl)methyl hydrogen (((4S)-4-(3-chlorophenyl)-2-oxido-1,3,2-dioxaphosphinan-2-yl)methyl)phosphonate

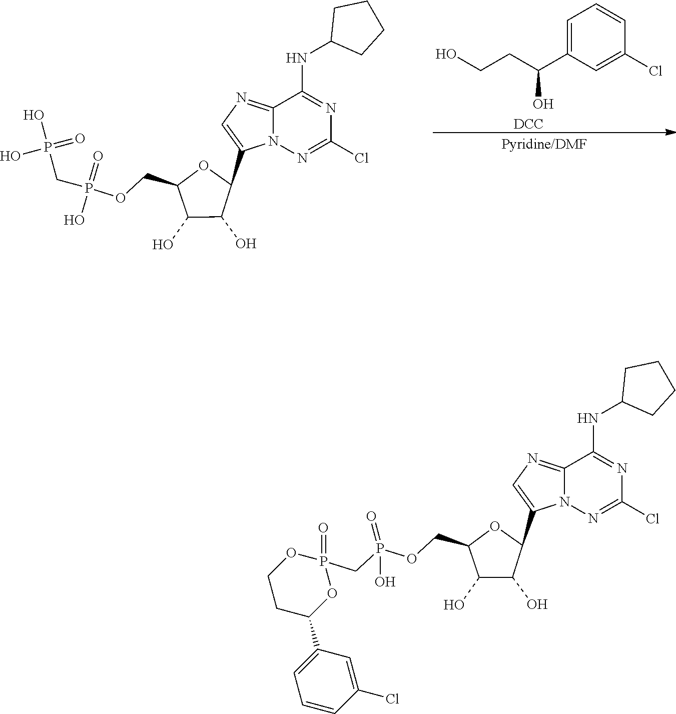 [SMILES: O=P(O)(O)CP(=O)(O)OC[C@H]1O[C@@H](c2cnc3c(NC4CCCC4)nc(Cl)nn23)[C@H](O)[C@@H]1O ; OCC[C@H](O)c1cccc(Cl)c1 ; "DCC" ; O=P(O)(CP1(=O)OCC[C@@H](c2cccc(Cl)c2)O1)OC[C@H]1O[C@@H](c2cnc3c(NC4CCCC4)nc(Cl)nn23)[C@H](O)[C@@H]1O]

To a solution of [(([(2R,3S,4R,5S)-5-[2-chloro-4-(cyclopentylamino) imidazo[2,1-f][1,2,4]triazin-7-yl]-3,4-dihydroxyoxolan-2-yl]methoxy)(hydroxy)phosphoryl)methyl] phosphonic acid (230 mg, 0.44 mmol) and (1S)-1-(3-chlorophenyl)propane-1,3-diol (238 mg, 1.28 mmol) in DMF (5 mL) and pyridine (1 mL) was added DCC (263 mg, 1.28 mmol), then the mixture was stirred at 70° C. for 4 h. The mixture was concentrated and purified by Prep-TLC (DCM/MeOH=10:1) to give ((2R,3S,4R,5S)-5-(2-chloro-4-(cyclopentylamino)imidazo[2,1-f][1,2,4]triazin-7-yl)-3,4-dihydroxytetrahydrofuran-2-yl)methyl hydrogen (((4S)-4-(3-chlorophenyl)-2-oxido-1,3,2-dioxaphosphinan-2-yl)methyl)phosphonate (6 mg, 2% yield) as a white solid.

¹H NMR (400 MHz, DMSO) δ 9.37 (d, J=7.5 Hz, 1H), 7.73-7.58 (m, 1H), 7.50-7.21 (m, 4H), 5.88-5.81 (m, 0.5H), 5.61-5.53 (m, 0.5H), 5.05-4.98 (m, 1H), 4.76-4.64 (m, 1H), 4.53-4.41 (m, 2H), 4.33-4.22 (m, 2H), 4.08-4.03 (m, 1H), 4.00-3.84 (m, 4H), 2.50-2.28 (m, 2H), 2.25-1.89 (m, 3H), 1.79-1.51 (m, 6H). Mass Spectrum (ESI) m/z=569.6 (M+1).

### Example S59

### Synthesis of ((2R,3S,4R,5S)-5-(2-chloro-4-(cyclopentylamino)imidazo[2,1-f][1,2,4]triazin-7-yl)-3,4-dihydroxytetrahydrofuran-2-yl)methyl methyl (((4S)-4-(3-chlorophenyl)-2-oxido-1,3,2-diaxaphosphinan-2-yl)methyl)phosphonate

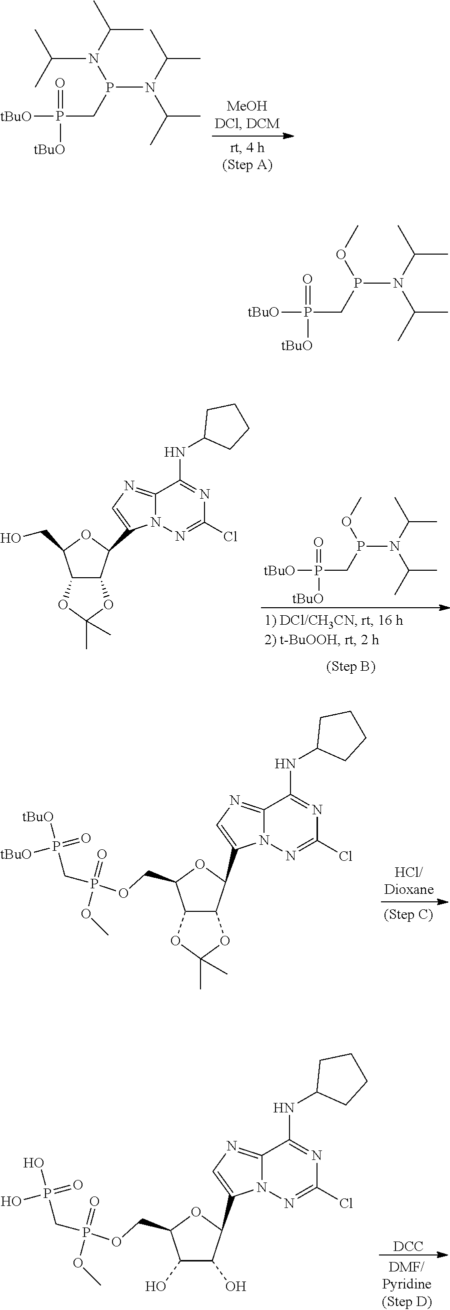

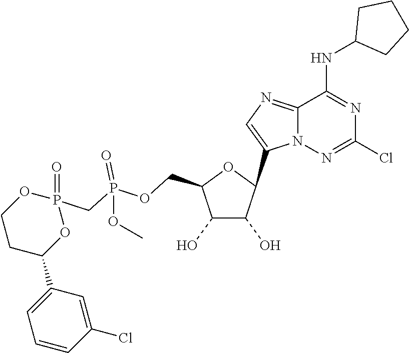 [SMILES: COP(=O)(CP1(=O)OCC[C@@H](c2cccc(Cl)c2)O1)OC[C@H]1O[C@@H](c2cnc3c(NC4CCCC4)nc(Cl)nn23)[C@H](O)[C@@H]1O]

Step A: To a solution of di-tert-butyl {[bis(diisopropylamino)phosphanyl]methyl} phosphonate (2 g, 4.56 mmol) in DCM (20 mL) was added MeOH (146.1 mg, 4.56 mmol) and DCI (323 mg, 2.74 mmol). Then the mixture was stirred at rt for 1 h and monitored by TLC. Once complete, the mixture was concentrated and purified by column chromatography on silica gel (EA/1% TEA in PE=5:1) to give di-tert-butyl {[(diisopropylamino)(methoxy)phosphanyl] methyl}phosphonate (1.5 g, 80% yield) as a colorless oil. Mass Spectrum (ESI) m/z=370.2 (M+1).

Step B: To a solution of [(3aR,4R,6S,6aS)-6-[2-chloro-4-(cyclopentylamino)imidazo[2,1-f][1,2,4]triazin-7-yl]-2,2-dimethyl-tetrahydrofuro[3,4-d][1,3]dioxol-4-yl]methanol (1 g, 2.44 mmol) and di-tert-butyl {[(diisopropylamino)(methoxy) phosphanyl]methyl}phosphonate (1.8 g, 4.88 mmol) in MeCN (10 mL) was added DCI (576 mg, 4.88 mmol). The mixture was stirred at rt overnight. Then t-BuOOH (10 eq) was added and the reaction was stirred for another 1 h. EA (20 mL) was added, and the organic layer was washed with aqueous Na₂CO₃ (20 mL×4), dried over Na₂SO₄, filtered and the filtrate was concentrated. The residue was purified by CombiFlash® (MeOH/DCM=0-3%) to give the product (1.2 g, 71% yield) as a colorless oil. Mass Spectrum (ESI) m/z=581.6 (M−111).

Step C: To a solution of [(3aR,4R,6S,6aS)-6-[2-chloro-4-(cyclopentylamino)imidazo[2,1-f][1,2,4]triazin-7-yl]-2,2-dimethyl-tetrahydrofuro[3,4-d][1,3]dioxol-4-yl]methyl methyl {[bis(tert-butoxy)phosphoryl]methyl}phosphonate (700 mg, 1.01 mmol) in 1,4-dioxane (7 mL) was added a solution of HCl in dioxane (4M, 1.75 mL). The mixture was stirred at rt for 2 h. Then it was concentrated and purified by Prep-HPLC (Daisogel-C18 250×50 mm, 10 um column, 0.2% FA in water/MeCN from 70% to 50%) to give (((((2R,3S,4R,5S)-5-(2-chloro-4-(cyclopentylamino)imidazo[2,1-f][1,2,4]triazin-7-yl)-3,4-dihydroxytetrahydrofuran-2-yl)methoxy)(methoxy)phosphoryl)methyl)phosphonic acid (130 mg, 24% yield) as a white solid. Mass Spectrum (ESI) m/z=541.6 (M+1).

Step D: To a solution of [({[(2R,3S,4R,5S)-5-[2-chloro-4-(cyclopentylamino)imidazo[2,1-f][1,2,4]triazin-7-yl]-3,4-dihydroxyoxolan-2-yl]methoxy}(methoxy)phosphoryl)methyl]phosphonic acid (100 mg, 0.18 mmol) and (1S)-1-(3-chlorophenyl)propane-1,3-diol (97 mg, 0.52 mmol) in DMF (5 mL) and pyridine (1 mL) was added DCC (107 mg, 0.06 mmol). Then the mixture was stirred at 70° C. for 4 h. The reaction was concentrated and purified by Prep-TLC (DCM/MeOH=10:1) to give ((2R,3S,4R,5S)-5-(2-chloro-4-(cyclopentylamino)imidazo[2,1-f][1,2,4]triazin-7-yl)-3,4-dihydroxytetrahydrofuran-2-yl)methyl methyl (((4S)-4-(3-chlorophenyl)-2-oxido-1,3,2-dioxaphosphinan-2-yl)methyl)phosphonate (30 mg, 23% yield) as an off-white solid.

¹H NMR (400 MHz, DMSO) δ 9.40 (m, 1H), 7.67 (d, J=7.8 Hz, 1H), 7.58 (d, J=6.6 Hz, 1H), 7.45-7.33 (m, 3H), 5.65 (m, 1H), 5.23 (d, J=20.6 Hz, 2H), 5.04 (d, J=5.9 Hz, 1H), 4.50 (m, 2H), 4.34 (m, 2H), 4.19 (m, 1H), 4.14-4.07 (m, 1H), 4.02 (m, 2H), 3.69-3.62 (m, 3H), 3.01-2.98 (m, 2H), 2.21-1.90 (m, 4H), 1.64 (m, 6H). Mass Spectrum (ESI) m/z=691.5 (M+1).

### Example S60

### Synthesis of (((((2R,3S,4R,1S)-5-(4-(cyclopentylamino)-2-cyclopropylimidazo[2,1-f][1,2,4]triazin-7-yl)-3,4-dihydroxytetrahydrofuran-2-yl)methoxy)(hydroxy)phosphoryl)methyl)phosphonic Acid

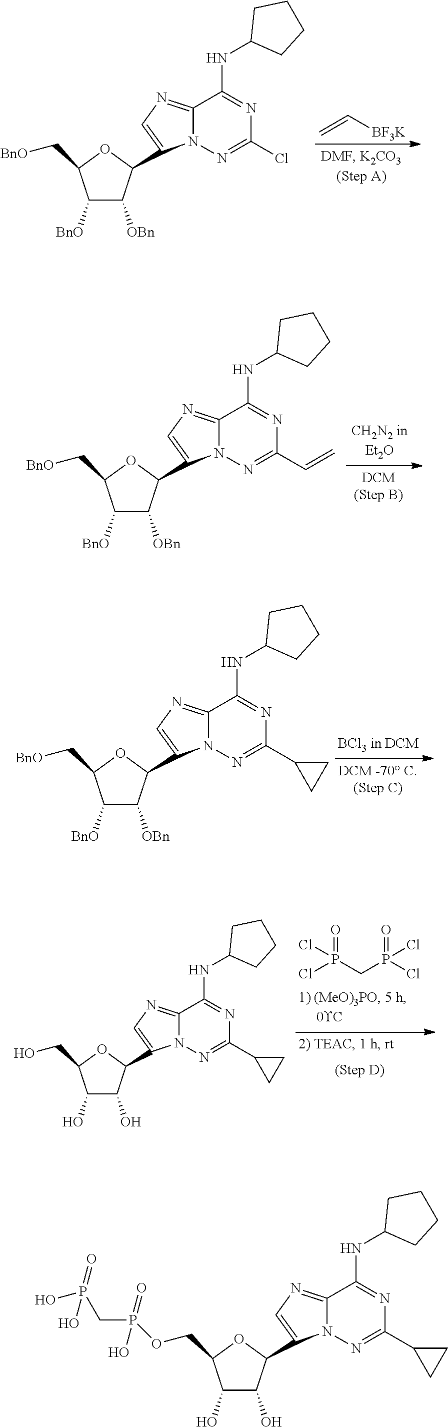

Step A: To a solution of potassium vinyltrifluoroborate (188 mg, 1.4 mmol), 7-[(2S,3S,4R,5R)-3,4-bis(benzyloxy)-5-[(benzyloxy)methyl]oxolan-2-yl]-2-chloro-N-cyclopentylimidazo[2,1-f][1,2,4]triazin-4-amine (0.6 g, 0.93 mmol) and K₂CO₃ (258 mg, 1.87 mmol) in DMF (2 mL) was added Pd(PPh₃)₄ (108 mg, 0.09 mmol). The reaction mixture was stirred at 120° C. for 18 hours under N₂ atmosphere. After cooling, water was added to the reaction, and the mixture was extracted with DCM (20 mL×2). The combined organic layers were washed with brine, dried, filtered and the filtrate was concentrated and purified by CombiFlash® (40 g, EA/PE=0-20%) to give 7-[(2S,3S,4R,5R)-3,4-bis(benzyloxy)-5-[(benzyloxy)methyl]oxolan-2-yl]-N-cyclopentyl-2-ethenylimidazo[2,1-f][1,2,4]triazin-4-amine (0.4 g, 60% yield) as a yellow oil. Mass Spectrum (ESI) m/z=631.9 (M+1).

Step B: To a solution of 7-[(2S,3S,4R,5R)-3,4-bis(benzyloxy)-5-[(benzyloxy)methyl]oxolan-2-yl]-N-cyclopentyl-2-ethenylimidazo[2,1-f][1,2,4]triazin-4-amine (250 mg, 0.39 mmol) in DCM (1 mL) was added CH₂N₂ in Et₂O (10 ml) portion wise. Then the mixture was stirred at rt for 15 h. The organic layer was washed with brine, dried, filtered and the filtrate was concentrated and purified by CombiFlash® (12 g, EA/PE=0-6%) to give 7-bromo-2,4-dichloroimidazo[2,1-f][1,2,4]triazine (130 mg, 50% yield) as na oil. Mass Spectrum (ESI) m/z=645.9 (M+1).

Step C and Step D: 7-[(2S,3S,4R,5R)-3,4-bis(benzyloxy)-5-[(benzyloxy)methyl]oxolan-2-yl]-N-cyclopentyl-2-cyclopropylimidazo[2,1-f][1,2,4]triazin-4-amine was converted to the title compound by procedures similar to the ones described in Example S5, Step I and Step J.

¹H NMR (400 MHz, D₂O) δ 7.54 (s, 1H), 5.19 (m, 1H), 4.56 (m, 1H), 4.45-4.37 (m, 1H), 4.32 (m, 1H), 4.17-4.10 (m, 1H), 4.00-3.90 (m, 2H), 2.10-1.87 (m, 5H), 1.71-1.48 (m, 6H), 1.02-0.95 (m, 2H), 0.93-0.85 (m, 2H). Mass Spectrum (ESI) m/z=532.1 (M−1).

### Example S61

### Synthesis of (((((2R,3S,4R,1S)-5-(4-(cyclopentyloxy)imidazo[2,1-f][1,2,4]triazin-7-yl)-3,4-dihydroxytetrahydrofuran-2-yl)methoxy)(hydroxy)phosphoryl)methyl)phosphonic Acid

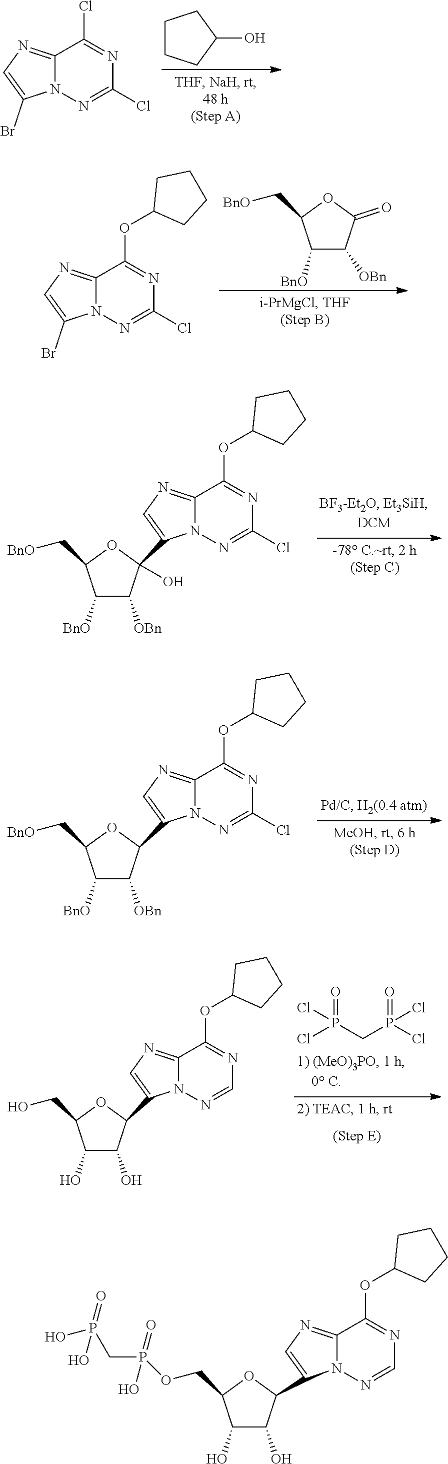

Step A: To a mixture of cyclopentanol (642.5 mg, 7.46 mmol) in THF (50 mL) was added sodium hydride (194 mg, 4.85 mmol) carefully at 0° C. under nitrogen. The reaction was stirred for 30 min at 0° C. Then a solution of 7-bromo-2,4-dichloroimidazo[2,1-f][1,2,4]triazine (1 g, 3.73 mmol) in THF (50 mL) was added carefully at 0° C. under nitrogen. The reaction was stirred for 48 h at 40° C. The reaction was quenched by sat. NH₄Cl solution and extracted with EA (100 mL×3). The organic layer was washed with brine and dried over Na₂SO₄, filtered and the filtrate was concentrated and purified by silica gel column chromatography (40 g, PE/EA=2:1) to give ethyl 7-bromo-2-chloro-4-(cyclopentyloxy)imidazo[2,1-f][1,2,4]triazine (750 mg, 57.1% yield) as a yellow solid. Mass Spectrum (ESI) m/z=319.0 (M+1).

Step B: To a mixture of 7-bromo-2-chloro-4-(cyclopentyloxy)imidazo[2,1-f][1,2,4]triazine (550 mg, 1.73 mmol) in THF (10 mL) was added isopropylmagnesium chloride—lithium chloride complex (1.3 M, 1.73 mL, 2.25 mmol) carefully at −10° C. under nitrogen. Then a solution of (3R,4R,5R)-3,4-bis(benzyloxy)-5-[(benzyloxy)methyl]oxolan-2-one (869 mg 2.08 mmol) in THF (5 mL) was added at −10° C. under nitrogen. The reaction was stirred for 2 h at rt. The reaction was quenched by addn. of sat. aq. NH₄Cl solution and the mixture was extracted with EA (25 mL×3). The organic layer was washed with brine and dried over Na₂SO₄, filtered and the filtrate was concentrated and purified by silica gel column chromatography (40 g, PE/EA=3:1) to give (2S,3R,4R,5R)-3,4-bis(benzyloxy)-5-[(benzyloxy)methyl]-2-[2-chloro-4-(cyclopentyloxy) imidazo[2,1-f][1,2,4]triazin-7-yl]oxolan-2-ol (500 mg, 39.3% yield) as a yellow solid. Mass Spectrum (ESI) m/z=657.1 (M+1).

Step C: To a mixture of (2S,3R,4R,5R)-3,4-bis(benzyloxy)-5-[(benzyloxy)methyl]-2-[2-chloro-4-(cyclopentyloxy)imidazo[2,1-f][1,2,4]triazin-7-yl]oxolan-2-ol (500 mg, 0.76 mmol) in DCM (15 mL) was added boron trifluoride etherate (230 mg, 7.6 mmol, 47%) carefully at −78° C. under nitrogen. Then triethylsilane (884 mg, 7.6 mmol) was added into the reaction at −78° C. under nitrogen. The reaction was stirred for 2 h at rt. The organic layer was washed with sat. NaHCO₃ solution and brine, dried over Na₂SO₄, filtered and the filtrate was concentrated and purified by silica gel column chromatography (40 g, PE/EA=2:1) to give 7-[(2S,3S,4R,5R)-3,4-bis(benzyloxy)-5-[(benzyloxy)methyl]oxolan-2-yl]-2-chloro-4-(cyclopentyloxy)imidazo[2,1-f][1,2,4]triazine (260 mg, 47% yield) as a yellow solid. Mass Spectrum (ESI) m/z=641.1 (M+1).

Step D: To a mixture of 7-[(2S,3S,4R,5R)-3,4-bis(benzyloxy)-5-[(benzyloxy)methyl]oxolan-2-yl]-2-chloro-4-(cyclopentyloxy)imidazo[2,1-f][1,2,4]triazine (120 mg, 0.36 mmol) in MeOH (20 mL) was added Pd/C (20 mg, 10%). The reaction was stirred for 6 h at rt under an atmosphere of hydrogen (0.4 atm). Then the reaction was filtered and the filtrate was concentrated to give (2S,3R,4S,5R)-2-[4-(cyclopentyloxy)imidazo[2,1-f][1,2,4]triazin-7-yl]-5-(hydroxymethyl)oxolane-3,4-diol (50 mg, 68%) as a yellow solid. Mass Spectrum (ESI) m/z=337.2 (M+1).

Step E: (2S,3R,4S,5R)-2-[4-(cyclopentyloxy)imidazo[2,1-f][1,2,4]triazin-7-yl]-5-(hydroxymethyl)oxolane-3,4-diol was converted to the title compound by a procedure similar to the one described in Example S5, Step J.

¹H NMR (400 MHz, D₂O) δ 8.30 (s, 1H), 7.82 (s, 1H), 5.65-5.59 (m, 1H), 5.28 (d, J=6.8 Hz, 1H), 4.66-4.61 (m, 1H), 4.38-4.33 (m, 1H), 4.21-4.18 (m, 1H), 4.03-3.96 (m, 2H), 2.09-1.87 (m, 6H), 1.79-1.72 (m, 2H), 1.67-1.59 (m, 2H). Mass Spectrum (ESI) m/z=495.0 (M+1).

### Example S62

### Synthesis of (((((2R,3S,4R,5S)-5-(2-cyano-4-(cyclopentylamino)imidazo[2,1-f][1,2,4]triazin-7-yl)-3,4-dihydroxytetrahydrofuran-2-yl)methoxy)(hydroxy)phosphoryl)methyl)phosphonic Acid

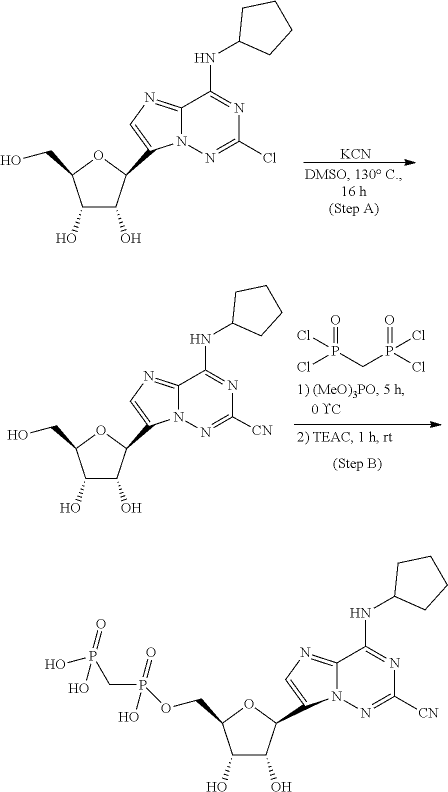

Step A: To a solution of (2S,3R,4S,5R)-2-[2-chloro-4-(cyclopentylamino)imidazo[2,1-f][1,2,4]triazin-7-yl]-5-(hydroxymethyl)oxolane-3,4-diol (105 mg, 0.28 mmol) in DMSO (1 mL) was added KCN (120 mg, 1.84 mmol). The reaction mixture was stirred at 130° C. for 16 hours under a N₂ atmosphere. After cooling to rt, water was added, and the mixture was extracted with EA (20 mL×2). The combined organic layers were washed with brine, dried, filtered and the filtrate was concentrated and purified by CombiFlash® (4 g, DCM/MeOH=0-10%) to give 4-(cyclopentylamino)-7-[(2S,3R,4S,5R)-3,4-dihydroxy-5-(hydroxymethyl)oxolan-2-yl]imidazo[2,1-f][1,2,4]triazine-2-carbonitrile (50 mg, 49% yield) as a yellow solid. Mass Spectrum (ESI) m/z=631.1 (M+1).

Step B: (4-(Cyclopentylamino)-7-[(2S,3R,4S,5R)-3,4-dihydroxy-5-(hydroxymethyl)oxolan-2-yl]imidazo[2,1-f][1,2,4]triazine-2-carbonitrile was converted to the title compound by a procedure similar to the one described in Example S5, Step J.

¹H NMR (400 MHz, D₂O) δ 7.72 (s, 1H), 5.19 (d, J=6.4 Hz, 1H), 4.57-4.51 (m, 1H), 4.43-4.37 (m, 1H), 4.33 (m, 1H), 4.19-4.13 (m, 1H), 4.02-3.93 (m, 2H), 2.10-1.90 (m, 4H), 1.79-1.49 (m, 6H). Mass Spectrum (ESI) m/z=517.1 (M−1).

### Example S63

### Synthesis of [({[(2R,3S,4R,1S)-5-{2-chloro-4-[(1S)-2,3-dihydro-1H-inden-1-ylamino]imidazo[2,1-f][1,2,4]triazin-7-yl}-3,4-dihydroxyaxolan-2-yl]methoxy}(hydroxy)phosphoryl)methyl]phosphonic Acid

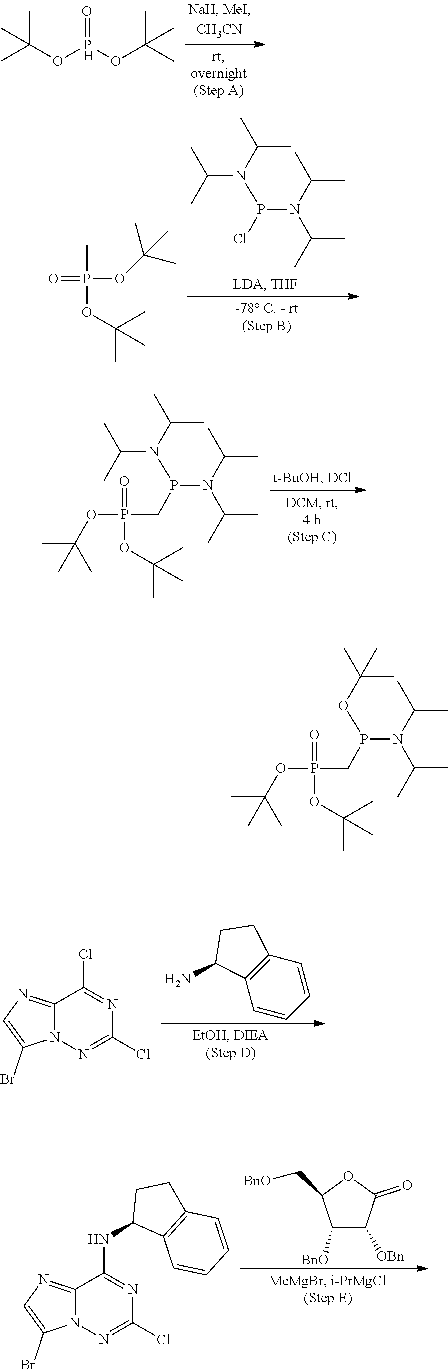

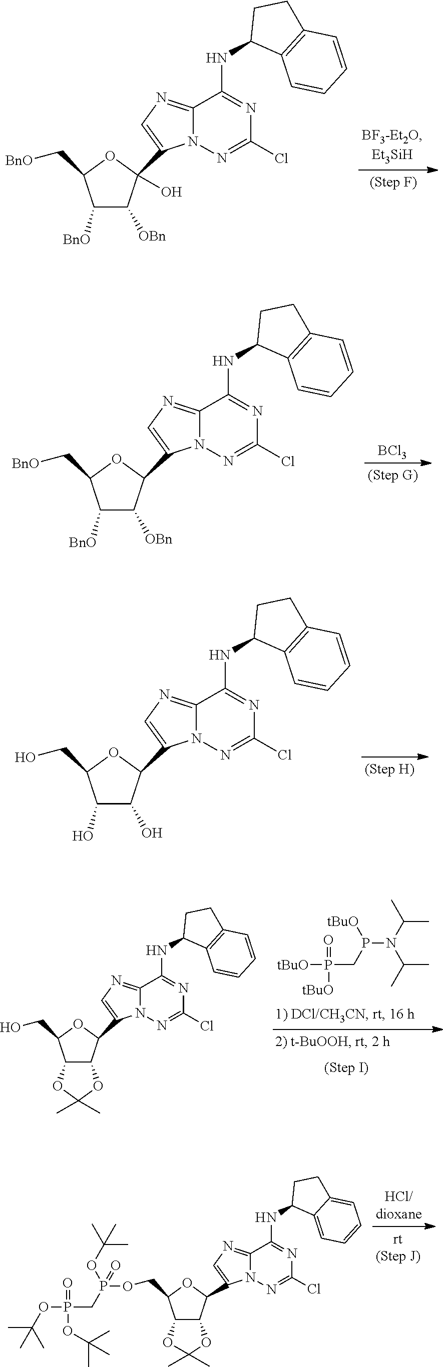

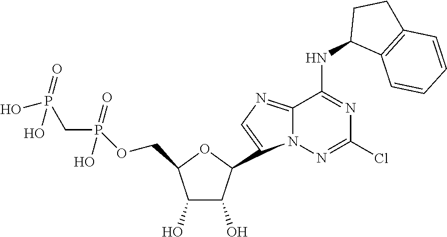 [SMILES: O=P(O)(O)CP(=O)(O)OC[C@H]1O[C@@H](c2cnc3c(N[C@H]4CCc5ccccc54)nc(Cl)nn23)[C@H](O)[C@@H]1O]

Step A: To a mixture of di-tert-butyl phosphonate (10.0 g, 51.5 mmol) in acetonitrile (40 mL) was added NaH (3.1 g, 77.2 mmol) carefully at 0° C. under nitrogen. The reaction was stirred for 30 min at rt. Then a solution of iodomethane (11.0 g, 77.2 mmol) in acetonitrile (10 mL) was added dropwise at rt under nitrogen. The reaction was stirred at rt overnight. The reaction was quenched by water (1 ml) and concentrated. The crude product was purified by column chromatography on silica gel (400 g, PE/EA=1:1) to give di-tert-butyl methylphosphonate (10 g, 84% yield) as a yellow oil. ¹H NMR (400 MHz, DMSO) δ 1.43 (s, 18H), 1.33 (d, J=17.2 Hz, 3H).

Step B: To a mixture of diisopropylamine (8.2 g, 80.7 mmol) in THF (30 mL) was added n-BuLi (33.6 mL, 80.7 mmol, 2.4M) at −78° C. under nitrogen. The reaction was stirred for 30 min at −78° C. Then di-tert-butyl methylphosphonate (8 g, 38.4 mmol) was added carefully at −78° C. under nitrogen. The reaction was stirred for 30 mi at −78° C. Then a solution of 1-chloro-N,N,N′,N′-tetraisopropylphosphanediamine (10.25 g, 38.4 mmol) in THF (70 mL) was added. The reaction was stirred and warmed to rt overnight. The reaction was quenched by sat. NaHCO₃ solution and extracted with EA (100 mL×3). The combined organic layers were washed with brine and dried over Na₂SO₄, filtered and the filtrate was concentrated. The crude product was purified by column chromatography on silica gel (800 g, eluting with PE/EA=20:1+1% TEA) to give di-tert-butyl ((bis(diisopropylamino)phosphanyl)methyl)phosphonate (12 g, 64.1% yield) as a yellow oil.

¹H NMR (400 MHz, CDCl₃) δ 3.47-3.31 (m, 4H), 2.18-2.04 (m, 2H), 1.56-1.49 (s, 18H), 1.19 (t, J=8.1 Hz, 24H).

Step C: To a mixture of di-tert-butyl {[bis(diisopropylamino)phosphanyl]methyl} phosphonate (4.0 g, 9.12 mmol) and 2-methylpropan-2-ol (676 mg, 9.12 mmol) in DCM (40 mL) was added 1H-imidazole-4,5-dicarbonitrile (646 mg, 5.47 mmol) carefully. The reaction was stirred for 4 h at rt. Then the reaction was concentrated. The crude product was purified by column chromatography on silica gel (400 g, eluting with PE/EA=10:1+1% TEA) to give di-tert-butyl ((tert-butoxy(diisopropylamino)phosphanyl)methyl)phosphonate (2.5 g, 60.0% yield) as a yellow oil. ¹H NMR (400 MHz, CDCl₃) δ 3.68-3.36 (m, 2H), 2.26-2.13 (m, 1H), 1.96-1.84 (m, 1H), 1.53 (d, J=1.4 Hz, 18H), 1.33 (s, 9H), 1.20 (d, J=6.7 Hz, 6H), 1.14 (d, J=6.8 Hz, 6H).

Step D: To a solution of 7-bromo-2,4-dichloroimidazo[2,1-f][1,2,4]triazine (8 g, 0.029 mol) and (1S)-2,3-dihydro-1H-inden-1-amine hydrochloride (5.6 g, 0.032 mol) in THF was added N,N-diisopropylethylamine (7.7 g, 0.059 mol). The reaction mixture was stirred at rt for 2 hours. The solvent was removed under reduced pressure, and the crude residue was purified by column chromatography on silica gel (PE/EA=3:1) to get 7-bromo-2-chloro-N-[(1S)-2,3-dihydro-1H-inden-1-yl]imidazo[2,1-f][1,2,4]triazin-4-amine (10 g, 83% yield) as a yellow solid. Mass Spectrum (ESI) m/z=364.6 (M+1)

Step E: To a solution of 7-bromo-2-chloro-N-[(1S)-2,3-dihydro-1H-inden-1-yl]imidazo[2,1-f][1,2,4]triazin-4-amine (4.5 g, 0.012 mol) in 50 mL anhydrous THF was added methyl magnesium bromide (3.0 mol/L, 5 mL, 0.015 mol), followed by isopropyl magnesium chloride-lithium chloride complex (16.2 mL, 0.020 mol). Then a solution of (3R,4R,5R)-3,4-bis(benzyloxy)-5-[(benzyloxy)methyl]oxolan-2-one (5.7 g, 0.013 mol) in THF (5 mL) was carefully added. The reaction mixture was stirred for 3 h at −78° C. After quenching with sat. aq. NH₄Cl solution, the mixture was extracted with EA. The organic layer was dried over anhydrous Na₂SO₄, filtered, and the filtrate was concentrated and purified by column chromatography on silica gel (PE/EA=4:6) to give (2S,3R,4R,5R)-3,4-bis(benzyloxy)-5-[(benzyloxy)methyl]-2-{2-chloro-4-[(1S)-2,3-dihydro-1H-inden-1-ylamino]imidazo[2,1-f][1,2,4]triazin-7-yl}oxolan-2-ol (4.3 g, 45% yield) as a yellow oil. Mass Spectrum (ESI) m/z=704.6 (M+1).

Step F: To a solution of (2S,3R,4R,5R)-3,4-bis(benzyloxy)-5-[(benzyloxy)methyl]-2-{2-chloro-4-[(1S)-2,3-dihydro-1H-inden-1-ylamino]-6H,7H-imidazo[2,1-f][1,2,4]triazin-7-yl}oxolan-2-ol (4.3 g, 6.1 mmol) in anhydrous DCM, triethylsilane (2.9 mL, 0.024 mol) and boron trifluoride diethyl etherate (3 mL, 0.024 mol) was added. The reaction was stirred for 1 h at −78° C. and allowed to warm up to rt. After quenching with sat. aq. NaHCO₃ solution, the mixture was extracted with EA. The organic layer was dried over anhydrous Na₂SO₄, filtered, and the filtrate was concentrated and purified by column chromatography on silica gel (PE/EA=20:80) to give 7-[(2S,3S,4R,5R)-3,4-bis(benzyloxy)-5-[(benzyloxy)methyl]oxolan-2-yl]-2-chloro-N-[(1S)-2,3-dihydro-1H-inden-1-yl]imidazo[2,1-f][1,2,4]triazin-4-amine (4.0 g, 85% yield) as a yellow oil. Mass Spectrum (ESI) m/z=687.6 (M+1).

Step G: To a solution of 7-[(2S,3S,4R,5R)-3,4-bis(benzyloxy)-5-[(benzyloxy)methyl]oxolan-2-yl]-2-chloro-N-[(1S)-2,3-dihydro-1H-inden-1-yl]imidazo[2,1-f][1,2,4]triazin-4-amine (4.3 g, 6.2 mmol) in DCM (50 mL) was added boron trichloride (1 M in DCM, 62.5 mL, 0.062 mol) at −78° C. The reaction was stirred at this temperature for 1.5 h. Then the reaction was quenched by adding a mixture of methanol:chloroform (2:1, 50 mL). After the reaction mixture reached to rt, it was neutralized with NH₃ in methanol (10%, 100 mL) and concentrated. The residue was purified by flash chromatography (MeOH/DCM=10:90) to give (2S,3R,4S,5R)-2-{2-chloro-4-[(1S)-2,3-dihydro-1H-inden-1-ylamino]imidazo[2,1-f][1,2,4]triazin-7-yl}-5-(hydroxymethyl)oxolane-3,4-diol (1.9 g, 66% yield) as a white solid. Mass Spectrum (ESI) m/z=417.8 (M+1).

Step H: To a solution of (2S,3R,4S,5R)-2-{2-chloro-4-[(1S)-2,3-dihydro-1H-inden-1-ylamino]imidazo[2,1-f][1,2,4]triazin-7-yl}-5-(hydroxymethyl)oxolane-3,4-diol (3 g, 7.19 mmol) in 25 mL acetone, 2,2-dimethoxypropane (15 g, 0.144 mol) and p-toluenesulfonic acid (1.54 g, 0.009 mol) were added. The reaction mixture was stirred for 16 h at rt. After quenching with sat. aq. NaHCO₃, the mixture was extracted with EA. The organic layer was dried over anhydrous Na₂SO₄, filtered, and the filtrate was concentrated and purified flash chromatography (PE/EA=1:1) to give [(3aR,4R,6S,6aS)-6-{2-chloro-4-[(1S)-2,3-dihydro-1H-inden-1-ylamino]imidazo[2,1-f][1,2,4]triazin-7-yl}-2,2-dimethyl-tetrahydrofuro[3,4-d][1,3]dioxol-4-yl]methanol (2.6 g, 71% yield) as a white solid. Mass Spectrum (ESI) m/z=457.8 (M+1).

Step I: To a mixture of [(3aR,4R,6S,6aS)-6-{2-chloro-4-[(1S)-2,3-dihydro-1H-inden-1-ylamino]imidazo[2,1-f][1,2,4]triazin-7-yl}-2,2-dimethyl-tetrahydrofuro[3,4-d][1,3]dioxol-4-yl]methanol (500 mg, 1.09 mmol) and di-tert-butyl {[(tert-butoxy)(diisopropylamino)phosphanyl]methyl}phosphonate (900 mg, 2.18 mmol) in acetonitrile (10 mL) was added 1H-imidazole-4,5-dicarbonitrile (257 mg, 2.18 mmol) carefully. The reaction was stirred at 20° C. for 12 h. Tert-butyl hydroperoxide (1.4 g, 10.9 mmol) was added into the mixture. The reaction was stirred for another 2 h. Then the reaction was diluted with EA (100 mL). The organic layer was washed with sat. Na₂CO₃ solution and brine, dried over Na₂SO₄, filtered and the filtrate was concentrated and purified by CombiFlash® (DCM/MeOH=100:3) to give di-tert-butyl [({[(3aR,4R,6S,6aS)-6-{2-chloro-4-[(1S)-2,3-dihydro-1H-inden-1-ylamino]imidazo[2,1-f][1,2,4]triazin-7-yl}-2,2-dimethyl-tetrahydrofuro[3,4-d][1,3]dioxol-4-yl]methoxy}(tert-butoxy)phosphoryl)methyl]phosphonate (800 mg, 84% yield) as a yellow oil. Mass Spectrum (ESI) m/z=615.5 (M+1).

Step J: To a mixture of di-tert-butyl [({[(3aR,4R,6S,6aS)-6-{2-chloro-4-[(1S)-2,3-dihydro-1H-inden-1-ylamino]imidazo[2,1-f][1,2,4]triazin-7-yl}-2,2-dimethyl-tetrahydrofuro[3,4-d][1,3]dioxol-4-yl]methoxy}(tert-butoxy)phosphoryl)methyl]phosphonate (1.25 g, 1.6 mmol) and ethylene glycol (0.5 g, 8 mmol) in dioxane (15 mL) was added hydrochloric acid (4 M in dioxane, 5 mL, 0.02 mol) carefully. The reaction was stirred at 20° C. for 1 h. The reaction mixture was concentrated, and the residue was purified by Prep-HPLC (Daisogel-C18 250×50 mm, 10 um column), using a gradient of 0.2% Formic acid in water/ACN from 80:20 to 60:40, to give [({[(2R,3S,4R,5S)-5-{2-chloro-4-[(1S)-2,3-dihydro-1H-inden-1-ylamino]imidazo[2,1-f][1,2,4]triazin-7-yl}-3,4-dihydroxyoxolan-2-yl]methoxy}(hydroxy)phosphoryl)methyl] phosphonic acid (500 mg, 56% yield) as a white solid. ¹H NMR (400 MHz, D₂O) δ 7.72 (s, 1H), 7.29-7.17 (m, 3H), 7.09 (m, 1H), 5.70 (t, J=7.2 Hz, 1H), 5.15 (d, J=6.3 Hz, 1H), 4.58-4.53 (m, 1H), 4.31 (t, J=4.8 Hz, 1H), 4.16 (m, 1H), 4.00-3.95 (m, 2H), 3.05-2.96 (m, 1H), 2.87 (m, 1H), 2.62-2.53 (m, 1H), 2.16-2.10 (m, 2H), 2.01-1.90 (m, 1H). Mass Spectrum (ESI) m/z=575.6 (M+1).

### Example S64

### Synthesis of (((((2R,3S,4R,1S)-5-(2-chloro-4-((S)-2-phenylpyrrolidin-1-yl)imidazo[2,1-f][1,2,4]triazin-7-yl)-3,4-dihydroxytetrahydrofuran-2-yl)methoxy)(hydroxy)phosphoryl)methyl)phosphonic Acid

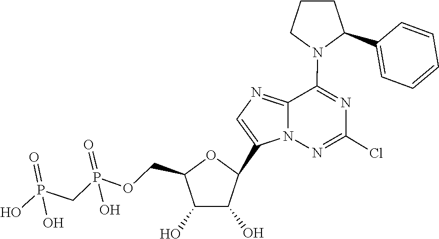 [SMILES: O=P(O)(O)CP(=O)(O)OC[C@H]1O[C@@H](c2cnc3c(N4CCC[C@H]4c4ccccc4)nc(Cl)nn23)[C@H](O)[C@@H]1O]

(((((2R,3S,4R,5S)-5-(2-chloro-4-((S)-2-phenylpyrrolidin-1-yl)imidazo[2,1-f][1,2,4]triazin-7-yl)-3,4-dihydroxytetrahydrofuran-2-yl)methoxyxhydroxy)phosphoryl)methyl) phosphonic acid was also by procedures similar to the one described in Example S63, replacing (1S)-2,3-dihydro-1H-inden-1-amine hydrochloride in step D with (S)-2-phenylpyrrolidine.

¹H NMR (400 MHz, D₂O) δ 7.81-7.41 (m, 1H), 7.29-7.14 (m, 5H), 6.30-5.48 (m, 1H), 5.19-5.10 (m, 1H), 4.68-4.49 (m, 2H), 4.25-4.21 (m, 1H), 4.19-4.13 (m, 1H), 4.07-3.98 (m, 2H), 3.81-3.77 (m, 1H), 2.49-2.25 (m, 1H), 2.21-1.77 (m, 5H). Mass Spectrum (ESI) m/z=589.7 (M+1).

### Example S65

### Synthesis of [({[(2R,3S,4R,5S)-5-[2-chloro-4-(pyrrolidin-1-yl)imidazo[2,1-f][1,2,4]triazin-7-yl]-3,4-dihydroxyoxolan-2-yl]methoxy}(hydroxy)phosphoryl)methyl]phosphonic Acid

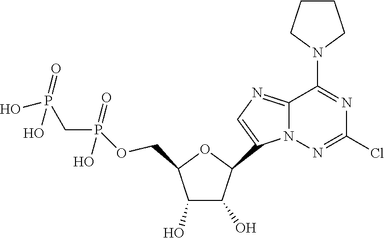 [SMILES: O=P(O)(O)CP(=O)(O)OC[C@H]1O[C@@H](c2cnc3c(N4CCCC4)nc(Cl)nn23)[C@H](O)[C@@H]1O]

[({[(2R,3S,4R,5S)-5-[2-chloro-4-(pyrrolidin-1-yl)imidazo[2,1-f][1,2,4]triazin-7-yl]-3,4-dihydroxyoxolan-2-yl]methoxy}(hydroxy)phosphoryl)methyl]phosphonic acid was also prepared by procedures similar to the one described in Example S63, replacing (1S)-2,3-dihydro-1H-inden-1-amine hydrochloride in step D with pyrrolidine.

¹H NMR (400 MHz, D₂O) δ 7.72 (s, 1H), 5.16-5.10 (m, 1H), 4.53-4.47 (m, 1H), 4.31-4.26 (m, 1H), 4.17-4.11 (m, 1H), 4.07-3.97 (m, 4H), 3.61-3.54 (m, 2H), 2.17 (t, J=18.4 Hz, 2H), 2.06-1.99 (m, 2H), 1.95-1.90 (m, 2H). Mass Spectrum (ESI) m/z=513.7 (M+1).

### Example S66

### Synthesis of (((((2R,3S,4R,5S)-5-(2-carbamoyl-4-(cyclopentylamino)imidazo[2,1-f][1,2,4]triazin-7-yl)-3,4-dihydroxytetrahydrofuran-2-yl)methoxy)(hydroxy)phosphoryl)methyl)phosphonic Acid

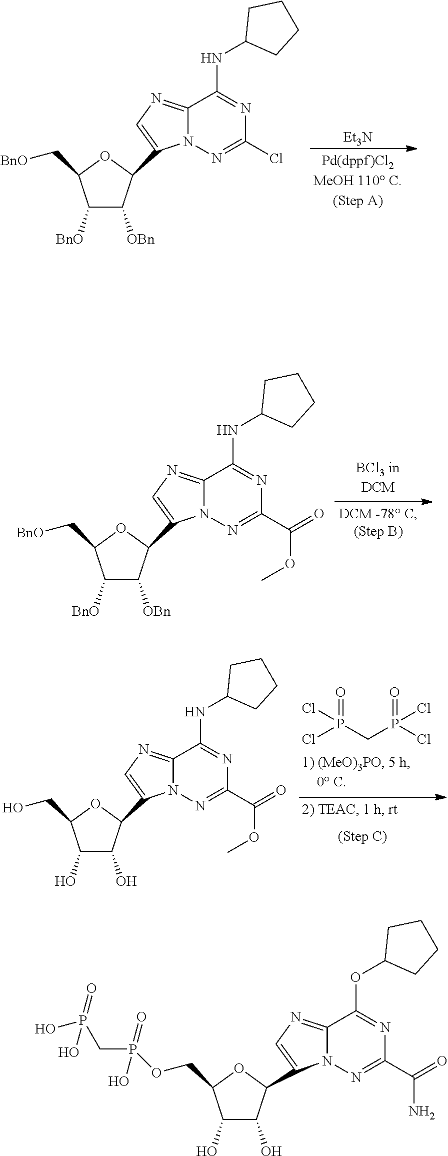

Step A: To a solution of 7-[(3S,4R,5R)-3,4-bis(benzyloxy)-5-[(benzyloxy)methyl]oxolan-2-yl]-2-chloro-N-cyclopentylimidazo[2,1-f][1,2,4]triazin-4-amine (0.6 g, 0.94 mmol) in MeOH (5 mL) was added Et₃N (0.19 g, 1.88 mmol) and Pd(dppf)Cl₂ (137 mg, 0.19 mmol). The mixture was stirred at 110° C. under a CO atmosphere for 16 h. Then the reaction mixture was concentrated and purified by CombiFlash® (20 g, EA/PE=0-20%) to give methyl 7-((2S,3S,4R,5R)-3,4-bis(benzyloxy)-5-((benzyloxy)methyl)tetrahydrofuran-2-yl)-4-(cyclopentylamino)imidazo[2,1-f][1,2,4]triazine-2-carboxylate (400 mg, 54% yield) as a yellow oil. Mass Spectrum (ESI) m/z=664.1 (M+1).

Step B and Step C: Methyl 7-[(2S,3S,4R,5R)-3,4-bis(benzyloxy)-5-[(benzyloxy)methyl]oxolan-2-yl]-4-(cyclopentylamino)imidazo[2,1-f][1,2,4]triazine-2-carboxylate was converted to the title compound by procedures similar to the ones described in Example S5, Step I and Step J.

¹H NMR (400 MHz, D₂O) δ 7.71 (s, 1H), 5.32 (d, J=6.2 Hz, 1H), 4.60-4.54 (m, 2H), 4.37-4.30 (m, 1H), 4.23-4.15 (m, 1H), 4.05-3.95 (m, 2H), 2.15-1.90 (m, 4H), 1.75-1.45 (m, 6H). Mass Spectrum (ESI) m/z=535.0 (M+1).

### Example S67

### Synthesis of (((((2R,3S,4R,1S)-5-(2-chloro-4-(cyclopentylamino)imidazo[2,1-f][1,2,4]triazin-7-yl)-3,4-dihydroxytetrahydrofuran-2-yl)methoxy)(2-(2-methoxyethoxy)ethoxy)phosphoryl)methyl)phosphonic Acid

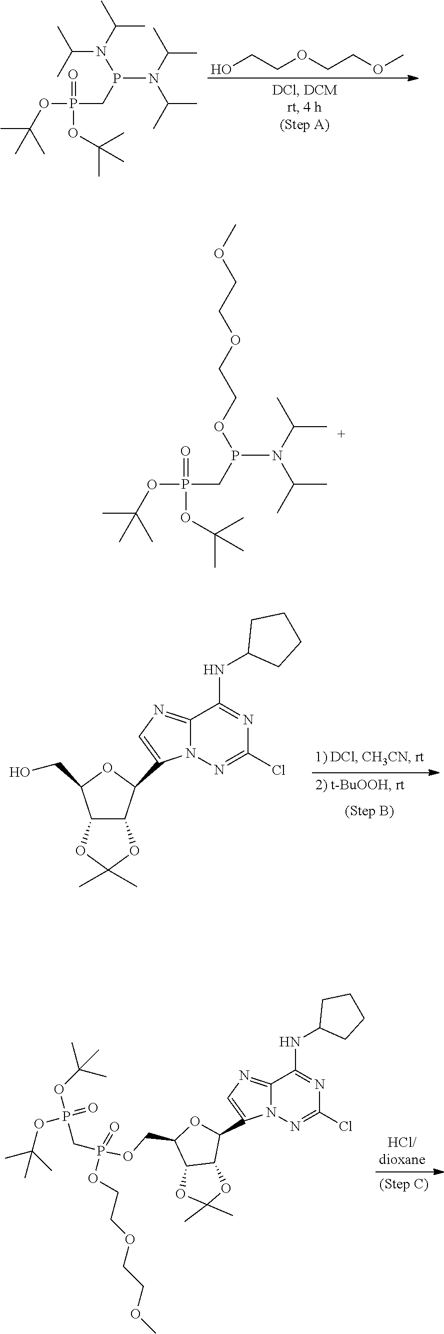

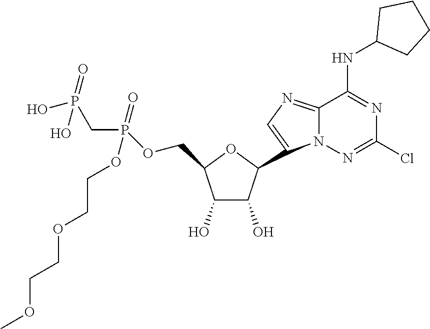 [SMILES: COCCOCCOP(=O)(CP(=O)(O)O)OC[C@H]1O[C@@H](c2cnc3c(NC4CCCC4)nc(Cl)nn23)[C@H](O)[C@@H]1O]

Step A: To a solution of di-tert-butyl {[bis(diisopropylamino)phosphanyl]methyl} phosphonate (500 mg, 1.14 mmol) in DCM (10 mL) was added 2-(2-methoxyethoxy)ethanol (123 mg, 1.03 mmol) and DCI (82 mg, 0.68 mmol), then the mixture was stirred at rt for 4 h, The resulting solution was concentrated and purified by column chromatography on silica gel (EA/l % TEA in PE=5:1) to give di-tert-butyl (((diisopropylamino)(2-(2-methoxyethoxy)ethoxy) phosphaneyl)methyl) phosphonate (400 mg, 73% yield) as a colorless oil. ¹H NMR (400 MHz, CDCl₃) δ 3.81-3.74 (m, 2H), 3.69-3.63 (m, 4H), 3.57-3.54 (m, 2H), 3.52-3.45 (m, 2H), 3.40 (s, 3H), 2.32-2.25 (m, 2H), 1.53 (d, J=1.3 Hz, 18H), 1.21 (d, J=6.7 Hz, 6H), 1.15 (d, J=6.8 Hz, 6H).

Step B: To a solution of [(3aR,4R,6S,6aS)-6-[2-chloro-4-(cyclopentylamino)imidazo[2,1-f][1,2,4]triazin-7-yl]-2,2-dimethyl-tetrahydrofuro[3,4-d][1,3]dioxol-4-yl]methanol (100 mg, 0.24 mmol) in MeCN (2 mL) was added DCI (43 mg, 0.37 mmol) and di-tert-butyl [2-(diisopropylamino)-3,6,9-trioxa-2-phosphadecan-1-yl]phosphonate (335 mg, 0.74 mmol). The mixture was stirred at rt overnight, then tert-butyl hydroperoxide (330 mg, 3.7 mmol) was added into the mixture. The reaction was stirred for another 2 h. Then the reaction was diluted with EA (100 mL) and washed with saturated aqueous Na₂CO₃. The organic layer was concentrated and purified by Prep-TLC (DCM/MeOH=20:1) to give ((3aR,4R,6S,6aS)-6-(2-chloro-4-(cyclopentylamino)imidazo[2,1-f][1,2,4]triazin-7-yl)-2,2-dimethyltetrahydrofuro[3,4-d][1,3]dioxol-4-yl)methyl (2-(2-methoxyethoxy)ethyl) ((di-tert-butoxyphosphoryl)methyl) phosphonate (30 mg, 15% yield) as a colorless oil. Mass Spectrum (ESI) m/z=670.0 (M-110+1).

Step C: A solution of di-tert-butyl [({[(3aR,4R,6S,6aS)-6-[2-chloro-4-(cyclopentylamino)imidazo[2,1-f][1,2,4]triazin-7-yl]-2,2-dimethyl-tetrahydrofuro[3,4-d][1,3]dioxol-4-yl]methoxy}[2-(2-methoxyethoxy)ethoxy]phosphoryl)methyl]phosphonate (30 mg, 0.03 mmol) in HCl/dioxane (2 mL) was stirred at rt overnight. Then the reaction was concentrated and purified by Prep-HPLC (Daisogel-C18 250×50 mm, 10 um column, 0.2% FA in water/MeCN, from 85% to 60%) to give (((((2R,3S,4R,5S)-5-(2-chloro-4-(cyclopentylamino)imidazo[2,1-f][1,2,4]triazin-7-yl)-3,4-dihydroxytetrahydrofuran-2-yl)methoxy)(2-(2-methoxyethoxy)ethoxy)phosphoryl)methyl)phosphonic acid (1 mg, 4% yield) as a white solid.

¹H NMR (400 MHz, D₂O) δ 7.58 (s, 1H), 5.13 (d, J=6.4 Hz, 1H), 4.60 (m, 1H), 4.42-4.28 (m, 3H), 4.20-4.16 (m, 3H), 3.98-3.92 (m, 2H), 3.48-3.38 (m, 5H), 3.21 (d, J=1.2 Hz, 3H), 2.21 (t, J=18.8 Hz, 2H), 2.00-1.95 (m, 2H), 1.80-1.58 (m, 6H). Mass Spectrum (ESI) m/z=630.0 (M+1).

### Example S68

### Synthesis of (((((2R,3S,4R,1S)-5-(2-chloro-4-(cyclopentylamino)imidazo[2,1-f][1,2,4]triazin-7-yl)-3,4-dihydroxytetrahydrofuran-2-yl)methoxy)(2-methoxyethoxy)phosphoryl)methyl)phosphonic Acid

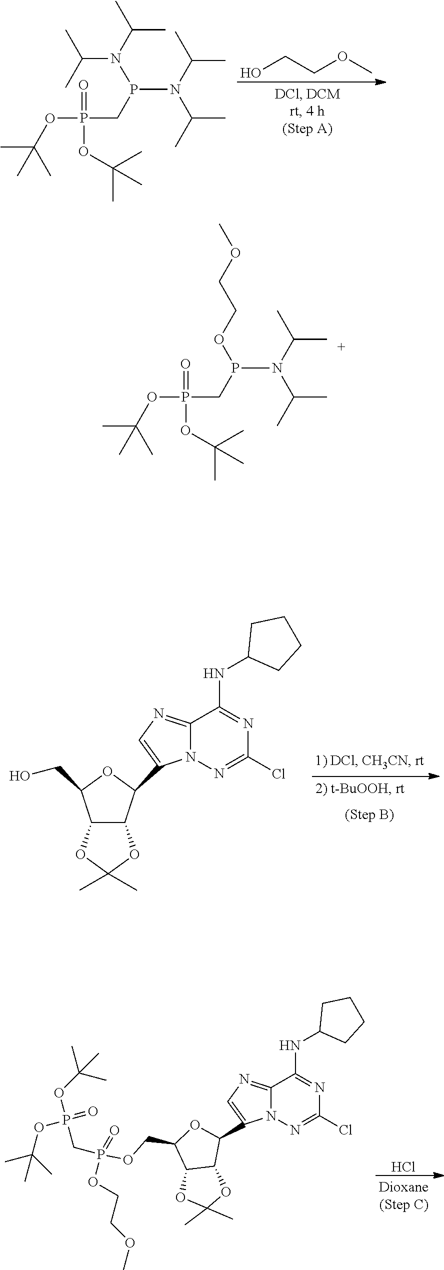

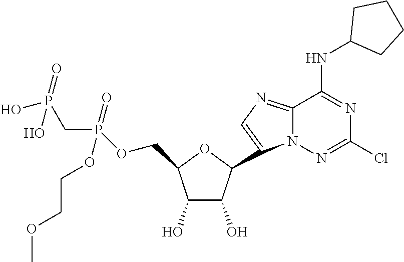 [SMILES: COCCOP(=O)(CP(=O)(O)O)OC[C@H]1O[C@@H](c2cnc3c(NC4CCCC4)nc(Cl)nn23)[C@H](O)[C@@H]1O]

Step A: To a solution of di-tert-butyl {[bis(diisopropylamino)phosphanyl]methyl} phosphonate (1 g, 2.28 mmol) in DCM (10 mL) was added 2-methoxyethanol (174 mg, 2.28 mmol) and 4,5-dihydro-1H-imidazole-4,5-dicarbonitrile (DCI, 162 mg, 1.37 mmol), then the mixture was stirred at rt for 4 h. The resulting solution was concentrated and purified by column chromatography on silica gel (EA/1% TEA in PE=5:1) to give di-tert-butyl (((diisopropylamino)(2-methoxyethoxy)phosphaneyl)methyl)phosphonate (800 mg, 76% yield) as a colorless oil. ¹H NMR (301 MHz, CDCl₃) δ 3.81-3.67 (m, 2H), 3.59-3.43 (m, 4H), 3.35 (s, 3H), 2.30-2.19 (m, 1H), 2.03-1.87 (m, 1H), 1.49 (d, J=1.2 Hz, 18H), 1.18 (d, J=6.7 Hz, 6H), 1.12 (d, J=6.8 Hz, 6H).

Step B: To a solution of [(3aR,4R,6S,6aS)-6-[2-chloro-4-(cyclopentylamino)imidazo[2,1-f][1,2,4]triazin-7-yl]-2,2-dimethyl-tetrahydrofuro[3,4-d][1,3]dioxol-4-yl]methanol (100 mg, 0.24 mmol) in MeCN (2 mL) was successively added 1H-imidazole-4,5-dicarbonitrile (43 mg, 0.36 mmol) and di-tert-butyl [(7-isopropyl-8-methyl-2,5-dioxa-7-aza-6-phosphanonan-6-yl)methyl]phosphonate (303 mg, 0.73 mmol). The mixture was stirred at rt overnight, then tert-butyl hydroperoxide (330 mg, 3.7 mmol) was added into the mixture. The reaction was stirred for another 2 h. Then the reaction was diluted with EA (100 mL) and washed with saturated aqueous Na₂CO₃. The organic layer was concentrated and purified by CombiFlash® (silica gel, 4 g, DCM/MeOH=0-6%) to give di-tert-butyl [({[(3aR,4R,6S,6aS)-6-[2-chloro-4-(cyclopentylamino)imidazo[2,1-f][1,2,4]triazin-7-yl]-2,2-dimethyl-tetrahydrofuro[3,4-d][1,3]dioxol-4-yl]methoxy}(2-methoxyethoxy)phosphoryl)methyl]phosphonate (250 mg, 44% yield) as a colorless oil. Mass Spectrum (ESI) m/z=626.0 (M+1).

Step C: A solution of di-tert-butyl [({[(3aR,4R,6S,6aS)-6-[2-chloro-4-(cyclopentylamino)imidazo[2,1-f][1,2,4]triazin-7-yl]-2,2-dimethyl-tetrahydrofuro[3,4-d][1,3]dioxol-4-yl]methoxy}(2-methoxyethoxy)phosphoryl)methyl]phosphonate (250 mg, 0.34 mmol) in HCl/dioxane (5 mL) was stirred overnight at rt. Then the mixture was concentrated and purified by Prep-HPLC (Daisogel-C18 250×50 mm, 10 um column, 0.2% FA in H₂O/MeCN=75%-55%) to give (((((2R,3S,4R,5S)-5-(2-chloro-4-(cyclopentylamino)imidazo[2,1-f][1,2,4]triazin-7-yl)-3,4-dihydroxytetrahydrofuran-2-yl)methoxy)(2-methoxyethoxy)phosphoryl)methyl)phosphonic acid (20 mg, 9% yield) as a white solid.

¹H NMR (400 MHz, D₂O) δ 7.60 (m, 1H), 5.14 (d, J=6.2 Hz, 1H), 4.62-4.55 (m, 1H), 4.39-4.28 (m, 2H), 4.22-4.12 (m, 3H), 4.00-3.88 (m, 2H), 3.45-3.38 (m, 2H), 3.20 (s, 3H), 2.40-2.25 (m, 2H), 2.01-1.93 (m, 2H), 1.70-1.54 (m, 6H). Mass Spectrum (ESI) m/z=586.0 (M+1).

### Example S69

### Synthesis of (((((2R,3S,4R,1S)-5-(2-chloro-4-(cyclopentylamino)imidazo[2,1-f][1,2,4]triazin-7-yl)-3,4-dihydroxytetrahydrofuran-2-yl)methoxy)(2-cyanoethoxy)phosphoryl)methyl) phosphonic Acid

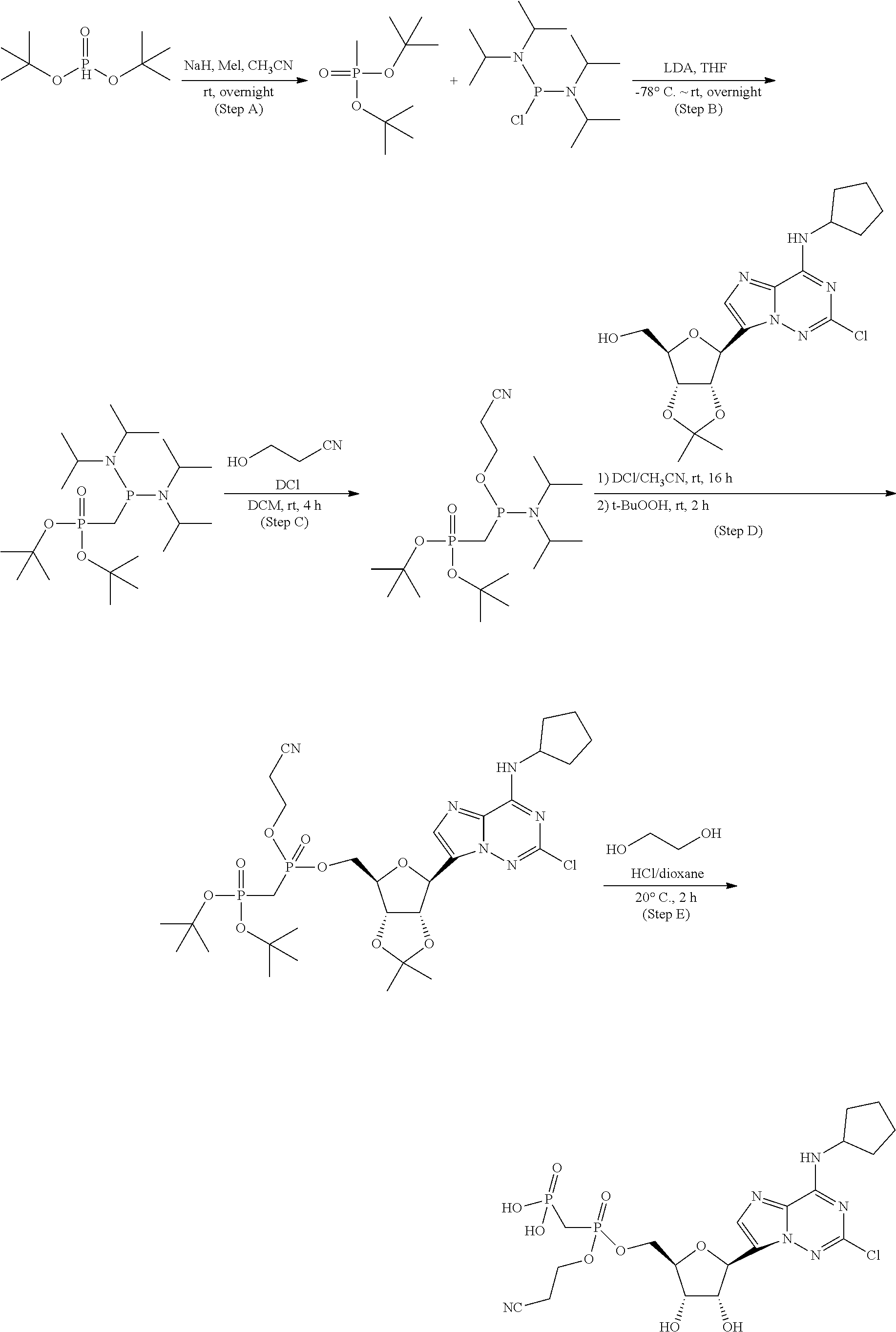

Step A: To a mixture of di-tert-butyl phosphonate (10 g, 51.5 mmol) in acetonitrile (40 mL) was added NaH (3.09 g, 77.2 mmol) carefully at 0° C. under nitrogen. The reaction was stirred for 30 min at rt. Then a solution of iodomethane (10.96 g, 77.24 mmol) in acetonitrile (10 mL) was added at rt under nitrogen. The reaction was stirred at rt overnight, then quenched by water (1 ml) and concentrated. The crude product was purified by silica gel column chromatography (400 g, PE/EA=1:1) to give di-tert-butyl methylphosphonate (10 g, 83.9% yield) as a yellow oil. ¹H NMR (400 MHz, DMSO) δ 1.43 (s, 18H), 1.33 (d, J=17.2 Hz, 3H).

Step B: To a mixture of diisopropylamine (8.2 g, 80.7 mmol) in THF (30 mL) was added n-BuLi (33.6 mL, 80.7 mmol, 2.4M) carefully at −78° C. under nitrogen. The reaction was stirred for 30 min at −78° C. Then di-tert-butyl methylphosphonate (8 g, 38.4 mmol) was added carefully at −78° C. under nitrogen. The reaction was stirred for 30 min at −78° C. Then a solution of 1-chloro-N,N,N′,N′-tetraisopropylphosphanediamine (10.25 g, 38.4 mmol) in THF (70 mL) was added at −78° C. under nitrogen. The reaction was stirred at rt overnight. The reaction was quenched by sat. NaHCO₃ solution and extracted with EA (100 mL×3). The combined organic layers were washed with brine, dried over Na₂SO₄, filtered and the filtrate was concentrated. The crude product was purified by silica gel column chromatography (800 g, PE/EA=20:1+1% TEA) to give di-tert-butyl ((bis(diisopropylamino)phosphanyl)methyl)phosphonate (12 g, 64.1% yield) as a yellow oil.

¹H NMR (400 MHz, CDCl₃) δ 3.47-3.31 (m, 4H), 2.18-2.04 (m, 2H), 1.56-1.49 (s, 18H), 1.19 (t, J=8.1 Hz, 24H).

Step C: To a mixture of di-tert-butyl {[bis(diisopropylamino)phosphanyl]methyl} phosphonate (800 mg, 1.84 mmol) and 3-hydroxypropanenitrile (131 mg, 1.84 mmol) in DCM (10 mL) was added 1H-imidazole-4,5-dicarbonitrile (130 mg, 1.1 mmol). The reaction was stirred for 4 h at rt. Then the mixture was concentrated and the residue was purified by silica gel column chromatography (12 g, PE/EA=3:1+1% TEA) to give di-tert-butyl {[(2-cyanoethoxy)(diisopropylamino)phosphanyl]methyl}phosphonate (680 mg, 80.2% yield) as a yellow oil. ¹H NMR (400 MHz, CDCl₃) δ 3.92-3.78 (m, 2H), 3.58-3.43 (m, 2H), 2.73-2.58 (m, 2H), 2.40-2.21 (m, 1H), 2.00-1.83 (m, 1H), 1.53 (d, J=2.2 Hz, 18H), 1.22 (d, J=6.7 Hz, 6H), 1.15 (d, J=6.8 Hz, 6H).

Step D: To a mixture of di-tert-butyl {[(2-cyanoethoxy)(diisopropylamino) phosphanyl]methyl} phosphonate (605 mg, 1.48 mmol) and [(3aR,4R,6S,6aS)-6-[2-chloro-4-(cyclopentylamino) imidazo[2,1-f][1,2,4]triazin-7-yl]-2,2-dimethyl-tetrahydrofuro[3,4-d][1,3]dioxol-4-yl] methanol (150 mg, 0.37 mmol) in MeCN (5 mL) was added DCI (66 mg, 0.55 mmol). The reaction was stirred for 16 h at rt. Then tert-butyl hydroperoxide (476 mg, 5.3 mmol) was added into the mixture. The reaction was stirred for 2 h at rt. Then the reaction was diluted with EA (30 mL), washed with sat. Na₂CO₃ solution and brine. The organic layer was dried over Na₂SO₄, filtered and the filtrate was concentrated and purified by CombiFlash® (12 g, eluting with DCM/MeOH=20:1) to give di-tert-butyl [({[(3aR,4R,6S,6aS)-6-[2-chloro-4-(cyclopentylamino)imidazo[2,1-f][1,2,4]triazin-7-yl]-2,2-dimethyl-tetrahydrofuro[3,4-d][1,3]dioxol-4-yl]methoxy}(2-cyanoethoxy)phosphoryl)methyl]phosphonate (250 mg, 93.2% yield) as a light yellow oil. Mass Spectrum (ESI) m/z=621.1 (M-110).

Step E: To a mixture of di-tert-butyl [({[(3aR,4R,6S,6aS)-6-[2-chloro-4-(cyclopentylamino) imidazo[2,1-f][1,2,4]triazin-7-yl]-2,2-dimethyl-tetrahydrofuro[3,4-d][1,3]dioxol-4-yl]methoxy} (2-cyanoethoxy)phosphoryl)methyl]phosphonate (100 mg, 0.14 mmol) and Ethylene glycol (85 mg, 1.36 mmol) in dioxane (2 mL) was added hydrochloric acid (0.25 mL, 3.00 mmol, 4M) carefully. The reaction was stirred for 2 h at 20° C. The reaction was concentrated and purified by Prep-HPLC (Daisogel-C18 250×50 mm, 10 um column) using a gradient of 20 mM TEAC in water/ACN from 80:20 to 60:40, and suitable fractions were pooled and lyophilized to give (((((2R,3S,4R,5S)-5-(2-chloro-4-(cyclopentylamino)imidazo[2,1-f][1,2,4] triazin-7-yl)-3,4-dihydroxytetrahydrofuran-2-yl)methoxy)(2-cyanoethoxy)phosphoryl)methyl) phosphonic acid (2.5 mg, 3% yield) as a white solid.

¹H NMR (400 MHz, D₂O) δ 7.65 (s, 1H), 5.16 (d, J=5.7 Hz, 1H), 4.60-4.56 (m, 1H), 4.42-4.29 (m, 2H), 4.29-4.16 (m, 3H), 4.12-4.06 (m, 2H), 2.79-2.66 (m, 2H), 2.39 (t, J=20.5 Hz, 2H), 2.02-1.96 (m, 2H), 1.74-1.53 (m, 6H). Mass Spectrum (ESI) m/z=581.0 (M+1).

### Example S70

### Synthesis of (((((((2R,3S,4R,5S)-5-(2-chloro-4-(cyclopentylamino)imidazo[2,1-f][1,2,4]triazin-7-yl)-3,4-dihydroxytetrahydrofuran-2-yl)methoxy)(hydroxy)phosphoryl)methyl)phosphoryl)bis(oxy)) bis(methylene) diisopropyl bis(carbonate)

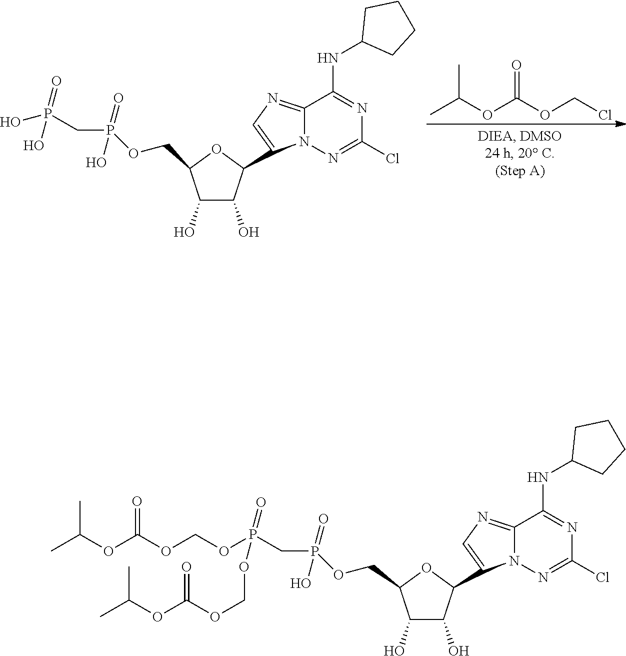

Step A: To a mixture of [({[(2R,3S,4R,5S)-5-[2-chloro-4-(cyclopentylamino)imidazo[2,1-f][1,2,4] triazin-7-yl]-3,4-dihydroxyoxolan-2 yl]methoxy}(hydroxy)phosphoryl)methyl]phosphonic acid (50 mg, 0.09 mmol, Example S5) and DIEA (367 mg, 2.8 mmol) in DMSO (2 mL) was added chloromethyl isopropyl carbonate (433 mg, 2.8 mmol) dropwise. The reaction was stirred for 24 h at 20° C. Then the mixture was purified by Prep-HPLC (Daisogel-C18 250×50 mm, 10 um column) using a gradient of 0.2% FA in water/ACN from 60:40 to 40:60 Suitable fractions were pooled and lyophilized to give (((((((2R,3S,4R,5S)-5-(2-chloro-4-(cyclopentylamino)imidazo[2,1-f][1,2,4]triazin-7-yl)-3,4-dihydroxytetrahydrofuran-2-yl)methoxyxhydroxy)phosphoryl)methyl)phosphoryl) bis(oxy))bis(methylene) diisopropyl bis(carbonate) (12 mg, 8% yield) as a white solid.

¹H NMR (400 MHz, D₂O) δ 7.58 (d, J=2.7 Hz, 1H), 5.52-5.44 (m, 1H), 5.44-5.32 (m, 3H), 5.13 (d, J=5.7 Hz, 1H), 4.78-4.73 (m, 2H), 4.63-4.58 (m, 1H), 4.40-4.26 (m, 2H), 4.18-4.12 (m, 3H), 2.47-2.33 (m, 2H), 2.02-1.92 (m, 2H), 1.74-1.64 (m, 2H), 1.62-1.58 (m, 4H), 1.27-1.11 (m, 12H). Mass Spectrum (ESI) m/z=760.0 (M+1).

### Example S71

### Synthesis of (((((2R,3S,4R,1S)-5-(2-chloro-4-(cyclopentylamino)imidazo[2,1-f][1,2,4]triazin-7-yl)-3,4-dihydroxytetrahydrofuran-2-yl)methoxy)(methoxy)phosphoryl)methy)phosphonic Acid

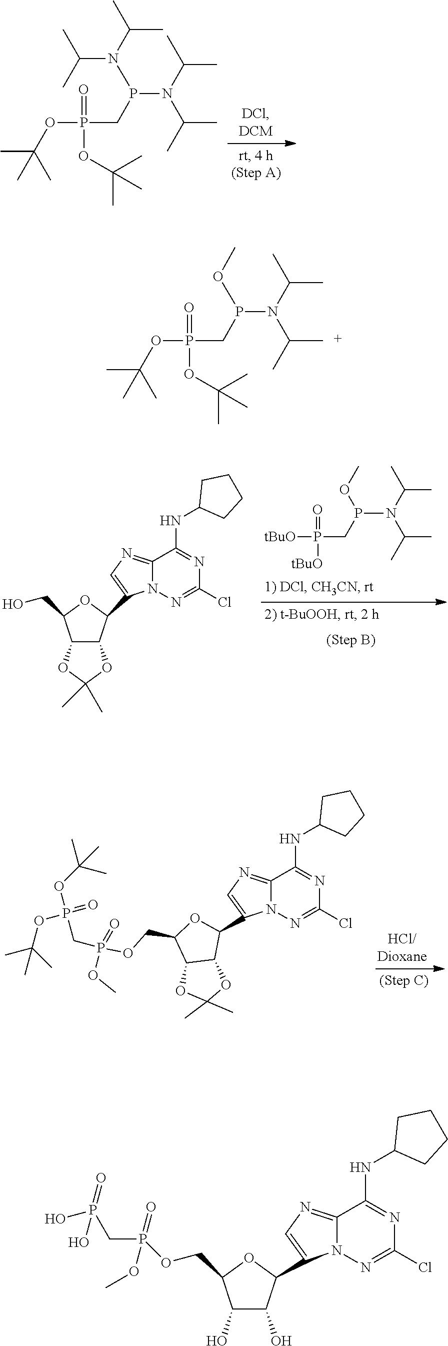

The title compound is obtained in Example S59, Step C, as a white solid.

¹H NMR (400 MHz, D₂O) δ 7.55 (s, 1H), 5.14 (d, J=5.9 Hz, 1H), 4.59-4.54 (m, 1H), 4.38-4.27 (m, 2H), 4.20-4.10 (m, 3H), 3.54 (dd, J=24.7, 11.4 Hz, 3H), 2.35-2.18 (m, 2H), 2.00-1.88 (m, 2H), 1.70-1.51 (in, 6H). Mass Spectrum (ESI) m/z=541.5 (M+1).

### Example S72

### Synthesis of (((((2R,3R,4S,5S)-5-(2-chloro-4-(hexahydrocyclopenta[c]pyrrol-2(1H)-yl)imidazo[2,1-f][1,2,4] triazin-7-yl)-4-fluoro-3-hydroxytetrahydrofuran-2-yl)methoxy)(hydroxy)phosphoryl)methyl) phosphonic Acid

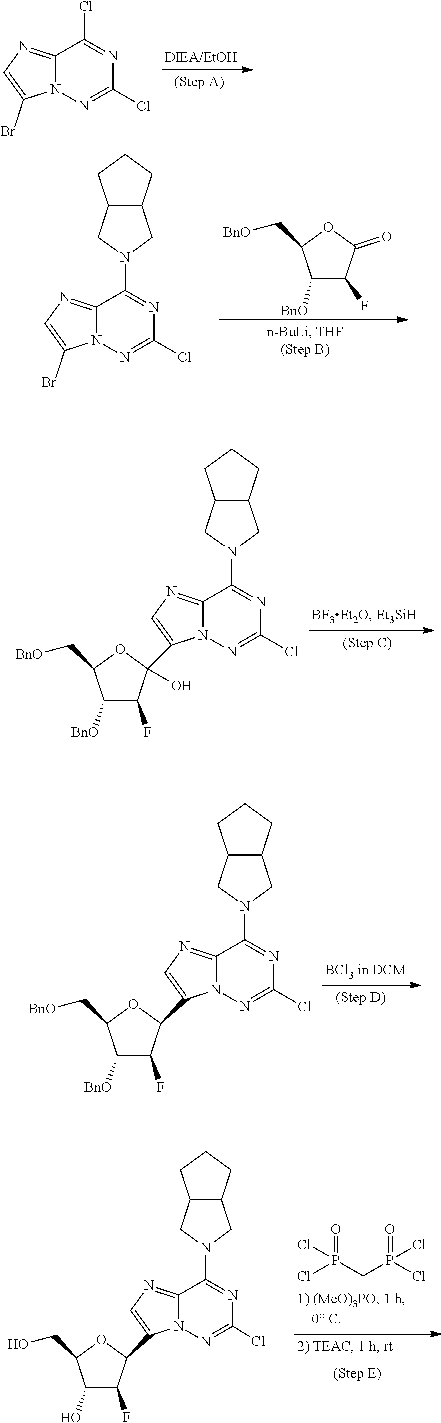

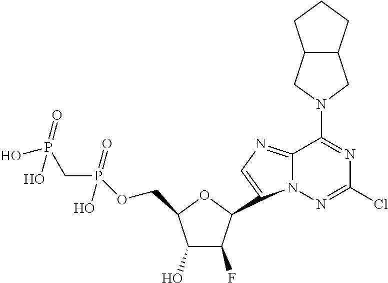 [SMILES: O=P(O)(O)CP(=O)(O)OC[C@H]1O[C@@H](c2cnc3c(N4CC5CCCC5C4)nc(Cl)nn23)[C@@H](F)[C@@H]1O]

Step A: To a mixture of 7-bromo-2,4-dichloroimidazo[2,1-f][1,2,4]triazine (1.5 g, 5.60 mmol) and DIEA (1.81 g, 14 mmol) in EtOH (25 mL) was added octahydrocyclopenta[c]pyrrole (910 mg, 6.16 mmol) carefully. The reaction was stirred for 16 h at 20° C. Then the reaction mixture was filtered and the solid was collected (1.6 g, 75.0%). The yellow solid obtained was used in the next step without further purification. Mass Spectrum (ESI) m/z=341.7 (M+1).

Step B: To a solution of 7-bromo-2-chloro-4-{hexahydro-1H-cyclopenta[c]pyrrol-2-yl}imidazo [2,1-f][1,2,4]triazine (1.4 g, 4.09 mmol) in THF (25 mL) was added n-BuLi (2.22 mL, 5.32 mmol, 2.4 M) carefully at −78° C. under nitrogen. The reaction mixture was stirred for 30 min at −78° C. Then a solution of (3S,4R,5R)-4-(benzyloxy)-5-[(benzyloxy)methyl]-3-fluorooxolan-2-one (1.49 g, 4.5 mmol) in THF (5 mL) was added at −78° C. under nitrogen. The reaction was stirred for another 2 h at −78° C., then quenched with sat. aq. NH₄Cl solution. The mixture extracted with EA (100 mL×3). The combined organic layers were washed with brine, dried over Na₂SO₄, filtered, and the filtrate was concentrated and purified by CombiFlash® (PE/EA=2:1) to give (3S,4R,5R)-4-(benzyloxy)-5-[(benzyloxy)methyl]-2-(2-chloro-4-{hexahydro-1H-cyclopenta[c]pyrrol-2-yl}imidazo[2,1-f][1,2,4]triazin-7-yl)-3-fluorooxolan-2-ol (1.8 g, 66.7% yield) as a yellow solid. Mass Spectrum (ESI) m/z=594.0 (M+1).

Step C: To a mixture of (3S,4R,5R)-4-(benzyloxy)-5-[(benzyloxy)methyl]-2-(2-chloro-4-{hexahydro-1H-cyclopenta[c]pyrrol-2-yl}imidazo[2,1-f][1,2,4]triazin-7-yl)-3-fluorooxolan-2-ol (1.6 g, 2.69 mmol) in DCM (30 mL) was added boron trifluoride etherate (3.82 g, 26.9 mmol) carefully at −78° C. under nitrogen. Then triethylsilane (3.1 g 26.9 mmol) was added at −78° C. under nitrogen. The reaction was stirred for 2 h at 20° C. The reaction was quenched by sat. NaHCO₃ solution and extracted with DCM (100 mL×3). The organic layer was washed with brine and dried over Na₂SO₄, filtered and the filtrate was concentrated. The crude product was purified by column chromatography to give 7-[(3R,4R,5R)-4-(benzyloxy)-5-[(benzyloxy)methyl]-3-fluorooxolan-2-yl]-2-chloro-4-(hexahydro-1H-cyclopenta[c]pyrrol-2-yl)imidazo[2,1-f][1,2,4]triazine (1.4 g, 81% yield) as a yellow oil. Mass Spectrum (ESI) m/z=577.9 (M+1).

Step D and Step E: 7-[(3R,4R,5R)-4-(benzyloxy)-5-[(benzyloxy)methyl]-3-fluorooxolan-2-yl]-2-chloro-4-{hexahydro-1H-cyclopenta[c]pyrrol-2-yl}imidazo[2,1-f][1,2,4]triazine was converted to the title compound by procedures similar to the ones described in Example S5, Step I and Step J.

¹H NMR (400 MHz, D₂O) δ 7.65 (s, 1H), 5.47-5.40 (m, 1H), 5.30-5.17 (m, 1H), 4.51-4.46 (m, 1H), 4.30-4.21 (m, 1H), 4.19-3.86 (m, 4H), 3.85-3.74 (m, 1H), 3.52-3.40 (m, 1H), 2.88-2.65 (m, 2H), 2.26 (t, J=20.3 Hz, 2H), 1.90-1.76 (m, 2H), 1.75-1.53 (m, 2H), 1.53-1.36 (m, 2H). Mass Spectrum (ESI) m/z=555.8 (M+1).

### Example S73

### Synthesis of (((((2R,3R,4S)-5-(2-chloro-4-(pyrrolidin-1-yl)imidazo[2,1-f][1,2,4]triazin-7-yl)-4-fluoro-3-hydroxytetrahydrofuran-2-yl)methoxy)(hydroxy)phosphoryl)methyl)phosphonic Acid

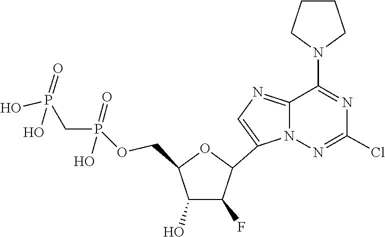 [SMILES: O=P(O)(O)CP(=O)(O)OC[C@H]1OC(c2cnc3c(N4CCCC4)nc(Cl)nn23)[C@@H](F)[C@@H]1O]

(((((2R,3R,4S)-5-(2-chloro-4-(pyrrolidin-1-yl)imidazo[2,1-f][1,2,4]triazin-7-yl)-4-fluoro-3-hydroxytetrahydrofuran-2-yl)methoxyxhydroxy)phosphoryl)methyl)phosphonic acid was prepared by procedures similar to the one described in S72, replacing octahydrocyclopenta[c] pyrrole in step A with pyrrolidine.

¹H NMR (400 MHz, D₂O) δ 7.66 (s, 1H), 5.49-5.37 (m, 1H), 5.31-5.18 (m, 1H), 4.51-4.46 (m, 1H), 4.16-3.96 (m, 5H), 3.58-3.49 (m, 2H), 2.25 (t, J=19.5 Hz, 2H), 2.07-1.97 (m, 2H), 1.96-1.91 (m, 2H). Mass Spectrum (ESI) m/z=515.6 (M+1).

### Example S74

### Synthesis of (((((2R,3R,4S)-5-(2-chloro-4-(((S)-1-(2-fluorophenyl)ethyl)amino)imidazo[2,1-f][1,2,4]triazin-7-yl)-4-fluoro-3-hydroxytetrahydrofuran-2-yl)methoxy)(hydroxy)phosphoryl)methyl)phosphonic Acid

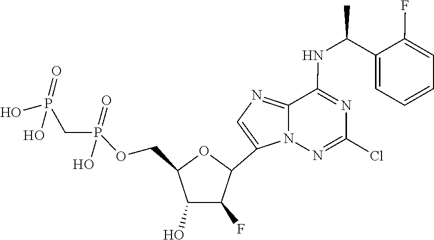 [SMILES: C[C@H](Nc1nc(Cl)nn2c(C3O[C@H](COP(=O)(O)CP(=O)(O)O)[C@@H](O)[C@@H]3F)cnc12)c1ccccc1F]

(((((2R,3R,4S)-5-(2-chloro-4-(((S)-1-(2-fluorophenyl)ethyl)amino)imidazo[2,1-f] [1,2,4]triazin-7-yl)-4-fluoro-3-hydroxytetrahydrofuran-2-yl)methoxy)(hydroxy)phosphoryl)methyl) phosphonic acid was prepared by procedures similar to the one described in S72, replacing octahydrocyclopenta[c]pyrrole in step A with (S)-1-(2-fluorophenyl)ethan-1l-amine.

¹H NMR (400 MHz, D₂O) δ 7.75-7.58 (m, 1H), 7.45-7.32 (m, 1H), 7.28-7.18 (m, 1H), 7.05-6.95 (m, 2H), 5.90-5.40 (m, 2H), 5.32-5.13 (m, 1H), 4.55-4.38 (m, 1H), 4.15-3.90 (m, 3H), 2.32-2.12 (m, 2H), 1.62-1.52 (m, 3H). Mass Spectrum (ESI) m/z=583.5 (M+1).

### Example S75

### Synthesis of (((((2R,3R,4S,5S)-5-(2-chloro-4-(3,3-difluoropyrrolidin-1-yl)imidazo[2,1-f][1,2,4]triazin-7-yl)-4-fluoro-3-hydroxytetrahydrofuran-2-yl)methoxy)(hydroxy)phosphoryl)methyl)phosphonic Acid

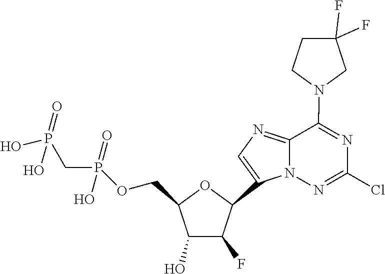 [SMILES: O=P(O)(O)CP(=O)(O)OC[C@H]1O[C@@H](c2cnc3c(N4CCC(F)(F)C4)nc(Cl)nn23)[C@@H](F)[C@@H]1O]

(((((2R,3R,4S,5S)-5-(2-chloro-4-(3,3-difluoropyrrolidin-1-yl)imidazo[2,1-f][1,2,4]triazin-7-yl)-4-fluoro-3-hydroxytetrahydrofuran-2-yl)methoxyxhydroxy)phosphoryl)methyl) phosphonic acid was prepared by procedures similar to the one described in S72, replacing octahydrocyclopenta[c]pyrrole in step A with 3,3-difluoropyrrolidine.

¹H NMR (400 MHz, DMSO) δ 7.70 (s, 1H), 5.53-5.45 (m, 1H), 5.21-5.08 (m, 1H), 4.70-4.65 (m, 1H), 4.56-4.53 (m, 1H), 4.32-4.29 (m, 1H), 4.18-4.00 (m, 4H), 3.96-3.93 (m, 1H), 2.73-2.57 (m, 2H), 2.25 (t, J=20.5 Hz, 2H). Mass Spectrum (ESI) m/z=551.5 (M+1).

### Example S76

### Synthesis of ((2R,3R,4S,5S)-5-(2-chloro-4-(hexahydrocyclopenta[c]pyrrol-2(1H)-yl)imidazo[2,1-f]J[1,2,4]triazin-7-yl)-4-fluoro-3-hydroxytetrahydrofuran-2-yl)methyl methyl ((dimethoxyphosphoryl)methyl)phosphonate

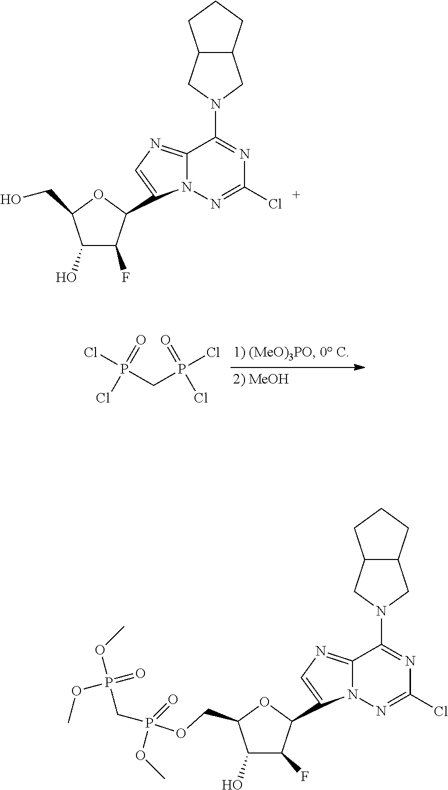 [SMILES: OC[C@H]1O[C@@H](c2cnc3c(N4CC5CCCC5C4)nc(Cl)nn23)[C@@H](F)[C@@H]1O ; O=P(Cl)(Cl)CP(=O)(Cl)Cl ; COP(=O)(CP(=O)(OC)OC[C@H]1O[C@@H](c2cnc3c(N4CC5CCCC5C4)nc(Cl)nn23)[C@@H](F)[C@@H]1O)OC]

Step A: To a mixture of (2R,3R,4S)-5-(2-chloro-4-{hexahydro-1H-cyclopenta[c]pyrrol-2-yl}imidazo[2,1-f][1,2,4]triazin-7-yl)-4-fluoro-2-(hydroxymethyl)oxolan-3-ol (Example S72, Step D, 150 mg, 0.38 mmol) in trimethyl phosphate (3 mL) was added [(dichlorophosphoryl)methyl]phosphonoyl dichloride (475 mg, 1.9 mmol) carefully at 0° C. under nitrogen. The reaction was stirred for 2 h at 0° C. Then methanol (1.2 g, 38 mmol) was added carefully at 0° C. under nitrogen. The reaction was stirred for 2 h at 0° C. Then purified by Prep-HPLC (Daisogel-C18 250×50 mm, 10 um column, 0.2% FA in water/MeCN, from 60% to 30%) to give ((2R,3R,4S,5S)-5-(2-chloro-4-(hexahydrocyclopenta[c]pyrrol-2(1H)-yl)imidazo[2,1-f][1,2,4]triazin-7-yl)-4-fluoro-3-hydroxytetrahydrofuran-2-yl)methyl methyl ((dimethoxyphosphoryl)methyl)phosphonate (22 mg, 14% yield) as a white solid.

¹H NMR (400 MHz, D₂O) δ 7.36 (s, 1H), 5.44-5.11 (m, 2H), 5.10-4.93 (m, 1H), 4.42-4.28 (m, 1H), 4.26-4.11 (m, 2H), 4.09-3.99 (m, 1H), 3.93-3.78 (m, 1H), 3.76-3.50 (m, 9H), 3.47-3.13 (m, 2H), 2.84-2.47 (m, 4H), 1.78-1.22 (m, 6H). Mass Spectrum (ESI) m/z=597.8 (M+1).

### Example S77

### Synthesis of (((((2R,3S,4R,1S)-5-(2-chloro-4-(((4-hydroxycyclohexyl)methyl)amino)imidazo[2,1-f][1,2,4]triazin-7-yl)-3,4-dihydroxytetrahydrofuran-2-yl)methoxy)(hydroxy)phosphoryl)methyl)phosphonic Acid

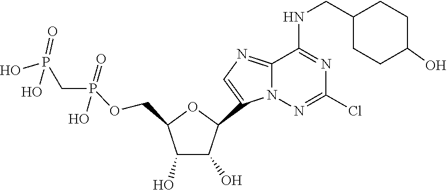 [SMILES: O=P(O)(O)CP(=O)(O)OC[C@H]1O[C@@H](c2cnc3c(NCC4CCC(O)CC4)nc(Cl)nn23)[C@H](O)[C@@H]1O]

(((((2R,3S,4R,5S)-5-(2-chloro-4-(((4-hydroxycyclohexyl)methyl)amino)imidazo[2,1-f][1,2,4]triazin-7-yl)-3,4-dihydroxytetrahydrofuran-2-yl)methoxyxhydroxy)phosphoryl)methyl) phosphonic acid was prepared by procedures similar to the one described in Example S5, replacing cyclopentanamine in Step F with 4-(aminomethyl)cyclohexan-1-ol.

¹H NMR (400 MHz, D₂O) δ 7.64 (s, 1H), 5.14 (d, J=6.6 Hz, 1H), 4.57-4.51 (m, 1H), 4.37-4.32 (m, 1H), 4.18-4.13 (m, 1H), 4.00-3.90 (m, 2H), 3.57-3.48 (m, 1H), 3.39-3.30 (m, 2H), 1.97 (t, J=19.6 Hz, 2H), 1.90-1.83 (m, 2H), 1.80-1.72 (m, 2H), 1.67-1.60 (m, 1H), 1.10-0.9 (m, 4H). Mass Spectrum (ESI) m/z=570.0 (M−1).

### Example S78

### Synthesis of (((((2R,3S,4R,1S)-5-(2-chloro-4-(((3-hydroxycyclohexyl)methyl)amino)imidazo[2,1-f][1,2,4]triazin-7-yl)-3,4-dihydroxytetrahydrofuran-2-yl)methoxy)(hydroxy)phosphoryl)methyl)phosphonic Acid

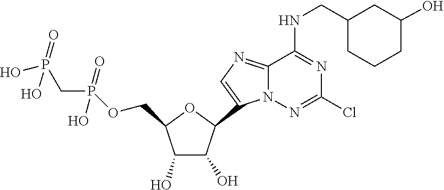 [SMILES: O=P(O)(O)CP(=O)(O)OC[C@H]1O[C@@H](c2cnc3c(NCC4CCCC(O)C4)nc(Cl)nn23)[C@H](O)[C@@H]1O]

(((((2R,3S,4R,5S)-5-(2-chloro-4-(((3-hydroxycyclohexyl)methyl)amino)imidazo[2,1-f][1,2,4]triazin-7-yl)-3,4-dihydroxytetrahydrofuran-2-yl)methoxy)(hydroxy)phosphoryl)methyl) phosphonic acid was prepared by procedures similar to the one described in Example S5, replacing cyclopentanamine in Step F with 3-(aminomethyl)cyclohexan-1-ol.

¹H NMR (400 MHz, D₂O) δ 7.66 (s, 1H), 5.15 (d, J=6.2 Hz, 1H), 4.57-4.52 (m, 1H), 4.37 (t, J=5.0 Hz, 1H), 4.17-4.14 (m, 1H), 4.04-3.93 (m, 2H), 3.58-3.51 (m, 1H), 3.41 (d, J=6.4 Hz, 2H), 2.00-1.76 (m, 5H), 1.74-1.62 (m, 3H), 0.98-0.81 (m, 3H). Mass Spectrum (ESI) m/z=570.0 (M−1).

### Example S79

### Synthesis of (((((2R,3S,4R,1S)-5-(2-chloro-4-(((1S,3S)-3-(piperidin-1-yl)cyclopentyl)amino)imidazo[2,1-f][1,2,4]triazin-7-yl)-3,4-dihydroxytetrahydrofuran-2-yl)methoxy)(hydroxy)phosphoryl)methyl)phosphonic Acid

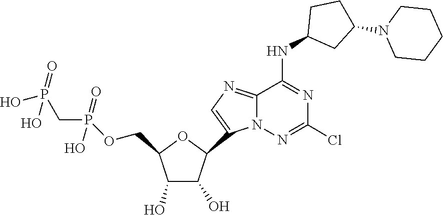 [SMILES: O=P(O)(O)CP(=O)(O)OC[C@H]1O[C@@H](c2cnc3c(N[C@H]4CC[C@H](N5CCCCC5)C4)nc(Cl)nn23)[C@H](O)[C@@H]1O]

(((((2R,3S,4R,5S)-5-(2-chloro-4-(((1S,3S)-3-(piperidin-1-yl)cyclopentyl)amino)imidazo[2,1-f][1,2,4]triazin-7-yl)-3,4-dihydroxytetrahydrofuran-2-yl)methoxyxhydroxy)phosphoryl)methyl)phosphonic acid was prepared by procedures similar to the one described in Example S5, replacing cyclopentanamine in Step F with (1S,3S)-3-(piperidin-1-yl)cyclopentan-1-amine.

¹H NMR (400 MHz, D₂O) δ 7.63 (s, 1H), 5.15-5.07 (m, 1H), 5.46-5.52 (m, 1H), 4.51-4.40 (m, 1H), 4.34-4.27 (m, 1H), 4.18-4.10 (m, 1H), 3.99-3.84 (m, 2H), 3.62-3.53 (m, 1H), 3.53-3.40 (m, 2H), 2.91-2.75 (m, 2H), 2.72-2.60 (m, 1H), 2.22-2.06 (m, 2H), 2.06-1.50 (m, 10H), 1.43-1.30 (m, 1H). Mass Spectrum (ESI) m/z=609.0 (M−1).

### Example S80

### Synthesis of (((((2R,3S,4R,1S)-5-(4-(cyclopentylamino)-2-vinylimidazo[2,1-f][1,2,4]triazin-7-yl)-3,4-dihydroxytetrahydrofuran-2-yl)methoxy)(hydroxy)phosphoryl)methyl)phosphonic Acid

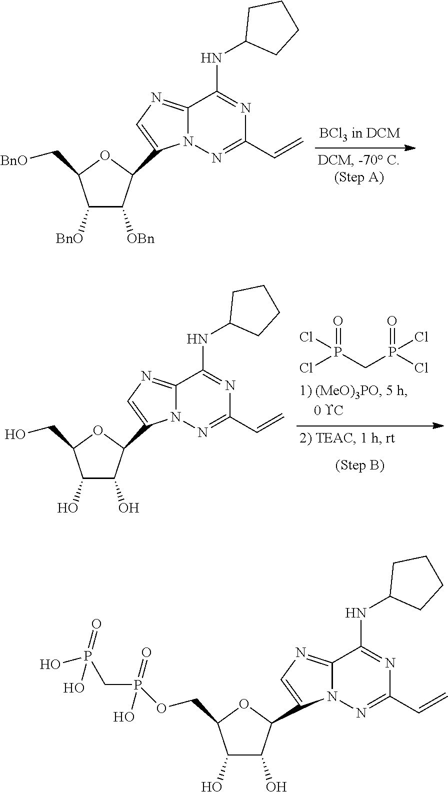

Step A and Step B: 7-[(2S,3S,4R,5R)-3,4-bis(benzyloxy)-5-[(benzyloxy)methyl]oxolan-2-yl]-N-cyclopentyl-2-ethenylimidazo[2,1-f][1,2,4]triazin-4-amine (obtained in Example S60, Step A) was converted to the title compound by procedures similar to the ones described in Example S5, Step I and Step J.

¹H NMR (400 MHz, D₂O) δ 7.60 (s, 1H), 6.66-6.57 (m, 1H), 6.44-6.35 (m, 1H), 5.73-5.65 (m, 1H), 5.23-5.15 (m, 1H), 4.61-4.53 (m, 2H), 4.34-4.30 (m, 1H), 4.18-4.13 (m, 1H), 4.01-3.90 (m, 2H), 2.15-1.95 (m, 4H), 1.78-1.50 (m, 6H). Mass Spectrum (ESI) m/z=518.1 (M−1).

### Example S81

### Synthesis of (((((2R,3S,4R,5R)—S-(4-(benzylamino)furo[3,2-d]pyrimidin-7-yl)-3,4-dihydroxytetrahydrofuran-2-yl)methoxy)(hydroxy)phosphoryl)methyl)phosphonic Acid

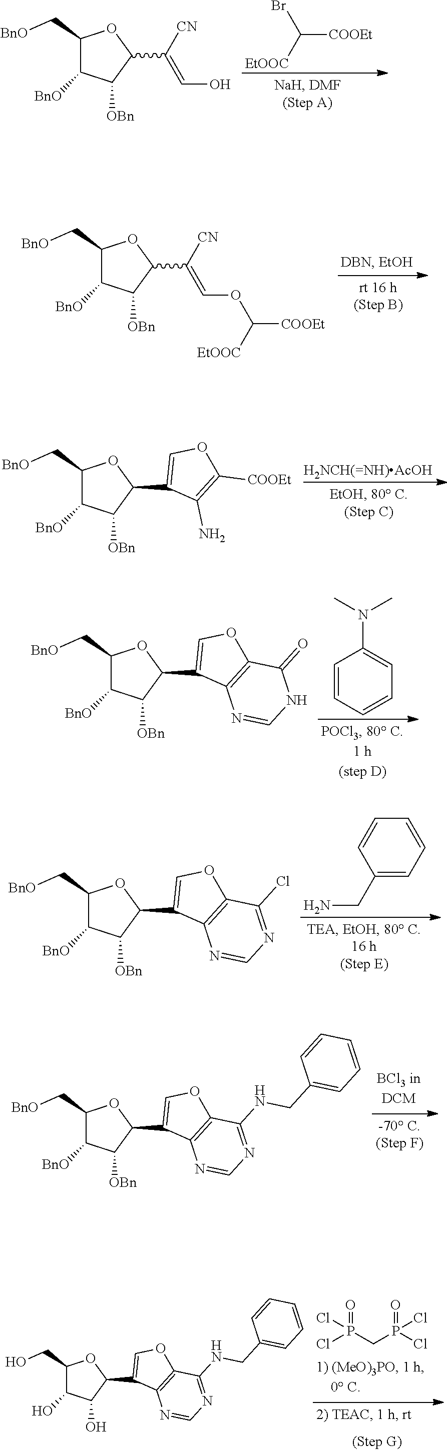

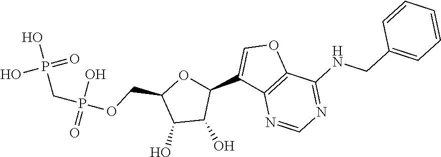 [SMILES: O=P(O)(O)CP(=O)(O)OC[C@H]1O[C@@H](c2coc3c(NCc4ccccc4)ncnc23)[C@H](O)[C@@H]1O]

Step A: To a solution of (2E)-2-[(4R,5R)-3,4-bis(benzyloxy)-5-[(benzyloxy)methyl]oxolan-2-yl]-3-hydroxyprop-2-enenitrile (5 g, 10.6 mmol) in DMF (40 ml) was added sodium hydride (60%, 528 mg, 13.2 mmol) at 0° C. The mixture was stirred at rt for 30 min, then 1,3-diethyl 2-bromopropanedioate (3.1 g, 13.2 mmol) was added dropwise at 0° C. The mixture was stirred at rt for another 16 h, then poured into ice-water and extracted with EA. The organic extracts were washed with water and brine, dried over Na₂SO₄ and filtered. The filtrate was concentrated to give 1,3-diethyl 2-{[(1E)-2-[(4R,5R)-3,4-bis(benzyloxy)-5-[(benzyloxy)methyl] oxolan-2-yl]-2-cyanoeth-1-en-1-yl]oxy} propanedioate (7 g, crude) as a yellow oil which was carried forward without further purification.

Step B: To a solution of 1,3-diethyl 2-{[(1E)-2-[(4R,5R)-3,4-bis(benzyloxy)-5-[(benzyloxy)methyl]oxolan-2-yl]-2-cyanoeth-1-en-1-yl]oxy}propanedioate (7 g, crude) in EtOH was added 2H,3H,4H,6H,7H,8H-pyrrolo[1,2-a]pyrimidine (1.39 g, 11.2 mmol) at rt and the mixture was stirred for 16 h. The mixture was concentrated and purified by CombiFlash® (PE:EA=3:1) to afford ethyl 3-amino-4-[(2S,4R,5R)-3,4-bis(benzyloxy)-5-[(benzyloxy)methyl]oxolan-2-yl]furan-2-carboxylate as a yellow oil (887 mg, 15% for two steps). Mass Spectrum (ESI) m/z=558.0 (M+1).

Step C: A solution of ethyl 3-amino-4-[(2S,4R,5R)-3,4-bis(benzyloxy)-5-[(benzyloxy)methyl]oxolan-2-yl]furan-2-carboxylate (887 mg, 1.59 mmol) and formamidine acetate (4.1 g, 39.77 mmol) in EtOH (20 mL) was stirred at 80° C. for 6 days. The mixture was diluted with DCM (100 mL), washed with water (200 mL) and brine (200 mL), dried over Na₂SO₄, filtered and the filtrate was concentrated and purified by CombiFlash® (PE/EA=5:1) to afford 7-[(2S,3S,4R,5R)-3,4-bis(benzyloxy)-5-[(benzyloxy)methyl]oxolan-2-yl]-3H-furo[3,2-d]pyrimidin-4-one as a white solid (152 mg, 18% yield). Mass Spectrum (ESI) m/z=538.5 (M+1).

Step D: To a suspension of 7-[(2S,3S,4R,5R)-3,4-bis(benzyloxy)-5-[(benzyloxy)methyl]oxolan-2-yl]-3H-furo[3,2-d]pyrimidin-4-one (152 mg, 0.28 mmol), benzyltriethylammonium chloride (128 mg, 0.57 mmol), and N,N-dimethylaniline (51 mg, 0.42 mmol) in ACN (5 mL) was added phosphorus oxychloride (260 mg, 1.69 mmol). The reaction mixture was stirred at 80° C. for 1 h. Then the solvent was removed under reduced pressure and the residue was dissolved in DCM. The solution was washed with sat. NaHCO₃ and brine and dried over Na₂SO₄. After filtration, the filtrate was concentrated and the residue was purified by CombiFlash® (PE/EA=3:1) to afford 7-[(2S,3S,4R,5R)-3,4-bis(benzyloxy)-5-[(benzyloxy)methyl] oxolan-2-yl]-4-chlorofuro[3,2-d]pyrimidine as a yellow oil (120 mg, 76% yield). Mass Spectrum (ESI) m/z=557.1 (M+1).

Step E: A solution of 7-[(2S,3S,4R,5R)-3,4-bis(benzyloxy)-5-[(benzyloxy)methyl]oxolan-2-yl]-4-chlorofuro[3,2-d]pyrimidine (120 mg, 0.22 mmol), benzylamine (24 mg, 0.23 mmol) and triethylamine (44 mg, 0.43 mmol) in EtOH (5 mL) was stirred at 80° C. for 16 hrs. The reaction mixture was concentrated and the residue was purified by CombiFlash® (PE/EA=1:1) to afford N-benzyl-7-[(2S,3S,4R,5R)-3,4-bis(benzyloxy)-5-[(benzyloxy)methyl]oxolan-2-yl]furo[3,2-d]pyrimidin-4-amine as a white solid (130 mg, 96% yield). Mass Spectrum (ESI) m/z=628.2 (M+1).

Step F: To a solution of N-benzyl-7-[(2S,3S,4R,5R)-3,4-bis(benzyloxy)-5-[(benzyloxy)methyl]oxolan-2-yl]furo[3,2-d]pyrimidin-4-amine (130 mg, 0.21 mmol) in DCM (3 mL) was added boron trichloride (1 M in DCM, 2 mL, 2.1 mmol) dropwise at −70° C. The mixture was stirred at −70° C. for 1 h. Then the reaction was brought to −30° C. over a period of 30 min, and quenched by adding a mixture of methanol:chloroform (2:1, 10 mL). After the reaction mixture had warmed to rt, it was neutralized with NH₃ in methanol (10%, 10 mL) and concentrated. The residue was purified by CombiFlash® (DCM/MeOH=100:0 to 9:1) to afford (2S,3R,4S,5R)-2-[4-(benzylamino)furo[3,2-d]pyrimidin-7-yl]-5-(hydroxymethyl)oxolane-3,4-diol (36 mg, 48% yield). Mass Spectrum (ESI) m/z=357.7 (M+1).

Step G: To a solution of (2S,3R,4S,5R)-2-[4-(benzylamino)furo[3,2-d]pyrimidin-7-yl]-5-(hydroxymethyl)oxolane-3,4-diol (36 mg, 0.1 mmol) in trimethylphosphate (1 mL) at 0° C. was added a cold solution of methylenebis(phosphonic dichloride) (125 mg, 0.5 mmol) in trimethylphosphate (1 mL) dropwise. Then the reaction solution was stirred at 0° C. for 1 h. TEAC (0.5 M, 0.7 mL) was added to the reaction carefully and the reaction was stirred at this temperature for 15 mins, then warmed to room temperature and continued to stir for 1 h. Trimethylphosphate was extracted using tert-butyl methyl ether (5 mL×3) and the aqueous layer was basified with ammonium hydroxide to pH ˜7-8, then purified by Prep-HPLC (Daisogel-C18 250×50 mm, 10 um column) using a gradient of 0.2% ammonium hydroxide in water/ACN from 100:0 to 95:5 to give (((((2R,3S,4R,5R)-5-(4-(benzylamino)furo[3,2-d]pyrimidin-7-yl)-3,4-dihydroxytetrahydrofuran-2-yl)methoxyxhydroxy)phosphoryl)methyl)phosphonic acid (2.2 mg, 4.2% yield) as a white solid. ¹H NMR (400 MHz, D₂O) δ 8.18 (s, 1H), 8.09 (s, 1H), 7.40-7.20 (m, 5H), 5.03 (d, J=6.3 Hz, 1H), 4.85-4.72 (m, 2H), 4.43-4.35 (m, 1H), 4.35-4.25 (m, 1H), 4.16-4.10 (m, 1H), 4.07-3.97 (m, 2H), 2.13-2.02 (m, 2H). Mass Spectrum (ESI) m/z=513.4 (M−1).

### Biological Examples

A variety of assays can be used to evaluate the inhibition of CD73 enzymatic activity. Compounds of the present disclosure display inhibition of CD73 in the following assays.

### Example B. CD73 Enzyme Assay

Soluble recombinant CD73 catalyzes the conversion of adenosine monophosphate (AMP) to adenosine and inorganic phosphate. The phosphate detection reagent, PiColorLock™ (Innova Bioscience, Cat #303-0125) is based on the change in absorbance of the dye malachite green in the presence of inorganic phosphate (Pi) and this property can be exploited to measure any enzyme that generates Pi. Recombinant Human 5′-Nucleotidase (CD73) (R&D #5795-EN, CHO derived CD73 (Trp27-Lys547), with a C-terminal 6-His tag) was used in the enzymatic assay. This assay was run in a 384-well plate format (Corning® NBS™ 384 well plates, Cat #3640). The basic assay procedure involves two steps: 1) Enzyme reaction: The CD73 enzyme (R&D #5795-EN) is incubated in the presence or absence of compounds. AMP (sigma, cat #01930) is added to start the kinase reaction. 2) Detection step: Gold mix is added to the assay system, then stabilizers are added. After incubation the absorbance of the solution is read at OD 635 nm. The recorded OD signal is proportional to the enzyme activity.

Briefly, 25 μl human CD73 (0.5 nM final concentration) in the enzymatic buffer solutions (20 mM Tris, 25 mM NaCl, 1 mM MgCl₂, pH 7.5, 0.005% Tween-20) were mixed with various concentrations of the test compound (dissolved in 100% DMSO). These solutions were incubated for 15 min at 25° C., and subsequently 25 μl AMP (30 μM final concentration) was added to start the reaction. The final reaction mixture of enzyme-substrate-compound was incubated for 20 min at 37° C. Meanwhile ‘Gold mix’ was prepared shortly before use by adding 1/100 vol. of accelerator to the PiColorLock™ Gold reagent. 12 μL/well of the ‘Gold mix’ was added to assay plate containing 50 μL enzyme reaction buffer and incubated at 25° C. for 5 min. 5 μL/well stabilizer was added to the assay plate and incubated at 25° C. for 30 min. The absorbance of the well solutions was measured at 635 nm on a Spark 10M instrument (TECAN).

The percent (%) inhibition at each concentration of a compound was calculated relative to the OD value in the Max and Min control wells contained within each assay plate. The Max control wells contained enzyme and substrate as 0% inhibition, and the M control wells only contained substrate without enzyme as 100% inhibition. The concentrations and percent inhibition values for a test compound are plotted and the concentration of the compound required to achieve 50% inhibition (IC₅₀) was determined with a four-parameter logistic dose response equation. Results for certain compounds are provided in the Table 2 below.

TABLE 2

| Com- | | Com- | | Com- | |
|---|---|---|---|---|---|
| pound | | pound | | pound | |
| No. | Potency | No. | Potency | No. | Potency |
| 1 | a | 2 | b | 3 | d |
| 4 | d | 5 | a | 6 | a |
| 7 | b | 8 | a | 9 | a |
| 10 | a | 11 | a | 12 | a |
| 13 | b | 14 | a | 15 | a |
| 16 | b | 17 | a | 18 | a |
| 19 | a | 20 | a | 21 | a |
| 22 | a | 23 | a | 24 | a |
| 25 | b | 26 | b | 27 | a |
| 28 | a | 29 | a | 30 | a |
| 31 | b | 32 | a | 33 | a |
| 34 | a | 35 | a | 36 | a |
| 37 | a | 38 | b | 39 | a |
| 40 | a | 41 | a | 42 | a |
| 43 | a | 44 | a | 45 | a |
| 46 | a | 47 | a | 48 | a |
| 49 | a | 50 | a | 51 | b |
| 52 | a | 53 | a | 54 | a |
| 55 | a | 56 | a | 57 | d |
| 58 | c | 59 | d | 60 | c |
| 61 | c | 62 | a | 63 | a |
| 64 | a | 65 | a | 66 | a |
| 67 | b | 68 | b | 69 | b |
| 70 | c | 71 | b | 72 | a |
| 73 | b | 74 | b | 75 | b |
| 76 | d | 77 | a | 78 | a |
| 79 | a | 80 | b | 81 | b |

“a” means an IC₅₀ of <10 nM; “b” means an IC₅₀ of 10-99 nM; “c” means an IC₅₀ of 100-999 nM; and “d” means IC₅₀ of >1000 nM

### Example B2. CD73 Cellular Assay

Cell surface CD73 catalyzes the conversion of adenosine monophosphate (AMP) to adenosine and inorganic phosphate. U87 MG human glioblastoma cells express high level of CD73. Cells are treated with compounds in a 96-well assay plate and supernatants are collected into a 384-well detection plate. The concentration of inorganic phosphate (Pi) in the supernatants is determined using the phosphate detection reagent, PiColorLock™ (Innova Bioscience, Cat #303-0125) following the manufacturer's instructions.

Briefly, on the day of the assay, U87 MG cells were harvested and resuspended in assay buffer which consisted of 20 mM HEPES, pH=7.4, 137 mM NaCl, 5.4 mM KCl, 1.3 mM CaCl₂, 4.2 mM NaHCO₃ and 0.1% glucose. To test the effect of compounds on cellular CD73 enzymatic activity, 500 nL/well of compounds dissolved in DMSO were added to a 96-well TC-treated microplate (Corning #3599). Next, 80 μL/well of U87 MG cells in assay buffer were added to assay plate. After 30 minutes of incubation in an atmosphere of 5% CO₂ at 37° C., 20 μL/well of 150 μM AMP (Adenosine 5′-monophosphate monohydrate, Sigma, Cat #01930) in assay buffer was added to assay plate. Final assay conditions consisted of 5000 cells per well in 0.5% DMSO and 30 μM AMP substrate. After 50 minutes of incubation in an atmosphere of 5% CO₂ at 37° C., 50 μL/well of supernatant was transferred to the 384-well detection plate (Corning® NBS™ 384 well plates, Cat #3640). Meanwhile “Gold mix” was prepared shortly before use by adding 1/100 volume of accelerator to the PiColorLock™ Gold reagent. 12 μL/well of the “Gold mix” was added to detection plate containing 50 μL/well of supernatant and incubated at 25° C. for 5 minutes. 5 μL/well stabilizer was added to the detection plate and incubated at 25° C. for 30 minutes. The absorbance at 635 nm was measured on a Spark 10M instrument (TECAN). The percent (%) inhibition at each concentration of a compound was calculated relative to the OD value in the Max and Min control wells contained within each assay plate. The Max control wells contained cells and substrate as 0% inhibition, and the Min control wells only contained cells as 100% inhibition. The concentrations and percent inhibition values for a test compound are plotted and the concentration of a the compound required to achieve 50% inhibition (IC50) was determined with a four-parameter logistic dose response equation. Results for certain compounds are provided in the Table 3 below.

TABLE 3

| Com- | | Com- | | Com- | |
|---|---|---|---|---|---|
| pound | | pound | | pound | |
| No. | Potency | No. | Potency | No. | Potency |
| 1 | a | 2 | b | 5 | a |
| 6 | a | 8 | a | 9 | a |
| 10 | a | 11 | a | 12 | a |
| 14 | a | 15 | a | 16 | b |
| 17 | a | 18 | b | 19 | a |
| 20 | a | 21 | a | 22 | a |
| 23 | a | 24 | a | 27 | a |
| 28 | a | 29 | a | 30 | a |
| 32 | a | 33 | b | 34 | b |
| 35 | a | 36 | a | 37 | a |
| 39 | a | 40 | b | 41 | a |
| 42 | a | 43 | a | 44 | a |
| 45 | a | 46 | a | 47 | b |
| 48 | b | 49 | a | 50 | a |
| 52 | a | 53 | a | 54 | a |
| 55 | a | 56 | a | 62 | a |
| 63 | a | 64 | a | 65 | a |
| 66 | a | 72 | b | 73 | b |
| 74 | c | 75 | b | | |

“a” means an IC₅₀ of <1 nM; “b” means an IC₅₀ of 1-9.9 nM; “c” means an IC₅₀ of 10-99.9 nM; and “d” means IC₅₀ of >100 nM

### Example B3. In Vivo Models

If desired, the compounds can be evaluated in vivo in appropriate animal models, such as, for example, syngeneic mouse models set forth in the art, for example in Sanmamed M. F., et al., Annals of Oncology, 27: 1190-1198, (2016).

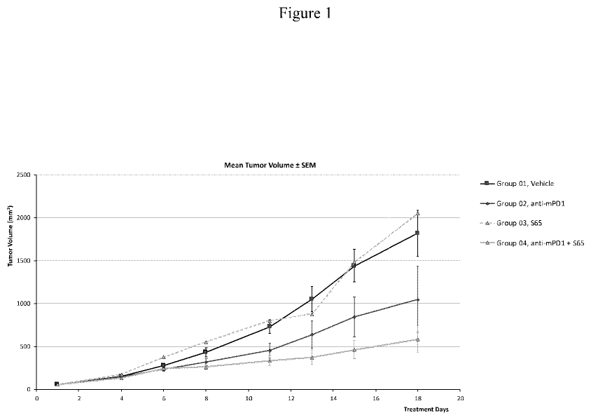

Some compounds were evaluated preclinically for in vivo therapeutic efficacy as single agents or in combination with other agents. Compounds of the invention show potent anti-tumor effects when dosed in combination with, for example, an anti-mPDI antibody as evidenced by reducing tumor volume in a subcutaneous CT-26 murine colon carcinoma syngeneic model in BALB/c female mice. CT-26 tumor cells were implanted subcutaneously into BALB/c female mice for each experiment. Anti-mPDl antibody (10 mg/kg) was dosed i.p. starting on study day 6 post implant twice weekly for a total of 4 doses. Compound No. 65 (10 mg/kg) or vehicle was administered iv once a day starting on day 6 post implant. FIG. 1 depicts the reduction in tumor volume in combination therapy.

All references throughout, such as publications, patents, patent applications and published patent applications, are incorporated herein by reference in their entireties.

1. A compound of formula (I): 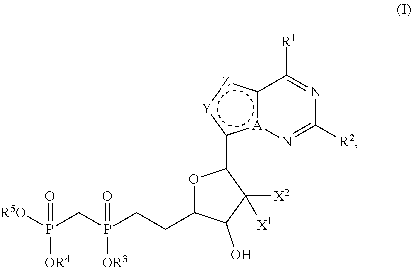
or a stereoisomer, tautomer, prodrug, or a pharmaceutically acceptable salt of any of the foregoing, wherein: [symbol]
means a fully saturated, partially saturated, or aromatic ring;
X¹ and X² are each independently H, —CN, C₁₋₆ alkyl, —OR′, or halogen, wherein R′ is H, C₁₋₆ alkyl, C₃₋₁₂ cycloalkyl, 3- to 12-membered heterocyclyl, 5- to 10-membered heteroaryl, or C₆₋₁₄ aryl;
Y is CH or N;
Z is CH, O, or N;
A is C or N;
R¹ is —NR^{1a}R^{1b} or —OR^{1a}, wherein R^{1a} and R^{1b} are each independently H, C₁₋₆ alkyl, C₃₋₁₂ cycloalkyl, 3- to 12-membered heterocyclyl, 5- to 10-membered heteroaryl, or C₆₋₁₄ aryl, wherein the C₁₋₆ alkyl, C₃₋₁₂ cycloalkyl, 3- to 12-membered heterocyclyl, 5- to 10-membered heteroaryl, and C₆₋₁₄ aryl are each independently optionally substituted with R⁶, or
R^{1a} and R^{1b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
R² is H, C₁₋₆ alkyl, C₂₋₆ alkenyl, C₂₋₆ alkynyl, halogen, —CN, —OR^{2a}, —SR^{2a}, —NR^{2a}R^{2b}, —OC(O)R^{2a}, —NR^{2a}C(O)R^{2b}, —NR^{2a}C(O)OR^{2b}, —NR^{2a}S(O)R^{2b}, —NR^{2a}aS(O)₂R^{2b}, —C(O)NR^{2a}R^{2b}, —C(O)NR^{2a}S(O)₂R^{2b}, C₃₋₁₂ cycloalkyl, 3- to 12-membered heterocyclyl, 5- to 10-membered heteroaryl, or C₆₋₁₄ aryl, wherein the C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, and C₆₋₁₄ aryl are each independently optionally substituted with R⁷, and wherein:
R^{2a} and R^{2b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{2a} and R^{2b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
R³, R⁴, and R⁵ are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl;
each R⁶ is independently oxo, C₁₋₆ alkyl, C₂₋₆ alkenyl, C₂₋₆ alkynyl, halogen, —CN, —OR^{6a}, —SR^{6a}, —NR^{6a}R^{6b}, —NO₂, —C═NH(OR^{6a}), —C(O)R^{6a}, —OC(O)R^{6a}, —C(O)OR^{6a}, —C(O)NR^{6a}R^{6a}, —OC(O)NR^{6a}R^{6b}, —NR^{6a}C(O)R^{6b}, —NR^{6a}C(O)OR^{6b}, —S(O)R^{6a}, —S(O)₂R^{6a}, —NR^{6a}S(O)R^{6b}, —C(O)NR^{6a}S(O)R^{6b}, —NR^{6a}S(O)₂R^{6b}, —C(O)NR^{6a}S(O)₂R^{6b}, —S(O)NR^{6a}R^{6b}, —S(O)₂NR^{6a}R^{6b}, —P(O)OR^{6a})(OR^{6b}), C₃₋₆ cycloalkyl, 3- to 12-membered heterocyclyl, 5- to 10-membered heteroaryl, or C₆₋₁₄ aryl, wherein the C₃₋₆ cycloalkyl, 3- to 12-membered heterocyclyl, 5- to 10-membered heteroaryl, and C₆₋₁₄ aryl are each independently optionally substituted with C₁₋₆ alkyl, C₂₋₆ alkenyl, C₂₋₆ alkynyl, halogen, hydroxyl, C₁₋₆ alkoxy, or —CN, and wherein:
R^{6a} and R^{6b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{6a} and R^{6b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN;
each R⁷ is independently oxo, C₁₋₆ alkyl, C₂₋₆ alkenyl, C₂₋₆ alkynyl, halogen, —CN, —OR^{7a}, —SR^{7a}, —NR^{7a}R^{7b}, —NO₂, —C═NH(OR^{7a}), —C(O)R^{7a}, —OC(O)R^{7a}, —C(O)OR^{7a}, —C(O)NR^{7a}R^{7b}, —OC(O)NR^{7a}R, —NR^{7a}C(O)R^{7b}, —NR^{7a}C(O)OR^{7b}, —S(O)R^{7a}, —S(O)₂R^{7a}, —NR^{7a}S(O)R^{7b}, —C(O)NR^{7a}S(O)R^{7b}, —NR^{7a}S(O)₂R^{7b}, —C(O)NR^{7a}S(O)₂R^{7b}, —S(O)NR^{7a}R^{7b}, —S(O)₂NR^{7a}R^{7b}, —P(O)(OR^{7a}) (OR^{7b}), C₃₋₆ cycloalkyl, 3- to 12-membered heterocyclyl, 5- to 10-membered heteroaryl, or C₆₋₁₄ aryl, wherein:
R^{7a} and R^{7b} are each independently H, C₁₋₆ alkyl, C₂₋₆ alkenyl, C₂₋₆ alkynyl, C₃₋₁₂ cycloalkyl, 3- to 12-membered heterocyclyl, 5- to 10-membered heteroaryl, or C₆₋₁₄ aryl, or
R^{7a} and R^{7b} are taken together with the nitrogen atom to which they attach to form a 3- to 12-membered heterocyclyl, which is optionally substituted with C₁₋₆ alkyl, C₂₋₆ alkenyl, C₂₋₆ alkynyl, halogen, hydroxyl, C₁₋₆ alkoxy, or —CN.

2. The compound of claim 1, or a stereoisomer, tautomer, prodrug, or a pharmaceutically acceptable salt of any of the foregoing, wherein Y is CH.

3. The compound of claim 1, or a stereoisomer, tautomer, prodrug, or a pharmaceutically acceptable salt of any of the foregoing, wherein Z is N.

4. The compound of claim 1, or a stereoisomer, tautomer, prodrug, or a pharmaceutically acceptable salt of any of the foregoing, wherein A is N.

5. The compound of claim 1, or a stereoisomer, tautomer, prodrug, or a pharmaceutically acceptable salt of any of the foregoing, wherein the compound is of formula (II):

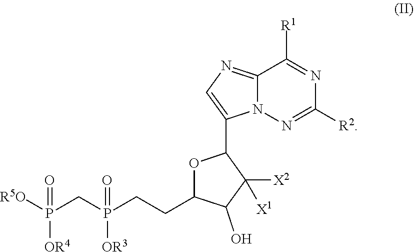

6. The compound of claim 1, or a stereoisomer, tautomer, prodrug, or a pharmaceutically acceptable salt of any of the foregoing, wherein X¹ is H or —OH.

7. The compound of claim 1, or a stereoisomer, tautomer, prodrug, or a pharmaceutically acceptable salt of any of the foregoing, wherein X² is H or halogen.

8. The compound of claim 1, or a stereoisomer, tautomer, prodrug, or a pharmaceutically acceptable salt of any of the foregoing, wherein R¹ is —NR^{1a}R^{1b}.

9. The compound of claim 1, or a stereoisomer, tautomer, prodrug, or a pharmaceutically acceptable salt of any of the foregoing, wherein R¹ is —OR^{1a}.

10. The compound of claim 1, or a stereoisomer, tautomer, prodrug, or a pharmaceutically acceptable salt of any of the foregoing, wherein R^{1a} is C₁₋₆ alkyl, C₃₋₁₂ cycloalkyl, or 3- to 12-membered heterocyclyl, each of which is independently optionally substituted with R.

11. The compound of claim 10, or a stereoisomer, tautomer, prodrug, or a pharmaceutically acceptable salt of any of the foregoing, wherein R⁶ is 3- to 12-membered heterocyclyl or C₆₋₁₄ aryl, each of which is independently optionally substituted with halogen.

12. The compound of claim 1, or a stereoisomer, tautomer, prodrug, or a pharmaceutically acceptable salt of any of the foregoing, wherein R^{1a} is K, or

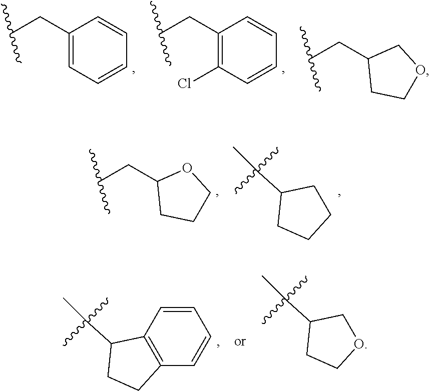

13. The compound of claim 1, or a stereoisomer, tautomer, prodrug, or a pharmaceutically acceptable salt of any of the foregoing, wherein R^{1b} is H or C₁₋₆ alkyl.

14. The compound of claim 1, or a stereoisomer, tautomer, prodrug, or a pharmaceutically acceptable salt of any of the foregoing, wherein R^{1a} and R^{1b} are taken together with the nitrogen atom to which they attach to form a 3- to 12-membered heterocyclyl.

15. The compound of claim 1, or a stereoisomer, tautomer, prodrug, or a pharmaceutically acceptable salt of any of the foregoing, wherein R^{1a} and R^{1b} are taken together with the nitrogen atom to which they attach to form

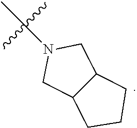 [SMILES: *N1CC2CCCC2C1]

16. The compound of claim 1, or a stereoisomer, tautomer, prodrug, or a pharmaceutically acceptable salt of any of the foregoing, wherein R² is H or halogen.

17. The compound of claim 1, or a stereoisomer, tautomer, prodrug, or a pharmaceutically acceptable salt of any of the foregoing, wherein R³ is H.

18. The compound of claim 1, or a stereoisomer, tautomer, prodrug, or a pharmaceutically acceptable salt of any of the foregoing, wherein R⁴ is H.

19. The compound of claim 1, or a stereoisomer, tautomer, prodrug, or a pharmaceutically acceptable salt of any of the foregoing, wherein R⁵ is H.

20. A compound selected from the group consisting of the compounds in Table 1, or a stereoisomer, tautomer, prodrug, or a pharmaceutically acceptable salt of any of the foregoing.

21. A pharmaceutical composition comprising at least one compound according to claim 1, or a stereoisomer, tautomer, prodrug, or a pharmaceutically acceptable salt of any of the foregoing, and a pharmaceutically acceptable excipient.

22. A kit comprising at least one compound according to claim 10, or a stereoisomer, tautomer, prodrug, or a pharmaceutically acceptable salt of any of the foregoing,

23. A method of treating a disease mediated by CD73 in an individual in need thereof, comprising administering to the individual a therapeutically effective amount of a compound according to claim 1, or a stereoisomer, tautomer, prodrug, or a pharmaceutically acceptable salt of any of the foregoing.

24. The method of claim 23, wherein the disease is cancer.

25. A method of inhibiting CD73, comprising contacting CD73 with a compound according to claim 1, or a stereoisomer, tautomer, prodrug, or a pharmaceutically acceptable salt of any of the foregoing.

26. (canceled)
